SEQUENCE LISTING (154 sequences)

SEQ ID NO: 1 - protein, 119 aa, Artificial Sequence
  synthesized
EVQLVESGGGLVQPGGSLRLSCKVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSS

SEQ ID NO: 2 - DNA, 357 bp, Artificial Sequence
  synthesized
gaagttcagctggtggaatccggcggaggattggttcaacctggcggctctctgagactgtcctgtaaggtgtctggcttctccctgaccaactacggcgtgcactgggtccgacaggcacctggaaaaggactggaatgggtcggagtgatttggagcggcggcaacaccgactacaacacccctttcaccagccggttcaccatctctcgggacacctccaagaacaccgtgtacctgcagatgaactccctgagagccgaggacaccgccgtgtactattgtgctagagccctgacctactatgactacgagttcgcctattggggccagggaaccctggtcacagtctcctct

SEQ ID NO: 3 - protein, 107 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVL

SEQ ID NO: 4 - DNA, 321 bp, Artificial Sequence
  synthesized
gagatcgtgatgacccagtctccttccacactgtccgcctctgtgggcgacagagtgatcatcacctgtagagccagccagtccatcggcaccaacatccactggtatcagcagaagcctggcaaggcccctaagctgctgatctactacgcctccgagtctatcagcggcatcccctccagattctccggctctggatctggcgctgagtttaccctgacaatctccagcctgcagcctgacgacttcgccacctactactgccagcagaacaacaactggcccaccacctttggccagggcaccaaactgacagttctt

SEQ ID NO: 5 - protein, 119 aa, Artificial Sequence
  synthesized
QVQLQQSGPGLVKPSQTLSITCTVSGFSLTNYGVHWIRQAPGKGLEWLGVIWSGGNTDYNTPFTSRFTISKDNSKNQVYFKLRSLRAEDTAIYYCARALTYYDYEFAYWGQGTLVTVSS

SEQ ID NO: 6 - DNA, 357 bp, Artificial Sequence
  synthesized
caagttcagttgcagcagtctggccctggcctcgtgaagccttctcagaccctgtctatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatctccaaggacaactccaagaaccaggtgtacttcaagctgcggtccctgagagccgaggacaccgccatctactactgtgctagagccctgacctactacgactacgagttcgcctattggggccagggcacactggtcacagtctcttct

SEQ ID NO: 7 - protein, 107 aa, Artificial Sequence
  synthesized
EIVLTQSPSILSVSPGERATFSCRASQSIGTNIHWYQQRPGKPPRLLIKYASESISGIPSRFSGSGSGTEFTLTITSVQSEDIAVYYCQQNNNWPTTFGPGTKLELK

SEQ ID NO: 8 - DNA, 321 bp, Artificial Sequence
  synthesized
gagatcgtgctgacccagtctccttccatcctgtctgtgtctcccggcgagagagccaccttcagctgtagagcctctcagtccatcggcaccaacatccactggtatcagcagcggcctggcaagcctcctcggctgctgattaagtacgcctccgagtctatcagcggcatcccctccagattctccggctctggatctggcaccgagttcaccctgaccatcacctccgtgcagtccgaggatatcgccgtgtactactgccagcagaacaacaactggcccaccacctttggacccggcaccaagctggaattgaaa

SEQ ID NO: 9 - protein, 119 aa, Artificial Sequence
  synthesized
QVQLVQSGPGLVKPSQTLSLTCTVSGFSLTNYGVHWIRQAPGKGLEWLGVIWSGGNTDYNTPFTSRFTITKDNSKNQVYFKLRSVRSEDTAVYYCARALTYYDYEFAYWGQGTLVTVSS

SEQ ID NO: 10 - DNA, 357 bp, Artificial Sequence
  synthesized
caagttcagctggttcagtctggccctggcctcgtgaagccttctcagaccctgtctctgacctgcaccgtgtctggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgagatccgtgcggagcgaggacaccgccgtgtactattgtgctagagccctgacctactacgactacgagttcgcctattggggccagggcacactggtcacagtctcttct

SEQ ID NO: 11 - protein, 107 aa, Artificial Sequence
  synthesized
EIVLTQSPSILSVSPGERVTFSCRASQSIGTNIHWYQQRPGKPPRLLIKYASESISGIPARFSGSGSGTEFTLTISSVQSEDFATYYCQQNNNWPTTFGPGTKLELK

SEQ ID NO: 12 - DNA, 321 bp, Artificial Sequence
  synthesized
gagatcgtgctgacccagtctccttccatcctgtctgtgtctcccggcgagagagtgaccttcagctgtagagcctctcagtccatcggcaccaacatccactggtatcagcagcggcctggcaagcctcctcggctgctgattaagtacgcctccgagtccatcagcggcatccctgccagattttccggctctggctctggcaccgagtttaccctgaccatctcctccgtgcagtccgaggatttcgccacctactactgccagcagaacaacaactggcccaccacctttggacccggcaccaagctggaattgaaa

SEQ ID NO: 13 - protein, 119 aa, Artificial Sequence
  synthesized
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSS

SEQ ID NO: 14 - DNA, 357 bp, Artificial Sequence
  synthesized
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttct

SEQ ID NO: 15 - protein, 107 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELK

SEQ ID NO: 16 - DNA, 321 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctggaattgaaa

SEQ ID NO: 17 - protein, 119 aa, Artificial Sequence
  synthesized
QVQLLQSGPGLVKPSETLSLTCTVSGFSLTNYGVHWIRQAPGKGLEWIGVIWSGGNTDYNTPFTSRFTISKDNSKNQVYFKLRSLTSEDTAIYYCARALTYYDYEFAYWGQGTLVTVSS

SEQ ID NO: 18 - DNA, 357 bp, Artificial Sequence
  synthesized
caagttcagctgttgcagtctggccctggcctggtcaagccttccgaaacactgtctctgacctgcaccgtgtccggcttctccctgaccaattatggcgtgcactggatcagacaggcccctggcaaaggcctggaatggatcggagtgatttggagcggcggcaacaccgactacaacacccctttcaccagccggttcaccatctccaaggacaactccaagaaccaggtgtacttcaagctgcggtccctgacctctgaggacaccgccatctactactgcgctagagccctgacctactacgactacgagttcgcctattggggccagggcacactggtcacagtctcttct

SEQ ID NO: 19 - protein, 107 aa, Artificial Sequence
  synthesized
EIQLTQSPSTLSVSPGERATFSCRASQSIGTNIHWYQQKTGQPPRLLIKYASESISGIPDRFSGSGSGTEFTLSITSVQSEDFAVYYCQQNNNWPTTFGPGTKLEIL

SEQ ID NO: 20 - DNA, 321 bp, Artificial Sequence
  synthesized
gagatccagctgacccagtctccttccacactgtctgtgtctcccggcgagagagccaccttcagctgtagagcctctcagtccatcggcaccaacatccactggtatcagcagaaaaccggccagcctcctcggctgctgattaagtacgcctccgagtccatcagcggcatccctgacagattctccggctctggctctggcaccgagttcaccctgtctatcacctccgtgcagtccgaggacttcgccgtgtactactgccagcagaacaacaactggcccaccacctttggacccggcaccaagctggaaattctt

SEQ ID NO: 21 - protein, 119 aa, Artificial Sequence
  synthesized
QVQLLQSGPGLVKPSETLSITCTVSGFSLTNYGVHWIRQAPGKGLEWIGVIWSGGNTDYNTPFTSRFTITKDNSKNQVFFKLRSVRAEDTALYYCARALTYYDYEFAYWGQGTLVTVSS

SEQ ID NO: 22 - DNA, 357 bp, Artificial Sequence
  synthesized
caagttcagctgttgcagtctggccctggcctggtcaagccttccgagacactgtccatcacctgtaccgtgtccggcttctccctgaccaattacggcgtgcactggatcagacaggcccctggcaaaggcctggaatggatcggagtgatttggagcggcggcaacaccgactacaacacccctttcaccagccggttcaccatcaccaaggacaactccaagaaccaggtgttcttcaagctgcggagcgtgcgcgctgaggataccgctctgtactattgcgctcgggccctgacctactacgactacgagtttgcttactggggccagggaaccctggtcaccgtttcttct

SEQ ID NO: 23 - protein, 107 aa, Artificial Sequence
  synthesized
EIVLTQSPSTLSVSPGERVSFSCRASQSIGTNIHWYQQRTGQPPRLLIKYASESISGIPARFSGSGSGTEFTLTITSVQSEDFAVYYCQQNNNWPTTFGQGTKLDIK

SEQ ID NO: 24 - DNA, 321 bp, Artificial Sequence
  synthesized
gagatcgtgctgacccagtctccttccacactgtctgtgtctcccggcgagagagtgtccttcagctgtagagcctctcagtccatcggcaccaacatccactggtatcagcagagaaccggccagcctcctcggctgctgattaagtacgcctccgagtccatcagcggcatccctgccagattttccggctctggctctggcaccgagttcaccctgaccatcacctccgtgcagtctgaggacttcgccgtgtactactgccagcagaacaacaactggcccaccacctttggccagggcaccaagctggatatcaaa

SEQ ID NO: 25 - protein, 119 aa, Artificial Sequence; identical to SEQ ID NO: 13
  synthesized

SEQ ID NO: 26 - DNA, 357 bp, Artificial Sequence; identical to SEQ ID NO: 14
  synthesized

SEQ ID NO: 27 - protein, 107 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVL

SEQ ID NO: 28 - DNA, 321 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttctt

SEQ ID NO: 29 - protein, 119 aa, Artificial Sequence
  synthesized
QVQLVESGPGLVQPSGSLSLTCTVSGFSLTNYGVHWVRQAPGKGLEWVGVIWSGGNTDYNTPFTSRFTISKDNSKNQVYLKMNSLRAEDTAVYYCARALTYYDYEFAYWGQGTLVTVSS

SEQ ID NO: 30 - DNA, 271 bp, Artificial Sequence
  synthesized
gaccaattacggcgtgcactgggttcgacaggctccaggcaaaggactggaatgggtcggagtgatttggagcggcggcaacaccgactacaacacccctttcaccagccggttcaccatctccaaggacaactccaagaaccaggtgtacctgaagatgaactccctgagagccgaggacaccgctgtgtattactgtgctagagccctgacctactacgactacgagttcgcctattggggccagggaaccctggtcacagtctcctct

SEQ ID NO: 31 - protein, 107 aa, Artificial Sequence
  synthesized
DIVLTQSPSSLSVSPGERVTISCRASQSIGTNIHWYQQKPGQAPRLLIKYASESISGIPSRFSGSGSGTDFTLTISSVESEDFAVYYCQQNNNWPTTFGQGTKLEIK

SEQ ID NO: 32 - DNA, 321 bp, Artificial Sequence
  synthesized
gacatcgtgctgacccagtctccatccagcctgtctgtgtctcctggcgagagagtgaccatctcttgccgggcctctcagagcatcggcaccaacatccactggtatcagcagaagcctggacaggcccctcggctgctgattaagtacgcctccgagtctatcagcggcatcccctccagattctccggctctggctctggcacagactttaccctgaccatcagctccgtggaatccgaggacttcgccgtgtactactgccagcagaacaacaactggcccaccacctttggccagggcaccaagctggaaatcaaa

SEQ ID NO: 33 - protein, 119 aa, Artificial Sequence; identical to SEQ ID NO: 29
  synthesized

SEQ ID NO: 34 - DNA, 357 bp, Artificial Sequence
  synthesized
caagttcagctggtggaatctggccctggcctggttcagccttctggctctctgtctctgacctgcaccgtgtctggcttctccctgaccaattacggcgtgcactgggttcgacaggctccaggcaaaggactggaatgggtcggagtgatttggagcggcggcaacaccgactacaacacccctttcaccagccggttcaccatctccaaggacaactccaagaaccaggtgtacctgaagatgaactccctgagagccgaggacaccgctgtgtattactgtgctagagccctgacctactacgactacgagttcgcctattggggccagggaaccctggtcacagtctcctct

SEQ ID NO: 35 - protein, 107 aa, Artificial Sequence
  synthesized
DIVLTQSPSSLSVSPGERVTISCRASQSIGTNIHWYQQKPGQAPRLLIKYASESISGIPSRFSGSGSGTDFTLTISSVESEDFAVYYCQQNNNWPTTFGQGTKLTVL

SEQ ID NO: 36 - DNA, 321 bp, Artificial Sequence
  synthesized
gacatcgtgctgacccagtctccatccagcctgtctgtgtctcctggcgagagagtgaccatctcttgccgggcctctcagagcatcggcaccaacatccactggtatcagcagaagcctggacaggcccctcggctgctgattaagtacgcctccgagtctatcagcggcatcccctccagattctccggctctggctctggcacagactttaccctgaccatcagctccgtggaatccgaggacttcgccgtgtactactgccagcagaacaacaactggcccaccacctttggccagggcaccaagctgacagttctt

SEQ ID NO: 37 - protein, 119 aa, Artificial Sequence
  synthesized
QVQLQESGPGLVKPSESLSLTCTVSGFSLTNYGVHWVRQPPGKGLEWIGVIWSGGNTDYNTPFTSRVTISKDNSKNQVSLKMNSLTAADTAVYYCARALTYYDYEFAYWGQGTLVTVSS

SEQ ID NO: 38 - DNA, 357 bp, Artificial Sequence
  synthesized
caagttcagctgcaagagtctggccctggcctggtcaagccttccgaatctctgtctctgacctgcaccgtgtccggcttctccctgaccaattatggcgtgcactgggttcgacagcctccaggcaaaggcctggaatggatcggagtgatttggagcggcggcaacaccgactacaacacccctttcacctctagagtgaccatcagcaaggacaactccaagaaccaggtgtccctgaagatgaacagcctgaccgctgccgacaccgctgtgtactattgtgctagagccctgacctactacgactacgagttcgcctattggggccagggaaccctggtcacagtctcctct

SEQ ID NO: 39 - protein, 107 aa, Artificial Sequence
  synthesized
DIVLTQSPATLSVSPGERATLSCRASQSIGTNIHWYQQKPGQAPRLLIKYASESISGIPSRFSGSGSGTDFTLTISSVQSEDFAVYYCQQNNNWPTTFGQGTKLEIK

SEQ ID NO: 40 - DNA, 321 bp, Artificial Sequence
  synthesized
gacatcgtgctgacccagtctccagccacactgagtgtgtctccaggcgagagagctaccctgtcctgtagagcctctcagtccatcggcaccaacatccactggtatcagcagaagcctggacaggcccctcggctgctgattaagtacgcctccgagtctatcagcggcatcccctccagattctccggctctggctctggcacagactttaccctgaccatctcctccgtgcagtccgaggatttcgccgtgtactactgccagcagaacaacaactggcccaccacctttggccagggcaccaagctggaaatcaaa

SEQ ID NO: 41 - protein, 119 aa, Artificial Sequence; identical to SEQ ID NO: 37
  synthesized

SEQ ID NO: 42 - DNA, 357 bp, Artificial Sequence; identical to SEQ ID NO: 38
  synthesized

SEQ ID NO: 43 - protein, 107 aa, Artificial Sequence
  synthesized
DIVLTQSPATLSVSPGERATLSCRASQSIGTNIHWYQQKPGQAPRLLIKYASESISGIPSRFSGSGSGTDFTLTISSVQSEDFAVYYCQQNNNWPTTFGQGTKLTVL

SEQ ID NO: 44 - DNA, 321 bp, Artificial Sequence
  synthesized
gacatcgtgctgacccagtctccagccacactgagtgtgtctccaggcgagagagctaccctgtcctgtagagcctctcagtccatcggcaccaacatccactggtatcagcagaagcctggacaggcccctcggctgctgattaagtacgcctccgagtctatcagcggcatcccctccagattctccggctctggctctggcacagactttaccctgaccatctcctccgtgcagtccgaggatttcgccgtgtactactgccagcagaacaacaactggcccaccacctttggccagggcaccaaactgacagttctt

SEQ ID NO: 45 - protein, 219 aa, Artificial Sequence
  synthesized
GSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 46 - DNA, 660 bp, Artificial Sequence
  synthesized
ggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 47 - protein, 329 aa, Artificial Sequence
  synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - DNA, 990 bp, Artificial Sequence
  synthesized
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttaa

SEQ ID NO: 49 - protein, 107 aa, Artificial Sequence [sequence omitted]

SEQ ID NO: 50 - DNA, 324 bp, Artificial Sequence
  synthesized
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 51 - protein, 20 aa, Artificial Sequence
  synthesized
GGGGSGGGGSGGGGSGGGGS

SEQ ID NO: 52 - DNA, 60 bp, Artificial Sequence
  synthesized
ggcggcggaggatctggcggaggtggaagcggaggcggaggaagcggtggcggcggatct

SEQ ID NO: 53 - protein, 8 aa, Artificial Sequence
  synthesized
GSHHHHHH

SEQ ID NO: 54 - DNA, 27 bp, Artificial Sequence
  synthesized
ggatcccatcatcaccatcaccattga

SEQ ID NO: 55 - protein, 254 aa, Artificial Sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSGSHHHHHH

SEQ ID NO: 56 - DNA, 765 bp, Artificial Sequence
  synthesized
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggtgctgggaccaagctggagctgaaaggcggtggcggtagtgggggaggcggttctggcggcggagggtccggcggtggaggatcacaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctcgagtggatcccatcatcaccatcaccattga

SEQ ID NO: 57 - protein, 254 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVLGGGGSGGGGSGGGGSGGGGSEVQLVESGGGLVQPGGSLRLSCTVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSSGSHHHHHH

SEQ ID NO: 58 - DNA, 765 bp, Artificial Sequence
  synthesized
gaaatcgttatgacacagtccccatccactcttagcgcttctgtaggggatcgagtgattatcacatgccgggcctcccaatccataggaaccaacatacactggtatcaacaaaaaccaggcaaagcgccaaaactgcttatctactacgcctccgagagtatttctggaatcccgagtcgcttctcaggttctggaagcggcgctgagtttaccctcacaatttcttcactccaaccggatgacttcgctacatattactgccaacaaaacaataattggccgacgacctttggccagggcacgaaacttacggtacttggcggtggcggtagtgggggaggcggttctggcggcggagggtccggcggtggaggatcagaagtacagcttgtcgagtccggtggggggcttgttcagccagggggttccttgaggctttcctgcaccgtctctgggtttagcttgacgaattacggcgttcactgggttagacaagcaccggggaaggggctggaatgggtcggtgtgatatggtccgggggtaatacggattacaatacacctttcacgtcacgctttacgattagcagggacacgtcaaaaaatacagtctacttgcagatgaactctcttagggcggaagatactgcagtttattactgcgcaagggctctgacatactacgattatgaatttgcatattggggccaggggactttggtcacggtctcgagtggatcccatcatcaccatcaccattga

SEQ ID NO: 59 - protein, 465 aa, Artificial Sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 60 - DNA, 1398 bp, Artificial Sequence
  synthesized
gacatcctgctgacccagtctccagtgatcctgtccgtgtctcctggcgagagagtgtccttcagctgcagagcctctcagtccatcggcaccaacatccactggtatcagcagcggaccaacggctcccctcggctgctgattaagtacgcctccgagtctatcagcggcatcccctccagattctccggctctggctctggcaccgacttcaccctgtccatcaactccgtggaatccgaggatatcgccgactactactgccagcagaacaacaactggcccaccacctttggcgctggcaccaagctggaattgaaaggcggcggaggatctggcggaggtggaagcggaggcggaggaagcggtggcggcggatctcaagttcagctcaagcagtctggccctggcctggttcagccttctcagagcctgagcatcacctgtaccgtgtccggcttctccctgaccaattacggcgtgcactgggttcgacagagccctggcaaaggactggaatggctgggagtgatttggagcggcggcaacaccgactacaacacccctttcacctctcggctgtctatcaacaaggacaactccaagagccaggtgttcttcaagatgaactccctgcagtccaacgacaccgccatctactactgtgctcgggccctgacctactacgactacgagtttgcttactggggccagggcaccctggtcacagtttctgctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 61 - protein, 465 aa, Artificial Sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 62 - DNA, 1398 bp, Artificial Sequence
  synthesized
gacatcctgctgacccagtctccagtgatcctgtccgtgtctcctggcgagagagtgtccttcagctgcagagcctctcagtccatcggcaccaacatccactggtatcagcagcggaccaacggctcccctcggctgctgattaagtacgcctccgagtctatcagcggcatcccctccagattctccggctctggctctggcaccgacttcaccctgtccatcaactccgtggaatccgaggatatcgccgactactactgccagcagaacaacaactggcccaccacctttggcgctggcaccaagctggaattgaaaggcggcggaggatctggcggaggtggaagcggaggcggaggaagcggtggcggcggatctcaagttcagctcaagcagtctggccctggcctggttcagccttctcagagcctgagcatcacctgtaccgtgtccggcttctccctgaccaattacggcgtgcactgggttcgacagagccctggcaaaggactggaatggctgggagtgatttggagcggcggcaacaccgactacaacacccctttcacctctcggctgtctatcaacaaggacaactccaagagccaggtgttcttcaagatgaactccctgcagtccgaggacaccgccatctactactgtgctcgggccctgacctactacgactacgagtttgcttactggggccagggcaccctggtcacagtttctgctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 63 - protein, 465 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVLGGGGSGGGGSGGGGSGGGGSEVQLVESGGGLVQPGGSLRLSCKVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 64 - DNA, 1398 bp, Artificial Sequence
  synthesized
gagatcgtgatgacccagtctccttccacactgtccgcctctgtgggcgacagagtgatcatcacctgtagagccagccagtccatcggcaccaacatccactggtatcagcagaagcctggcaaggcccctaagctgctgatctactacgcctccgagtctatcagcggcatcccctccagattctccggctctggatctggcgctgagtttaccctgacaatctccagcctgcagcctgacgacttcgccacctactactgccagcagaacaacaactggcccaccacctttggccagggcaccaaactgacagttcttggcggcggaggatctggcggaggtggaagcggaggcggaggaagcggtggcggcggatctgaagttcagctggtggaatccggcggaggattggttcaacctggcggctctctgagactgtcctgtaaggtgtctggcttctccctgaccaactacggcgtgcactgggtccgacaggcacctggaaaaggactggaatgggtcggagtgatttggagcggcggcaacaccgactacaacacccctttcaccagccggttcaccatctctcgggacacctccaagaacaccgtgtacctgcagatgaactccctgagagccgaggacaccgccgtgtactattgtgctagagccctgacctactatgactacgagttcgcctattggggccagggaaccctggtcacagtctcctctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 65 - protein, 465 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELKGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 66 - DNA, 1398 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctggaattgaaaggcggcggaggatctggcggaggtggaagcggaggcggaggaagcggtggcggcggatctcaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 67 - protein, 465 aa, Artificial Sequence
  synthesized
DIVLTQSPSSLSVSPGERVTISCRASQSIGTNIHWYQQKPGQAPRLLIKYASESISGIPSRFSGSGSGTDFTLTISSVESEDFAVYYCQQNNNWPTTFGQGTKLEIKGGGGSGGGGSGGGGSGGGGSQVQLVESGPGLVQPSGSLSLTCTVSGFSLTNYGVHWVRQAPGKGLEWVGVIWSGGNTDYNTPFTSRFTISKDNSKNQVYLKMNSLRAEDTAVYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 68 - DNA, 1398 bp, Artificial Sequence
  synthesized
gacatcgtgctgacccagtctccatccagcctgtctgtgtctcctggcgagagagtgaccatctcttgccgggcctctcagagcatcggcaccaacatccactggtatcagcagaagcctggacaggcccctcggctgctgattaagtacgcctccgagtctatcagcggcatcccctccagattctccggctctggctctggcacagactttaccctgaccatcagctccgtggaatccgaggacttcgccgtgtactactgccagcagaacaacaactggcccaccacctttggccagggcaccaagctggaaatcaaaggcggcggaggatctggcggaggtggaagcggaggcggaggaagcggtggcggcggatctcaagttcagctggtggaatctggccctggcctggttcagccttctggctctctgtctctgacctgcaccgtgtctggcttctccctgaccaattacggcgtgcactgggttcgacaggctccaggcaaaggactggaatgggtcggagtgatttggagcggcggcaacaccgactacaacacccctttcaccagccggttcaccatctccaaggacaactccaagaaccaggtgtacctgaagatgaactccctgagagccgaggacaccgctgtgtattactgtgctagagccctgacctactacgactacgagttcgcctattggggccagggaaccctggtcacagtctcctctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 69 - protein, 465 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 70 - DNA, 1398 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgacagttcttggcggcggaggatctggcggaggtggaagcggaggcggaggaagcggtggcggcggatctcaagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 71 - protein, 465 aa, Artificial Sequence
  synthesized
DIVLTQSPSSLSVSPGERVTISCRASQSIGTNIHWYQQKPGQAPRLLIKYASESISGIPSRFSGSGSGTDFTLTISSVESEDFAVYYCQQNNNWPTTFGQGTKLTVLGGGGSGGGGSGGGGSGGGGSQVQLVESGPGLVQPSGSLSLTCTVSGFSLTNYGVHWVRQAPGKGLEWVGVIWSGGNTDYNTPFTSRFTISKDNSKNQVYLKMNSLRAEDTAVYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 72 - DNA, 1398 bp, Artificial Sequence
  synthesized
gacatcgtgctgacccagtctccatccagcctgtctgtgtctcctggcgagagagtgaccatctcttgccgggcctctcagagcatcggcaccaacatccactggtatcagcagaagcctggacaggcccctcggctgctgattaagtacgcctccgagtctatcagcggcatcccctccagattctccggctctggctctggcacagactttaccctgaccatcagctccgtggaatccgaggacttcgccgtgtactactgccagcagaacaacaactggcccaccacctttggccagggcaccaagctgacagttcttggcggcggaggatctggcggaggtggaagcggaggcggaggaagcggtggcggcggatctcaagttcagctggtggaatctggccctggcctggttcagccttctggctctctgtctctgacctgcaccgtgtctggcttctccctgaccaattacggcgtgcactgggttcgacaggctccaggcaaaggactggaatgggtcggagtgatttggagcggcggcaacaccgactacaacacccctttcaccagccggttcaccatctccaaggacaactccaagaaccaggtgtacctgaagatgaactccctgagagccgaggacaccgctgtgtattactgtgctagagccctgacctactacgactacgagttcgcctattggggccagggaaccctggtcacagtctcctctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 73 - protein, 465 aa, Artificial Sequence
  synthesized
DIVLTQSPATLSVSPGERATLSCRASQSIGTNIHWYQQKPGQAPRLLIKYASESISGIPSRFSGSGSGTDFTLTISSVQSEDFAVYYCQQNNNWPTTFGQGTKLEIKGGGGSGGGGSGGGGSGGGGSQVQLQESGPGLVKPSESLSLTCTVSGFSLTNYGVHWVRQPPGKGLEWIGVIWSGGNTDYNTPFTSRVTISKDNSKNQVSLKMNSLTAADTAVYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 74 - DNA, 1398 bp, Artificial Sequence
  synthesized
gacatcgtgctgacccagtctccagccacactgagtgtgtctccaggcgagagagctaccctgtcctgtagagcctctcagtccatcggcaccaacatccactggtatcagcagaagcctggacaggcccctcggctgctgattaagtacgcctccgagtctatcagcggcatcccctccagattctccggctctggctctggcacagactttaccctgaccatctcctccgtgcagtccgaggatttcgccgtgtactactgccagcagaacaacaactggcccaccacctttggccagggcaccaagctggaaatcaaaggcggcggaggatctggcggaggtggaagcggaggcggaggaagcggtggcggcggatctcaagttcagctgcaagagtctggccctggcctggtcaagccttccgaatctctgtctctgacctgcaccgtgtccggcttctccctgaccaattatggcgtgcactgggttcgacagcctccaggcaaaggcctggaatggatcggagtgatttggagcggcggcaacaccgactacaacacccctttcacctctagagtgaccatcagcaaggacaactccaagaaccaggtgtccctgaagatgaacagcctgaccgctgccgacaccgctgtgtactattgtgctagagccctgacctactacgactacgagttcgcctattggggccagggaaccctggtcacagtctcctctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 75 - protein, 465 aa, Artificial Sequence
  synthesized
EIVLTQSPSTLSVSPGERVSFSCRASQSIGTNIHWYQQRTGQPPRLLIKYASESISGIPARFSGSGSGTEFTLTITSVQSEDFAVYYCQQNNNWPTTFGQGTKLDIKGGGGSGGGGSGGGGSGGGGSQVQLLQSGPGLVKPSETLSITCTVSGFSLTNYGVHWIRQAPGKGLEWIGVIWSGGNTDYNTPFTSRFTITKDNSKNQVFFKLRSVRAEDTALYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 76 - DNA, 1398 bp, Artificial Sequence
  synthesized
gagatcgtgctgacccagtctccttccacactgtctgtgtctcccggcgagagagtgtccttcagctgtagagcctctcagtccatcggcaccaacatccactggtatcagcagagaaccggccagcctcctcggctgctgattaagtacgcctccgagtccatcagcggcatccctgccagattttccggctctggctctggcaccgagttcaccctgaccatcacctccgtgcagtctgaggacttcgccgtgtactactgccagcagaacaacaactggcccaccacctttggccagggcaccaagctggatatcaaaggcggcggaggatctggcggaggtggaagcggaggcggaggaagcggtggcggcggatctcaagttcagctgttgcagtctggccctggcctggtcaagccttccgagacactgtccatcacctgtaccgtgtccggcttctccctgaccaattacggcgtgcactggatcagacaggcccctggcaaaggcctggaatggatcggagtgatttggagcggcggcaacaccgactacaacacccctttcaccagccggttcaccatcaccaaggacaactccaagaaccaggtgttcttcaagctgcggagcgtgcgcgctgaggataccgctctgtactattgcgctcgggccctgacctactacgactacgagtttgcttactggggccagggaaccctggtcaccgtttcttctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 77 - protein, 465 aa, Artificial Sequence
  synthesized
EIVLTQSPSILSVSPGERVTFSCRASQSIGTNIHWYQQRPGKPPRLLIKYASESISGIPARFSGSGSGTEFTLTISSVQSEDFATYYCQQNNNWPTTFGPGTKLELKGGGGSGGGGSGGGGSGGGGSQVQLVQSGPGLVKPSQTLSLTCTVSGFSLTNYGVHWIRQAPGKGLEWLGVIWSGGNTDYNTPFTSRFTITKDNSKNQVYFKLRSVRSEDTAVYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 78 - DNA, 1398 bp, Artificial Sequence
  synthesized
gagatcgtgctgacccagtctccttccatcctgtctgtgtctcccggcgagagagtgaccttcagctgtagagcctctcagtccatcggcaccaacatccactggtatcagcagcggcctggcaagcctcctcggctgctgattaagtacgcctccgagtccatcagcggcatccctgccagattttccggctctggctctggcaccgagtttaccctgaccatctcctccgtgcagtccgaggatttcgccacctactactgccagcagaacaacaactggcccaccacctttggacccggcaccaagctggaattgaaaggcggcggaggatctggcggaggtggaagcggaggcggaggaagcggtggcggcggatctcaagttcagctggttcagtctggccctggcctcgtgaagccttctcagaccctgtctctgacctgcaccgtgtctggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgagatccgtgcggagcgaggacaccgccgtgtactattgtgctagagccctgacctactacgactacgagttcgcctattggggccagggcacactggtcacagtctcttctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 79 - protein, 465 aa, Artificial Sequence
  synthesized
EIQLTQSPSTLSVSPGERATFSCRASQSIGTNIHWYQQKTGQPPRLLIKYASESISGIPDRFSGSGSGTEFTLSITSVQSEDFAVYYCQQNNNWPTTFGPGTKLEILGGGGSGGGGSGGGGSGGGGSQVQLLQSGPGLVKPSETLSLTCTVSGFSLTNYGVHWIRQAPGKGLEWIGVIWSGGNTDYNTPFTSRFTISKDNSKNQVYFKLRSLTSEDTAIYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 80 - DNA, 1398 bp, Artificial Sequence
  synthesized
gagatccagctgacccagtctccttccacactgtctgtgtctcccggcgagagagccaccttcagctgtagagcctctcagtccatcggcaccaacatccactggtatcagcagaaaaccggccagcctcctcggctgctgattaagtacgcctccgagtccatcagcggcatccctgacagattctccggctctggctctggcaccgagttcaccctgtctatcacctccgtgcagtccgaggacttcgccgtgtactactgccagcagaacaacaactggcccaccacctttggacccggcaccaagctggaaattcttggcggcggaggatctggcggaggtggaagcggaggcggaggaagcggtggcggcggatctcaagttcagctgttgcagtctggccctggcctggtcaagccttccgaaacactgtctctgacctgcaccgtgtccggcttctccctgaccaattatggcgtgcactggatcagacaggcccctggcaaaggcctggaatggatcggagtgatttggagcggcggcaacaccgactacaacacccctttcaccagccggttcaccatctccaaggacaactccaagaaccaggtgtacttcaagctgcggtccctgacctctgaggacaccgccatctactactgcgctagagccctgacctactacgactacgagttcgcctattggggccagggcacactggtcacagtctcttctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 81 - protein, 465 aa, Artificial Sequence
  synthesized
EIVLTQSPSILSVSPGERATFSCRASQSIGTNIHWYQQRPGKPPRLLIKYASESISGIPSRFSGSGSGTEFTLTITSVQSEDIAVYYCQQNNNWPTTFGPGTKLELKGGGGSGGGGSGGGGSGGGGSQVQLQQSGPGLVKPSQTLSITCTVSGFSLTNYGVHWIRQAPGKGLEWLGVIWSGGNTDYNTPFTSRFTISKDNSKNQVYFKLRSLRAEDTAIYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 82 - DNA, 1398 bp, Artificial Sequence
  synthesized
gagatcgtgctgacccagtctccttccatcctgtctgtgtctcccggcgagagagccaccttcagctgtagagcctctcagtccatcggcaccaacatccactggtatcagcagcggcctggcaagcctcctcggctgctgattaagtacgcctccgagtctatcagcggcatcccctccagattctccggctctggatctggcaccgagttcaccctgaccatcacctccgtgcagtccgaggatatcgccgtgtactactgccagcagaacaacaactggcccaccacctttggacccggcaccaagctggaattgaaaggcggcggaggatctggcggaggtggaagcggaggcggaggaagcggtggcggcggatctcaagttcagttgcagcagtctggccctggcctcgtgaagccttctcagaccctgtctatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatctccaaggacaactccaagaaccaggtgtacttcaagctgcggtccctgagagccgaggacaccgccatctactactgtgctagagccctgacctactacgactacgagttcgcctattggggccagggcacactggtcacagtctcttctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 83 - protein, 465 aa, Artificial Sequence
  synthesized
DIVLTQSPATLSVSPGERATLSCRASQSIGTNIHWYQQKPGQAPRLLIKYASESISGIPSRFSGSGSGTDFTLTISSVQSEDFAVYYCQQNNNWPTTFGQGTKLTVLGGGGSGGGGSGGGGSGGGGSQVQLQESGPGLVKPSESLSLTCTVSGFSLTNYGVHWVRQPPGKGLEWIGVIWSGGNTDYNTPFTSRVTISKDNSKNQVSLKMNSLTAADTAVYYCARALTYYDYEFAYWGQGTLVTVSSGSGGSPVAGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 84 - DNA, 1398 bp, Artificial Sequence
  synthesized
gacatcgtgctgacccagtctccagccacactgagtgtgtctccaggcgagagagctaccctgtcctgtagagcctctcagtccatcggcaccaacatccactggtatcagcagaagcctggacaggcccctcggctgctgattaagtacgcctccgagtctatcagcggcatcccctccagattctccggctctggctctggcacagactttaccctgaccatctcctccgtgcagtccgaggatttcgccgtgtactactgccagcagaacaacaactggcccaccacctttggccagggcaccaaactgacagttcttggcggcggaggatctggcggaggtggaagcggaggcggaggaagcggtggcggcggatctcaagttcagctgcaagagtctggccctggcctggtcaagccttccgaatctctgtctctgacctgcaccgtgtccggcttctccctgaccaattatggcgtgcactgggttcgacagcctccaggcaaaggcctggaatggatcggagtgatttggagcggcggcaacaccgactacaacacccctttcacctctagagtgaccatcagcaaggacaactccaagaaccaggtgtccctgaagatgaacagcctgaccgctgccgacaccgctgtgtactattgtgctagagccctgacctactacgactacgagttcgcctattggggccagggaaccctggtcacagtctcctctggatccggcggctctcccgtcgctggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgagatgccacgtcaaaggcttctatcccagcgacatcgccgtggagtgggagagcaatgggcagccggagaacaactacaagaccacgaagcccgtgctggactccgacggctccttcttcctctacagcaccctcaccgtggacaagagcaggtggcagcaggggaacgtcttctcatgctccgtgctccatgaggctctgcacaaccactacacacagaagagcctctccctgtctccgggcaaatga

SEQ ID NO: 85 - protein, 1217 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVLGGGGSGGGGSGGGGSGGGGSEVQLVESGGGLVQPGGSLRLSCSVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 86 - DNA, 3654 bp, Artificial Sequence
  synthesized
gaaatcgttatgacacagtccccatccactcttagcgcttctgtaggggatcgagtgattatcacatgccgggcctcccaatccataggaaccaacatacactggtatcaacaaaaaccaggcaaagcgccaaaactgcttatctactacgcctccgagagtatttctggaatcccgagtcgcttctcaggttctggaagcggcgctgagtttaccctcacaatttcttcactccaaccggatgacttcgctacatattactgccaacaaaacaataattggccgacgacctttggccagggcacgaaacttacggtacttggcggtggcggtagtgggggaggcggttctggcggcggagggtccggcggtggaggatcagaagtacagcttgtcgagtccggtggggggcttgttcagccagggggttccttgaggctttcctgctccgtctctgggtttagcttgacgaattacggcgttcactgggttagacaagcaccggggaaggggctggaatgggtcggtgtgatatggtccgggggtaatacggattacaatacacctttcacgtcacgctttacgattagcagggacacgtcaaaaaatacagtctacttgcagatgaactctcttagggcggaagatactgcagtttattactgcgcaagggctctgacatactacgattatgaatttgcatattggggccaggggactttggtcacggtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 87 - protein, 473 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDPVLTQSPSSLSASVGDRVTISCQSSQSVAKNNNLAWFQQKPGQAPKLLIYSASTLAAGVPSRFSGSGSGTDFTLTISSVQPEDFATYYCSARDSGNIQSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 1422 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgacggtactgggtggaggcggttcaggcggaggtggttccggcggtggcggctccggtggaggcggctctcaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggcggtggagggtccggcggtggtggatccgatccagttctgacacaaagtccatccagcctgtctgcctcagtcggcgacagagtgaccatcagttgccagagctcacagtctgtggctaagaacaacaacttggcgtggttccaacagaaacctggacaggctccgaaattgctgatctattctgcttccacgcttgctgctggtgttccttcccgcttttcaggtagtggtagcgggacagacttcactttgactataagcagcgtgcagcctgaagattttgcgacctactattgttctgctagagacagtggaaatattcagtcctttggggggggaacgaaggtcgaaataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 89 - protein, 1533 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVLGGGGSGGGGSGGGGSGGGGSEVQLVESGGGLVQPGGSLRLSCSVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 90 - DNA, 4602 bp, Artificial Sequence
  synthesized
gaaatcgttatgacacagtccccatccactcttagcgcttctgtaggggatcgagtgattatcacatgccgggcctcccaatccataggaaccaacatacactggtatcaacaaaaaccaggcaaagcgccaaaactgcttatctactacgcctccgagagtatttctggaatcccgagtcgcttctcaggttctggaagcggcgctgagtttaccctcacaatttcttcactccaaccggatgacttcgctacatattactgccaacaaaacaataattggccgacgacctttggccagggcacgaaacttacggtacttggcggtggcggtagtgggggaggcggttctggcggcggagggtccggcggtggaggatcagaagtacagcttgtcgagtccggtggggggcttgttcagccagggggttccttgaggctttcctgctccgtctctgggtttagcttgacgaattacggcgttcactgggttagacaagcaccggggaaggggctggaatgggtcggtgtgatatggtccgggggtaatacggattacaatacacctttcacgtcacgctttacgattagcagggacacgtcaaaaaatacagtctacttgcagatgaactctcttagggcggaagatactgcagtttattactgcgcaagggctctgacatactacgattatgaatttgcatattggggccaggggactttggtcacggtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacgagagggccccgagctgtctcctgatgacccagcaggcctcttggacttgcggcagggtatgttcgctcaacttgtggctcagaatgttctgctcattgatggaccactctcttggtatagtgaccccggtctggccggggtgagtctgaccggcgggctctcttataaagaggatactaaggaactggtcgtagcaaaagcgggcgtttattacgttttttttcagctggagctcaggcgcgtggtggccggcgagggcagtggctctgtgtccctggccctgcacttacagcccttgagaagcgctgcaggtgctgccgcactggctttaactgttgacctccctccggcctcttctgaagctagaaacagcgctttcggcttccaagggcgcctgctgcacctgagcgcaggccagcgcttaggtgtgcaccttcatacagaggccagggcccgacacgcttggcagctcacacagggtgccacggttctcggacttttccgcgttactcccgagatccccgctggcctcggaagtactggttctgggtctaaacccggttccggcgaaggtagtactaaaggacgagaagggccagagttaagtccagatgaccctgctgggcttttggacctgcggcagggcatgttcgctcaactggtggctcagaacgtgctgctgatcgatggccccctgagttggtacagcgatcccgggctggcaggcgtgtcacttacagggggcctctcttacaaggaagacaccaaggagttagtggtcgctaaggctggcgtgtattacgtgttcttccaactggagctgagaagggttgtggcaggagagggtagcggcagcgtgtctttagcccttcacttgcagcccctgaggtctgctgcaggtgcagccgctctcgcgctcaccgtggatctccccccagcctcatctgaagctaggaacagtgcatttggctttcagggacgcttgctgcacctctccgctggacagaggctgggcgtgcaccttcacacagaggcccgtgccaggcatgcatggcagctcactcagggggcaacagtgctgggtctcttccgcgtgactcctgaaataccagctggacttggcggtggaggcagcggcggaggaggatctcgtgaggggccagaactgtcccccgatgacccagccggactgctcgatctcagacagggcatgttcgctcagcttgtagcccaaaatgtcctcctgattgacggccctttgagctggtatagtgatcccggcttggccggggtatctctgaccggaggcctctcctacaaggaagacaccaaagagctggtggtggcaaaagcgggggtgtattatgtgttctttcagctcgagctgcggagagttgtggccggggaagggtctgggagcgtatctcttgcacttcacctgcagcccctgcgcagcgccgctggagccgccgcccttgctcttactgtggatctgcctcctgcttcctcagaagcacgcaacagcgccttcggctttcaaggacgtctcctgcacttgtccgcaggacagaggttgggcgtccatttacacactgaggcacgggcacggcacgcttggcagcttacccagggagccaccgtgctgggactctttagagtgacacccgagatccccgctggcttgtga

SEQ ID NO: 91 - protein, 473 aa, Artificial Sequence; identical to SEQ ID NO: 87
  synthesized

SEQ ID NO: 92 - DNA, 1422 bp, Artificial Sequence; identical to SEQ ID NO: 88
  synthesized

SEQ ID NO: 93 - protein, 1217 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELKGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 94 - DNA, 3654 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagttggaactgaaaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 95 - protein, 473 aa, Artificial Sequence; identical to SEQ ID NO: 87
  synthesized

SEQ ID NO: 96 - DNA, 1422 bp, Artificial Sequence; identical to SEQ ID NO: 88
  synthesized

SEQ ID NO: 97 - protein, 1533 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELKGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 98 - DNA, 4602 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagttggaactgaaaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacgagagggccccgagctgtctcctgatgacccagcaggcctcttggacttgcggcagggtatgttcgctcaacttgtggctcagaatgttctgctcattgatggaccactctcttggtatagtgaccccggtctggccggggtgagtctgaccggcgggctctcttataaagaggatactaaggaactggtcgtagcaaaagcgggcgtttattacgttttttttcagctggagctcaggcgcgtggtggccggcgagggcagtggctctgtgtccctggccctgcacttacagcccttgagaagcgctgcaggtgctgccgcactggctttaactgttgacctccctccggcctcttctgaagctagaaacagcgctttcggcttccaagggcgcctgctgcacctgagcgcaggccagcgcttaggtgtgcaccttcatacagaggccagggcccgacacgcttggcagctcacacagggtgccacggttctcggacttttccgcgttactcccgagatccccgctggcctcggaagtactggttctgggtctaaacccggttccggcgaaggtagtactaaaggacgagaagggccagagttaagtccagatgaccctgctgggcttttggacctgcggcagggcatgttcgctcaactggtggctcagaacgtgctgctgatcgatggccccctgagttggtacagcgatcccgggctggcaggcgtgtcacttacagggggcctctcttacaaggaagacaccaaggagttagtggtcgctaaggctggcgtgtattacgtgttcttccaactggagctgagaagggttgtggcaggagagggtagcggcagcgtgtctttagcccttcacttgcagcccctgaggtctgctgcaggtgcagccgctctcgcgctcaccgtggatctccccccagcctcatctgaagctaggaacagtgcatttggctttcagggacgcttgctgcacctctccgctggacagaggctgggcgtgcaccttcacacagaggcccgtgccaggcatgcatggcagctcactcagggggcaacagtgctgggtctcttccgcgtgactcctgaaataccagctggacttggcggtggaggcagcggcggaggaggatctcgtgaggggccagaactgtcccccgatgacccagccggactgctcgatctcagacagggcatgttcgctcagcttgtagcccaaaatgtcctcctgattgacggccctttgagctggtatagtgatcccggcttggccggggtatctctgaccggaggcctctcctacaaggaagacaccaaagagctggtggtggcaaaagcgggggtgtattatgtgttctttcagctcgagctgcggagagttgtggccggggaagggtctgggagcgtatctcttgcacttcacctgcagcccctgcgcagcgccgctggagccgccgcccttgctcttactgtggatctgcctcctgcttcctcagaagcacgcaacagcgccttcggctttcaaggacgtctcctgcacttgtccgcaggacagaggttgggcgtccatttacacactgaggcacgggcacggcacgcttggcagcttacccagggagccaccgtgctgggactctttagagtgacacccgagatccccgctggcttgtga

SEQ ID NO: 99 - protein, 473 aa, Artificial Sequence; identical to SEQ ID NO: 87
  synthesized

SEQ ID NO: 100 - DNA, 1422 bp, Artificial Sequence; identical to SEQ ID NO: 88
  synthesized

SEQ ID NO: 101 - protein, 1224 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 102 - DNA, 3675 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 103 - protein, 475 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLRLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - DNA, 1428 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgacggtactgggtggaggcggttcaggcggaggtggttccggcggtggcggctccggtggaggcggctctcaggtcacattgaaggaatctggccccggccttgttcagccaggacagacccttaggctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagcggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaattcactctcaccatcagcagcctgcagcctgatgattttgcaacttattactgccaaggctatttttattttattagtcgtacttatgtaaattctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 105 - protein, 1229 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLGGGGSGGGGSGGGGSGGGGSEVQLVESGGGLVQPGGSLRLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 106 - DNA, 3687 bp, Artificial Sequence
  synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttggtggcggaggcagtggtggcgggggcagcggaggtggtggttcagggggtggtgggagcgaagtccaattggtagaaagtggcggtggtctggtgcaacctggtggatctcttcgcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagtggcggtggagggtccggcggtggtggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaa

SEQ ID NO: 107 - protein, 470 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - DNA, 1413 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggcggtggagggtccggcggtggtggatcagaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 109 - protein, 1229 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLGGGGSGGGGSGGGGSGGGGSEVQLVESGGGLVQPGGSLRLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSQVQLKQSGPGLVQPSQSLSITCTVSGFSLTNYGVHWVRQSPGKCLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 110 - DNA, 3690 bp, Artificial Sequence
  synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttggtggcggaggcagtggtggcgggggcagcggaggtggtggttcagggggtggtgggagcgaagtccaattggtagaaagtggcggtggtctggtgcaacctggtggatctcttcgcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagtggcggtggagggtccggcggtggtggatcacaggtgcagctgaagcagtcaggacctggcctagtgcagccctcacagagcctgtccatcacctgcacagtctctggtttctcattaactaactatggtgtacactgggttcgccagtctccaggaaagtgtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 111 - protein, 734 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGCGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 112 - DNA, 2205 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggattccttccaggtttagtggcagtggatcagggacagattttactcttagcatcaacagtgtggagtctgaagatattgcagattattactgtcaacaaaataataactggccaaccacgttcggttgtgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagctga

SEQ ID NO: 113 - protein, 1234 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSGGGGSGGGGSEVQLVESGGGLVQPGGSLRLSCAASGFTISTNAMSWVREAPGKCLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 114 - DNA, 3705 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggtggaggcggatctggcggaggtggttccggcggtggcggctccggtggaggcggctctgaggtgcagctggtggagtctgggggaggcttggtccagcctggggggtccctgagactctcctgtgcagcctctggattcaccatcagtaccaatgcaatgagctgggtccgcgaggctccagggaagtgtctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 115 - protein, 739 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 116 - DNA, 2220 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggctgtgggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagctga

SEQ ID NO: 117 - protein, 1229 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLGGGGSGGGGSGGGGSGGGGSEVQLVESGGGLVQPGGSLRLSCAASGFTISTNAMSWVRQAPGKGLEWVGVITGRDITYYASWAKGRFTISRDTSKNTVYLQMNSLRAEDTAVYYCARDGGSSAITSNNIWGQGTLVTVSSGGGGSGGGGSQVQLQQSGPGLVKPSETLSITCTVSGFSLTNYGVHWIRQAPGKCLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSGRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 118 - DNA, 3690 bp, Artificial Sequence
  synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttggtggcggaggcagtggtggcgggggcagcggaggtggtggttcagggggtggtgggagcgaagtccaattggtagaaagtggcggtggtctggtgcaacctggtggatctcttcgcctctcatgcgccgctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgagggcggaagacacagctgtgtattactgcgcgagggatggaggtagttccgccataacttccaacaacatatggggacaaggcacgctggttactgtctcgagtggcggtggagggtccggcggtggtggatcacaagtacagttgcagcaatccggtcccggtctcgtcaaaccgagtgagacgcttagtataacgtgtactgtttcaggctttagccttacgaactatggagttcactggattcggcaggcacccggcaaatgtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtgtcgagcggtggaggcggatctggcggaggtggttccggcggtggcggctccggtggaggcggctctgacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaaggcggtggagggtccggcggtggtggctccggacggtcgctggtggagtctgggggaggcttggtccagcctggggggtccctgagactctcctgtactgcctctggattcaccatcagtagctaccacatgcagtgggtccgccaggctccagggaaggggctggagtacatcggaaccattagtagtggtggtaatgtatactacgcaagctccgctagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcttcaggcggtggcggtagtgggggaggcggttctggcggcggagggtccggcggtggaggatcagacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaatga

SEQ ID NO: 119 - protein, 734 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGCGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGGGGSGGGGSGGGGSGGGGSQVQLQESGGGLVKPGGSLSLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSS

SEQ ID NO: 120 - DNA, 2205 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgctcggatggaactctggtcttactactttgattattgggggcaggggactctcgtcacggtctcgagtggcggtggagggtccggcggtggtggatcagaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggatgcggtacaaagctgaccgttttacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcggtagcggtggcggcggaagtggtggcggaggatcccagtctgccctgactcagcctgcctccgtgtctgggtctcctggacagtcgatcaccatctcctgcactggaaccagcagtgacgttggtggttataactttgtctcctggtaccaacaacacccaggcaaagcccccaaactcatgatctatgatgtcagtgatcggccctcaggggtgtctgatcgcttctccggctccaagtctggcaacacggcctccctgatcatctctggcctccaggctgacgacgaggctgattattactgcagctcatatgggagcagcagcactcatgtgattttcggcggagggaccaaggtgaccgtcctaggtggaggcggttcaggcggaggtggttccggcggtggcggctccggtggaggcggctctcaggtgcaattgcaggagtcggggggaggcctggtcaagcctggagggtccctgagtctctcctgtgcagcctctggattcacctttagtagttattggatgagctgggtccgccaggctccagggaaggggctggagtgggtggccaacataaaccgcgatggaagtgcgagttactatgtggactctgtgaagggccgattcaccatctccagagacgacgccaagaactcactgtatctgcaaatgaacagcctgagagctgaggacacggctgtgtattactgtgcgagagatcgtggggtgggctacttcgatctctggggccgtggcaccctggtcaccgtctctagctga

SEQ ID NO: 121 - protein, 1270 aa, Artificial Sequence
  synthesized
EIVMTQSPSTLSASVGDRVIITCRASQSIGTNIHWYQQKPGKAPKLLIYYASESISGIPSRFSGSGSGAEFTLTISSLQPDDFATYYCQQNNNWPTTFGQGTKLTVLGGGGSGGGGSGGGGSGGGGSEVQLVESGGGLVQPGGSLRLSCSVSGFSLTNYGVHWVRQAPGKGLEWVGVIWSGGNTDYNTPFTSRFTISRDTSKNTVYLQMNSLRAEDTAVYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGSTGSGSKPGSGEGSTKGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 122 - DNA, 3813 bp, Artificial Sequence
  synthesized
gaaatcgttatgacacagtccccatccactcttagcgcttctgtaggggatcgagtgattatcacatgccgggcctcccaatccataggaaccaacatacactggtatcaacaaaaaccaggcaaagcgccaaaactgcttatctactacgcctccgagagtatttctggaatcccgagtcgcttctcaggttctggaagcggcgctgagtttaccctcacaatttcttcactccaaccggatgacttcgctacatattactgccaacaaaacaataattggccgacgacctttggccagggcacgaaacttacggtacttggcggtggcggtagtgggggaggcggttctggcggcggagggtccggcggtggaggatcagaagtacagcttgtcgagtccggtggggggcttgttcagccagggggttccttgaggctttcctgctccgtctctgggtttagcttgacgaattacggcgttcactgggttagacaagcaccggggaaggggctggaatgggtcggtgtgatatggtccgggggtaatacggattacaatacacctttcacgtcacgctttacgattagcagggacacgtcaaaaaatacagtctacttgcagatgaactctcttagggcggaagatactgcagtttattactgcgcaagggctctgacatactacgattatgaatttgcatattggggccaggggactttggtcacggtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtataccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggcggcggcggttcaggtggtgggggatcccgagagggccccgagctgtctcctgatgacccagcaggcctcttggacttgcggcagggtatgttcgctcaacttgtggctcagaatgttctgctcattgatggaccactctcttggtatagtgaccccggtctggccggggtgagtctgaccggcgggctctcttataaagaggatactaaggaactggtcgtagcaaaagcgggcgtttattacgttttttttcagctggagctcaggcgcgtggtggccggcgagggcagtggctctgtgtccctggccctgcacttacagcccttgagaagcgctgcaggtgctgccgcactggctttaactgttgacctccctccggcctcttctgaagctagaaacagcgctttcggcttccaagggcgcctgctgcacctgagcgcaggccagcgcttaggtgtgcaccttcatacagaggccagggcccgacacgcttggcagctcacacagggtgccacggttctcggacttttccgcgttactcccgagatccccgctggcctcggaagtactggttctgggtctaaacccggttccggcgaaggtagtactaaaggacgagaagggccagagttaagtccagatgaccctgctgggcttttggacctgcggcagggcatgttcgctcaactggtggctcagaacgtgctgctgatcgatggccccctgagttggtacagcgatcccgggctggcaggcgtgtcacttacagggggcctctcttacaaggaagacaccaaggagttagtggtcgctaaggctggcgtgtattacgtgttcttccaactggagctgagaagggttgtggcaggagagggtagcggcagcgtgtctttagcccttcacttgcagcccctgaggtctgctgcaggtgcagccgctctcgcgctcaccgtggatctccccccagcctcatctgaagctaggaacagtgcatttggctttcagggacgcttgctgcacctctccgctggacagaggctgggcgtgcaccttcacacagaggcccgtgccaggcatgcatggcagctcactcagggggcaacagtgctgggtctcttccgcgtgactcctgaaataccagctggacttggcggtggaggcagcggcggaggaggatctcgtgaggggccagaactgtcccccgatgacccagccggactgctcgatctcagacagggcatgttcgctcagcttgtagcccaaaatgtcctcctgattgacggccctttgagctggtatagtgatcccggcttggccggggtatctctgaccggaggcctctcctacaaggaagacaccaaagagctggtggtggcaaaagcgggggtgtattatgtgttctttcagctcgagctgcggagagttgtggccggggaagggtctgggagcgtatctcttgcacttcacctgcagcccctgcgcagcgccgctggagccgccgcccttgctcttactgtggatctgcctcctgcttcctcagaagcacgcaacagcgccttcggctttcaaggacgtctcctgcacttgtccgcaggacagaggttgggcgtccatttacacactgaggcacgggcacggcacgcttggcagcttacccagggagccaccgtgctgggactctttagagtgacacccgagatccccgctggcttgtga

SEQ ID NO: 123 - protein, 963 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDPQLTQSPSSLSATVGQRVTINCQSSQSVAKNNNLAWFQQKPGKPPKLLIYSASTLAAGVPSRFSGSGSGTQFTLTITRVQSEDFATYYCSARDSGNIQSFGGGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVTVLGGGGSGGGGSGGGGSGGGGSEVQLLESGGGLVQPGGSLSLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSS

SEQ ID NO: 124 - DNA, 2892 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggcggtggagggtccggcggtggtggatcagatcctcaattgacccagtcccctagtagcctttctgcaaccgtgggccagagggtaacaatcaattgccagtcaagccagtccgtagcgaagaataataatttggcatggttccaacaaaaaccgggtaaacctcctaaactccttatctactcagcttcaacccttgccgcgggtgtgcccagccggttctcaggctcaggttccgggactcaatttacacttaccatcactcgcgtccaaagtgaggattttgcaacctactattgttctgcacgggattccgggaacatacagtcctttgggggtggaactcgggtggagataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggaggcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccaagccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaacagattcacgcagaagagcctctccctgtctccgggtggcggtggagggtccggcggtggtggatccgacattcagatgacacagtcaccgtctacgctctcagcctcagtaggcgatagggtgactatcacctgccaagcctcccaatctataagttcacacctcaactggtatcaacagaaacccggtaaagcaccgaagctgctcatctacaaggcatcaactcttgcctcaggtgtgcctagtcgcttctccgggagcggctccggtactgagtttactttgacgatatcttccctccaacccgacgacttcgccacatactattgtcagcagggttatagttggggaaatgttgacaatgtgtttggagggggaacgaaagtgactgtgttgggagggggaggaagtggcggcggcggaagcggaggtggtggctccggcgggggtggaagcgaagtgcaattgctcgaaagtggtggcggacttgtgcaacctggtggaagcctctcactctcttgcgcggcgagtggttttagcttcagtagcggctacgatatgtgctgggtaagacaggctcctggtaaaggccttgagtggattgcgtgtatcgccgccgggtctgctggtataacatacgatgcaaactgggcgaaagggcgcttcacaatctccagggacaactcaaaaaatacattgtacctccagatgaatagcctgagagccgaagacaccgcagtctactattgcgcaaggtcagcatttagcttcgattatgcaatggatctttggggccaggggacgctcgtcaccgtctcatcttga

SEQ ID NO: 125 - protein, 955 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELKGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSQVQLQESGGRLVQPGEPLSLTCKTSGIDLSSNAIGWVRQAPGKGLEWIGVIFGSGNTYYASWAKGRFTISRSTSTVYLKMNSLRSEDTAIYYCARGGYSSDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKLTVLGGGGSGGGGSGGGGSGGGGSEVQLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSS

SEQ ID NO: 126 - DNA, 2868 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagttggaactgaaaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcacaagtgcagttgcaagaaagtggtggtagactggttcagcctggtgaacccttgtcactgacgtgtaaaacaagcggcattgatctgtcctctaacgccatcggatgggtccgacaggccccaggaaaaggtctggagtggatcggagttatcttcgggagcggcaatacatactacgcaagctgggcaaaagggcgatttacgatatcacggagcacctctacagtttatttgaaaatgaactccctccggtccgaggataccgcgatatattactgtgccagaggggggtactcctctgatatctgggggcagggtacactggttacagtttcatccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtataccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggcggcggcggttcaggtggtgggggatccgacgttgtgatgacgcagtctcctagttcagtatcagcgtctgttggggatcgagtcacgattacatgccaggcgagccaaaatattaggacgtatctgtcttggtatcaacaaaagcctggtaaggctccaaaactcctcatttacgccgctgccaacttggctagtggagtaccttcacgctttagtgggtcaggttctggaacagattttacgttgacgatctccgatttggaaccaggggacgctgccacgtattattgccagtccacctacctcgggactgactacgttggtggggcgtttggcggaggaacaaaactcactgtacttggtggtggtggttcaggcggggggggttccggcggaggcggttctgggggtggaggttccgaggttcagttggttgaaagtggggggggactcgttcagcctggaggctctttgcgactttcttgtaccgcttctgggttcactataagttcatatcatatgcaatgggtaagacaggcgcctggtaagggacttgaatacataggaacaataagctctggtggtaacgtctattacgcctcatccgcgcgggggaggtttacaatttccaggccttctagcaaaaacacggtagatctgcaaatgaactctcttcgcgctgaagatacagcagtctactactgcgccagggattcagggtattctgaccccatgtgggggcagggcacattggttaccgtgtcctcttga

SEQ ID NO: 127 - protein, 963 aa, Artificial Sequence; identical to SEQ ID NO: 123
  synthesized

SEQ ID NO: 128 - DNA, 2892 bp, Artificial Sequence; identical to SEQ ID NO: 124
  synthesized

SEQ ID NO: 129 - protein, 962 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLGGGGSGGGGSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKLTVLGGGGSGGGGSGGGGSGGGGSEVQLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSS

SEQ ID NO: 130 - DNA, 2889 bp, Artificial Sequence
  synthesized
gaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttaggcggtggcggtagtgggggaggcggttctggcggcggagggtccggcggtggaggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctcgagcggcggtggagggtccggcggtggtggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtgtcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtataccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggcggcggcggttcaggtggtgggggatccgacgttgtgatgacgcagtctcctagttcagtatcagcgtctgttggggatcgagtcacgattacatgccaggcgagccaaaatattaggacgtatctgtcttggtatcaacaaaagcctggtaaggctccaaaactcctcatttacgccgctgccaacttggctagtggagtaccttcacgctttagtgggtcaggttctggaacagattttacgttgacgatctccgatttggaaccaggggacgctgccacgtattattgccagtccacctacctcgggactgactacgttggtggggcgtttggcggaggaacaaaactcactgtacttggtggtggtggttcaggcggggggggttccggcggaggcggttctgggggtggaggttccgaggttcagttggttgaaagtggggggggactcgttcagcctggaggctctttgcgactttcttgtaccgcttctgggttcactataagttcatatcatatgcaatgggtaagacaggcgcctggtaagggacttgaatacataggaacaataagctctggtggtaacgtctattacgcctcatccgcgcgggggaggtttacaatttccaggccttctagcaaaaacacggtagatctgcaaatgaactctcttcgcgctgaagatacagcagtctactactgcgccagggattcagggtattctgaccccatgtgggggcagggcacattggttaccgtgtcctcttga

SEQ ID NO: 131 - protein, 965 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVTVLGGGGSGGGGSGGGGSGGGGSEVQLLESGGGLVQPGGSLSLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSS

SEQ ID NO: 132 - DNA, 2898 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagcggcggtggagggtccggcggtggtggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaattcactctcaccatcagcagcctgcagcctgatgattttgcaacttattactgccaaggctatttttattttattagtcgtacttatgtaaattctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggaggcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccaagccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaacagattcacgcagaagagcctctccctgtctccgggtggcggtggagggtccggcggtggtggatccgacattcagatgacacagtcaccgtctacgctctcagcctcagtaggcgatagggtgactatcacctgccaagcctcccaatctataagttcacacctcaactggtatcaacagaaacccggtaaagcaccgaagctgctcatctacaaggcatcaactcttgcctcaggtgtgcctagtcgcttctccgggagcggctccggtactgagtttactttgacgatatcttccctccaacccgacgacttcgccacatactattgtcagcagggttatagttggggaaatgttgacaatgtgtttggagggggaacgaaagtgactgtgttgggagggggaggaagtggcggcggcggaagcggaggtggtggctccggcgggggtggaagcgaagtgcaattgctcgaaagtggtggcggacttgtgcaacctggtggaagcctctcactctcttgcgcggcgagtggttttagcttcagtagcggctacgatatgtgctgggtaagacaggctcctggtaaaggccttgagtggattgcgtgtatcgccgccgggtctgctggtataacatacgatgcaaactgggcgaaagggcgcttcacaatctccagggacaactcaaaaaatacattgtacctccagatgaatagcctgagagccgaagacaccgcagtctactattgcgcaaggtcagcatttagcttcgattatgcaatggatctttggggccaggggacgctcgtcaccgtctcatcttga

SEQ ID NO: 133 - protein, 967 aa, Artificial Sequence
  synthesized
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKGGGGSGGGGSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSGGGGSGGGGSQVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKLTVLGGGGSGGGGSGGGGSGGGGSEVQLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSS

SEQ ID NO: 134 - DNA, 2904 bp, Artificial Sequence
  synthesized
gacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagaatttactctcaccatcagcagcctgcagcctgatgattttgcaacttattactgccaaggctatttttattttattagtcgtacttatgtaaattctttcggcggagggaccaaggtggagatcaaaggcggtggcggtagtgggggaggcggttctggcggcggagggtccggcggtggaggatcagaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgagagacggtggttcttctgctattactagtaacaacatttggggccagggaaccctggtcaccgtctcgagtggcggtggagggtccggcggtggtggatcacaagtacagttgcagcaatccggtcccggtctcgtcaaaccgagtgagacgcttagtataacgtgtactgtttcaggctttagccttacgaactatggagttcactggattcggcaggcacccggcaaaggtttggaatggctgggtgttatttggtcaggtggaaatacagactataacaccccctttacaagtcggttcacaattacgaaagataattccaaaaatcaagtttatttcaagttgagatccgtccgcgcggacgacactgcgatctactattgtgcgagggcactgacctactacgattacgaatttgcgtattgggggcaagggactcttgtaacagtctccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtataccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggcggcggcggttcaggtggtgggggatccgacgttgtgatgacgcagtctcctagttcagtatcagcgtctgttggggatcgagtcacgattacatgccaggcgagccaaaatattaggacgtatctgtcttggtatcaacaaaagcctggtaaggctccaaaactcctcatttacgccgctgccaacttggctagtggagtaccttcacgctttagtgggtcaggttctggaacagattttacgttgacgatctccgatttggaaccaggggacgctgccacgtattattgccagtccacctacctcgggactgactacgttggtggggcgtttggcggaggaacaaaactcactgtacttggtggtggtggttcaggcggggggggttccggcggaggcggttctgggggtggaggttccgaggttcagttggttgaaagtggggggggactcgttcagcctggaggctctttgcgactttcttgtaccgcttctgggttcactataagttcatatcatatgcaatgggtaagacaggcgcctggtaagggacttgaatacataggaacaataagctctggtggtaacgtctattacgcctcatccgcgcgggggaggtttacaatttccaggccttctagcaaaaacacggtagatctgcaaatgaactctcttcgcgctgaagatacagcagtctactactgcgccagggattcagggtattctgaccccatgtgggggcagggcacattggttaccgtgtcctcttga

SEQ ID NO: 135 - protein, 960 aa, Artificial Sequence
  synthesized
ENVLTQSPASLSASPGERVTITCSASSSVSYMHWYQQKPGQAPKLWIYDTSKLASGVPSRFSGSGSGNDHTLTISSMEPEDFATYYCFQGSVYPFTFGQGTKVTVLGGGGSGGGGSGGGGSGGGGSQVTLKESGPGLVQPGQTLSLTCAFSGFSLSTSGMGVGWIRQPPGKGLEWLAHIWWDDDKRYNPALKSRLTISKDTSKNQVYLQMNSLDAEDTAVYYCARMELWSYYFDYWGQGTLVTVSSGGGGSGGGGSEIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVTVLGGGGSGGGGSGGGGSGGGGSEVQLLESGGGLVQPGGSLSLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSS

SEQ ID NO: 136 - DNA, 2883 bp, Artificial Sequence
  synthesized
gaaaatgtattgacacagagccccgcctccctcagtgcctcacctggggaaagggtaactatcacttgctctgcatcaagcagcgtctcatacatgcattggtatcaacaaaagcctggacaggcccccaagctctggatatacgatacgagcaagctggcttccggcgtacctagccgcttcagtggttccggctcaggcaacgatcacacccttacgatttccagtatggaacccgaagattttgcaacttattattgtttccaggggagcgtgtacccattcactttcgggcaggggacaaaagtgaccgtcctaggcggtggcggtagtgggggaggcggttctggcggcggagggtccggcggtggaggatcacaggtcacattgaaggaatctggccccggccttgttcagccaggacagacccttagcctcacctgtgccttcagtggtttttctcttagcactagcggtatgggggtcggctggattcggcagcctcccggcaaaggtcttgagtggttggctcacatttggtgggacgacgacaaacggtataatcctgccttgaaaagtcggctgaccattagtaaggatacctcaaaaaatcaagtgtacttgcaaatgaatagccttgacgccgaggatacggctgtatattattgcgcgcggatggaactctggtcttactactttgattattgggggcaggggactctcgtcacggtctcgagtggcggtggagggtccggcggtggtggatcagaaatcgtccttacacaatctcctagcacactgagtgtgagccccggcgaacgcgcgactttctcttgcagggcaagtcaatccatagggactaatatacattggtatcaacaaaagccaggtaaaccacccaggcttttgattaagtatgcaagtgagtctatttccggtatccctgaccgcttctctggatcaggcagtggcacagagttcacactcaccatatctagtgtgcaatcagaggacttcgccgtgtattactgccaacagaataataactggccgactaccttcggacccggtacaaagctgaccgttttacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggaggcgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccaagccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaacagattcacgcagaagagcctctccctgtctccgggtggcggtggagggtccggcggtggtggatccgacattcagatgacacagtcaccgtctacgctctcagcctcagtaggcgatagggtgactatcacctgccaagcctcccaatctataagttcacacctcaactggtatcaacagaaacccggtaaagcaccgaagctgctcatctacaaggcatcaactcttgcctcaggtgtgcctagtcgcttctccgggagcggctccggtactgagtttactttgacgatatcttccctccaacccgacgacttcgccacatactattgtcagcagggttatagttggggaaatgttgacaatgtgtttggagggggaacgaaagtgactgtgttgggagggggaggaagtggcggcggcggaagcggaggtggtggctccggcgggggtggaagcgaagtgcaattgctcgaaagtggtggcggacttgtgcaacctggtggaagcctctcactctcttgcgcggcgagtggttttagcttcagtagcggctacgatatgtgctgggtaagacaggctcctggtaaaggccttgagtggattgcgtgtatcgccgccgggtctgctggtataacatacgatgcaaactgggcgaaagggcgcttcacaatctccagggacaactcaaaaaatacattgtacctccagatgaatagcctgagagccgaagacaccgcagtctactattgcgcaaggtcagcatttagcttcgattatgcaatggatctttggggccaggggacgctcgtcaccgtctcatcttga

SEQ ID NO: 137 - protein, 448 aa, Artificial Sequence
  synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 138 - DNA, 1347 bp, Artificial Sequence
  synthesized
caagttcagctcaagcagtctggccctggcctggttcagccttctcagagcctgagcatcacctgtaccgtgtccggcttctccctgaccaattacggcgtgcactgggttcgacagagccctggcaaaggactggaatggctgggagtgatttggagcggcggcaacaccgactacaacacccctttcacctctcggctgtctatcaacaaggacaactccaagagccaggtgttcttcaagatgaactccctgcagtccaacgacaccgccatctactactgtgctcgggccctgacctactacgactacgagtttgcttactggggccagggcaccctggtcacagtttctgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgatgcatgaggctctgcacaaccactacacgcagaagagcctctccctgtctccgggttga

SEQ ID NO: 139 - protein, 214 aa, Artificial Sequence
  synthesized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - DNA, 645 bp, Artificial Sequence
  synthesized
gacatcctgctgacccagtctccagtgatcctgtccgtgtctcctggcgagagagtgtccttcagctgcagagcctctcagtccatcggcaccaacatccactggtatcagcagcggaccaacggctcccctcggctgctgattaagtacgcctccgagtctatcagcggcatcccctccagattctccggctctggctctggcaccgacttcaccctgtccatcaactccgtggaatccgaggatatcgccgactactactgccagcagaacaacaactggcccaccacctttggcgctggcaccaagctggaattgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 141 - protein, 448 aa, Artificial Sequence
  synthesized
QVQLQQSGPGLVKPSETLSITCTVSGFSLTNYGVHWIRQAPGKGLEWLGVIWSGGNTDYNTPFTSRFTITKDNSKNQVYFKLRSVRADDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 142 - DNA, 1347 bp, Artificial Sequence
  synthesized
caagttcagttgcagcagtctggccctggcctggtcaagccttctgagacactgtccatcacctgtaccgtgtccggcttctccctgaccaattacggcgtgcactggatcagacaggcccctggcaaaggactggaatggctgggagtgatttggagcggcggcaacaccgactacaacacccctttcaccagccggttcaccatcaccaaggacaactccaagaaccaggtgtacttcaagctgcggagcgtgcgggctgatgacaccgccatctactactgtgctcgggccctgacctactacgactacgagtttgcttactggggccagggcaccctggtcacagtttcttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgatgcatgaggctctgcacaaccactacacgcagaagagcctctccctgtctccgggttga

SEQ ID NO: 143 - protein, 214 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - DNA, 645 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctggaattgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 145 - protein, 449 aa, Artificial Sequence
  synthesized
EVQLVESGGGLVQPGGSLRLSCTASGFTISTNAMSWVRQAPGKGLEWVGVITGRDITYYASWAKGRFTISRDTSKNTVYLQMNSLRAEDTAVYYCARDGGSSAITSNNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 146 - DNA, 1350 bp, Artificial Sequence
  synthesized
gaagtccaattggtagaaagtggcggtggtctggtgcaacctggtggatctcttcgcctctcatgcacggctagtggctttactatttcaactaatgcgatgagctgggttcgccaggcccccggcaaaggacttgagtgggtcggcgtcatcaccggcagggacattacatactatgcgagttgggcaaagggcaggttcacgattagccgcgatacttcaaagaataccgtttaccttcaaatgaatagcttgcgcgcggaagacacagctgtgtattactgcgcgcgggatggaggtagttccgccataacttccaacaacatatggggacaaggcacgctggttactgtgtcgtcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctgctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 147 - protein, 219 aa, Artificial Sequence
  synthesized
EIVMTQSPSTLSASVGDRVIITCQASESISSWLAWYQQKPGKAPKLLIYEASKLASGVPSRFSGSGSGAEFTLTISSLQPDDFATYYCQGYFYFISRTYVNSFGQGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - DNA, 660 bp, Artificial Sequence
  synthesized
gaaatcgttatgacgcagagtccctccacgctctccgctagtgtcggggatcgcgtcattatcacatgccaggcctccgagtcaatcagcagctggcttgcatggtatcaacagaagccgggaaaagctcctaaattgctgatctatgaagcgtcaaaattggcgtctggtgtcccatctaggttctccggctctgggtctggtgcggaatttactttgacaatctccagtcttcaaccagacgatttcgctacctactactgccaagggtatttctattttataagccggacatatgtaaactccttcggccaaggaacaaagttgactgttcttcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 149 - protein, 214 aa, Artificial Sequence
  synthesized
EIVLTQSPSTLSVSPGERATFSCRASQSIGTNIHWYQQKPGKPPRLLIKYASESISGIPDRFSGSGSGTEFTLTISSVQSEDFAVYYCQQNNNWPTTFGPGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - DNA, 645 bp, Artificial Sequence
  synthesized
gagatcgtgctgacccagtctccttccacactgtctgtgtctcccggcgagagagccaccttcagctgtagagcctctcagtccatcggcaccaacatccactggtatcagcagaagcccggcaagcctcctcggctgctgattaagtacgcctccgagtccatcagcggcatccctgacagattctccggctctggctctggcaccgagtttaccctgaccatctcctccgtgcagtccgaggatttcgccgtgtactactgccagcagaacaacaactggcccaccacctttggacccggcaccaagctgaccgtgctgcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 151 - protein, 448 aa, Artificial Sequence
  synthesized
QVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 152 - DNA, 1347 bp, Artificial Sequence
  synthesized
caagttcagctcaagcagtctggccctggcctggttcagccttctcagagcctgagcatcacctgtaccgtgtccggcttctccctgaccaattacggcgtgcactgggttcgacagagccctggcaaaggactggaatggctgggagtgatttggagcggcggcaacaccgactacaacacccctttcacctctcggctgtctatcaacaaggacaactccaagagccaggtgttcttcaagatgaactccctgcagtccgaggacaccgccatctactactgtgctcgggccctgacctactacgactacgagtttgcttactggggccagggcaccctggtcacagtttctgctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgatgcatgaggctctgcacaaccactacacgcagaagagcctctccctgtctccgggttga

SEQ ID NO: 153 - protein, 119 aa, Artificial Sequence
  synthesized
  misc_feature (92)..(92): Xaa can be any naturally occurring amino acid
QVQLQQSGPGLVKPSESLSLTCTVSGFSLTNYGVHWVRQAPGKGLEWLGVIWSGGNTDYNTPFTSRFTISKDNSKNQVYFKMNSLRAEDTAXYYCARALTYYDYEFAYWGQGTLVTVSS

SEQ ID NO: 154 - protein, 107 aa, Artificial Sequence
  synthesized
EIVLTQSPSTLSVSPGERVTFSCRASQSIGTNIHWYQQKPGQPPRLLIKYASESISGIPSRFSGSGSGTEFTLTISSVQSEDFAVYYCQQNNNWPTTFGQGTKLELK

(19) United States
(12) Patent Application Publication
(10) Pub. No.: US 2025/0353917 A1
(43) Pub. Date: Nov. 20, 2025

(54) EGFR BINDING COMPLEX AND METHOD OF MAKING AND USING THEREOF

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Dennis R. GOULET, Redmond, WA (US); Nga Sze Amanda MAK, Mukilteo, WA (US); Hai ZHU, Bothell, WA (US); Yi ZHU, Redmond, WA (US)

(21) Appl. No.: 18/027,583

(22) PCT Filed: Sep. 21, 2021
(86) PCT No.: PCT/US2021/051165

§ 371 Date: Mar. 21, 2023

Related U.S. Application Data
(60) Provisional application No. 63/081,315, filed on Sep. 21, 2020.
(60) Provisional application No. 63/109,877, filed on Nov. 5, 2020.

(51) Int. Cl. C07K 16/28; C07K 16/46

(52) U.S. Cl. CPC C07K 16/2863; C07K 16/2809; C07K 16/468; C07K 2317/24; C07K 2317/31; C07K 2317/55; C07K 2317/569; C07K 2317/92

(57) ABSTRACT

A binding domain having a binding specificty to human EGFR (epithelium growth factor receptor) comprises a VH domain and a VL domain, wherein the VH and VL domain each independently comprises a sequence having at least 90% sequence identify to an amino acid sequence as disclosed thereof. The application further provides antibodies comprising the binding domain.

### CROSS REFERENCE TO RELATED APPLICATIONS

This application claims the benefit of the filing date of U.S. Provisional Application Ser. No. 63/081,315 filed Sep. 21, 2020, and U.S. Provisional Application Ser. No. 63/109,877 filed Nov. 5, 2020 under 35 U.S.C. 119(e), the entire disclosures of which are incorporated by reference herein.

### TECHNICAL FIELD

The present disclosure generally relates to the technical field of cancer therapy using antibodies, and more particularly relates to making and using multi-specific antibodies.

### BACKGROUND

Cetuximab is a chimeric (mouse/human) monoclonal antibody targeting the human epidermal growth factor receptor (EGFR). It was approved in the US and EU in 2004 for treatment of colorectal cancer and is also used to treat head and neck cancer.¹⁻³ In addition to the mAb, cetuximab has also been used in T-cell redirecting bispecific antibodies,⁴ antibody-peptide fusions, and antibody-drug conjugates.⁵

Cetuximab can bind to domain III of the extracellular domain of EGFR that is often overly expressed on tumor cells. The binding of cetuximab on tumor cells competitively inhibits binding of EGF and other ligands, prevents EGFR from dimerization, and prohibits receptor tyrosine autophosphorylation. As a result of inhibition and reduced EGFR-mediated signaling, the binding of cetuximab effectively downregulates tumor cell proliferation, angiogenesis, and metastasis while inducing apoptosis. In addition to targeting EGFR, the Fc domain of cetuximab can bind to CD16a and other Fc receptors and recruit and activate immune mechanisms, such as antibody-dependent cellular cytotoxicity.⁴ These anti-tumor properties of cetuximab are highly desirable for developing combinational therapies, either as a single agent or a component of regimen. However, there is a point of concern that cetuximab's variable regions (VH/Vk) remain on a mouse framework since its isolation from mouse hybridoma. It has been shown that the use of mouse sequences, such as mouse VH/Vk, can increase incidence of immunogenicity when proteins are administered to human patients. Thus, a protein therapeutics with humanized VH/Vk regions may decrease the risk of cetuximab-derived immunogenicity in humans.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The application provides, among others, binding domains and peptides having binding specificity to human epithelium growth factor receptor (EGFR), antibody-like proteins incorporating the anti-EGFR binding domains and peptides as disclosed herein, immunoconjugates and pharmaceutical compositions incorporating the anti-EGFR binding domains and peptides as disclosed herein, methods of making and using such anti-EGFR binding domains, peptides and antibody-like proteins. In one embodiment, the anti-EGFR antibody-like proteins including antibodies, monoclonal antibodies, humanized antibodies, or chimeric antibodies. In one embodiment, the anti-EGFR antibody may be monospecific or multi-specific. In one embodiment, the multi-specific anti-EGFR antibody may be bispecific, tri-specific, tetra-specific, penta-specific, or hexa-specific. In one embodiment, the anti-EGFR antibody may be symmetric or asymmetric.

In one aspect, the application provides human EGFR binding peptide having a binding specificity to human EGFR. The peptide may include an amino acid sequence having at least 70%, 80%, 85%, 90%, 92%, 95%, 98%, 99%, or 100% sequence identity to SEQ ID NO. 1, 3, 5, 7, 9, 11, 13, 15, 17, 19, 21, 23, 25, 27, 29, 31, 33, 35, 37, 39, 41, 43, 57, 61, 63, 65, 67, 69, 71, 73, 75, 77, 79, 81, 83, a combination thereof.

In one embodiment, the EGFR binding peptide includes a variable heavy (VH) chain and a variable light (VL) chain. In one embodiment, the VH chains comprises an amino acid sequence having at least 70%, 80%, 85%, 90%, 92%, 95%, 98%, 99%, or 100% sequence identify to SEQ ID NO. 1, 5, 9, 13, 17, 21, 25, 29, 33, 37, 41. In one embodiment, the VL chain comprises an amino acid sequence having at least 70%, 80%, 85%, 90%, 92%, 95%, 98%, 99%, or 100% sequence identity to SEQ ID NO. 3, 7, 11, 15, 19, 23, 27, 31, 35, 39, 43.

In one embodiment, the EGFR binding peptide includes a scFv domain and the scFv domain comprises the VH chain and VL chain as disclosed herein.

In one embodiment, the application provides an anti-EGFR scFv domain or peptides forming such scFv domain. In one embodiment, the scFv domain comprises an amino acid sequence having at least 70%, 80%, 85%, 90%, 92%, 95%, 98%, 99%, or 100% sequence identify to SEQ ID NO. 57, 61, 63, 65, 67, 69, 71, 73, 75, 77, 79, 81, 83. In one embodiment, the scFv domain comprises the VH chain and the VL chain as disclosed herein.

In one embodiment, the EGFR binding peptide may include a histidine residue linked to at least one end of the scFv domain (for example, ScFV-HIS). In one embodiment, the EGFR binding peptide may include an amino acid sequence having at least 70%, 80%, 85%, 90%, 92%, 95%, 98%, 99%, or 100% sequence identity to 57.

In one embodiment, the EGFR binding peptide may include a Fab domain, and the Fab domain comprises the VH chain and the VL chain as disclosed herein. In one embodiment, the EGFR binding peptide may further include a Fc domain linked to the Fab domain to provide a Fab-monoFc fusion protein. In one embodiment, the Fc domain comprises a sequence having at least 70%, 80%, 85%, 90%, 92%, 95%, 98%, 99%, or 100% sequence identity to an amino acid sequence selected from SEQ ID NO. 45 and 47.

In another aspect, the application provides an antibody-like protein having a binding specificity to human EGFR. The antibody-like protein may include an EGFR binding domain having a variable heavy (VH) chain and a variable light (VL) chain. In one embodiment, the VH chains comprises an amino acid sequence having at least 70%, 80%, 85%, 90%, 92%, 95%, 98%, 99%, or 100% sequence identify to SEQ ID NO. 1, 5, 9, 13, 17, 21, 25, 29, 33, 37, or 41. In one embodiment, the VL chain may include an amino acid sequence having at least 70%, 80%, 85%, 90%, 92%, 95%, 98%, 99%, or 100% sequence identity to SEQ ID NO. 3, 7, 11, 15, 19, 23, 27, 31, 35, 39, or 43.

In one embodiment, the antibody-like protein may include a scFv domain having an amino acid sequence having at least 70%, 80%, 85%, 90%, 92%, 95%, 98%, 99%, or 100% sequence identity to SEQ ID NO. 57, 59, 61, 63, 65, 67, 69, 71, 73, 75, 77, 79, 81, or 83.

In one embodiment, the antibody-like protein may a monospecific antibody. In one embodiment, the antibody may include an amino acid sequence having at least 70%, 80%, 85%, 90%, 92%, 95%, 98%, 99%, or 100% sequence identity to SEQ ID NO. 137, 139, 141, 143; 141, 149, 151, 139, 145, 147 or a combination thereof. In one embodiment, the monospecific antibody may include pairs of light chains and heavy chains, or fragments thereof selected from the sequence combinations of SEQ ID NO. 137 and 139; 141 and 143; 141 and 149; 151 and 139; 145 and 147.

In one embodiment, the antibody-like protein may have a binding specificity to at least 2 different antigens selected from a tumor antigen, an immune signaling antigen, or a combination thereof.

In one embodiment, the antibody-like protein may be a bispecific antibody. In one embodiment, the bispecific antibody may include an amino acid sequence having at least 70%, 80%, 85%, 90%, 92%, 95%, 98%, 99%, or 100% sequence identity to SEQ ID NO. 137, 145, 139, 147, 141, 145, 143, 147, 141, 145, 149, 147, 151, 145, 139, 147 or a combination thereof. In one embodiment, the bispecific antibody may include the combinations of light chains and heavy chains (or fragments thereof) selected from the sequence combinations of SEQ ID NO. 137 and 145 and 139 and 147; 141 and 145 and 143 and 147; 141 and 145 and 149 and 147; 151 and 145 and 139 and 147.

In one embodiment, the antibody-like protein may have a binding specificity to at least 3 different antigens selected from a tumor antigen, an immune signaling antigen, or a combination thereof.

In one embodiment, the antibody-like protein may have a binding specificity to at least 4 different antigens selected from a tumor antigen, an immune signaling antigen, or a combination thereof.

In one embodiment, the antibody-like protein may have a binding specificity to at least 5 different antigens selected from a tumor antigen, an immune signaling antigen, or a combination thereof. In one embodiment, the antibody-like protein may be a penta-specific antibody.

In one embodiment, the penta-specific antibody may include an amino acid sequence having at least 70%, 80%, 85%, 90%, 92%, 95%, 98%, 99%, or 100% sequence identity to SEQ ID NO. 85, 87, 89, 91, 93, 95, 97, 99, 101, 103, 105, 107 or a combination thereof. In one embodiment, the penta-specific antibody may include pairs of light chains and heavy chains or fragments thereof selected from the sequence combinations of SEQ ID NO. 85 and 87; 89 and 91; 93 and 95; 97 and 99; 101 and 103; 105 and 107.

In on embodiment, the antibody-like protein may have a binding specificity to at least 6 different antigens selected from a tumor antigen, an immune signaling antigen, or a combination thereof. In one embodiment, the antibody-like protein may be a hexa-specific antibody.

In one embodiment, the hexa-specific antibody may include an amino acid sequence having at least 70%, 80%, 85%, 90%, 92%, 95%, 98%, 99%, or 100% sequence identity to SEQ ID NO. 109, 111, 113, 115, 117, 119 or a combination thereof. In one embodiment, the hexa-specific antibody may include a combination of light chains and heavy chain or fragments thereof selected from the combination of SEQ ID NO. 109 and 111; 113 and 115; 117 and 119.

In one embodiment, the antibody-like protein may include a heavy chain (HC) and a light chain (LC). In one embodiment, the HC comprises an amino acid sequence having at least 98%, 95%, or 92% of sequence identity to SEQ ID NO. 85, 89, 93, 97, 101, 105, 109, 113, 117, 137, 141, 145, 151; and the LC comprises an amino acid sequence having at least 70%, 80%, 85%, 90%, 92%, 95%, 98%, 99%, or 100% of sequence identity to SEQ ID NO. 87, 91, 95, 99, 103, 107, 111, 115, 119, 139, 143, 147, 149.

In one embodiment, the antibody-like protein may include a heavy chain monomer and a light chain monomer, wherein the heavy chain monomer having a N-terminus and a C-terminus, comprising in tandem from the N-terminal to the C-terminal, an optional first binding domain (D1) at the N-terminal, a Fab region as a second binding domain (D2) comprising a light chain, a Fc domain, an optional third binding domain (D3), and an optional fourth binding domain (D4) at the C-terminal. The light chain may comprise an optional fifth binding domain (D5) covalently attached to the C-terminus, an optional sixth binding domain (D6) covalently attached to the N-terminus, or a combination thereof. At least one of D1, D2, D3, D4, D5 and D6 comprises the EGFR binding domain as disclosed herein.

In one embodiment, the D1 comprises the EGFR binding domain. In one embodiment, the D2 comprises the EGFR binding domain. In one embodiment, each of the D3, D4, D5 and D6 comprises the EGFR binding domain. In one embodiment, the D1, D2, D3, D4, D5 and D6 each has a binding specificity to a different antigen, wherein the antigen is a tumor antigen, an immune signaling antigen, or a combination thereof.

In one embodiment, the antibody-like protein may be a bispecific antibody. In one embodiment, the bispecific antibody is asymmetric with the D2 comprising the EGFR binding domain and the D3 has a binding specificity to CD3.

In one embodiment, bispecific antibody may include an amino acid sequence having at least 70%, 80%, 85%, 90%, 92%, 95%, 98%, 99%, or 100% sequence identity to SEQ ID NO. 137, 145, 139, 147, 141, 145, 143, 147, 141, 145, 149, 147, 151, 145, 139, 147 or a combination thereof. In one embodiment, the bispecific antibody may include the combinations of light chains and heavy chains, or fragments thereof selected from the sequence combinations of 147; 141 and 145 and 149 and 147; 151 and 145 and 139 and 147.

In one embodiment, the antibody-like protein may include an amino acid sequence having at least 70%, 80%, 85%, 90%, 92%, 95%, 98%, 99%, or 100% sequence identity to SEQ ID NO. 137, 139, 141, 143, 141, 149, 151, 139, 145, 147 or a combination thereof. In one embodiment, the bispecific antibody may include the combinations of light chains and heavy chains, or fragments thereof selected from the sequence combinations of SEQ ID NO. 137 and 139, 141 and 143, 141 and 149, 151 and 139, 145 and 147.

In one aspect, the application provides the antibody-like protein having a Fab-Fc structure with one or more binding domains attached to the Fab-Fc structure. In one embodiment, the antibody-like protein has a N-terminus and a C-terminus and include a first monomer and a second monomer. The first monomer includes, from the N-terminus to the C-terminus, a first binding domain (mD1), a variable heavy (VH) chain, a CH1 domain, a first hinge, a first CH2 domain, a first CH3 domain, and a fourth binding domain (mD4). The second monomer includes from the N-terminus to the C-terminus, a second binding domain (mD2), a variable light (VL) chain, a CL domain, a second hinge, a second CH2 domain, and a second CH3 domain, and a fifth binding domain (mD5). The CH chain and CL chain forms a third binding domain (mD3). The first monomer and the second monomer may be covalently paired through at least one disulfide bond between the CH1 domain and the CL domain and at least one disulfide bond between the first hinge and the second hinge, and the antibody-like protein is at least bi-specific.

In one embodiment, at least one of the mD1, mD2, mD3, mD5, and mD5 in the antibody-like protein may include the EGFR binding domain as disclosed herein. In one embodiment, the mD3 domain comprises the EGFR binding domain. In one embodiment, the mD2 domain comprises the EGFR binding domain. In one embodiment, the mD2, mD4, mD5 each comprises the EGFR binding domain.

In one embodiment, the antibody-like protein may include an amino acid sequence having at least 70%, 80%, 85%, 90%, 92%, 95%, 98%, 99%, or 100% sequence identity to SEQ ID NO 121, 123, 125, 127, 129, 131, 133, 135 or a combination thereof. In one embodiment, the antibody-like protein may include the combinations of peptides or fragments thereof selected from the sequence combinations of 121 and 123; 125 and 127; 129 and 131; 133 and 135.

In one aspect, the application provides heavy chains. In one embodiment, the heavy chain may include an amino acid sequence having at least 70%, 80%, 85%, 90%, 92%, 95%, 98%, 99%, or 100% sequence identity to SEQ ID NO. 121, 125, 129, 133.

In one aspect, the application provides light chain. In one embodiment, the light chain may include an amino acid sequence having at least 70%, 80%, 85%, 90%, 92%, 95%, 98%, 99%, or 100% sequence identity to SEQ ID NO. 123, 127, 131, 135.

In one aspect, the application provides isolated nucleic acid sequence encoding the antibody-like protein, light chains, heavy chains, and peptide sequences as disclosed herein.

In one aspect, the application provides expression vector comprising the isolated nucleic acid sequences as disclosed herein.

In one aspect, the application provides host cell for producing the antibody-like protein, light chains, heavy chains, or combinations thereof. In one embodiment, the host cell includes the isolated nucleic acid sequence as disclosed herein. In one embodiment, the host cells may be prokaryotic or eukaryotic.

In one aspect, the application may include an immunoconjugate. In one embodiment, the immunoconjugate may include the antibody-like protein, the antibodies, anti-EGFR binding domains or peptides as disclosed herein, and a cytotoxic agent. In one embodiment, the cytotoxic agent may include a chemotherapeutic agent, a growth inhibitory agent, a toxin, or a radioactive isotope.

In one aspect, the application provides pharmaceutical composition for treating diseases or health conditions. In one embodiment, the pharmaceutical composition may include the antibody-like protein, antibodies, immunoconjugates, anti-EGFR binding domains or peptides as disclosed herein, and a pharmaceutically acceptable carrier.

In one embodiment, the pharmaceutical composition may further comprise a therapeutic agent. In one embodiment, the therapeutic agent may be a chemotherapeutic agent, a growth inhibitory agent, a toxin, a radioactive isotope, or a combination thereof. In one embodiment, the therapeutic agent may be, for example, capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof.

In one aspect, the application provides methods for treating or preventing a cancer, an autoimmune disease, or an infectious disease in a subject. In one embodiment, the method may include the step of administering to the subject a pharmaceutical composition comprising a purified antibody-like protein, the antibody, the immunoconjugates, the anti-EGFR domains, or peptides as disclosed herein. In one embodiment, the subject is a mammal. In one embodiment, the subject is a human.

In one embodiment, the method may further include the step of co-administering an effective amount of a therapeutic agent. In one embodiment, the therapeutic agent may be an antibody, a chemotherapy agent, an enzyme, or a combination thereof.

In one embodiment, the cancer may include cells expressing HER3 or EGFR. In one embodiment, the cancer may be, for example, breast cancer, colorectal cancer, pancreatic cancer, head and neck cancer, melanoma, ovarian cancer, prostate cancer, non-small lung cell cancer, small cell lung cancer, glioma, esophageal cancer, nasopharyngeal cancer, kidney cancer, gastric cancer, liver cancer, bladder cancer, cervical cancer, brain cancer, lymphoma, leukaemia, myeloma

In one aspect, the application provides method for producing the antibody-like protein, antibody, the anti-EGFR domains, or peptides as disclosed herein. In one embodiment, the method may include the steps of culturing a host cell such that the DNA sequence encoding the antibody-like protein, the anti-EGFR domains or peptides as disclosed herein, is expressed, and purifying said multi-specific antibody-like protein, the anti-EGFR domains or peptides as disclosed herein.

In one aspect, the application provides a solution comprising an effective concentration of the antibody-like protein, the antibody, the immunoconjugates, the anti-EGFR domains or peptides as disclosed herein. In one embodiment, the solution is blood plasma in a subject. In one embodiment, the subject is a human.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 depicts the alignment of cetuximab-derived VH (A) and VL (B) sequences in Kabat numbering, where N85E is aglycosylated cetuximab and H1 through H11 are humanized variants, the consensus sequences of VH and VL are generated by Geneious software (geneious.com), in which the consensus sequence is displayed above the alignment or assembly, and shows which residues are conserved (are always the same), and which residues are variable. A consensus is constructed from the most frequent residues at each site (alignment column), so that the total fraction of rows represented by the selected residues in that column reaches at least a specified threshold;

FIG. 2 displays T20 humanness scores for cetuximab-derived VH (A) and Vk (B) domains, and (C) the predicted number of MHCII-binding peptides in cetuximab-derived variable regions, based on the MixMHC2pred algorithm;

FIG. 3 shows the SEC profile of His-tagged anti-EGFR scFv domains indicating humanizing SI-79R1 (cetuximab) to SI-79R2 (H1) resulted in lower aggregation;

FIG. 4 shows the Octet binding analysis of His-tagged anti-EGFR scFv domains, demonstrating that humanized cetuximab, H1, has similar binding to human EGFR as mouse scFv;

FIG. 5 shows the result of thermal stability analysis of His-tagged anti-EGFR scFv domains, indicating that humanized cetuximab, SI-79R2, is significantly more stable than SI-79R1 as measured by DLS (unfolding at higher temperature);

FIG. 6 shows the results of chemical denaturation stability analysis of His-tagged anti-EGFR scFv domains, indicating that humanized Cetuximab, SI-79R2, is significantly more stable than SI-79R1 as measured by guanidine and urea denaturation (higher concentration of guanidine/urea required to unfold);

FIG. 7 shows analytical size-exclusion chromatograms for scFv-monoFc (A) and mAb proteins (B) immediately after first step protein A purification, and non-reducing SDS-PAGE of purified scFv-monoFc proteins (C), where data are representative of two independent expressions and purifications, N85E is aglycosylated cetuximab, and H1 through H11 are humanized variants;

FIG. 8 shows the binding kinetics of cetuximab-derived scFv-monoFc proteins (A) and mAbs (B) as determined by biolayer interferometry using anti-human Fc (AHC) sensors and soluble recombinant extracellular domain of human EGFR, where data are representative of two independent experiments, K_{D} values are shown in Tables 6-8, N85E is aglycosylated cetuximab, and H1 through H11 are humanized variants;

FIG. 9 shows thermal stability of cetuximab-derived scFv-monoFc proteins (A) and mAbs (B) as determined by dynamic light scattering, where data are representative of two independent experiments, unfolding temperatures (the point at which the radius surpassed 10 nm) are shown in Tables 7-8, N85E is aglycosylated cetuximab, and H1 through H11 are humanized variants;

FIG. 10 shows cation exchange chromatography of αCD3×αEGFR bispecific antibodies and their parental αCD3 and αEGFR antibodies showing their characteristic retention times;

FIG. 11 shows T cell-dependent cellular cytotoxicity (TDCC) of αCD3×αEGFR bispecific antibodies, using luciferized EGFR-bearing B×PC-3 cell line as target cells incubated with activated T cells and the αEGFR arm, including wild-type cetuximab, aglycosylated cetuximab (N85E), and humanized version H7, and luminescence signals as the endpoint of viable BxPC-3 cells after 72 hours (EC50 values are shown in Table 8);

FIG. 12 demonstrates GNC antibodies in a schematic diagram showing the configuration: 1) the variable regions of Fab in black (D2), both the constant regions of Fab and the Fc region in white; 2) additional scFv antigen binding domains in shaded boxes (each replaceable by a receptor-ligand binding); 3) a heavy chain monomer linking D1 to its N-terminus and/or D3 and/or D4 tandemly to its C-terminus through D4; and 4) a light chain monomer linking D5 and/or D6 to its N- and C-terminus, resulting in hexa-GNC and penta-GNC antibodies, respectively;

FIG. 13 shows the analytical SEC profile of anti-huEGFR penta-GNC antibodies, comprising either a humanizing anti-EGFR scFv (SI-55P3, H1 scFv; SI-55P4, H1 scFv; SI-79P2, H4 scFv; SI-79P3, H4 scFv; and SI-55P9, H7 scFv) or a Fab region (SI-77P1, H7 Fab), have low aggregation after protein A purification.

FIG. 14 shows the Octet binding analysis of anti-huEGFR penta-GNC antibodies, indicating that the penta-GNC antibodies having either a humanizing anti-EGFR scFv (SI-55P3, H1 scFv; SI-79P2, H4 scFv; and SI-79P3, H4 scFv) or a Fab region (SI-77P1, H7 Fab) retain tight binding;

FIG. 15 demonstrates that the penta-GNC antibodies having humanized anti-EGFR scFv (SI-55P9, H7 scFv) or Fab (SI-77P1, H7 Fab) elicit potent TDCC to EGFR-expressing tumor cells;

FIG. 16 shows analytical SEC profiles of anti-huEGFR hexa-GNC antibodies, indicating that the hexa-GNC antibodies having either a humanized anti-EGFR scFv (SI-55H11, H7 scFv) or a Fab region (SI-77H4, H7 Fab) have lower aggregation than the hexa-GNC having anti-EGFR domain derived from Cetuximab;

FIG. 17 shows the Octet binding analysis of anti-huEGFR hexa-GNC antibodies indicates that the hexa-GNC antibodies having either a humanizing anti-EGFR scFv (SI-55H11, H7 scFv) or a Fab region (SI-77H4, H7 Fab) retain binding to EGFR comparable to the Cetuximab-derived hexa-GNC antibody, SI-77H4;

FIG. 18 shows that the hexa-GNC antibodies having humanized anti-EGFR scFv (SI-55H11, H7 scFv) elicit potent TDCC to EGFR-expressing tumor cells;

FIG. 19 depicts (A) a schematic diagram of asymmetric bispecific antibodies, of which the αEGFR Fab is derived from one of three cetuximab Fabs (wild-type with or without N85E, or with humanized VH/VL; the second Fab is &CD3 Fab; and the CH3 domain contains the K409R mutation; and (B) miniGNC antibody-like proteins in a schematic diagram showing the heterodimeric configuration: 1) the variable regions of a single Fab in black (mD3), both the constant regions of Fab and the Fc region in white; 2) additional scFv antigen binding domains in shaded boxes (each replaceable by a receptor-ligand binding); 3) Chain A monomer linking mD1 to its N-terminus and mD4 to its C-terminus; and 4) Chain B monomer linking mD2 to its N-terminus and mD5 to its C-terminus;

FIG. 20 shows the analytical SEC profiles of anti-huEGFR penta-miniGNC antibodies, indicating that the penta-miniGNC antibodies having either a humanizing anti-EGFR scFv (SI-68P7, H1 scFv; SI-79P1, H4 scFv; and SI-68P13, H7 scFv) or a Fab domain (SI-68P17, H7 Fab) have low aggregation;

FIG. 21 shows the Octet binding analysis of anti-huEGFR penta-miniGNC antibodies, indicating that the penta-miniGNC antibodies having either a humanized anti-EGFR scFv (SI-709P1, H4 scFv; SI-68P13, H7 scFv) or a Fab region (SI-68P17, H7 Fab) retain binding to EGFR; and

FIG. 22 shows that the penta-miniGNC antibodies having a humanized anti-EGFR scFv (SI-68P13, H7 scFv) or a Fab region (SI-68P17, H7 Fab) elicit potent TDCC to EGFR-expressing tumor cells.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

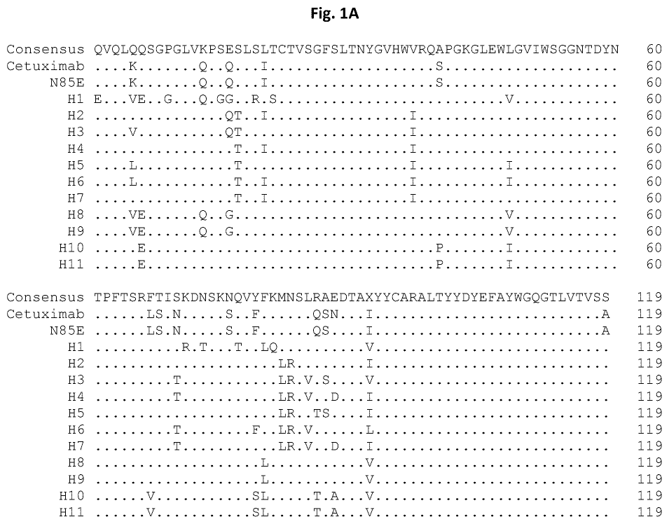

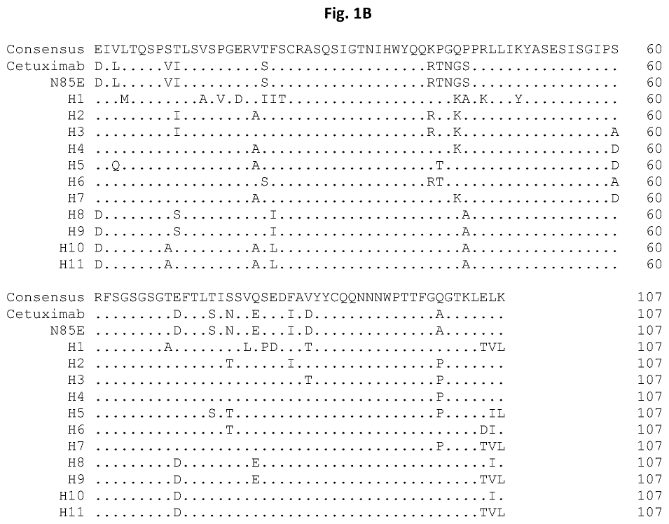

The present disclosure provides, among others, isolated antibodies, methods of making such antibodies, monoclonal and/or recombinant monospecific antibodies, multi-specific antibodies, antibody-drug conjugates and/or immuno-conjugates composed from such antibodies or antigen binding fragments, pharmaceutical compositions containing the antibodies, monoclonal and/or recombinant monospecific antibodies, multi-specific antibodies, antibody-drug conjugates and/or immuno-conjugates, the methods for making the antibodies and compositions, and the methods for treating cancer using the antibodies and compositions disclosed herein. Specifically, the present disclosure provides isolated monoclonal antibodies (mAb) or antigen-binding fragments thereof having a binding specificity to human EGFR (Table 1, FIG. 1), wherein the isolated mAb or antigen-binding fragments comprise an amino acid sequence having an identity with a sequence selected from SEQ ID NO. 1 and 3; 5 and 7; 9 and 11; 13 and 15; 17 and 19; 21 and 23; 25 and 27; 29 and 31; 33 and 35; 37 and 39; 41 and 43; 55; 57; 59; 61; 63; 65; 67; 69; 71; 73; 75; 77; 79; 81; 83; 85 and 87; 89 and 91; 93 and 95; 97 and 99; 101 and 103; 105 and 107; 109 and 111; 113 and 115; 117 and 119; 121 and 123; 125 and 127; 129 and 131; 133 and 135; 137 and 139; 141 and 143; 141 and 149; 151 and 139; 145 and 147; 137, 145, 139 and 147; 141, 145, 143 and 147; 141, 145, 149 and 147; 151, 145, 139 and 147.

The terms “a”, “an” and “the” as used herein are defined to mean “one or more” and include the plural unless the context is inappropriate.

The terms “polypeptide”, “peptide”, and “protein”, as used herein, are interchangeable and are defined to mean a biomolecule composed of amino acids linked by a peptide bond.

The term “antigen” refers to an entity or fragment thereof which can induce an immune response in an organism, particularly an animal, more particularly a mammal including a human. The term includes immunogens and regions thereof responsible for antigenicity or antigenic determinants.

The terms “antigen- or epitope-binding portion or fragment”, “variable region”, “variable region sequence”, or “binding domain” refer to fragments of an antibody that are capable of binding to an antigen (such as EGFR in this application). The antigen-binding fragment (Fab) is a region (Fab region) on an antibody that binds to antigens. These fragments may be capable of the antigen-binding function and additional functions of the intact antibody. Examples of binding fragments include, but are not limited to, a single-chain Fv fragment (scFv) consisting of the variable light chain (VL) and variable heavy chain (VH) domains of a single arm of an antibody connected in a single polypeptide chain by a synthetic linker, or a Fab fragment which is a monovalent fragment consisting of the VL, constant light (CL), VH and constant heavy 1 (CH1) domains.

Antibody fragments can be even smaller sub-fragments and can consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments can be screened for utility using the same techniques employed with intact antibodies. The “antigen- or epitope-binding portion or fragment”, “variable region”, “variable region sequence”, or “binding domain” may be derived from an antibody of the present disclosure by a number of art-known techniques. For example, purified monoclonal antibodies can be cleaved with an enzyme, such as pepsin, and subjected to HPLC gel filtration. Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

The term “antibody” is used in the broadest sense and specifically covers single monoclonal antibodies and/or recombinant antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, multi-specific or multi-effective, human and humanized antibodies, as well as active fragments thereof. Examples of active fragments of molecules that bind to known antigens include Fab, F(ab′)₂, scFv and Fv fragments, including the products of a Fab immunoglobulin expression library and epitope-binding fragments of any of the antibodies and fragments mentioned above.

The term “Fv” refers to the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

In some embodiments, antibody may include immunoglobulin molecules and immunologically active portions of immunoglobulin molecules, i.e. molecules that contain a binding site and that immunospecifically bind an antigen. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The light chains of antibodies (immunoglobulins) from any vertebrate species can be assigned to one of two clearly distinct types, called kappa and lambda, based on the amino acid sequences of their constant domains. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567). “Recombinant” means the antibodies are generated using recombinant nucleic acid techniques in exogeneous host cells.

Monoclonal antibodies can be produced using various methods, including without limitation, mouse hybridoma, phage display, recombinant DNA, molecular cloning of antibodies directly from primary B cells, and antibody discovery methods (see Siegel. Transfus. Clin. Biol. 2002; Tiller. New Biotechnol. 2011; Seeber et al. PLOS One. 2014). Monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

The term “multi-specific” antibody as used herein denotes an antibody that has at least two binding sites each having a binding affinity to an epitope of an antigen. The term “bi-specific, tri-specific, tetra-specific, penta-specific, or hexa-specific” antibody as used herein denotes an antibody that has two, three, four, five, or six antigen-binding sites. For example, the antibodies disclosed herein with five binding sites are penta-specific, with six binding sites are hexa-specific.

The term “guidance and navigation control (GNC)” protein refers to a multi-specific protein capable of binding to at least one effector cell (such as immune cell) antigen and at least one target cell (such as tumor cell, immune cell, or microbial cell) antigen (WO2019191120A1, incorporated herein by reference in its entirety). The GNC protein may adopt an antibody-core structure including a Fab region and Fc region with various binding domains attached to the antibody-core, in which case the GNC protein is also termed GNC antibody. The GNC protein may adopt an antibody-like structure, in which case the Fv fragment may be replaced with a non-antibody based binding domain, such as NKG2D, 4-1BBL (a 4-1BB receptor ligand), 4-1BBL trimer for 4-1BB, or a receptor.

The term “GNC antibody” refers to a GNC protein had an antibody structure that is capable of binding to at least one effector cell (such as an immune cell) and at least one target cell (such as a tumor cell, an immune cell, or a microbial cell) simultaneously. The term “biGNC, triGNC, tetraGNC, pentaGNC, or hexaGNC” antibody as used herein denotes a GNC antibody that has two, three, four, five, or six antigen-binding sites, of which at least one antigen-binding site has the binding affinity to an immune cell and at least one antigen-binding site has the binding affinity to a tumor cell. In one embodiment, the GNC antibodies disclosed herein have four to six binding sites (or binding domain) and are tetraGNC, pentaGNC, and hexaGNC antibodies, respectively. In some embodiments, the GNC antibodies include antibody binding domains (such as Fab and scFv) without the requirement for additional protein engineering in the Fc region. In one embodiment, the GNC antibodies additionally have the advantage of retaining bivalency for each targeted antigen. Further in one embodiment, the GNC antibodies have the advantage of avidity effects that result in higher affinity for antigens and slower dissociation rates. This bivalency for each antigen is in contrast to many multi-specific platforms that are monovalent for each targeted antigen, and thus often lose the beneficial avidity effects that make antibody binding so strong.

The term “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “isolated” or “purified” refers to a biological molecule free from at least some of the components with which it naturally occurs. Either “Isolated” or “purified,” when used to describe the various polypeptides disclosed herein, means a polypeptide that has been identified and separated and/or recovered from a cell or cell culture from which it was expressed. Ordinarily, a purified polypeptide will be prepared by at least one purification step. An “isolated” or a “purified” antibody refers to an antibody which is substantially free of other antibodies having different antigenic a binding specificity.

The term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated. While the immunogenic response generally includes both cellular (T cell) and humoral (antibody) arms of the immune response, antibodies directed against therapeutic proteins (anti-drug antibodies, ADA) may consist of IgM, IgG, IgE, and/or lgA isotypes.

The terms “specific binding”, “specifically binds to”, or “is specific for a particular antigen or an epitope” means that the binding is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “affinity” refers to a measure of the attraction between two polypeptides, such as antibody/antigen, receptor/ligand, etc. The intrinsic attraction between two polypeptides can be expressed as the binding affinity equilibrium dissociation constant (KD) of a particular interaction. A KD binding affinity constant can be measured, e.g., by Bio-Layer Interferometry, where KD is the ratio of kdis (the dissociation rate constant) to kon (the association rate constant), as KD=kdis/kon.

Specific binding for a particular antigen or an epitope can be exhibited, for example, by an antibody having a KD for an antigen or epitope of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater, where KD refers to the equilibrium dissociation constant of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

A potential shortcoming of cetuximab is that its variable regions were discovered in mice, and these regions retain non-human sequences. It has been demonstrated that chimeric antibodies may have increased capacity for immunogenicity when compared to humanized or human antibodies.⁶ On the other hand, humanization can increase the stability of antibodies by making the framework regions more compatible.⁷ Another concern is the occupied glycan site at VH N85 (Kabat), where Fab glycosylation could affect the biological properties of the antibody, as well as introduce glycan heterogeneity that must be well-controlled during manufacturing.^{8,9} While immunogenicity of cetuximab appears low based on low incidence of anti-cetuximab IgG response (5%), hypersensitivity is a common occurrence due largely to pre-existing IgE antibodies against the galactose-α-1,3-galactose oligosaccharide that modifies the VH when expressed in SP2/0 cells.¹⁰⁻¹²

To overcome these liabilities, cetuximab was humanized with the goals of removing post-translational modification sites, stabilizing the antibody, and reducing the potential for immunogenicity while retaining high affinity for EGFR. Strategies for humanization included a straight CDR graft onto a stable human framework, sequence-guided grafting onto the most similar germline or consensus framework, and a structure-guided approach based on predicted stability effects of humanizing mutations. The result is a panel of humanized cetuximab sequences with superior biophysical properties, where the structural modeling approach was the most successful in generating stable binders with no loss in EGFR affinity.

Humanization of antibodies discovered in non-human species is a common practice not only to decrease the immunogenicity, but also to increase stability and remove sequence liabilities. In this study, cetuximab scFv was humanized using three distinct strategies including unbiased CDR grafting, sequence-guided humanization, and model-guided humanization (Table 2). Although each approach was successful in generating EGFR binders with increased humanness, there was a clear trend in stability and affinity retention across the humanization strategies. Whereas the simple CDR graft resulted in destabilization and the largest (4-fold) loss in antigen affinity, the sequence homology approach resulted in stabilization and a more modest (2-fold) decrease in affinity, and the model-guided approach was the most successful with significant stabilization and no change in antigen affinity.

When expressed as a mAb, the humanized version H7 had increased titer and thermal stability relative to cetuximab, unchanged binding kinetics, and similar TDCC potency when transformed to bispecific αEGFR×αCD3 format (Table 8).

In addition to superior biophysical properties, the humanized versions removed sequence liabilities associated with the mouse variable regions of cetuximab. The humanness of VH and VL were significantly increased for all humanizations, and the presence of immunogenic peptides appeared to be reduced based on predicted affinity for MHCII alleles (Table 1). Although it is difficult to predict immunogenicity of therapeutic antibodies, the increased humanness and decreased number of T cell epitopes could feasibly reduce incidence of immunogenicity.^{17,18,20} Furthermore, the removal of glycosylation and deamidation sites reduces the complexity of lot-to-lot characterization and eliminates the potential for immunogenic Fab saccharides, even when expressed in non-human cells.

After humanization, additional modification of the three C-terminal residues of VL was attempted as a means to decrease aggregation of scFvs.^{13,15,16} Due to absence of the CH1/CL domains, scFvs have surfaces unnaturally exposed to the solvent. As human Vλ has a more hydrophobic C-terminus with fewer charged residues than V_{K} (LTVL vs. LEIK), it may have superior packing of its last beta sheet. Indeed, this modification decreased aggregation in all three cases (on average by 8%), and increased titer and thermal stability in two of three cases (Table 6).

Furthermore, IgG antibodies have a conserved N-glycosylation site at N297, located in the CH2 domain within the upper Fc domain. In addition, a minority of antibodies are glycosylated in the Fab region, due to N-glycosylation motifs that are sometimes present within the variable regions (8). The glycan profile of antibody therapeutics must be routinely characterized batch-to-batch in order to ensure consistent and homogeneous protein is obtained during large-scale expression and purification. A challenge arises when glycan sites are located both within the Fab and the Fc of an antibody, as the molecule must be digested or otherwise deconvoluted to clearly identify the glycan profiles at each site. However, removal of the glycan within the Fab domain often compromises its affinity for the targeted antigen, as the glycan can stabilize the active antigen-binding conformation. In the case of cetuximab, the antibody is glycosylated at N99 (AHo) of the VH domain (9), glycosylation increases the cost of goods due to the need for additional quality control steps to characterize its glycan sites. Removal of the Fab glycan while retaining the original affinity for EGFR represents a major leap forward for the generation of antibodies that are easier to characterize and still retain full efficacy.

Cetuximab has been humanized previously using other strategies. One CDR grafting study of cetuximab generated an antibody that could bind to cells overexpressing EGFR, though the affinity was decreased 9-fold.^{21,22} This increase in K_{D} mirrors the affinity change observed for CDR grafting in the present study. Also, cetuximab has been glycoengineered to remove α-1,3-galactose epitopes, demonstrating an alternate approach to decrease the immunogenicity of this antibody.²³ Collectively, the data presented here demonstrate that protein engineering of cetuximab can improve its stability and immunogenicity properties, and more generally suggest that sequence-, and especially structure-guided methods can be used to generate humanized antibodies with superior stability and binding properties.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1. Humanized EGFR Binding Sequences

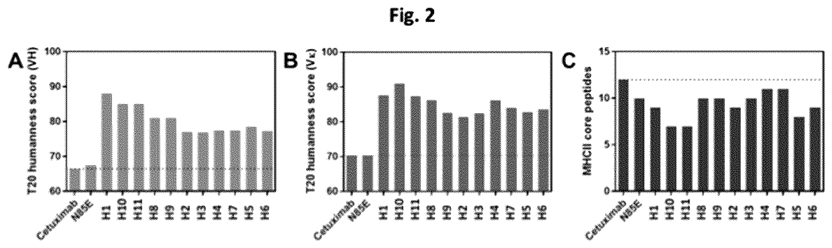

The humanness of the sequences was calculated using the Lake Pharma Antibody Analyzer (https://dm.lakepharma.com/bioinformatics/), which provides a T20 score (range 0 to 100, with 100 being the most human) (Table 1, FIG. 2C). Notably, the wild-type mouse sequences had low scores of 66.44 (VH) and 70.38 (VK) when calculating a score for the framework regions only. In contrast, the sequences for humanized variants (e.g. H1-H11) had significantly higher T20 humanness scores, ranging from 76.95 to 88.10 (VH) and from 81.44 to 91.04 (VK). Thus, the sequences for humanized variants are predicted to have lower immunogenicity than the mouse sequences due to lower MHCII binding and higher degree of humanness.

To increase the humanness of the cetuximab variable regions and decrease the potential for immunogenicity, the mouse VH and Vk domains were converted to a more human framework (FIG. 1). Version H1 was based on a simple graft of Kabat CDR residues onto a stable human framework.¹³ Versions H8, H9, H10, and H11 were designed based on sequence homology to human germline sequences. In particular, for versions H10 and H11, the framework residues were mutated in order to match the most similar human germline sequences. For versions H8 and H9, the framework residues were mutated to the consensus residue in human antibodies. The rest of the humanized versions (H2, H3, H4, H5, H6, H7) were designed based on structural analysis of cetuximab, by mutating framework residues to those residues occurring with a frequency of at least 5% in the human germline that caused the most stable structure in silico. Because the energy analysis for this type of humanization depends on the input model, several input structures were examined. Version H2 used the cetuximab crystal structure 1YY9.¹⁴ Versions H3, H4, H5, H6, and H7 used scFv models generated from the antibody modeling feature of Discovery Studio, based on the sequence of cetuximab variable domains. Versions H4, H5, H6, and H7 incorporated changes in the input sequence to increase similarity of the VH C-terminus to the consensus sequence in humans, or to make the V_{K} C-terminus more Vλ-like. After humanization in Discovery Studio, H7, H9, and H11 were further modified by converting the last three residues of the V_{K} domain into their corresponding residues from the λJ-gene. This change was evaluated due to the known importance of the last VL beta strand in determining scFv stability and aggregation propensity, and the more hydrophobic nature of the Vλ terminus, which could provide packing energy to stabilize the interaction.^{13,15,16} All humanization strategies are summarized in Table 2.

For humanized version H1, the cetuximab Kabat CDRs were grafted onto a stable framework described previously.¹³ All other humanized versions were designed using Discovery Studio 2020 suite. Versions H8-H11 were designed using the Predict Humanizing Mutations protocol based exclusively on the amino acid sequence of cetuximab as the Query Sequence. Identity Threshold was set to 50, Frequent Residue Substitution Tolerance was set to 20, Germline Substitution Tolerance was set to 0, and substitutions of Kabat CDR residues, IMGT CDR residues, Vernier Zone residues, and human germline residues were excluded. Versions H10 and H11 were generated based on Germline substitutions, while versions H8 and H9 used Frequent Residue Substitutions. Versions H2-H7 were designed using different input models for cetuximab with Calculate Mutation Energy set to True (CHARMm forcefield) in order to generate Best Single Mutations sequences. The Query Structure was various models for cetuximab, as shown in Table 2. Version H2 used the cetuximab component of PDB 1YY9 (cetuximab in complex with EGFR) in order to capture the poses of CDRs in the bound state. Versions H3-H7 used cetuximab models generated by Discovery Studio's Antibody Modeling Cascade. The Input Sequences were cetuximab VH and VL for H3, cetuximab VH (ending TVSS instead of TVSA) and VL for H4 and H7, cetuximab VH and VL (ending LTVL instead of LELK) for H5, and cetuximab VH (ending TVSS instead of TVSA) and VL (ending LTVL instead of LELK) for H6. The Top 5 Framework Templates were used with Sequence Similarity Cutoff of 10. CDR loop definition was set to Honegger and Maximum Templates Per Loop was set to 3 with Optimization Level set to High. After generating humanized sequences, versions H10, H8, and H4 were modified to H11, H9, and H7, respectively, by substituting the last four residues of the VL to LTVL to mimic the stable FR4 of lambda antibodies.

Sequences for cetuximab variable domains and their humanized versions are shown in (FIG. 1). Panels A and B show alignments of the VH and VL sequences, respectively. Vernier zone residues flanking the CDR region, and in structurally important framework regions, were also conserved to maintain antigen binding. Examination of amino acid identity between the sequences (Table 3) revealed that the humanized VH sequences had 84-87% identity with cetuximab, and 79-100% identity with each other. Excluding comparison of versions with modified lambda J region, which by definition have 100% VH identity to their corresponding unmodified humanization, the maximum identity between humanized VH sequences was 95%. The humanized VL sequences had 79-86% identity with cetuximab, and 76-98% identity with each other. Notably, the sequence identity was reduced when comparing only the framework regions (70-82% identity for cetuximab VH and humanized VH, and 60-82% identity for cetuximab VL and humanized VL).

Besides the Fc glycans, wild-type cetuximab has 2 potential glycosylation sites in the VH domain (Kabat N85, known to be glycosylated) and the VK domain (Kabat N41, part of NGS glycosylation motif), respectively. In addition, this same VK N49 may be deamidated, as it forms an NG deamidation motif. In order to remove liabilities associated with stability and manufacturing assessment of these post-translational modification sites, all 3 liabilities (2 glycosylation and 1 deamidation) were removed in all humanized sequences, H1-H11 (FIG. 1). In particular, Kabat residue VH N85, comprising an occupied NDT glycosylation motif in cetuximab, was modified to A, D, or E amino acids in the humanizations, eliminating this known glycan site. Similarly, VL N41, comprising an NGS glycosylation motif in cetuximab, was changed to the more typical G residue in all humanizations

The humanness of wild-type cetuximab and humanized variable regions was calculated using the T20 humanness score based on the sequence of the framework regions.¹⁷ Cetuximab, which is a chimeric antibody with mouse variable regions, had low T20 scores of 66.44 (VH) and 70.38 (V_{K}). The T20 score of humanized VH domains increased from 66.44 to a range of 76.95-88.10 (Table 1, FIG. 2A), while the score of humanize V_{K} domains increased from 70.38 to a range of 81.44-91.04 (Table 1, FIG. 2B). Thus, humanization of cetuximab variable regions significantly improved the humanness of these sequences, which could reduce immunogenicity based on increased sequence homology to human germlines.

While presence of non-human sequences in biologics can cause immunogenicity in the form of anti-drug antibodies (ADAs), robust high-affinity ADA can only occur if the offending B cell is activated to undergo class-switch recombination to the IgG subtype. This B cell activation requires binding of presented MHCII-peptide to a compatible T cell receptor on CD4+ T cells. Thus, an undesired ADA response is more likely to occur if the therapeutic antibody contains peptides that bind stably to MHC class II.

The MixMHC2pred algorithm (https://github.com/GfellerLab/MixMHC2pred) was used to predict MHCII-binding ligands within the antibody sequences.¹⁸ The algorithm detects the number of ‘core’ peptides in a given amino acid sequence that will bind to MHCII with sufficient affinity to form a stable T cell epitope. The higher the number of MHCII-binding peptides identified in a sequence, the more potential T cell epitopes the sequence contains. Notably, the algorithm cannot distinguish immunogenic versus tolerogenic peptides; however, a high number of core peptides increases the likelihood of containing some peptides that are pro-immunogenic. The MixMHC2pred algorithm was purchased and downloaded from its GitHub repository. After running the algorithm on VL/VH scFv sequences containing (G₄S)₄ linker, the number of core peptides was calculated and tabulated for the different sequences. Scoring was performed across multiple alleles, allowing sequences to be evaluated for the presence of strong ligands to any allele of MHCII. The number of core peptides was calculated based on the number of peptides in the sequence that could bind to any MHCII allele with a score in the top 0.2% of interactions.

In order to evaluate the presence of MHCII epitopes within cetuximab variable regions, the VH and VL sequences were run through a calculator that predicts MHCII binding affinity. The algorithm, MixMHC2pred, is based on binding of ˜100,000 peptides to different HLA-II alleles.¹⁸ Based on an input sequence, MixMHC2pred evaluates binding of each peptide within the sequence to each HLA-II allele, and returns a ranked score for each residue based on its strongest interaction with any allele. The number of core peptides binding to MHC was calculated based on the number of unique peptides ranking within the top 0.2% of all interactions. To simplify the scoring system into a single value per VH-VL pair, sequences were run as scFv [VL-(G₄S)₃-VH], which accounts for peptides within both VH and VL. Using this system, the number of MHCII core peptides was calculated for cetuximab and the humanized sequences (Table 1, FIG. 2C). Although MHCII binding was not used as a criterion for humanization, all of the humanized sequences had a reduced number of peptides scoring within 0.2% of interactions. While cetuximab had 12 core peptides, the humanized sequences had 7-11 core peptides. This reduction in MHCII binding, combined with more human sequence, may lessen the likelihood of immunogenicity for the humanized variable regions. Additionally, 12 residues within CDRs were identified in the mouse sequence that are predicted to be part of MHCII-binding peptides. In many humanized variants, including H2, H3, H4, H5, H6, H7, H10, and H11, the number of CDR residues in MHCII-binding peptides was reduced.

### Example 2. Methods of Making and Characterizing Humanized EGFR Binding Peptides, Domains, Antibodies, and Antibody-Like Proteins

To characterize humanized anti-EGFR variable regions in the form of various single therapeutics, i.e. EGFR binding complex, αhuEGFR variants (αhuEGFR), His-tagged scFv protein (scFv-6His), recombinant scFv-monoFc monomer (scFv-monoFc), monoclonal antibody (mAb), bispecific antibody (bispecific), penta-GNC antibody (pentaGNC), hexa-GNC antibody (HexaGNC), and penta-miniGNC antibody (miniGNC) were generated and characterized, as listed in Table 4, by the following methods.

2a. Expression and Purification of EGFR Binding Complex

Protein stability is a key parameter defined by the difference in free energy between the folded and unfolded states. For protein therapeutics, stability may impact immunogenicity, pharmacokinetics, and even efficacy (7), and reduction of aggregation can help to develop therapeutics that are easier to manufacture and safer for patients. In addition, expression efficiency and protein yield directly determine the cost of protein therapeutics. If proteins can be more efficiently expressed to reach higher titers and increased yield of purified protein, manufacturing costs can be reduced significantly.

Proteins were expressed by transfecting the expression plasmids for His-tagged scFv or scFv-monoFc (single plasmid) or co-transfecting heavy and light chains (for other formats) in the ExpiCHO system (Thermo Fisher), collectively called EGFR binding complex. Briefly, 10 μg of each expression plasmid (or 20 μg of an unpaired plasmid) was brought to 1 ml with OptiPRO SFM medium. 1 ml of OptiPRO SFM medium containing 80 ul Expifectamine CHO reagent was added to the DNA and incubated at room temperature for 2.5 minutes. The resulting mixture was then added to 25 ml ExpiCHO cells at 6×10⁶ cells/ml in a 125 ml Erlenmeyer flask and incubated at 37° C., 5% CO₂, 150 rpm. Cells were fed with 8.75 ml ExpiCHO feed and 150 μl of CHO enhancer at 24 hours post-transfection and shifted to 32° C., 5% CO₂, 150 rpm. Cells were fed again at 48 hours post-transfection with 8.75 ml ExpiCHO feed. Culture supernatant was harvested 8 days post-transfection, spun for 1 hour at 4500 rpm to pellet the cells and then passed through a 0.2 mm filter.

Fc-containing proteins were purified from the harvested supernatant using a 1-ml MabSelect PrismA protein A column (GE Healthcare). The column was equilibrated with phosphate-buffered saline. The supernatant was then passed through the column at a flow rate of 2 ml/min. The column was washed with 10 ml PBS+0.1% Triton X-100, followed by 10 ml PBS+300 mM NaCl, and finally 10 ml PBS. Protein was then eluted by passing 5 ml of 50 mM sodium acetate, pH 3.5 through the column. The eluted protein was immediately neutralized by addition of 0.5 ml 1M Tris-Cl, pH8.0.

His-tagged scFv proteins were purified from the harvested supernatant using a 1-ml HisTrap HP column or 1-ml protein L (CaptoL) column (GE). The column was equilibrated with phosphate-buffered saline containing 0.5 M NaCl and 20 mM imidazole, pH 7.4 (HisTrap) or PBS (protein L). The supernatant was spiked with 10× binding buffer to reach 0.5 M NaCl and 20 mM imidazole (His trap only) and run over the column at a flow rate of 2 ml/min. The column was washed with 10 column volumes of PBS containing 0.5 M NaCl and 20 mM imidazole (HisTrap) or PBS (protein L), and the protein was eluted using PBS containing 0.5 M NaCl and 500 mM imidazole, pH 7.4 (HisTrap) or 50 mM sodium acetate pH 3.5, later neutralized with 0.5 ml 1M Tris pH 8.0 (protein L).

Immediately after first-step protein A or His tag purification, scFv-monoFc proteins were analyzed by analytical SEC using using Waters Acquity UPLC H-Class with ACQUITY UPLC® Protein BEH SEC 200 Å, 4.6 mm×150 mm, 1.7 μm column. PBS (125 mM sodium phosphate, 137 mM sodium chloride, pH 6.8) was used as mobile phase for 10-minute runs at 0.3 ml/min, injecting 10 μg protein. For higher resolution, mAbs were instead analyzed by analytical SEC using an Acquity Arc Waters HPLC with XBridge BEH SEC 300 Å, 7.8×300 mm, 3.5 μm column. PBS (150 mM sodium phosphate, 100 mM sodium chloride, pH 6.8) was used as mobile phase for 20-minute runs at 0.714 ml/min, injecting 50 g protein. Two separate purifications were assessed for each protein, with % peak of interest values reported as average±standard deviation.

2b. Assays for Characterizing the Binding Specificity and Affinity of EGFR Binding Complex

Biolayer interferometry (Octet) binding assays were performed on an Octet96 or Octet384 instrument to ensure that proteins containing humanized cetuximab binding domains retain binding to their cognate antigens. Fc-containing protein was captured to anti-human Fc (AHC) sensor tips by loading for 180 seconds at 10 μg/ml. Alternatively, His-tagged proteins were covalently coupled at 10 ug/ml to AR2G tips using manufacturer protocol. After a 60-second baseline step, a 180- to 300-second association phase with serial dilutions (0-200 nM; 1:2.5 dilution factor) or a single 100 nM concentration of purified human EGFR in assay buffer (phosphate-buffered saline containing 0.1% BSA, 0.05% Tween20) was performed, followed by a 300- to 600—second dissociation phase in assay buffer. Regeneration was achieved using 10 mM glycine, pH 1.5. Binding curves were globally fit to a 1:1 model to extract the dissociation constants, K_{D}. Binding kinetics for each protein were assessed in duplicate, with tabulated values reported as average±standard deviation.

2c. Assays for Characterizing TDCC of EGFR Binding Complex

The tumor-targeting properties of the humanized anti-EGFR domain in multi-specific antibodies, collectively known as GNC antibodies, were evaluated by testing their ability to induce tumor-specific cytotoxicity while engaging T-cell activation, redirecting T-cell mediated cytolysis, and ultimately killing the target cells. A luminescence-based T cell-dependent cellular cytotoxicity (TDCC) assay was used to measure the extent of antibody-induced cellular cytotoxicity by quantification of cell viability via constitutive expression of luciferase.

Luciferized BXPC3 tumor cells (ATCC) were cultured at 37° C., 5% CO2 in the RPMI 1640 media containing 10% fetal bovine serum. Cell viability was monitored with a Vi-CELL automated cell counter (Beckman Coulter). 500 tumor cells (20 L) per well were plated into a 384-well, white, flat-bottom polystyrene TC-treated microplate (Corning) and incubated at 37° C., 5% CO2. After 24 hours, human pan T-cells were added to reach an effector-to-target (E-T) ratio of 5:1 and antibody was added with a 5-fold dilution series (0-30 nM). Cells were dispensed using a Multidrop bulk liquid dispenser (BIOTEK). Antibody dilutions were added (10 μL/well) and plates were incubated for an additional 72 hrs at 37° C., 5% CO2 before luminescence-based cell viability quantification.

To quantify the luminescence produced by constitutively expressed firefly luciferase, the Bright-Glo Luciferase Assay System (Promega) was used. BrightGlo reagent was added (20 μL per well) at room temperature and luminescence was quantified with a luminescence detecting plate reader (BMG Labtech). Antibody EC50 was determined by transforming the data in Microsoft Excel and analyzing with GraphPad Prism 6 software “log (agonist) vs. response—variable slope (four parameters)”. The resulting EC50 value is reported. The TDCC assay was done in quadruplicate with good inter-plate reproducibility, and no significant variability was seen from different locations on the plate.

### Example 3. His-Tagged-EGFR Binding scFv Proteins

The sequences encoding humanized (H1) anti-EGFR binding domains were cloned into His-tagged scFv expression format containing the residues GSHHHHHH at the C-terminal of the scFv. The expression vectors were transfected into 25 ml of ExpiCHO and expressed for 8 days before harvesting and purifying via protein L affinity chromatography. The H1 variant had significantly higher titer than the mouse version (Table 5).

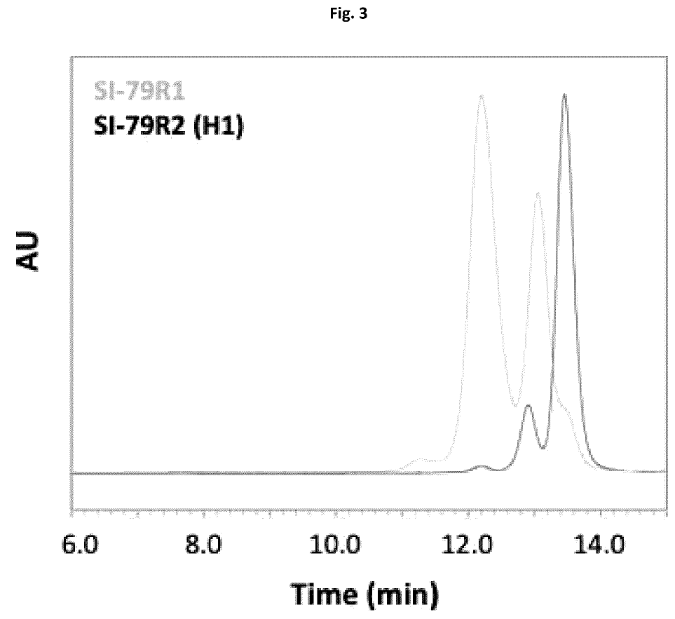

Analytical SEC data after protein L purification demonstrates that the humanized scFv had significantly less aggregation than the scFv encoded by wild-type mouse sequence (FIG. 3, Table 5). Additionally, the main peak was shifted to the right, consistent with modification of the glycosylation site and resulting aglycosylation of the VH.

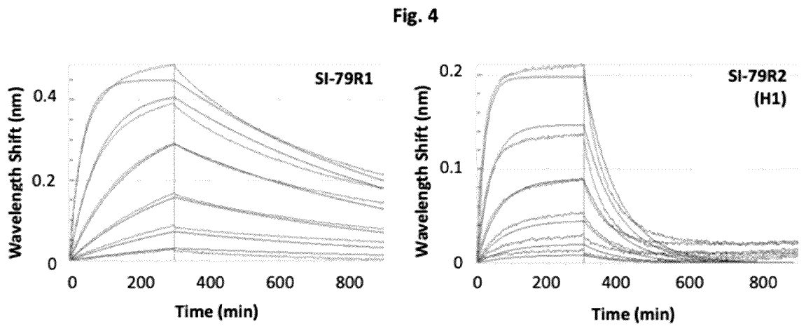

Octet was used to verify that the humanized scFv protein can bind to human EGFR (FIG. 4). His-tagged scFv proteins were loaded via covalent coupling onto AR2G sensors at 10 ug/ml and bound to a serial dilution (highest 200 nM, 1:2.5 dilutions) of His-tagged human EGFR. The resulting global fit to a 1:1 binding model demonstrated that both wild-type mouse scFv and humanized scFv proteins bind to EGFR with affinities in the low nanomolar range (Table 5).

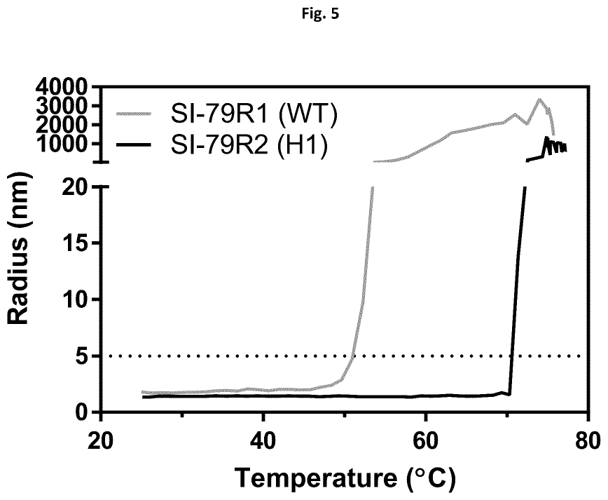

Dynamic light scattering was used to compare thermal stability of the scFv proteins (FIG. 5). The temperature was ramped from 25° C. to 75° C. at 0.5° C./min while the radius of the scFv proteins (1 mg/ml) was monitored by a Wyatt DynaPro Plate Reader III. The data revealed that the H1 version has a significantly higher Tm value (Table 5), consistent with increased stability.

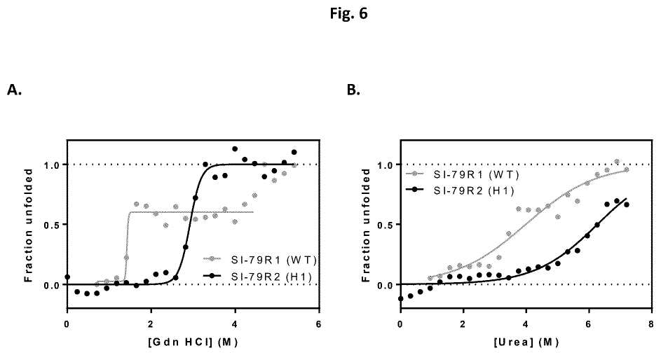

The stability toward chemical denaturation was also examined using guanidine and urea unfolding assays (FIG. 6). Proteins at a concentration of 0.1 mg/ml were incubated with 24 guanidine HCl concentrations from 0 to 5.4 M or urea concentration from 0 to 7.2 M overnight. Fluorescence intensity (excitation 295 nm, emission 360 nm) was measured on a CLARIOstar plate reader, fluorescence intensity was normalized to represent fraction unfolded protein, and sigmoidal fits were used to extract EC50 values for stability comparison. The humanized variant H1 was more resistant than wild-type to unfolding by guanidine denaturation according to resulting EC50 vales (Table 5).

### Example 4. Humanized EGFR Binding scFv-monoFc Fusion Proteins

To assess of the biophysical properties of humanized cetuximab VH and VL domains, sequences were cloned into scFv format and fused to a monoFc to facilitate purification and Octet analysis.¹⁹ The scFv domain was in VL-VH orientation, and included a (G₄S)₄ linker between VH and VL domains. Notably, generation of the scFv panel was more efficient than generating the corresponding mAb panel, which requires separate cloning for heavy and light chains. As controls, the wild-type cetuximab scFv and an aglycosylated version generated by mutation of the modified asparagine residue (VH N85E) were also generated.

Plasmids encoding wild-type, aglycosylated (N85E), and humanized scFv-monoFc proteins were transiently transfected in ExpiCHO cells, and protein was purified from the cell supernatant using protein A affinity chromatography. As shown in Table 6, the majority of humanized proteins had superior expression titer to the wild-type and simple aglycosylated versions of cetuximab despite containing the same monoFc domain and using the same algorithm for codon optimization. While the average titers for wild-type and aglycosylated cetuximab were 163 and 116 μg/ml, respectively, the average titer for the humanized versions ranged from 220 to 506 μg/ml for H8 and H7, respectively.

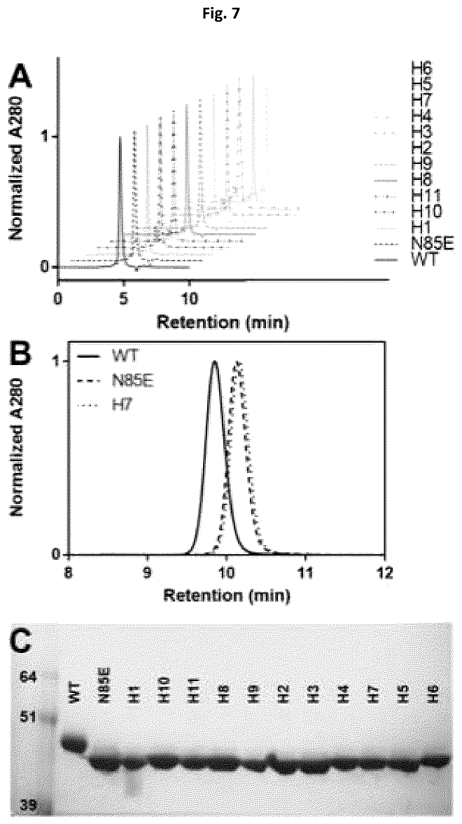

After the first step protein A purification, analytical size-exclusion chromatography (SEC) was used to assess aggregation of the scFv-monoFc proteins (FIG. 7A, Table 6). The wild-type and aglycosylated cetuximab had 93.6% and 94.9% protein of interest, respectively, due to a small amount of aggregation, and the humanized versions had on average similar levels of aggregation. The version with the least aggregation (H9) had 97.4% protein of interest, while the most aggregated version (H4) had 82.6% protein of interest. The modification of V_{K} C-terminus to include the sequence from Vλ appeared to decrease aggregation. Humanized versions containing these Vλ residues (H11, H9, and H7) had less aggregation than the corresponding versions where the original V_{K} residues were used (H10, H8, and H4, respectively). Preparative SEC was performed for all proteins in order to remove aggregate, exchange into storage buffer, and ensure accuracy of subsequent biophysical assays which benefit from using highly pure protein. SDS-PAGE of purified scFv-monoFc proteins demonstrated increased mobility of N85E and all humanized versions relative to wild-type cetuximab, confirming lack of glycosylation for these variants (FIG. 7C).

The scFv-monoFc proteins were analyzed by SDS-PAGE using NuPAGE 4-12% Bis-Tris gels (Thermo Fisher, NP0323BOX) and MES running buffer (Thermo Fisher, NP0002). 3 μg of each protein was prepared in LDS sample buffer (Thermo Fisher, NP0007) with or without 10 mM DTT and heated for 10 min at 70° C. Gels were run for 50 minutes at 150 V, stained with SimplyBlue (Thermo Fisher, LC6065), and destained with water before imaging.

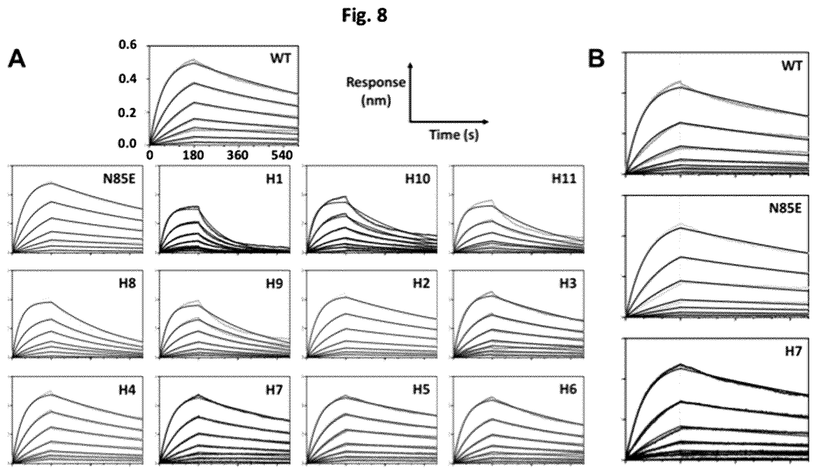

Binding of scFv-monoFc proteins to human EGFR was assessed by biolayer interferometry to reveal whether the humanization process altered binding kinetics (Table 6, FIG. 8A). The monoFc domain was used to load proteins onto anti-human Fc (AHC) sensors, followed by binding of scFv to serial dilutions of the extracellular domain of human EGFR. Wild-type cetuximab scFv had an affinity of 3.18 nM, consistent with previous reports. The aglycosylated variant (N85E) had very similar binding kinetics with a KD of 3.16 nM, indicating that glycosylation is not imperative for antigen binding.

The KD values for the humanized versions fell into three main categories. For the humanized version using a straight CDR graft onto a stable human framework (H1), there was a 4-fold decrease in binding affinity which was driven by increased rate of dissociation. For humanizations based on sequence homology to a single human germline (H10, H11) or the global dataset of human germlines (H8, H9), there was a consistent 2-fold decrease in binding affinity where faster dissociation was again the kinetic determinant. Finally, for humanizations based on structural homology (H2 through H7), there was no significant decrease in binding affinity.

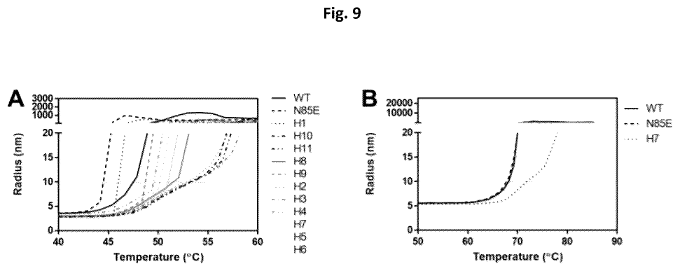

Thermal stability of scFv-monoFc proteins was assessed by dynamic light scattering (Table 6, FIG. 9A) by observing the increase in hydrodynamic radius as the temperature was ramped from 25° C. to 85° C.

Since the shapes of the unfolding curves were complex and not uniform for the different samples, the temperature at which the radius surpassed 10 nm was used to objectively compare protein stabilities. Using this metric, the wild-type cetuximab protein unfolded at 47.2° C., while the aglycosylated N85E variant appeared slightly less stable, unfolding at 44.5° C. Thus, the occupied glycosylation site may help to stabilize the folded conformation of wild-type cetuximab scFv.

Similar to the binding results, three categories of stability were observed. For the humanization based on CDR grafting to an unrelated human framework, the stability was slightly decreased relative to wild-type cetuximab. Five humanized versions showed similar or slightly enhanced stability relative to cetuximab. Two of these were based on sequence homology to the global dataset of human germlines (H8, H9) while three were based on structural modeling (H4, H5, H7). Lastly, five humanized versions appeared to be significantly more stable than the other proteins. H10 and H11 were generated by CDR grafting onto the most sequentially homologous human framework, while H2, H3, and H6 were based on homology models. In contrast to SEC data showing systematic reduction in aggregation by using C-terminal residues from λ J genes, the DLS data did not show a consistent impact of these residues on stability. Whereas versions H11 and H7 showed subtle increases in stability relative to H10 and H4, respectively, H9 actually appeared less stable than its relative H8.

### Example 5. Humanized Anti-EGFR Monoclonal Antibodies

To understand if the properties of scFv-monoFc proteins would translate to IgG format, mAbs were generated for wild-type cetuximab, the aglycosylated variant N85E, and a humanized version of cetuximab. Based on the highest protein expression, low aggregation, improved thermal stability, and unchanged binding affinity, humanized version H7 was selected for conversion to mAb format. The three mAb proteins were produced by transient transfection in ExpiCHO cells and harvested after 9 days of expression.

Mirroring the scFv-monoFc results, the expression titer of humanized H7 was increased relative to that of wild-type or aglycosylated cetuximab (Table 8), although the difference in titer was not as pronounced as for the scFv-mFc format. After protein A purification, all proteins were >99% pure as assessed by analytical SEC (Table 8, FIG. 7B). Notably, there was significantly less aggregation of the mAbs than the corresponding scFv-mFc proteins, which could be attributed to the intrinsic stability of the IgG backbone relative to scFv and monoFc domains. The SEC data also demonstrates that wild-type cetuximab had a significantly shorter retention time than either the aglycosylated N85E or humanized H7 versions. This difference in apparent molecular size can be attributed to the glycosylation of cetuximab, which is absent in N85E and humanized versions.

Binding kinetics of mAbs to human EGFR were assessed by biolayer interferometry (Table 8, FIG. 8B) and demonstrated no difference in binding affinity or kinetics between versions. These results confirmed the results of the scFv-monoFc proteins, which demonstrated that the aglycosylating mutation N85E and the humanization mutations of H7 did not disrupt the interaction of cetuximab CDRs with its antigen. Binding affinity of the mAbs was similar to that of the corresponding scFv-monoFc proteins.

Finally, the DLS experiment was repeated to characterize the stability of the mAbs (Table 8, FIG. 9B). Whereas wild-type and aglycosylated cetuximab had very similar stabilities (unfolding at 68.3 and 68.1° C., respectively), the H7 version had an elevated unfolding temperature of 72.5° C. Thus, humanized version H7 appears to be more stable than wild-type cetuximab whether in scFv or mAb format.

### Example 6: Bispecific Antibodies with T Cell Engager and Humanized EGFR Binding

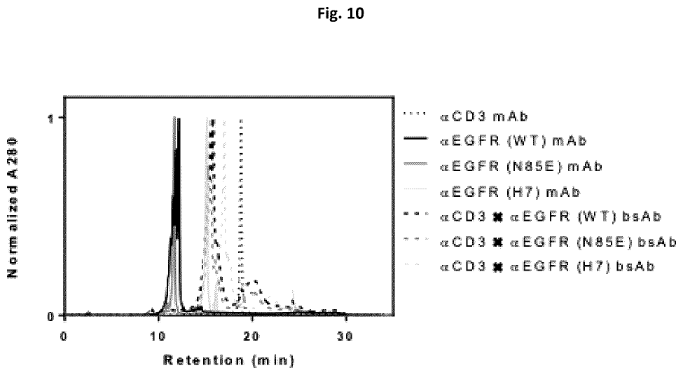

As a final evaluation of functional activity, bispecific versions of cetuximab were generated and used in a T cell-dependent cellular cytotoxicity (TDCC) assay. The three versions of cetuximab mAb (wild-type, N85E, H7) contained the K409R mutation in the CH3 domain, which allowed for controlled Fab-arm exchange to occur when incubated with an anti-CD3 antibody containing the complementary F405L mutation. The formation of αEGFR×αCD3 bispecific antibodies from the complementary anti-EGFR and anti-CD3 mAbs was confirmed by cation exchange chromatography (FIG. 10). Antibodies were analyzed by cation exchange chromatography using Agilent 1260 Infinity Quaternary HPLC with Thermo Scientific ProPac™ SCX-10 HPLC Column, 4×250 mm, 10 μm at 35° C. Thermo Scientific CX-1 pH Gradient Buffers were used as mobile phases (Table 7 contains gradient steps). 50 μg of protein sample was loaded and separated with flow rate of 0.5 ml/min, eluted at gradient shown in table below over 35 minutes.

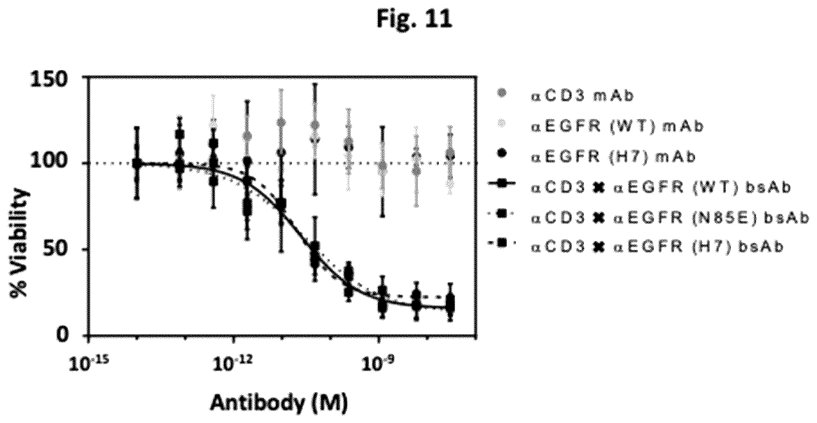

To assess TDCC activity, serial dilutions of bispecific antibodies and control mAbs were incubated with activated T cells and luciferized EGFR-bearing BxPC3 target cells at an effector:target ratio of 5:1 in a 384-well plate (FIG. 11, Table 8). After 3-day incubation at 37° C., BrightGlo reagent was added to read out luminescence, which is proportional to the number of remaining target cells. The bispecific cetuximab×αCD3 antibody showed potent tumor cell killing with an EC50 value of 24.6 nM. Agycosylated N85E and humanized H7 showed similar EC50 values (30.7 and 20.3 pM, respectively) with overlapping 95% confidence intervals. In contrast, none of the control mAbs (αCD3, cetuximab, or cetuximab H7) showed any BxPC3 killing up to 30 nM, indicating that cytotoxicity required simultaneous targeting of both tumor cells and T cells. Thus, the humanized version of cetuximab retained the biological functionality of cetuximab when tested in a TDCC assay.

### Example 7. Penta-GNC Antibodies Having a Humanized Anti-EGFR scFv or Fab Domain

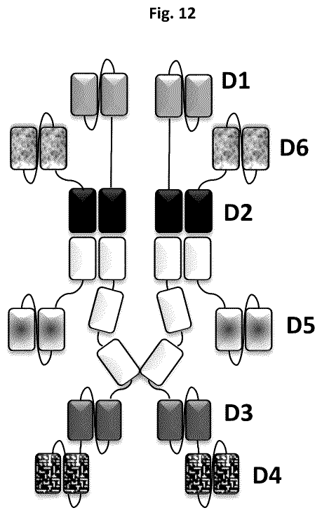

Humanized EGFR binding variants, H1, H4, and H7, were configured and cloned into PentaGNC format in either one of four scFv positions or the Fab position (FIG. 12, D1 or D2 position). Proteins were transfected into 25 mL of ExpiCHO and expressed for 8 days before harvesting and purifying via protein A affinity chromatography. The proteins were expressed with good titer (Table 9).

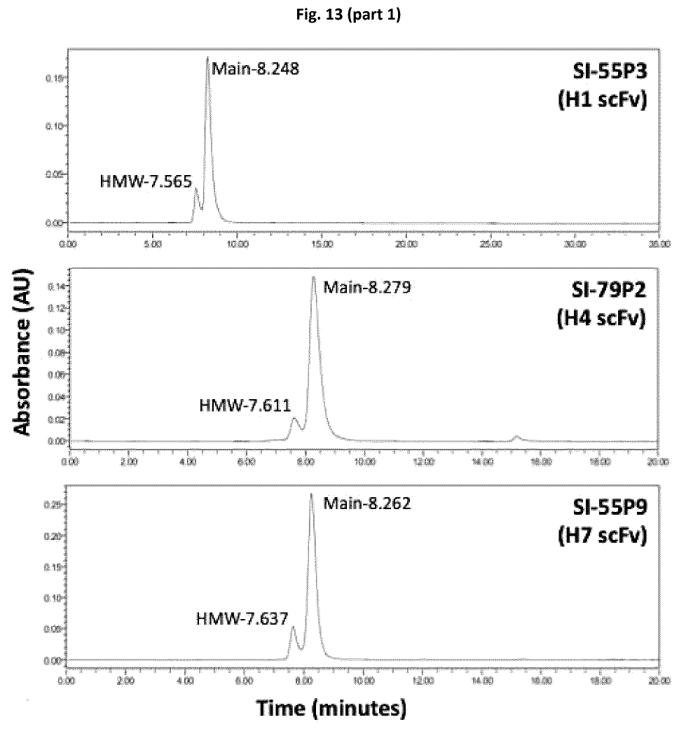

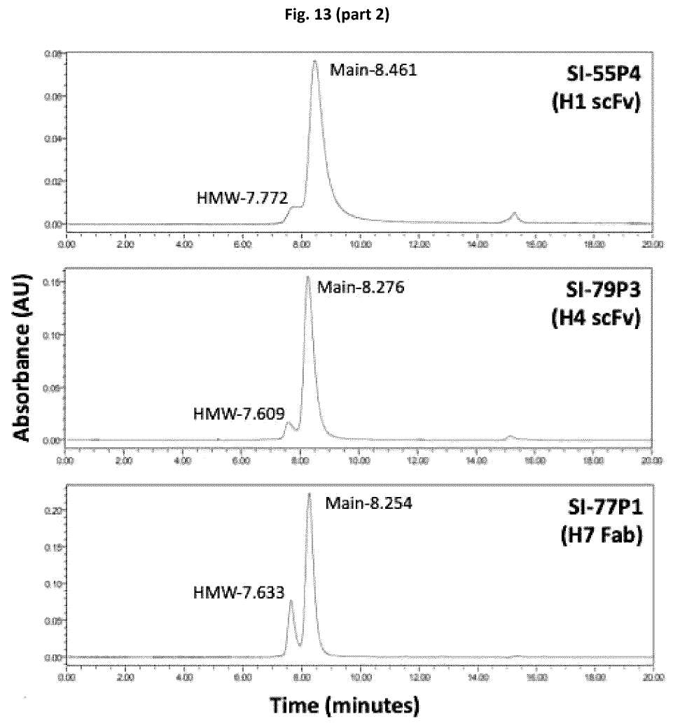

Analytical SEC data after protein A purification demonstrates that the penta-GNC antibodies comprising a humanized anti-EGFR domain, as either a scFv or Fab, can be expressed with low aggregation (FIG. 13, Table 9).

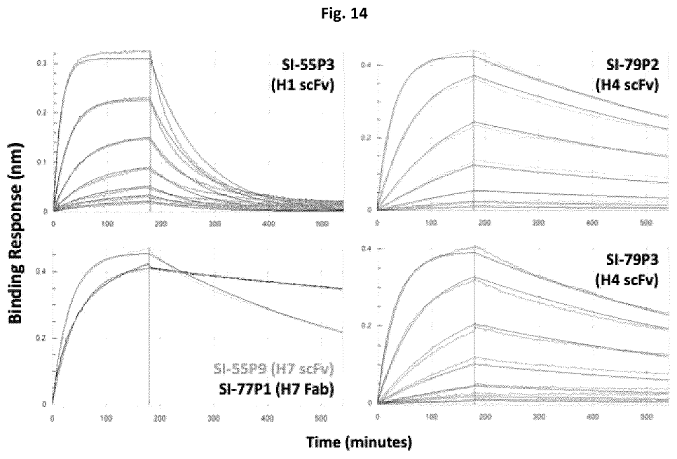

Octet was used to verify that the penta-GNC antibodies having a humanized anti-EGFR domain (e.g. H1, H4, H7) can bind to human EGFR (FIG. 14). The penta-GNC antibodies were loaded via AHC sensors at 10 μg/ml and bound to a serial dilution (highest 200 nM, 1:2.5 dilutions) or a single 100-nM concentration of His-tagged human EGFR. The resulting global fit to a 1:1 binding model demonstrated that the penta-GNC antibodies bind to EGFR with affinities in the low nanomolar range (Table 10).

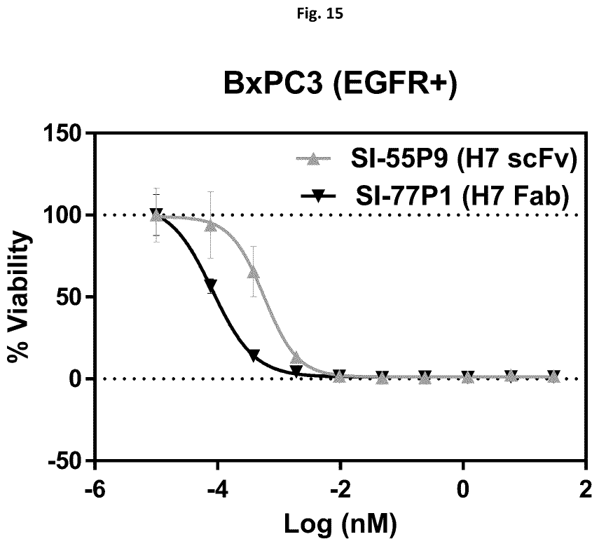

Two penta-GNC antibodies were tested for their TDCC activity using luciferized BXPC3 cells as target cells (FIG. 15). 5-fold serial dilutions (0-30 nM) of pentaGNC antibodies were dosed to a mixture of 500 BxPC3 cells and 2500 activated T cells (effector:target at 5:1), which were incubated for 72 hours before measuring the luminescence readout corresponding to viability of the target cells. Resulting fits to a sigmoidal function revealed that the EGFR-binding domains (H7) of the penta-GNC antibodies efficiently targeted the BxPC3 tumor cells for killing by co-incubated T cells, as demonstrated by EC50 values in the sub-picomolar range (Table 9).

### Example 8. Hexa-GNC Antibodies Having a Humanized Anti-EGFR scFv or Fab Domain

The humanized anti-EGFR binding variant, H7, was configured and cloned into the hexa-GNC format in either one of five scFv positions or the Fab position (FIG. 12, D1 or D2 position). Proteins were transfected into 25 mL of ExpiCHO and expressed for 8 days before harvesting and purifying via protein A affinity chromatography. The proteins were expressed with good titer (Table 11).

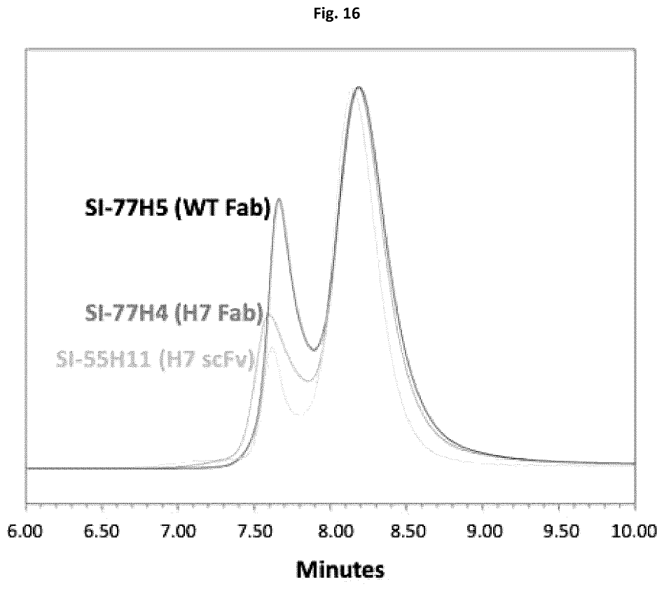

Analytical SEC data after protein A purification demonstrates that the hexa-GNC molecules containing a humanized anti-EGFR domain, either a scFv or a Fab can be expressed with low aggregation (FIG. 16, Table 11). Notably, both proteins containing version H7 had significantly less aggregation than the protein with cetuximab.

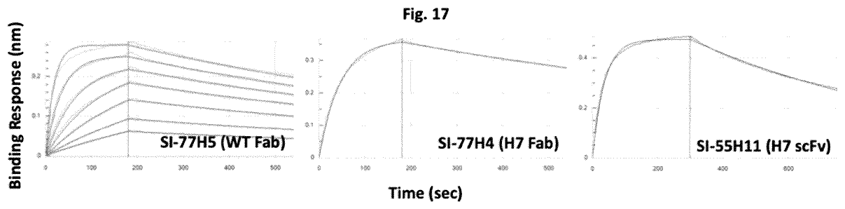

Octet was used to verify that the hexa-GNC antibodies containing a humanized anti-EGFR domain can bind to human EGFR (FIG. 17). The hexa-GNC proteins were loaded via AHC sensors at 10 μg/ml and bound to a serial dilution (highest 200 nM, 1:2.5 dilutions) or a single 100-nM concentration of His-tagged human EGFR. The resulting global fit to a 1:1 binding model demonstrated that the hexaGNC antibodies bind to EGFR with affinities in the low nanomolar range (Table 11).

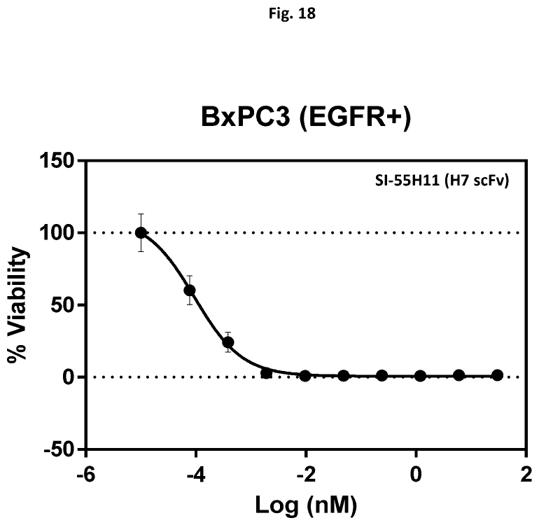

One HexaGNC was tested for activity in a TDCC bioassay using luciferized BXPC3 cells as target cells (FIG. 18). 5-fold serial dilutions (0-30 nM) of the hexa-GNC antibodies were dosed to a mixture of 500 BxPC3 cells and 2500 activated T cells, which were incubated for 72 hours before measuring the luminescence readout corresponding to viability of the target cells. Resulting fits to a sigmoidal function revealed that the EGFR-binding domain (H7) of the hexa-GNC antibody efficiently targeted the BxPC3 tumor cells for killing by co-incubated T cells, as demonstrated by an EC50 value in the sub-picomolar range (Table 11).

### Example 9. Penta-miniGNC Antibodies Having a Humanized Anti-EGFR scFv or Fab Domain

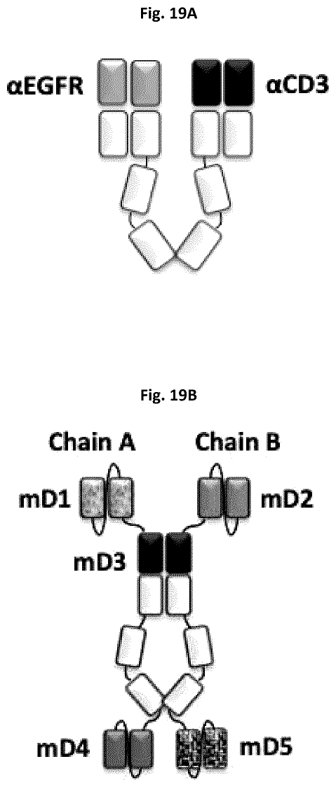

Humanized EGFR binding variants, H1, H4, and H7, were configured and cloned into the penta-miniGNC format (PCT/US2021/022847, incorporated herein by reference in its entirety) at either one of four scFv positions (mD1, mD2, mD4, mD5) or the Fab (mD3) position (FIG. 19). Proteins were transfected into 25 mL of ExpiCHO and expressed for 8 days before harvesting and purifying via protein A affinity chromatography. The proteins were expressed with good titer (Table 12).

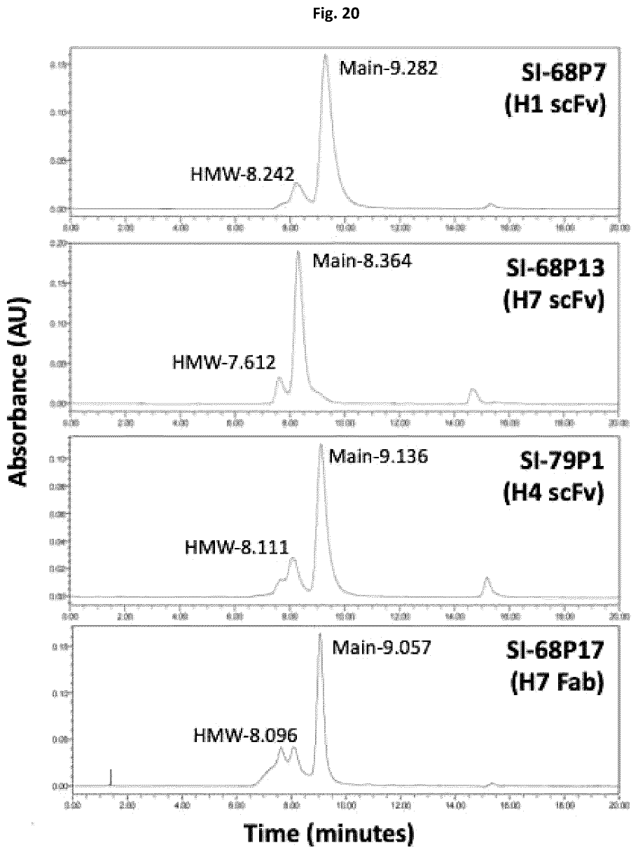

Analytical SEC data after protein A purification demonstrates that penta-miniGNC molecules containing a humanized anti-EGFR domain can be expressed with low aggregation (FIG. 20, Table 12).

Octet was used to verify that the penta-miniGNC antibodies containing humanized anti-EGFR domains (H4, H7) can bind to human EGFR (FIG. 21). The penta-miniGNC antibodies were loaded via AHC sensors at 10 μg/ml and bound to a serial dilution (highest 200 nM, 1:2.5 dilutions) or a single 100-nM concentration of His-tagged human EGFR. The resulting global fit to a 1:1 binding model demonstrated that the penta-miniGNC antibodies bind to EGFR with affinities in the low nanomolar range (Table 12).

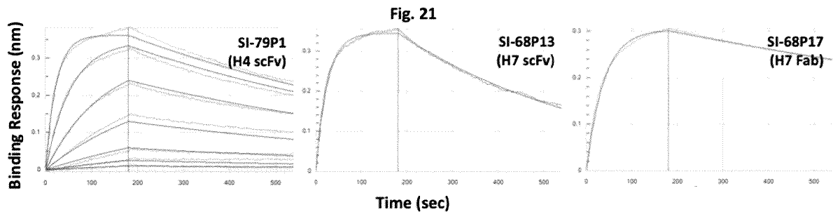

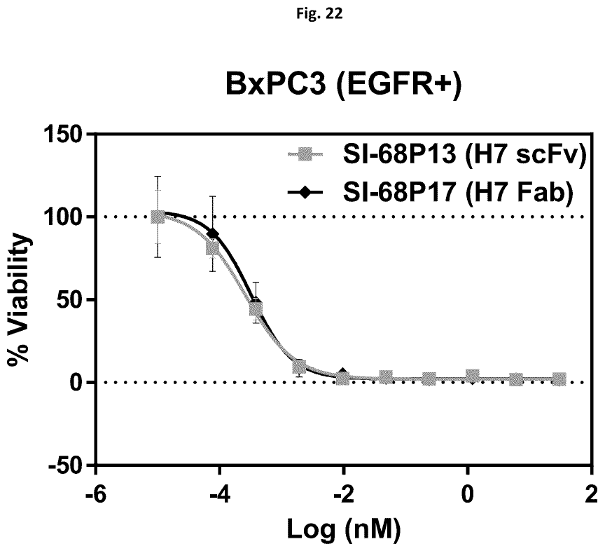

Two penta-miniGNC antibodies were tested for TDCC activity using luciferized BXPC3 cells as target cells (FIG. 22). 5-fold serial dilutions (0-30 nM) of penta-miniGNC antibodies were dosed to a mixture of 500 BxPC3 cells and 2500 activated T cells (effector:target at 5:1), which were incubated for 72 hours before measuring the luminescence readout corresponding to viability of the target cells. Resulting fits to a sigmoidal function revealed that the EGFR-binding variant, H7, of the penta-miniGNC antibody efficiently targeted the BxPC3 tumor cells for killing by co-incubated T cells, as demonstrated by EC50 values in the sub-picomolar range (Table 12).

### Tables

TABLE 1
Humanization of VH/VK regions predicts reduced immunogenicity
and increased humanness scores (framework regions only)

| | Core | CDR residues in | Sequence | VH T20 | VK T20 |
|---|---|---|---|---|---|
| Version | peptides | core peptide | length | score | score |
| Cetuximab | 13 | 12 | 241 | 66.44 | 70.38 |
| H1 | 9 | 12 | 241 | 88.10 | 87.69 |
| H2 | 10 | 6 | 241 | 77.07 | 81.44 |
| H3 | 11 | 6 | 241 | 76.95 | 82.56 |
| H4 | 11 | 6 | 241 | 77.47 | 86.26 |
| H5 | 8 | 10 | 241 | 78.56 | 82.88 |
| H6 | 9 | 10 | 241 | 77.30 | 83.63 |
| H7 | 11 | 6 | 241 | 77.47 | 84.04 |
| H8 | 11 | 12 | 241 | 81.09 | 86.31 |
| H9 | 11 | 12 | 241 | 81.09 | 82.63 |
| H10 | 7 | 10 | 241 | 85.11 | 91.04 |
| H11 | 7 | 10 | 241 | 85.11 | 87.44 |

TABLE 2
Methods for generating humanized cetuximab variants

| Humanized | | | Starting | |
|---|---|---|---|---|
| version | Method | Basis | sequence/model | Modification |
| H1 | CDR graft onto stable framework | Sequence | Cetuximab, FW1.4gen | None |
| H2 | DS Best Single Mutations | Model | PDB 1YY9 | None |
| H3 | DS Best Single Mutations | Model | Cetuximab model | None |
| H4 | DS Best Single Mutations | Model | Cetuximab model (TVSS) | None |
| H5 | DS Best Single Mutations | Model | Cetuximab model (LTVL) | None |
| H6 | DS Best Single Mutations | Model | Cetuximab model (TVSS-LTVL) | None |
| H7 | DS Best Single Mutations | Model | Cetuximab model (TVSS) | Lambda J |
| H8 | DS Frequent Residue Substitutions | Sequence | Cetuximab | None |
| H9 | DS Frequent Residue Substitutions | Sequence | Cetuximab | Lambda J |
| H10 | DS Germline Substitutions | Sequence | Cetuximab | None |
| H11 | DS Germline Substitutions | Sequence | Cetuximab | Lambda J |

TABLE 3
The sequence identity matrices of the entire VH domain (A), VH framework regions (entire
VH domain, except Kabat CDR residues) (B), the entire VL domain (C), and the VL framework
regions (entire VL domain, except Kabat CDR residues) (D), for wild-type cetuximab, aglycosylated
cetuximab (N85E), and humanized cetuximab versions (H1-H11), respectively.

| | | | | | | | | | | | | |
|---|---|---|---|---|---|---|---|---|---|---|---|---|
| WT | N85E | H1 | H10 | H11 | H8 | H9 | H2 | H3 | H4 | H7 | H5 | H6 |

VH identity matrix -- all residues

| | | | | | | | | | | | | | |
|---|---|---|---|---|---|---|---|---|---|---|---|---|---|
| WT | | 99.2 | 78.2 | 84.9 | 84.9 | 85.7 | 85.7 | 86.6 | 84.9 | 84.9 | 84.9 | 84.9 | 84.0 |
| N85E | 99.2 | | 79.0 | 84.9 | 84.9 | 86.6 | 86.6 | 87.4 | 85.7 | 84.9 | 84.9 | 85.7 | 84.9 |
| H1 | 78.2 | 79.0 | | 84.9 | 84.9 | 92.4 | 92.4 | 82.4 | 82.4 | 79.8 | 79.8 | 81.5 | 79.8 |
| H10 | 84.9 | 84.9 | 84.9 | | 100.0 | 92.4 | 92.4 | 87.4 | 85.7 | 86.6 | 86.6 | 89.1 | 86.6 |
| H11 | 84.9 | 84.9 | 84.9 | 100.0 | | 92.4 | 92.4 | 87.4 | 85.7 | 86.6 | 86.6 | 89.1 | 86.6 |
| H8 | 85.7 | 86.6 | 92.4 | 92.4 | 92.4 | | 100.0 | 89.9 | 89.9 | 87.4 | 87.4 | 89.1 | 87.4 |
| H9 | 85.7 | 86.6 | 92.4 | 92.4 | 92.4 | 100.0 | | 89.9 | 89.9 | 87.4 | 87.4 | 89.1 | 87.4 |
| H2 | 86.6 | 87.4 | 82.4 | 87.4 | 87.4 | 89.9 | 89.9 | | 95.0 | 96.6 | 96.6 | 95.0 | 94.1 |
| H3 | 84.9 | 85.7 | 82.4 | 85.7 | 85.7 | 89.9 | 89.9 | 95.0 | | 95.0 | 95.0 | 94.1 | 94.1 |
| H4 | 84.9 | 84.9 | 79.8 | 86.6 | 86.6 | 87.4 | 87.4 | 96.6 | 95.0 | | 100.0 | 93.3 | 95.8 |
| H7 | 84.9 | 84.9 | 79.8 | 86.6 | 86.6 | 87.4 | 87.4 | 96.6 | 95.0 | 100.0 | | 93.3 | 95.8 |
| H5 | 84.9 | 85.7 | 81.5 | 89.1 | 89.1 | 89.1 | 89.1 | 95.0 | 94.1 | 93.3 | 93.3 | | 94.1 |
| H6 | 84.0 | 84.9 | 79.8 | 86.6 | 86.6 | 87.4 | 87.4 | 94.1 | 94.1 | 95.8 | 95.8 | 94.1 | |

VH identity matrix -- framework residues

| | | | | | | | | | | | | | |
|---|---|---|---|---|---|---|---|---|---|---|---|---|---|
| WT | | 98.9 | 70.1 | 79.3 | 79.3 | 80.5 | 80.5 | 81.6 | 79.3 | 79.3 | 79.3 | 79.3 | 78.2 |
| N85E | 98.9 | | 71.3 | 79.3 | 79.3 | 81.6 | 81.6 | 82.8 | 80.5 | 79.3 | 79.3 | 80.5 | 79.3 |
| H1 | 70.1 | 71.3 | | 79.3 | 79.3 | 89.7 | 89.7 | 75.9 | 75.9 | 72.4 | 72.4 | 74.7 | 72.4 |
| H10 | 79.3 | 79.3 | 79.3 | | 100.0 | 89.7 | 89.7 | 82.8 | 80.5 | 81.6 | 81.6 | 85.1 | 81.6 |
| H11 | 79.3 | 79.3 | 79.3 | 100.0 | | 89.7 | 89.7 | 82.8 | 80.5 | 81.6 | 81.6 | 85.1 | 81.6 |
| H8 | 80.5 | 81.6 | 89.7 | 89.7 | 89.7 | | 100.0 | 86.2 | 86.2 | 82.8 | 82.8 | 85.1 | 82.8 |
| H9 | 80.5 | 81.6 | 89.7 | 89.7 | 89.7 | 100.0 | | 86.2 | 86.2 | 82.8 | 82.8 | 85.1 | 82.8 |
| H2 | 81.6 | 82.8 | 75.9 | 82.8 | 82.8 | 86.2 | 86.2 | | 93.1 | 95.4 | 95.4 | 93.1 | 92.0 |
| H3 | 79.3 | 80.5 | 75.9 | 80.5 | 80.5 | 86.2 | 86.2 | 93.1 | | 93.1 | 93.1 | 92.0 | 92.0 |
| H4 | 79.3 | 79.3 | 72.4 | 81.6 | 81.6 | 82.8 | 82.8 | 95.4 | 93.1 | | 100.0 | 90.8 | 94.3 |
| H7 | 79.3 | 79.3 | 72.4 | 81.6 | 81.6 | 82.8 | 82.8 | 95.4 | 93.1 | 100.0 | | 90.8 | 94.3 |
| H5 | 79.3 | 80.5 | 74.7 | 85.1 | 85.1 | 85.1 | 85.1 | 93.1 | 92.0 | 90.8 | 90.8 | | 92.0 |
| H6 | 78.2 | 79.3 | 72.4 | 81.6 | 81.6 | 82.8 | 82.8 | 92.0 | 92.0 | 94.3 | 94.3 | 92.0 | |

VL identity matrix -- all residues

| | | | | | | | | | | | | | |
|---|---|---|---|---|---|---|---|---|---|---|---|---|---|
| WT | | 100.0 | 70.1 | 83.2 | 81.3 | 85.0 | 83.2 | 86.0 | 85.0 | 82.2 | 79.4 | 82.2 | 84.1 |
| N85E | 100.0 | | 70.1 | 83.2 | 81.3 | 85.0 | 83.2 | 86.0 | 85.0 | 82.2 | 79.4 | 82.2 | 84.1 |
| H1 | 70.1 | 70.1 | | 79.4 | 82.2 | 80.4 | 83.2 | 77.6 | 80.4 | 80.4 | 83.2 | 76.6 | 78.5 |
| H10 | 83.2 | 83.2 | 79.4 | | 97.2 | 95.3 | 92.5 | 88.8 | 87.9 | 91.6 | 89.7 | 88.8 | 88.8 |
| H11 | 81.3 | 81.3 | 82.2 | 97.2 | | 92.5 | 95.3 | 86.9 | 86.0 | 89.7 | 92.5 | 87.9 | 86.9 |
| H8 | 85.0 | 85.0 | 80.4 | 95.3 | 92.5 | | 97.2 | 87.9 | 88.8 | 89.7 | 87.9 | 86.9 | 88.8 |
| H9 | 83.2 | 83.2 | 83.2 | 92.5 | 95.3 | 97.2 | | 86.0 | 86.9 | 87.9 | 90.7 | 86.0 | 86.9 |
| H2 | 86.0 | 86.0 | 77.6 | 88.8 | 86.9 | 87.9 | 86.0 | | 95.3 | 95.3 | 92.5 | 90.7 | 90.7 |
| H3 | 85.0 | 85.0 | 80.4 | 87.9 | 86.0 | 88.8 | 86.9 | 95.3 | | 95.3 | 92.5 | 88.8 | 91.6 |
| H4 | 82.2 | 82.2 | 80.4 | 91.6 | 89.7 | 89.7 | 87.9 | 95.3 | 95.3 | | 97.2 | 93.5 | 90.7 |
| H7 | 79.4 | 79.4 | 83.2 | 89.7 | 92.5 | 87.9 | 90.7 | 92.5 | 92.5 | 97.2 | | 93.5 | 89.7 |
| H5 | 82.2 | 82.2 | 76.6 | 88.8 | 87.9 | 86.9 | 86.0 | 90.7 | 88.8 | 93.5 | 93.5 | | 91.6 |
| H6 | 84.1 | 84.1 | 78.5 | 88.8 | 86.9 | 88.8 | 86.9 | 90.7 | 91.6 | 90.7 | 89.7 | 91.6 | |

VL identity matrix -- framework residues

| | | | | | | | | | | | | | |
|---|---|---|---|---|---|---|---|---|---|---|---|---|---|
| WT | | 100.0 | 60.0 | 77.5 | 75.0 | 80.0 | 77.5 | 81.3 | 80.0 | 76.3 | 72.5 | 76.3 | 78.8 |
| N85E | 100.0 | | 60.0 | 77.5 | 75.0 | 80.0 | 77.5 | 81.3 | 80.0 | 76.3 | 72.5 | 76.3 | 78.8 |
| H1 | 60.0 | 60.0 | | 72.5 | 76.3 | 73.8 | 77.5 | 70.0 | 73.8 | 73.8 | 77.5 | 68.8 | 71.3 |
| H10 | 77.5 | 77.5 | 72.5 | | 96.3 | 93.8 | 90.0 | 85.0 | 83.8 | 88.8 | 86.3 | 85.0 | 85.0 |
| H11 | 75.0 | 75.0 | 76.3 | 96.3 | | 90.0 | 93.8 | 82.5 | 81.3 | 86.3 | 90.0 | 83.8 | 82.5 |
| H8 | 80.0 | 80.0 | 73.8 | 93.8 | 90.0 | | 96.3 | 83.8 | 85.0 | 86.3 | 83.8 | 82.5 | 85.0 |
| H9 | 77.5 | 77.5 | 77.5 | 90.0 | 93.8 | 96.3 | | 81.3 | 82.5 | 83.8 | 87.5 | 81.3 | 82.5 |
| H2 | 81.3 | 81.3 | 70.0 | 85.0 | 82.5 | 83.8 | 81.3 | | 93.8 | 93.8 | 90.0 | 87.5 | 87.5 |
| H3 | 80.0 | 80.0 | 73.8 | 83.8 | 81.3 | 85.0 | 82.5 | 93.8 | | 93.8 | 90.0 | 85.0 | 88.8 |
| H4 | 76.3 | 76.3 | 73.8 | 88.8 | 86.3 | 86.3 | 83.8 | 93.8 | 93.8 | | 96.3 | 91.3 | 87.5 |
| H7 | 72.5 | 72.5 | 77.5 | 86.3 | 90.0 | 83.8 | 87.5 | 90.0 | 90.0 | 96.3 | | 91.3 | 86.3 |
| H5 | 76.3 | 76.3 | 68.8 | 85.0 | 83.8 | 82.5 | 81.3 | 87.5 | 85.0 | 91.3 | 91.3 | | 88.8 |
| H6 | 78.8 | 78.8 | 71.3 | 85.0 | 82.5 | 85.0 | 82.5 | 87.5 | 88.8 | 87.5 | 86.3 | 88.8 | |

TABLE 4
EGFR binding complex in the forms of humanized EGFR binding sequence variants (variable
regions H1-H11), His-tagged scFv protein (scFv-6His), recombinant scFv-monoFc monomer
(scFv-monoFc), monoclonal antibody (mAb), bispecific antibody (bispecific), penta-GNC
antibody (pentaGNC), hexa-GNC antibody (hexaGNC), and penta-miniGNC antibody (miniGNC).

| Cetuximab | | | | | | | |
|---|---|---|---|---|---|---|---|
| version | scFv-6His | scFv-monoFc | mAb | Bispecific | PentaGNC | HexaGNC | miniGNC |
| Wild-type | SI-79R1 | SI-79SF1 | SI-79C1 | SI-79X1 | | SI-77H5 | |
| Aglycosylated | | SI-79SF2 | SI-79C5 | SI-79X5 | | | |
| (N85E) | | | | | | | |
| H1 | SI-79R2 | SI-79SF3 | | | SI-55P3 | | SI-68P7 |
| | | | | | SI-55P4 | | |
| H2 | | SI-79SF12 | | | | | |
| H3 | | SI-79SF10 | | | | | |
| H4 | | SI-79SF4 | SI-79C2 | SI-79X2 | SI-79P2 | | SI-79P1 |
| | | | | | SI-79P3 | | |
| H5 | | SI-79SF11 | | | | | |
| H6 | | SI-79SF9 | | | | | |
| H7 | | SI-79SF6 | SI-79C3 | SI-79X3 | SI-55P9 | SI-55H11 | SI-68P13 |
| | | | | | SI-77P1 | SI-77H4 | SI-68P17 |
| H8 | | SI-79SF5 | | | | | |
| H9 | | SI-79SF7 | | | | | |
| H10 | | SI-79SF8 | | | | | |
| H11 | | SI-79SF13 | | | | | |

TABLE 5
Characterization of His-tagged humanized anti-EGFR scFv.

| | | | | DLS | Gdn | huEGFR |
|---|---|---|---|---|---|---|
| | | Titer | aSEC | Tm | EC50 | KD |
| Protein | αhuEGFR | (ug/ml) | % POI | (° C.) | (M) | (nM) |
| SI-79R1 | Cetuximab | 13.8 | 39.5 | 47.87 | 1.42 | 2.08 |
| SI-79R2 | H1 | 108.2 | 84.5 | 71.37 | 2.92 | 12.0 |

TABLE 6
Biophysical properties of cetuximab-derived scFv-monoFc proteins. Values
are average and standard deviation of two independent experiments.

| | Titer | aSEC | DLS Temp | EGFR KD | ka [1/(M · | kd (1/ |
|---|---|---|---|---|---|---|
| Version | (μg/ml) | % POI | (° C.) | (nM) | s)] ×10⁵ | s) ×10⁻³ |
| WT | 163 ± 39 | 93.6 ± 2.6 | 47.2 ± 1.0 | 3.18 ± 0.18 | 4.44 ± 1.13 | 1.42 ± 0.44 |
| N85E | 116 ± 6 | 94.9 ± 3.1 | 44.5 ± 1.3 | 3.16 ± 0.35 | 4.44 ± 0.91 | 1.42 ± 0.44 |
| H1 | 309 ± 26 | 91.5 ± 7.1 | 46.2 ± 0.9 | 13.1 ± 1.2 | 7.18 ± 1.68 | 9.27 ± 1.34 |
| H2 | 411 ± 59 | 86.8 ± 2.1 | 52.4 ± 2.1 | 3.08 ± 0.68 | 4.81 ± 0.91 | 1.51 ± 0.61 |
| H3 | 418 ± 8 | 88.1 ± 2.7 | 52.3 ± 2.0 | 3.45 ± 0.81 | 5.04 ± 0.92 | 1.77 ± 0.73 |
| H4 | 412 ± 5 | 82.6 ± 5.5 | 49.7 ± 1.4 | 3.05 ± 0.62 | 4.50 ± 0.54 | 1.39 ± 0.44 |
| H5 | 394 ± 23 | 85.6 ± 6.2 | 50.2 ± 1.5 | 2.27 ± 0.38 | 4.74 ± 1.31 | 1.10 ± 0.48 |
| H6 | 372 ± 73 | 88.0 ± 3.4 | 52.2 ± 2.0 | 3.42 ± 0.36 | 4.76 ± 1.33 | 1.65 ± 0.62 |
| H7 | 506 ± 14 | 92.7 ± 2.3 | 50.5 ± 2.4 | 3.14 ± 0.53 | 4.47 ± 0.97 | 1.43 ± 0.54 |
| H8 | 220 ± 3 | 92.2 ± 0.0 | 51.3 ± 1.1 | 6.79 ± 1.35 | 5.71 ± 0.78 | 3.93 ± 1.30 |
| H9 | 211 ± 22 | 97.4 ± 0.9 | 48.7 ± 1.3 | 6.92 ± 1.58 | 5.63 ± 0.67 | 3.95 ± 1.35 |
| H10 | 308 ± 3 | 83.7 ± 1.9 | 52.6 ± 1.3 | 6.77 ± 2.10 | 6.65 ± 0.73 | 4.58 ± 1.89 |
| H11 | 376 ± 12 | 88.6 ± 2.4 | 53.0 ± 1.3 | 6.74 ± 1.61 | 6.61 ± 1.11 | 4.54 ± 1.81 |

TABLE 7
Gradient method for cation exchange separation
of αEGFR and αCD3 antibodies.

| | Time (min) | % B |
|---|---|---|
| Gradient | 0.00 | 0.0 |
| | 3.00 | 0.0 |
| | 5.00 | 25.0 |
| | 20.00 | 70.0 |
| | 20.01 | 100.0 |
| | 25.00 | 100.0 |
| | 25.01 | 0.0 |
| | 35.00 | 0.0 |

TABLE 8
Biophysical properties of cetuximab-derived monoclonal antibodies. Values
are average and standard deviation of two independent experiments.

| | Titer | aSEC | DLS Temp | EGFR KD | ka [1/(M · | kd (1/ | *EC50 |
|---|---|---|---|---|---|---|---|
| Version | (μg/ml) | % POI | (° C.) | (nM) | s)] ×105 | s) ×10−4 | (pM) |
| WT | 203 ± 13 | 99.9 ± 0.2 | 68.3 ± 0.1 | 2.68 ± 0.07 | 3.46 ± 0.27 | 9.26 ± 0.48 | 24.6 |
| | | | | | | | [10.4-58.4] |
| N85E | 209 ± 9 | 99.4 ± 0.3 | 68.1 ± 0.9 | 2.46 ± 0.28 | 3.26 ± 0.41 | 7.96 ± 0.09 | 30.7 |
| | | | | | | | [11.3-83.8] |
| H7 | 249 ± 1 | 99.1 ± 0.4 | 72.5 ± 1.2 | 2.41 ± 0.15 | 3.37 ± 0.20 | 8.12 ± 0.05 | 20.3 |
| | | | | | | | [13.9-29.9] |

*EC50 value for depleting EGFR+ BxPC3 cells using αCD3 × αEGFR bispecific antibody, with 95% confidence interval shown in brackets.

TABLE 9
Characterization of the penta-GNC antibodies comprising a humanized
anti-EGFR scFv domain or a humanized anti-EGFR Fab region.

| | αEGFR | GNC | | Titer | aSEC | huEGFR K_{D} | TDCC EC50 |
|---|---|---|---|---|---|---|---|
| GNC Ab | Variant | Domain | GNC binding domains | (μg/ml) | % POI | (nM) | (pM) |
| SI-55P3 | H1 | scFv (1) | αCD3 (2), αPD-L1 (3), | 175.9 | 86.13 | 18.1 | nd |
| | | | α4-1BB (4), αCD19 (6) | | | | |
| SI-55P4 | H1 | scFv (1) | αCD3 (2), αPD-L1 (3), | 59.6 | 93.22 | nd | nd |
| | | | 41BBLT (4), αCD19 (6) | | | | |
| SI-79P2 | H4 | scFv (1) | αCD3 (2), αPD-L1 (3), | 68.9 | 87.74 | 4.30 | nd |
| | | | α4-1BB (4), αCD19 (6) | | | | |
| SI-79P3 | H4 | scFv (1) | αCD3 (2), αPD-L1 (3), | 70.3 | 92.80 | 5.21 | nd |
| | | | 41BBLT (4), αCD19 (6) | | | | |
| SI-55P9 | H7 | scFv (1) | αCD3 (2), αPD-L1 (3), | 118.0 | 85.96 | 6.88 | 0.5727 |
| | | | α4-1BB (4), αCD19 (6) | | | | |
| SI-77P1 | H7 | Fab (2) | αCD3 (1), αPD-L1 (3), | 98.1 | 77.94 | 2.17 | 0.0826 |
| | | | α4-1BB (4), αCD19 (6) | | | | |

TABLE 10
Octet binding analysis of EGFR binding complex.

| | Octet Binding Kinetics | | |
|---|---|---|---|
| Protein | K_{D} (nM) | kₐ (M⁻¹s⁻¹) | k_{d} (s⁻¹) |
| SI-79R1 | 2.08 | 657000 | 0.00137 |
| SI-79R2 | 12.00 | 894000 | 0.01070 |
| SI-55P3 | 18.10 | 601000 | 0.01090 |
| SI-79P2 | 4.30 | 328000 | 0.00141 |
| SI-79P3 | 5.21 | 284000 | 0.00148 |
| SI-55P9 | 6.88 | 297000 | 0.00204 |
| SI-77P1 | 2.17 | 212000 | 0.00046 |
| SI-77H5 | 3.39 | 286000 | 0.00097 |
| SI-55H11 | 4.65 | 268000 | 0.00125 |
| SI-77H4 | 3.29 | 214000 | 0.00122 |
| SI-79P1 | 3.03 | 425000 | 0.00158 |
| SI-68P13 | 5.64 | 389000 | 0.00219 |
| SI-68P17 | 2.46 | 259000 | 0.00064 |

TABLE 11
Characterization of the hexaGNC antibodies comprising a humanized
anti-EGFR scFv domain or a humanized anti-EGFR Fab region.

| | αEGFR | GNC | | Titer | aSEC | huEGFR K_{D} | TDCC EC50 |
|---|---|---|---|---|---|---|---|
| GNC Ab | Variant | Domain | GNC binding domains | (μg/ml) | % POI | (nM) | (pM) |
| SI-77H5 | Cetuximab | Fab (2) | αCD3 (1), αPD-L1 (3), α4-1BB | 35.0 | 72.02 | 3.39 | nd |
| | | | (4), αHER3 (5), αCD19 (6) | | | | |
| SI-77H4 | H7 | Fab (2) | αCD3 (1), αPD-L1 (3), α4-1BB | 61.1 | 77.25 | 3.29 | nd |
| | | | (4), αHER3 (5), αCD19 (6) | | | | |
| SI-55H11 | H7 | scFv (1) | αCD3 (2), aPD-L1 (3), α4-1BB | 30.0 | 84.42 | 4.65 | 0.09387 |
| | | | (4), αHER3 (5), αCD19 (6) | | | | |

TABLE 12
Characterization of the penta-miniGNC antibodies comprising a humanized
anti-EGFR scFv domain or a humanized anti-EGFR Fab domain.

| | αEGFR | miniGNC | | Titer | aSEC | huEGFR K_{D} | TDCC EC50 |
|---|---|---|---|---|---|---|---|
| GNC Ab | Variant | Domain | GNC binding domains | (μg/ml) | % POI | (nM) | (pM) |
| SI-68P7 | H1 | scFv (1) | αCD19 (2), αCD3 (3), | 85.2 | 84.60 | nd | nd |
| | | | 41BBLT (4), αPD-L1 (5) | | | | |
| SI-79P1 | H4 | scFv (1) | αCD19 (2), αCD3 (3), α4- | 75.3 | 72.61 | 3.03 | nd |
| | | | 1BB (4), αPD-L1 (5) | | | | |
| SI-68P13 | H7 | scFv (1) | αCD19 (2), αCD3 (3), α4- | 76.6 | 87.69 | 5.64 | 0.259 |
| | | | 1BB (4), αPD-L1 (5) | | | | |
| SI-68P17 | H7 | Fab (3) | αCD3 (1), αCD19 (2), α4- | 62.0 | 54.87 | 2.46 | 0.322 |
| | | | 1BB (4), αPD-L1 (5) | | | | |

SEQUENCE LISTING
Sequences of humanized EGFR binding sequence variants (H1-H11)

| Sequence | Amino acid seq. ID | Nucleotide seq. ID |
|---|---|---|
| H1 VH | 1 | 2 |
| H1 VL | 3 | 4 |
| H2 VH | 5 | 6 |
| H2 VL | 7 | 8 |
| H3 VH | 9 | 10 |
| H3 VL | 11 | 12 |
| H4 VH | 13 | 14 |
| H4 VL | 15 | 16 |
| H5 VH | 17 | 18 |
| H5 VL | 19 | 20 |
| H6 VH | 21 | 22 |
| H6 VL | 23 | 24 |
| H7 VH | 25 | 26 |
| H7 VL | 27 | 28 |
| H8 VH | 29 | 30 |
| H8 VL | 31 | 32 |
| H9 VH | 33 | 34 |
| H9 VL | 35 | 36 |
| H10 VH | 37 | 38 |
| H10 VL | 39 | 40 |
| H11 VH | 41 | 42 |
| H11 VL | 43 | 44 |

Sequences of antibody constant regions, linker motifs, and tags

| Sequence | Amino acid seq. ID | Nucleotide seq. ID |
|---|---|---|
| MonoFc | 45 | 46 |
| Human IgG1 | 47 | 48 |
| Human C kappa | 49 | 50 |
| (G4S)4 linker | 51 | 52 |
| His tag | 53 | 54 |

Sequences of αEGFR scFv-His proteins

| Sequence | Amino acid seq. ID | Nucleotide seq. ID |
|---|---|---|
| SI-79R1 | 55 | 56 |
| SI-79R2 | 57 | 58 |

Sequences of αEGFR scFv-monoFc proteins

| Sequence | Amino acid seq. ID | Nucleotide seq. ID |
|---|---|---|
| SI-79SF1 | 59 | 60 |
| SI-79SF2 | 61 | 62 |
| SI-79SF3 | 63 | 64 |
| SI-79SF4 | 65 | 66 |
| SI-79SF5 | 67 | 68 |
| SI-79SF6 | 69 | 70 |
| SI-79SF7 | 71 | 72 |
| SI-79SF8 | 73 | 74 |
| SI-79SF9 | 75 | 76 |
| SI-79SF10 | 77 | 78 |
| SI-79SF11 | 79 | 80 |
| SI-79SF12 | 81 | 82 |
| SI-79SF13 | 83 | 84 |

Sequences of pentaGNC proteins containing a humanized EGFR binding domain

| Sequence | Amino acid seq. ID | Nucleotide seq. ID |
|---|---|---|
| SI-55P3 HC | 85 | 86 |
| SI-55P3 LC | 87 | 88 |
| SI-55P4 HC | 89 | 90 |
| SI-55P4 LC | 91 | 92 |
| SI-79P2 HC | 93 | 94 |
| SI-79P2 LC | 95 | 96 |
| SI-79P3 HC | 97 | 98 |
| SI-79P3 LC | 99 | 100 |
| SI-55P9 HC | 101 | 102 |
| SI-55P9 LC | 103 | 104 |
| SI-77P1 HC | 105 | 106 |
| SI-77P1 LC | 107 | 108 |

Sequences of hexaGNC proteins containing a humanized EGFR binding domain

| Sequence | Amino acid seq. ID | Nucleotide seq. ID |
|---|---|---|
| SI-77H5 HC | 109 | 110 |
| SI-77H5 LC | 111 | 112 |
| SI-55H11 HC | 113 | 114 |
| SI-55H11 LC | 115 | 116 |
| SI-77H4 HC | 117 | 118 |
| SI-77H4 LC | 119 | 120 |

Sequences of penta-miniGNC proteins containing a humanized EGFR binding domain

| Sequence | Amino acid seq. ID | Nucleotide seq. ID |
|---|---|---|
| SI-68P7 HC | 121 | 122 |
| SI-68P7 LC | 123 | 124 |
| SI-79P1 HC | 125 | 126 |
| SI-79P1 LC | 127 | 128 |
| SI-68P13 HC | 129 | 130 |
| SI-68P13 LC | 131 | 132 |
| SI-68P17 HC | 133 | 134 |
| SI-68P17 LC | 135 | 136 |

Sequences of αEGFR mAbs and αCD3 mAbs

| Sequence | Amino acid seq. ID | Nucleotide seq. ID |
|---|---|---|
| SI-79C1 HC | 137 | 138 |
| SI-79C1 LC | 139 | 140 |
| SI-79C2 HC | 141 | 142 |
| SI-79C2 LC | 143 | 144 |
| SI-79C3 HC | 141 | 142 |
| SI-79C3 LC | 149 | 150 |
| SI-79C5 HC | 151 | 152 |
| SI-79C5 LC | 139 | 140 |
| SI-9C21 HC | 145 | 146 |
| SI-9C21 LC | 147 | 148 |

Sequences of αEGFR x αCD3 bispecific antibodies

| Sequence | Amino acid seq. ID | Nucleotide seq. ID |
|---|---|---|
| SI-79X1 HC1 | 137 | 138 |
| SI-79X1 HC2 | 145 | 146 |
| SI-79X1 LC1 | 139 | 140 |
| SI-79X1 LC2 | 147 | 148 |
| SI-79X2 HC1 | 141 | 142 |
| SI-79X2 HC2 | 145 | 146 |
| SI-79X2 LC1 | 143 | 144 |
| SI-79X2 LC2 | 147 | 148 |
| SI-79X3 HC1 | 141 | 142 |
| SI-79X3 HC2 | 145 | 146 |
| SI-79X3 LC1 | 149 | 150 |
| SI-79X3 LC2 | 147 | 148 |
| SI-79X5 HC1 | 151 | 152 |
| SI-79X5 HC2 | 145 | 146 |
| SI-79X5 LC1 | 139 | 140 |
| SI-79X5 LC2 | 147 | 148 |

>Sequence ID 1: Cetuximab H1 VH amino acid sequence
EVQLVESGGGLVQPGGSLRLSCKVSGFSLTNYGVHWVRQAPGKGLEWVGVIWSGGNTDYNTPFTSRFTISRDTSKNTVYLQMNSLR
AEDTAVYYCARALTYYDYEFAYWGQGTLVTVSS
>Sequence ID 2: Cetuximab H1 VH nucleotide sequence
GAAGTTCAGCTGGTGGAATCCGGCGGAGGATTGGTTCAACCTGGCGGCTCTCTGAGACTGTCCTGTAAGGTGTCTGGCTTCTCCCT
GACCAACTACGGCGTGCACTGGGTCCGACAGGCACCTGGAAAAGGACTGGAATGGGTCGGAGTGATTTGGAGCGGCGGCAACACCG
ACTACAACACCCCTTTCACCAGCCGGTTCACCATCTCTCGGGACACCTCCAAGAACACCGTGTACCTGCAGATGAACTCCCTGAGA
GCCGAGGACACCGCCGTGTACTATTGTGCTAGAGCCCTGACCTACTATGACTACGAGTTCGCCTATTGGGGCCAGGGAACCCTGGT
CACAGTCTCCTCT
>Sequence ID 3: Cetuximab H1 VL amino acid sequence
EIVMTQSPSTLSASVGDRVIITCRASQSIGTNIHWYQQKPGKAPKLLIYYASESISGIPSRFSGSGSGAEFTLTISSLQPDDFATY
YCQQNNNWPTTFGQGTKLTVL
>Sequence ID 4: Cetuximab H1 VL nucleotide sequence
GAGATCGTGATGACCCAGTCTCCTTCCACACTGTCCGCCTCTGTGGGCGACAGAGTGATCATCACCTGTAGAGCCAGCCAGTCCAT
CGGCACCAACATCCACTGGTATCAGCAGAAGCCTGGCAAGGCCCCTAAGCTGCTGATCTACTACGCCTCCGAGTCTATCAGCGGCA
TCCCCTCCAGATTCTCCGGCTCTGGATCTGGCGCTGAGTTTACCCTGACAATCTCCAGCCTGCAGCCTGACGACTTCGCCACCTAC
TACTGCCAGCAGAACAACAACTGGCCCACCACCTTTGGCCAGGGCACCAAACTGACAGTTCTT
>Sequence ID 5: Cetuximab H2 VH amino acid sequence
QVQLQQSGPGLVKPSQTLSITCTVSGFSLTNYGVHWIRQAPGKGLEWLGVIWSGGNTDYNTPFTSRFTISKDNSKNQVYFKLRSLR
AEDTAIYYCARALTYYDYEFAYWGQGTLVTVSS
>Sequence ID 6: Cetuximab H2 VH nucleotide sequence
CAAGTTCAGTTGCAGCAGTCTGGCCCTGGCCTCGTGAAGCCTTCTCAGACCCTGTCTATCACCTGTACCGTGTCCGGCTTCTCCCT
GACCAATTACGGCGTGCACTGGATCAGACAGGCCCCTGGCAAAGGACTGGAATGGCTGGGAGTGATTTGGAGCGGCGGCAACACCG
ACTACAACACCCCTTTCACCAGCCGGTTCACCATCTCCAAGGACAACTCCAAGAACCAGGTGTACTTCAAGCTGCGGTCCCTGAGA
GCCGAGGACACCGCCATCTACTACTGTGCTAGAGCCCTGACCTACTACGACTACGAGTTCGCCTATTGGGGCCAGGGCACACTGGT
CACAGTCTCTTCT
>Sequence ID 7: Cetuximab H2 VL amino acid sequence
EIVLTQSPSILSVSPGERATFSCRASQSIGTNIHWYQQRPGKPPRLLIKYASESISGIPSRFSGSGSGTEFTLTITSVQSEDIAVY
YCQQNNNWPTTFGPGTKLELK
>Sequence ID 8: Cetuximab H2 VL nucleotide sequence
GAGATCGTGCTGACCCAGTCTCCTTCCATCCTGTCTGTGTCTCCCGGCGAGAGAGCCACCTTCAGCTGTAGAGCCTCTCAGTCCAT
CGGCACCAACATCCACTGGTATCAGCAGCGGCCTGGCAAGCCTCCTCGGCTGCTGATTAAGTACGCCTCCGAGTCTATCAGCGGCA
TCCCCTCCAGATTCTCCGGCTCTGGATCTGGCACCGAGTTCACCCTGACCATCACCTCCGTGCAGTCCGAGGATATCGCCGTGTAC
TACTGCCAGCAGAACAACAACTGGCCCACCACCTTTGGACCCGGCACCAAGCTGGAATTGAAA
>Sequence ID 9: Cetuximab H3 VH amino acid sequence
QVQLVQSGPGLVKPSQTLSLTCTVSGFSLTNYGVHWIRQAPGKGLEWLGVIWSGGNTDYNTPFTSRFTITKDNSKNQVYFKLRSVR
SEDTAVYYCARALTYYDYEFAYWGQGTLVTVSS
>Sequence ID 10: Cetuximab H3 VH nucleotide sequence
CAAGTTCAGCTGGTTCAGTCTGGCCCTGGCCTCGTGAAGCCTTCTCAGACCCTGTCTCTGACCTGCACCGTGTCTGGCTTCTCCCT
GACCAATTACGGCGTGCACTGGATCAGACAGGCCCCTGGCAAAGGACTGGAATGGCTGGGAGTGATTTGGAGCGGCGGCAACACCG
ACTACAACACCCCTTTCACCAGCCGGTTCACCATCACCAAGGACAACTCCAAGAACCAGGTGTACTTCAAGCTGAGATCCGTGCGG
AGCGAGGACACCGCCGTGTACTATTGTGCTAGAGCCCTGACCTACTACGACTACGAGTTCGCCTATTGGGGCCAGGGCACACTGGT
CACAGTCTCTTCT
>Sequence ID 11: Cetuximab H3 VL amino acid sequence
EIVLTQSPSILSVSPGERVTFSCRASQSIGTNIHWYQQRPGKPPRLLIKYASESISGIPARFSGSGSGTEFTLTISSVQSEDFATY
YCQQNNNWPTTFGPGTKLELK
>Sequence ID 12: Cetuximab H3 VL nucleotide sequence
GAGATCGTGCTGACCCAGTCTCCTTCCATCCTGTCTGTGTCTCCCGGCGAGAGAGTGACCTTCAGCTGTAGAGCCTCTCAGTCCAT
CGGCACCAACATCCACTGGTATCAGCAGCGGCCTGGCAAGCCTCCTCGGCTGCTGATTAAGTACGCCTCCGAGTCCATCAGCGGCA
TCCCTGCCAGATTTTCCGGCTCTGGCTCTGGCACCGAGTTTACCCTGACCATCTCCTCCGTGCAGTCCGAGGATTTCGCCACCTAC
TACTGCCAGCAGAACAACAACTGGCCCACCACCTTTGGACCCGGCACCAAGCTGGAATTGAAA
>Sequence ID 13: Cetuximab H4 VH amino acid sequence
QVQLQQSGPGLVKPSETLSITCTVSGFSLTNYGVHWIRQAPGKGLEWLGVIWSGGNTDYNTPFTSRFTITKDNSKNQVYFKLRSVR
ADDTAIYYCARALTYYDYEFAYWGQGTLVTVSS
>Sequence ID 14: Cetuximab H4 VH nucleotide sequence
CAAGTTCAGTTGCAGCAGTCTGGCCCTGGCCTGGTCAAGCCTTCTGAGACACTGTCCATCACCTGTACCGTGTCCGGCTTCTCCCT
GACCAATTACGGCGTGCACTGGATCAGACAGGCCCCTGGCAAAGGACTGGAATGGCTGGGAGTGATTTGGAGCGGCGGCAACACCG
ACTACAACACCCCTTTCACCAGCCGGTTCACCATCACCAAGGACAACTCCAAGAACCAGGTGTACTTCAAGCTGCGGAGCGTGCGG
GCTGATGACACCGCCATCTACTACTGTGCTCGGGCCCTGACCTACTACGACTACGAGTTTGCTTACTGGGGCCAGGGCACCCTGGT
CACAGTTTCTTCT
>Sequence ID 15: Cetuximab H4 VL amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLELK
>Sequence ID 16: Cetuximab H4 VL nucleotide sequence
GAGATCGTGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAGCCCGGCAAGCCTCCTCGGCTGCTGATTAAGTACGCCTCCGAGTCCATCAGCGGCA
TCCCTGACAGATTCTCCGGCTCTGGCTCTGGCACCGAGTTTACCCTGACCATCTCCTCCGTGCAGTCCGAGGATTTCGCCGTGTAC
TACTGCCAGCAGAACAACAACTGGCCCACCACCTTTGGACCCGGCACCAAGCTGGAATTGAAA
>Sequence ID 17: Cetuximab H5 VH amino acid sequence
QVQLLQSGPGLVKPSETLSLTCTVSGFSLTNYGVHWIRQAPGKGLEWIGVIWSGGNTDYNTPFTSRFTISKDNSKNQVYFKLRSLT
SEDTAIYYCARALTYYDYEFAYWGQGTLVTVSS
>Sequence ID 18: Cetuximab H5 VH nucleotide sequence
CAAGTTCAGCTGTTGCAGTCTGGCCCTGGCCTGGTCAAGCCTTCCGAAACACTGTCTCTGACCTGCACCGTGTCCGGCTTCTCCCT
GACCAATTATGGCGTGCACTGGATCAGACAGGCCCCTGGCAAAGGCCTGGAATGGATCGGAGTGATTTGGAGCGGCGGCAACACCG
ACTACAACACCCCTTTCACCAGCCGGTTCACCATCTCCAAGGACAACTCCAAGAACCAGGTGTACTTCAAGCTGCGGTCCCTGACC
TCTGAGGACACCGCCATCTACTACTGCGCTAGAGCCCTGACCTACTACGACTACGAGTTCGCCTATTGGGGCCAGGGCACACTGGT
CACAGTCTCTTCT
>Sequence ID 19: Cetuximab H5 VL amino acid sequence
EIQLTQSPSTLSVSPGERATFSCRASQSIGTNIHWYQQKTGQPPRLLIKYASESISGIPDRFSGSGSGTEFTLSITSVQSEDFAVY
YCQQNNNWPTTFGPGTKLEIL
>Sequence ID 20: Cetuximab H5 VL nucleotide sequence
GAGATCCAGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAAACCGGCCAGCCTCCTCGGCTGCTGATTAAGTACGCCTCCGAGTCCATCAGCGGCA
TCCCTGACAGATTCTCCGGCTCTGGCTCTGGCACCGAGTTCACCCTGTCTATCACCTCCGTGCAGTCCGAGGACTTCGCCGTGTAC
TACTGCCAGCAGAACAACAACTGGCCCACCACCTTTGGACCCGGCACCAAGCTGGAAATTCTT
>Sequence ID 21: Cetuximab H6 VH amino acid sequence
QVQLLQSGPGLVKPSETLSITCTVSGFSLTNYGVHWIRQAPGKGLEWIGVIWSGGNTDYNTPFTSRFTITKDNSKNQVFFKLRSVR
AEDTALYYCARALTYYDYEFAYWGQGTLVTVSS
>Sequence ID 22: Cetuximab H6 VH nucleotide sequence
CAAGTTCAGCTGTTGCAGTCTGGCCCTGGCCTGGTCAAGCCTTCCGAGACACTGTCCATCACCTGTACCGTGTCCGGCTTCTCCCT
GACCAATTACGGCGTGCACTGGATCAGACAGGCCCCTGGCAAAGGCCTGGAATGGATCGGAGTGATTTGGAGCGGCGGCAACACCG
ACTACAACACCCCTTTCACCAGCCGGTTCACCATCACCAAGGACAACTCCAAGAACCAGGTGTTCTTCAAGCTGCGGAGCGTGCGC
GCTGAGGATACCGCTCTGTACTATTGCGCTCGGGCCCTGACCTACTACGACTACGAGTTTGCTTACTGGGGCCAGGGAACCCTGGT
CACCGTTTCTTCT
>Sequence ID 23: Cetuximab H6 VL amino acid sequence
EIVLTQSPSTLSVSPGERVSFSCRASQSIGTNIHWYQQRTGQPPRLLIKYASESISGIPARFSGSGSGTEFTLTITSVQSEDFAVY
YCQQNNNWPTTFGQGTKLDIK
>Sequence ID 24: Cetuximab H6 VL nucleotide sequence
GAGATCGTGCTGACCCAGTCTCCTTCCACACTGTCTGTGTCTCCCGGCGAGAGAGTGTCCTTCAGCTGTAGAGCCTCTCAGTCCAT
CGGCACCAACATCCACTGGTATCAGCAGAGAACCGGCCAGCCTCCTCGGCTGCTGATTAAGTACGCCTCCGAGTCCATCAGCGGCA
TCCCTGCCAGATTTTCCGGCTCTGGCTCTGGCACCGAGTTCACCCTGACCATCACCTCCGTGCAGTCTGAGGACTTCGCCGTGTAC
TACTGCCAGCAGAACAACAACTGGCCCACCACCTTTGGCCAGGGCACCAAGCTGGATATCAAA
>Sequence ID 25: Cetuximab H7 VH amino acid sequence
QVQLQQSGPGLVKPSETLSITCTVSGFSLTNYGVHWIRQAPGKGLEWLGVIWSGGNTDYNTPFTSRFTITKDNSKNQVYFKLRSVR
ADDTAIYYCARALTYYDYEFAYWGQGTLVTVSS
>Sequence ID 26: Cetuximab H7 VH nucleotide sequence
CAAGTTCAGTTGCAGCAGTCTGGCCCTGGCCTGGTCAAGCCTTCTGAGACACTGTCCATCACCTGTACCGTGTCCGGCTTCTCCCT
GACCAATTACGGCGTGCACTGGATCAGACAGGCCCCTGGCAAAGGACTGGAATGGCTGGGAGTGATTTGGAGCGGCGGCAACACCG
ACTACAACACCCCTTTCACCAGCCGGTTCACCATCACCAAGGACAACTCCAAGAACCAGGTGTACTTCAAGCTGCGGAGCGTGCGG
GCTGATGACACCGCCATCTACTACTGTGCTCGGGCCCTGACCTACTACGACTACGAGTTTGCTTACTGGGGCCAGGGCACCCTGGT
CACAGTTTCTTCT
>Sequence ID 27: Cetuximab H7 VL amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVL
>Sequence ID 28: Cetuximab H7 VL nucleotide sequence
GAGATCGTGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAGCCCGGCAAGCCTCCTCGGCTGCTGATTAAGTACGCCTCCGAGTCCATCAGCGGCA
TCCCTGACAGATTCTCCGGCTCTGGCTCTGGCACCGAGTTTACCCTGACCATCTCCTCCGTGCAGTCCGAGGATTTCGCCGTGTAC
TACTGCCAGCAGAACAACAACTGGCCCACCACCTTTGGACCCGGCACCAAGCTGACAGTTCTT
>Sequence ID 29: Cetuximab H8 VH amino acid sequence
QVQLVESGPGLVQPSGSLSLTCTVSGFSLTNYGVHWVRQAPGKGLEWVGVIWSGGNTDYNTPFTSRFTISKDNSKNQVYLKMNSLR
AEDTAVYYCARALTYYDYEFAYWGQGTLVTVSS
>Sequence ID 30: Cetuximab H8 VH nucleotide sequence
CAAGTTCAGCTGGTGGAATCTGGCCCTGGCCTGGTTCAGCCTTCTGGCTCTCTGTCTCTGACCTGCACCGTGTCTGGCTTCTCCCT
GACCAATTACGGCGTGCACTGGGTTCGACAGGCTCCAGGCAAAGGACTGGAATGGGTCGGAGTGATTTGGAGCGGCGGCAACACCG
ACTACAACACCCCTTTCACCAGCCGGTTCACCATCTCCAAGGACAACTCCAAGAACCAGGTGTACCTGAAGATGAACTCCCTGAGA
GCCGAGGACACCGCTGTGTATTACTGTGCTAGAGCCCTGACCTACTACGACTACGAGTTCGCCTATTGGGGCCAGGGAACCCTGGT
CACAGTCTCCTCT
>Sequence ID 31: Cetuximab H8 VL amino acid sequence
DIVLTQSPSSLSVSPGERVTISCRASQSIGTNIHWYQQKPGQAPRLLIKYASESISGIPSRFSGSGSGTDFTLTISSVESEDFAVY
YCQQNNNWPTTFGQGTKLEIK
>Sequence ID 32: Cetuximab H8 VL nucleotide sequence
GACATCGTGCTGACCCAGTCTCCATCCAGCCTGTCTGTGTCTCCTGGCGAGAGAGTGACCATCTCTTGCCGGGCCTCTCAGAGCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCAGCTCCGTGGAATCCGAGGACTTCGCCGTGTAC
TACTGCCAGCAGAACAACAACTGGCCCACCACCTTTGGCCAGGGCACCAAGCTGGAAATCAAA
>Sequence ID 33: Cetuximab H9 VH amino acid sequence
QVQLVESGPGLVQPSGSLSLTCTVSGFSLTNYGVHWVRQAPGKGLEWVGVIWSGGNTDYNTPFTSRFTISKDNSKNQVYLKMNSLR
AEDTAVYYCARALTYYDYEFAYWGQGTLVTVSS
>Sequence ID 34: Cetuximab H9 VH nucleotide sequence
CAAGTTCAGCTGGTGGAATCTGGCCCTGGCCTGGTTCAGCCTTCTGGCTCTCTGTCTCTGACCTGCACCGTGTCTGGCTTCTCCCT
GACCAATTACGGCGTGCACTGGGTTCGACAGGCTCCAGGCAAAGGACTGGAATGGGTCGGAGTGATTTGGAGCGGCGGCAACACCG
ACTACAACACCCCTTTCACCAGCCGGTTCACCATCTCCAAGGACAACTCCAAGAACCAGGTGTACCTGAAGATGAACTCCCTGAGA
GCCGAGGACACCGCTGTGTATTACTGTGCTAGAGCCCTGACCTACTACGACTACGAGTTCGCCTATTGGGGCCAGGGAACCCTGGT
CACAGTCTCCTCT
>Sequence ID 35: Cetuximab H9 VL amino acid sequence
DIVLTQSPSSLSVSPGERVTISCRASQSIGTNIHWYQQKPGQAPRLLIKYASESISGIPSRFSGSGSGTDFTLTISSVESEDFAVY
YCQQNNNWPTTFGQGTKLTVL
>Sequence ID 36: Cetuximab H9 VL nucleotide sequence
GACATCGTGCTGACCCAGTCTCCATCCAGCCTGTCTGTGTCTCCTGGCGAGAGAGTGACCATCTCTTGCCGGGCCTCTCAGAGCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCAGCTCCGTGGAATCCGAGGACTTCGCCGTGTAC
TACTGCCAGCAGAACAACAACTGGCCCACCACCTTTGGCCAGGGCACCAAGCTGACAGTTCTT
>Sequence ID 37: Cetuximab H10 VH amino acid sequence
QVQLQESGPGLVKPSESLSLTCTVSGFSLTNYGVHWVRQPPGKGLEWIGVIWSGGNTDYNTPFTSRVTISKDNSKNQVSLKMNSLT
AADTAVYYCARALTYYDYEFAYWGQGTLVTVSS
>Sequence ID 38: Cetuximab H10 VH nucleotide sequence
CAAGTTCAGCTGCAAGAGTCTGGCCCTGGCCTGGTCAAGCCTTCCGAATCTCTGTCTCTGACCTGCACCGTGTCCGGCTTCTCCCT
GACCAATTATGGCGTGCACTGGGTTCGACAGCCTCCAGGCAAAGGCCTGGAATGGATCGGAGTGATTTGGAGCGGCGGCAACACCG
ACTACAACACCCCTTTCACCTCTAGAGTGACCATCAGCAAGGACAACTCCAAGAACCAGGTGTCCCTGAAGATGAACAGCCTGACC
GCTGCCGACACCGCTGTGTACTATTGTGCTAGAGCCCTGACCTACTACGACTACGAGTTCGCCTATTGGGGCCAGGGAACCCTGGT
CACAGTCTCCTCT
>Sequence ID 39: Cetuximab H10 VL amino acid sequence
DIVLTQSPATLSVSPGERATLSCRASQSIGTNIHWYQQKPGQAPRLLIKYASESISGIPSRFSGSGSGTDFTLTISSVQSEDFAVY
YCQQNNNWPTTFGQGTKLEIK
>Sequence ID 40: Cetuximab H10 VL nucleotide sequence
GACATCGTGCTGACCCAGTCTCCAGCCACACTGAGTGTGTCTCCAGGCGAGAGAGCTACCCTGTCCTGTAGAGCCTCTCAGTCCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCTCCTCCGTGCAGTCCGAGGATTTCGCCGTGTAC
TACTGCCAGCAGAACAACAACTGGCCCACCACCTTTGGCCAGGGCACCAAGCTGGAAATCAAA
>Sequence ID 41: Cetuximab H11 VH amino acid sequence
QVQLQESGPGLVKPSESLSLTCTVSGFSLTNYGVHWVRQPPGKGLEWIGVIWSGGNTDYNTPFTSRVTISKDNSKNQVSLKMNSLT
AADTAVYYCARALTYYDYEFAYWGQGTLVTVSS
>Sequence ID 42: Cetuximab H11 VH nucleotide sequence
CAAGTTCAGCTGCAAGAGTCTGGCCCTGGCCTGGTCAAGCCTTCCGAATCTCTGTCTCTGACCTGCACCGTGTCCGGCTTCTCCCT
GACCAATTATGGCGTGCACTGGGTTCGACAGCCTCCAGGCAAAGGCCTGGAATGGATCGGAGTGATTTGGAGCGGCGGCAACACCG
ACTACAACACCCCTTTCACCTCTAGAGTGACCATCAGCAAGGACAACTCCAAGAACCAGGTGTCCCTGAAGATGAACAGCCTGACC
GCTGCCGACACCGCTGTGTACTATTGTGCTAGAGCCCTGACCTACTACGACTACGAGTTCGCCTATTGGGGCCAGGGAACCCTGGT
CACAGTCTCCTCT
>Sequence ID 43: Cetuximab H11 VL amino acid sequence
DIVLTQSPATLSVSPGERATLSCRASQSIGTNIHWYQQKPGQAPRLLIKYASESISGIPSRFSGSGSGTDFTLTISSVQSEDFAVY
YCQQNNNWPTTFGQGTKLTVL
>Sequence ID 44: Cetuximab H11 VL nucleotide sequence
GACATCGTGCTGACCCAGTCTCCAGCCACACTGAGTGTGTCTCCAGGCGAGAGAGCTACCCTGTCCTGTAGAGCCTCTCAGTCCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCTCCTCCGTGCAGTCCGAGGATTTCGCCGTGTAC
TACTGCCAGCAGAACAACAACTGGCCCACCACCTTTGGCCAGGGCACCAAACTGACAGTTCTT
>Sequence ID 45: MonoFc amino acid sequence
GSGGSPVAGPSVFLFPPKPKDTLMISRTPEVTCVVVDVSHEDPEVKFNWYVDGVEVHNAKTKPREEQYASTYRVVSVLTVLHQDWL
NGKEYKCKVSNKALPAPIEKTISKAKGQPREPQVYTLPPSRDELTKNQVSLRCHVKGFYPSDIAVEWESNGQPENNYKTTKPVLDS
DGSFFLYSTLTVDKSRWQQGNVFSCSVLHEALHNHYTQKSLSLSPGK
>Sequence ID 46: MonoFc nucleotide sequence
GGATCCGGCGGCTCTCCCGTCGCTGGACCGTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCAACTGGTACGTGGACGGCGTGGAGGTGCATA
ATGCCAAGACAAAGCCGCGGGAGGAGCAGTACGCTAGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCCATCGAGAAAACCATCTCCAAAGCCAAAGGGCAGCC
CCGAGAACCACAGGTGTACACCCTGCCCCCATCCCGGGATGAGCTGACCAAGAACCAGGTCAGCCTGAGATGCCACGTCAAAGGCT
TCTATCCCAGCGACATCGCCGTGGAGTGGGAGAGCAATGGGCAGCCGGAGAACAACTACAAGACCACGAAGCCCGTGCTGGACTCC
GACGGCTCCTTCTTCCTCTACAGCACCCTCACCGTGGACAAGAGCAGGTGGCAGCAGGGGAACGTCTTCTCATGCTCCGTGCTCCA
TGAGGCTCTGCACAACCACTACACACAGAAGAGCCTCTCCCTGTCTCCGGGCAAATGA
>Sequence ID 47: Human IgG1 amino acid sequence
ASTKGPSVFPLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPG
>Sequence ID 48: Human IgG1 nucleotide sequence
GCTAGCACCAAGGGCCCATCGGTCTTCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTGCCCAGCACC
TGAACTCCTGGGGGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAATGCCAAGACA
AAGCCGCGGGAGGAGCAGTACAACAGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAGCCAAAGGGCAGCCCCGAGAACCAC
AGGTGTACACCCTGCCCCCATCCCGGGATGAGCTGACCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAAGACCACGCCTCCCGTGCTGGACTCCGACGGCTCCTT
CTTCCTCTATAGCAAGCTCACCGTGGACAAGAGCAGGTGGCAGCAGGGGAACGTCTTCTCATGCTCCGTGATGCATGAGGCTCTGC
ACAACCACTACACGCAGAAGAGCCTCTCCCTGTCTCCGGGTTAA
>Sequence ID 49: Human C kappa amino acid sequence
RTVAAPSVFIFPPSDEQLKSGTASVVCLLNNFYPREAKVQWKVDNALQSGNSQESVTEQDSKDSTYSLSSTLTLSKADYEKHKVYA
CEVTHQGLSSPVTKSENRGEC
>Sequence ID 50: Human C kappa amino acid sequence
CGTACGGTGGCTGCACCATCTGTCTTCATCTTCCCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCCCTCCAATCGGGTAACTCCCAGGAGAGTGTCACAG
AGCAGGACAGCAAGGACAGCACCTACAGCCTCAGCAGCACCCTGACGCTGAGCAAAGCAGACTACGAGAAACACAAAGTCTACGCC
TGCGAAGTCACCCATCAGGGCCTGAGCTCGCCCGTCACAAAGAGCTTCAACAGGGGAGAGTGTTAG
>Sequence ID 51: (G4S) 4 linker amino acid sequence
GGGGSGGGGSGGGGSGGGGS
>Sequence ID 52: (G4S) 4 linker nucleotide sequence
GGCGGCGGAGGATCTGGCGGAGGTGGAAGCGGAGGCGGAGGAAGCGGTGGCGGCGGATCT
>Sequence ID 53: His tag amino acid sequence
GSHHHHHH
>Sequence ID 54: His tag nucleotide sequence
GGATCCCATCATCACCATCACCATTGA
>Sequence ID 55: SI-79R1 amino acid sequence
DILLTQSPVILSVSPGERVSFSCRASQSIGTNIHWYQQRTNGSPRLLIKYASESISGIPSRFSGSGSGTDFTLSINSVESEDIADY
YCQQNNNWPTTFGAGTKLELKGGGGSGGGGSGGGGSGGGGSQVQLKQSGPGLVQPSQSLSITCTVSGFSLTNYGVHWVRQSPGKGL
EWLGVIWSGGNTDYNTPFTSRLSINKDNSKSQVFFKMNSLQSNDTAIYYCARALTYYDYEFAYWGQGTLVTVSSGSHHHHHH
>Sequence ID 56: SI-79R1 nucleotide sequence
GACATCTTGCTGACTCAGTCTCCAGTCATCCTGTCTGTGAGTCCAGGAGAAAGAGTCAGTTTCTCCTGCAGGGCCAGTCAGAGTAT
TGGCACAAACATACACTGGTATCAGCAAAGAACAAATGGTTCTCCAAGGCTTCTCATAAAGTATGCTTCTGAGTCTATCTCTGGGA
TTCCTTCCAGGTTTAGTGGCAGTGGATCAGGGACAGATTTTACTCTTAGCATCAACAGTGTGGAGTCTGAAGATATTGCAGATTAT
TACTGTCAACAAAATAATAACTGGCCAACCACGTTCGGTGCTGGGACCAAGCTGGAGCTGAAAGGCGGTGGCGGTAGTGGGGGAGG
CGGTTCTGGCGGCGGAGGGTCCGGCGGTGGAGGATCACAGGTGCAGCTGAAGCAGTCAGGACCTGGCCTAGTGCAGCCCTCACAGA
GCCTGTCCATCACCTGCACAGTCTCTGGTTTCTCATTAACTAACTATGGTGTACACTGGGTTCGCCAGTCTCCAGGAAAGGGTCTG
GAGTGGCTGGGAGTGATATGGAGTGGTGGAAACACAGACTATAATACACCTTTCACATCCAGACTGAGCATCAACAAGGACAATTC
CAAGAGCCAAGTTTTCTTTAAAATGAACAGTCTGCAATCTAATGACACAGCCATATATTACTGTGCCAGAGCCCTCACCTACTATG
ATTACGAGTTTGCTTACTGGGGCCAAGGGACTCTGGTCACTGTCTCGAGTGGATCCCATCATCACCATCACCATTGA
>Sequence ID 57: SI-79R2 amino acid sequence
EIVMTQSPSTLSASVGDRVIITCRASQSIGTNIHWYQQKPGKAPKLLIYYASESISGIPSRFSGSGSGAEFTLTISSLQPDDFATY
YCQQNNNWPTTFGQGTKLTVLGGGGSGGGGSGGGGSGGGGSEVQLVESGGGLVQPGGSLRLSCTVSGFSLTNYGVHWVRQAPGKGL
EWVGVIWSGGNTDYNTPFTSRFTISRDTSKNTVYLQMNSLRAEDTAVYYCARALTYYDYEFAYWGQGTLVTVSSGSHHHHHH
>Sequence ID 58: SI-79R2 nucleotide sequence
GAAATCGTTATGACACAGTCCCCATCCACTCTTAGCGCTTCTGTAGGGGATCGAGTGATTATCACATGCCGGGCCTCCCAATCCAT
AGGAACCAACATACACTGGTATCAACAAAAACCAGGCAAAGCGCCAAAACTGCTTATCTACTACGCCTCCGAGAGTATTTCTGGAA
TCCCGAGTCGCTTCTCAGGTTCTGGAAGCGGCGCTGAGTTTACCCTCACAATTTCTTCACTCCAACCGGATGACTTCGCTACATAT
TACTGCCAACAAAACAATAATTGGCCGACGACCTTTGGCCAGGGCACGAAACTTACGGTACTTGGCGGTGGCGGTAGTGGGGGAGG
CGGTTCTGGCGGCGGAGGGTCCGGCGGTGGAGGATCAGAAGTACAGCTTGTCGAGTCCGGTGGGGGGCTTGTTCAGCCAGGGGGTT
CCTTGAGGCTTTCCTGCACCGTCTCTGGGTTTAGCTTGACGAATTACGGCGTTCACTGGGTTAGACAAGCACCGGGGAAGGGGCTG
GAATGGGTCGGTGTGATATGGTCCGGGGGTAATACGGATTACAATACACCTTTCACGTCACGCTTTACGATTAGCAGGGACACGTC
AAAAAATACAGTCTACTTGCAGATGAACTCTCTTAGGGCGGAAGATACTGCAGTTTATTACTGCGCAAGGGCTCTGACATACTACG
ATTATGAATTTGCATATTGGGGCCAGGGGACTTTGGTCACGGTCTCGAGTGGATCCCATCATCACCATCACCATTGA
>Sequence ID 59: SI-79SF1 amino acid sequence
DILLTQSPVILSVSPGERVSFSCRASQSIGTNIHWYQQRTNGSPRLLIKYASESISGIPSRFSGSGSGTDFTLSINSVESEDIADY
YCQQNNNWPTTFGAGTKLELKGGGGGGGGSGGGGSGGGGSQVQLKQSGPGLVQPSQSLSITCTVSGFSLTNYGVHWVRQSPGKGL
EWLGVIWSGGNTDYNTPFTSRLSINKDNSKSQVFFKMNSLQSNDTAIYYCARALTYYDYEFAYWGQGTLVTVSAGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 60: SI-79SF1 nucleotide sequence
GACATCCTGCTGACCCAGTCTCCAGTGATCCTGTCCGTGTCTCCTGGCGAGAGAGTGTCCTTCAGCTGCAGAGCCTCTCAGTCCAT
CGGCACCAACATCCACTGGTATCAGCAGCGGACCAACGGCTCCCCTCGGCTGCTGATTAAGTACGCCTCCGAGTCTATCAGCGGCA
TCCCCTCCAGATTCTCCGGCTCTGGCTCTGGCACCGACTTCACCCTGTCCATCAACTCCGTGGAATCCGAGGATATCGCCGACTAC
TACTGCCAGCAGAACAACAACTGGCCCACCACCTTTGGCGCTGGCACCAAGCTGGAATTGAAAGGCGGCGGAGGATCTGGCGGAGG
TGGAAGCGGAGGCGGAGGAAGCGGTGGCGGCGGATCTCAAGTTCAGCTCAAGCAGTCTGGCCCTGGCCTGGTTCAGCCTTCTCAGA
GCCTGAGCATCACCTGTACCGTGTCCGGCTTCTCCCTGACCAATTACGGCGTGCACTGGGTTCGACAGAGCCCTGGCAAAGGACTG
GAATGGCTGGGAGTGATTTGGAGCGGCGGCAACACCGACTACAACACCCCTTTCACCTCTCGGCTGTCTATCAACAAGGACAACTC
CAAGAGCCAGGTGTTCTTCAAGATGAACTCCCTGCAGTCCAACGACACCGCCATCTACTACTGTGCTCGGGCCCTGACCTACTACG
ACTACGAGTTTGCTTACTGGGGCCAGGGCACCCTGGTCACAGTTTCTGCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 61: SI-79SF2 amino acid sequence
DILLTQSPVILSVSPGERVSFSCRASQSIGTNIHWYQQRTNGSPRLLIKYASESISGIPSRFSGSGSGTDFTLSINSVESEDIADY
YCQQNNNWPTTFGAGTKLELKGGGGSGGGGSGGGGSGGGGSQVQLKQSGPGLVQPSQSLSITCTVSGFSLTNYGVHWVRQSPGKGL
EWLGVIWSGGNTDYNTPFTSRLSINKDNSKSQVFFKMNSLQSEDTAIYYCARALTYYDYEFAYWGQGTLVTVSAGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 62: SI-79SF2 nucleotide sequence
GACATCCTGCTGACCCAGTCTCCAGTGATCCTGTCCGTGTCTCCTGGCGAGAGAGTGTCCTTCAGCTGCAGAGCCTCTCAGTCCAT
CGGCACCAACATCCACTGGTATCAGCAGCGGACCAACGGCTCCCCTCGGCTGCTGATTAAGTACGCCTCCGAGTCTATCAGCGGCA
TCCCCTCCAGATTCTCCGGCTCTGGCTCTGGCACCGACTTCACCCTGTCCATCAACTCCGTGGAATCCGAGGATATCGCCGACTAC
TACTGCCAGCAGAACAACAACTGGCCCACCACCTTTGGCGCTGGCACCAAGCTGGAATTGAAAGGCGGCGGAGGATCTGGCGGAGG
TGGAAGCGGAGGCGGAGGAAGCGGTGGCGGCGGATCTCAAGTTCAGCTCAAGCAGTCTGGCCCTGGCCTGGTTCAGCCTTCTCAGA
GCCTGAGCATCACCTGTACCGTGTCCGGCTTCTCCCTGACCAATTACGGCGTGCACTGGGTTCGACAGAGCCCTGGCAAAGGACTG
GAATGGCTGGGAGTGATTTGGAGCGGCGGCAACACCGACTACAACACCCCTTTCACCTCTCGGCTGTCTATCAACAAGGACAACTC
CAAGAGCCAGGTGTTCTTCAAGATGAACTCCCTGCAGTCCGAGGACACCGCCATCTACTACTGTGCTCGGGCCCTGACCTACTACG
ACTACGAGTTTGCTTACTGGGGCCAGGGCACCCTGGTCACAGTTTCTGCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 63: SI-79SF3 amino acid sequence
EIVMTQSPSTLSASVGDRVIITCRASQSIGTNIHWYQQKPGKAPKLLIYYASESISGIPSRFSGSGSGAEFTLTISSLQPDDFATY
YCQQNNNWPTTFGQGTKLTVLGGGGSGGGGSGGGGSGGGGSEVQLVESGGGLVQPGGSLRLSCKVSGFSLTNYGVHWVRQAPGKGL
EWVGVIWSGGNTDYNTPFTSRFTISRDTSKNTVYLQMNSLRAEDTAVYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 64: SI-79SF3 nucleotide sequence
GAGATCGTGATGACCCAGTCTCCTTCCACACTGTCCGCCTCTGTGGGCGACAGAGTGATCATCACCTGTAGAGCCAGCCAGTCCAT
CGGCACCAACATCCACTGGTATCAGCAGAAGCCTGGCAAGGCCCCTAAGCTGCTGATCTACTACGCCTCCGAGTCTATCAGCGGCA
TCCCCTCCAGATTCTCCGGCTCTGGATCTGGCGCTGAGTTTACCCTGACAATCTCCAGCCTGCAGCCTGACGACTTCGCCACCTAC
TACTGCCAGCAGAACAACAACTGGCCCACCACCTTTGGCCAGGGCACCAAACTGACAGTTCTTGGCGGCGGAGGATCTGGCGGAGG
TGGAAGCGGAGGCGGAGGAAGCGGTGGCGGCGGATCTGAAGTTCAGCTGGTGGAATCCGGCGGAGGATTGGTTCAACCTGGCGGCT
CTCTGAGACTGTCCTGTAAGGTGTCTGGCTTCTCCCTGACCAACTACGGCGTGCACTGGGTCCGACAGGCACCTGGAAAAGGACTG
GAATGGGTCGGAGTGATTTGGAGCGGCGGCAACACCGACTACAACACCCCTTTCACCAGCCGGTTCACCATCTCTCGGGACACCTC
CAAGAACACCGTGTACCTGCAGATGAACTCCCTGAGAGCCGAGGACACCGCCGTGTACTATTGTGCTAGAGCCCTGACCTACTATG
ACTACGAGTTCGCCTATTGGGGCCAGGGAACCCTGGTCACAGTCTCCTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 65: SI-79SF4 amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLELKGGGGGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 66: SI-79SF4 nucleotide sequence
GAGATCGTGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAGCCCGGCAAGCCTCCTCGGCTGCTGATTAAGTACGCCTCCGAGTCCATCAGCGGCA
TCCCTGACAGATTCTCCGGCTCTGGCTCTGGCACCGAGTTTACCCTGACCATCTCCTCCGTGCAGTCCGAGGATTTCGCCGTGTAC
TACTGCCAGCAGAACAACAACTGGCCCACCACCTTTGGACCCGGCACCAAGCTGGAATTGAAAGGCGGCGGAGGATCTGGCGGAGG
TGGAAGCGGAGGCGGAGGAAGCGGTGGCGGCGGATCTCAAGTTCAGTTGCAGCAGTCTGGCCCTGGCCTGGTCAAGCCTTCTGAGA
CACTGTCCATCACCTGTACCGTGTCCGGCTTCTCCCTGACCAATTACGGCGTGCACTGGATCAGACAGGCCCCTGGCAAAGGACTG
GAATGGCTGGGAGTGATTTGGAGCGGCGGCAACACCGACTACAACACCCCTTTCACCAGCCGGTTCACCATCACCAAGGACAACTC
CAAGAACCAGGTGTACTTCAAGCTGCGGAGCGTGCGGGCTGATGACACCGCCATCTACTACTGTGCTCGGGCCCTGACCTACTACG
ACTACGAGTTTGCTTACTGGGGCCAGGGCACCCTGGTCACAGTTTCTTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 67: SI-79SF5 amino acid sequence
DIVLTQSPSSLSVSPGERVTISCRASQSIGTNIHWYQQKPGQAPRLLIKYASESISGIPSRFSGSGSGTDFTLTISSVESEDFAVY
YCQQNNNWPTTFGQGTKLEIKGGGGSGGGGSGGGGSGGGGSQVQLVESGPGLVQPSGSLSLTCTVSGFSLTNYGVHWVRQAPGKGL
EWVGVIWSGGNTDYNTPFTSRFTISKDNSKNQVYLKMNSLRAEDTAVYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 68: SI-79SF5 nucleotide sequence
GACATCGTGCTGACCCAGTCTCCATCCAGCCTGTCTGTGTCTCCTGGCGAGAGAGTGACCATCTCTTGCCGGGCCTCTCAGAGCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCAGCTCCGTGGAATCCGAGGACTTCGCCGTGTAC
TACTGCCAGCAGAACAACAACTGGCCCACCACCTTTGGCCAGGGCACCAAGCTGGAAATCAAAGGCGGCGGAGGATCTGGCGGAGG
TGGAAGCGGAGGCGGAGGAAGCGGTGGCGGCGGATCTCAAGTTCAGCTGGTGGAATCTGGCCCTGGCCTGGTTCAGCCTTCTGGCT
CTCTGTCTCTGACCTGCACCGTGTCTGGCTTCTCCCTGACCAATTACGGCGTGCACTGGGTTCGACAGGCTCCAGGCAAAGGACTG
GAATGGGTCGGAGTGATTTGGAGCGGCGGCAACACCGACTACAACACCCCTTTCACCAGCCGGTTCACCATCTCCAAGGACAACTC
CAAGAACCAGGTGTACCTGAAGATGAACTCCCTGAGAGCCGAGGACACCGCTGTGTATTACTGTGCTAGAGCCCTGACCTACTACG
ACTACGAGTTCGCCTATTGGGGCCAGGGAACCCTGGTCACAGTCTCCTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 69: SI-79SF6 amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVLGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 70: SI-79SF6 nucleotide sequence
GAGATCGTGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAGCCCGGCAAGCCTCCTCGGCTGCTGATTAAGTACGCCTCCGAGTCCATCAGCGGCA
TCCCTGACAGATTCTCCGGCTCTGGCTCTGGCACCGAGTTTACCCTGACCATCTCCTCCGTGCAGTCCGAGGATTTCGCCGTGTAC
TACTGCCAGCAGAACAACAACTGGCCCACCACCTTTGGACCCGGCACCAAGCTGACAGTTCTTGGCGGCGGAGGATCTGGCGGAGG
TGGAAGCGGAGGCGGAGGAAGCGGTGGCGGCGGATCTCAAGTTCAGTTGCAGCAGTCTGGCCCTGGCCTGGTCAAGCCTTCTGAGA
CACTGTCCATCACCTGTACCGTGTCCGGCTTCTCCCTGACCAATTACGGCGTGCACTGGATCAGACAGGCCCCTGGCAAAGGACTG
GAATGGCTGGGAGTGATTTGGAGCGGCGGCAACACCGACTACAACACCCCTTTCACCAGCCGGTTCACCATCACCAAGGACAACTC
CAAGAACCAGGTGTACTTCAAGCTGCGGAGCGTGCGGGCTGATGACACCGCCATCTACTACTGTGCTCGGGCCCTGACCTACTACG
ACTACGAGTTTGCTTACTGGGGCCAGGGCACCCTGGTCACAGTTTCTTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 71: SI-79SF7 amino acid sequence
DIVLTQSPSSLSVSPGERVTISCRASQSIGTNIHWYQQKPGQAPRLLIKYASESISGIPSRFSGSGSGTDFTLTISSVESEDFAVY
YCQQNNNWPTTFGQGTKLTVLGGGGSGGGGSGGGGSGGGGSQVQLVESGPGLVQPSGSLSLTCTVSGFSLTNYGVHWVRQAPGKGL
EWVGVIWSGGNTDYNTPFTSRFTISKDNSKNQVYLKMNSLRAEDTAVYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 72: SI-79SF7 nucleotide sequence
GACATCGTGCTGACCCAGTCTCCATCCAGCCTGTCTGTGTCTCCTGGCGAGAGAGTGACCATCTCTTGCCGGGCCTCTCAGAGCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCAGCTCCGTGGAATCCGAGGACTTCGCCGTGTAC
TACTGCCAGCAGAACAACAACTGGCCCACCACCTTTGGCCAGGGCACCAAGCTGACAGTTCTTGGCGGCGGAGGATCTGGCGGAGG
TGGAAGCGGAGGCGGAGGAAGCGGTGGCGGCGGATCTCAAGTTCAGCTGGTGGAATCTGGCCCTGGCCTGGTTCAGCCTTCTGGCT
CTCTGTCTCTGACCTGCACCGTGTCTGGCTTCTCCCTGACCAATTACGGCGTGCACTGGGTTCGACAGGCTCCAGGCAAAGGACTG
GAATGGGTCGGAGTGATTTGGAGCGGCGGCAACACCGACTACAACACCCCTTTCACCAGCCGGTTCACCATCTCCAAGGACAACTC
CAAGAACCAGGTGTACCTGAAGATGAACTCCCTGAGAGCCGAGGACACCGCTGTGTATTACTGTGCTAGAGCCCTGACCTACTACG
ACTACGAGTTCGCCTATTGGGGCCAGGGAACCCTGGTCACAGTCTCCTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 73: SI-79SF8 amino acid sequence
DIVLTQSPATLSVSPGERATLSCRASQSIGTNIHWYQQKPGQAPRLLIKYASESISGIPSRFSGSGSGTDFTLTISSVQSEDFAVY
YCQQNNNWPTTFGQGTKLEIKGGGGSGGGGSGGGGSGGGGSQVQLQESGPGLVKPSESLSLTCTVSGFSLTNYGVHWVRQPPGKGL
EWIGVIWSGGNTDYNTPFTSRVTISKDNSKNQVSLKMNSLTAADTAVYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 74: SI-79SF8 nucleotide sequence
GACATCGTGCTGACCCAGTCTCCAGCCACACTGAGTGTGTCTCCAGGCGAGAGAGCTACCCTGTCCTGTAGAGCCTCTCAGTCCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCTCCTCCGTGCAGTCCGAGGATTTCGCCGTGTAC
TACTGCCAGCAGAACAACAACTGGCCCACCACCTTTGGCCAGGGCACCAAGCTGGAAATCAAAGGCGGCGGAGGATCTGGCGGAGG
TGGAAGCGGAGGCGGAGGAAGCGGTGGCGGCGGATCTCAAGTTCAGCTGCAAGAGTCTGGCCCTGGCCTGGTCAAGCCTTCCGAAT
CTCTGTCTCTGACCTGCACCGTGTCCGGCTTCTCCCTGACCAATTATGGCGTGCACTGGGTTCGACAGCCTCCAGGCAAAGGCCTG
GAATGGATCGGAGTGATTTGGAGCGGCGGCAACACCGACTACAACACCCCTTTCACCTCTAGAGTGACCATCAGCAAGGACAACTC
CAAGAACCAGGTGTCCCTGAAGATGAACAGCCTGACCGCTGCCGACACCGCTGTGTACTATTGTGCTAGAGCCCTGACCTACTACG
ACTACGAGTTCGCCTATTGGGGCCAGGGAACCCTGGTCACAGTCTCCTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 75: SI-79SF9 amino acid sequence
EIVLTQSPSTLSVSPGERVSFSCRASQSIGTNIHWYQQRTGQPPRLLIKYASESISGIPARFSGSGSGTEFTLTITSVQSEDFAVY
YCQQNNNWPTTFGQGTKLDIKGGGGSGGGGSGGGGSGGGGSQVQLLQSGPGLVKPSETLSITCTVSGFSLTNYGVHWIRQAPGKGL
EWIGVIWSGGNTDYNTPFTSRFTITKDNSKNQVFFKLRSVRAEDTALYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 76: SI-79SF9 nucleotide sequence
GAGATCGTGCTGACCCAGTCTCCTTCCACACTGTCTGTGTCTCCCGGCGAGAGAGTGTCCTTCAGCTGTAGAGCCTCTCAGTCCAT
CGGCACCAACATCCACTGGTATCAGCAGAGAACCGGCCAGCCTCCTCGGCTGCTGATTAAGTACGCCTCCGAGTCCATCAGCGGCA
TCCCTGCCAGATTTTCCGGCTCTGGCTCTGGCACCGAGTTCACCCTGACCATCACCTCCGTGCAGTCTGAGGACTTCGCCGTGTAC
TACTGCCAGCAGAACAACAACTGGCCCACCACCTTTGGCCAGGGCACCAAGCTGGATATCAAAGGCGGCGGAGGATCTGGCGGAGG
TGGAAGCGGAGGCGGAGGAAGCGGTGGCGGCGGATCTCAAGTTCAGCTGTTGCAGTCTGGCCCTGGCCTGGTCAAGCCTTCCGAGA
CACTGTCCATCACCTGTACCGTGTCCGGCTTCTCCCTGACCAATTACGGCGTGCACTGGATCAGACAGGCCCCTGGCAAAGGCCTG
GAATGGATCGGAGTGATTTGGAGCGGCGGCAACACCGACTACAACACCCCTTTCACCAGCCGGTTCACCATCACCAAGGACAACTC
CAAGAACCAGGTGTTCTTCAAGCTGCGGAGCGTGCGCGCTGAGGATACCGCTCTGTACTATTGCGCTCGGGCCCTGACCTACTACG
ACTACGAGTTTGCTTACTGGGGCCAGGGAACCCTGGTCACCGTTTCTTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 77: SI-79SF10 amino acid sequence
EIVLTQSPSILSVSPGERVTFSCRASQSIGTNIHWYQQRPGKPPRLLIKYASESISGIPARFSGSGSGTEFTLTISSVQSEDFATY
YCQQNNNWPTTFGPGTKLELKGGGGSGGGGSGGGGSGGGGSQVQLVQSGPGLVKPSQTLSLTCTVSGFSLTNYGVHWIRQAPGKGL
EWLGVIWSGGNTDYNTPFTSRFTITKDNSKNQVYFKLRSVRSEDTAVYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 78: SI-79SF10 nucleotide sequence
GAGATCGTGCTGACCCAGTCTCCTTCCATCCTGTCTGTGTCTCCCGGCGAGAGAGTGACCTTCAGCTGTAGAGCCTCTCAGTCCAT
CGGCACCAACATCCACTGGTATCAGCAGCGGCCTGGCAAGCCTCCTCGGCTGCTGATTAAGTACGCCTCCGAGTCCATCAGCGGCA
TCCCTGCCAGATTTTCCGGCTCTGGCTCTGGCACCGAGTTTACCCTGACCATCTCCTCCGTGCAGTCCGAGGATTTCGCCACCTAC
TACTGCCAGCAGAACAACAACTGGCCCACCACCTTTGGACCCGGCACCAAGCTGGAATTGAAAGGCGGCGGAGGATCTGGCGGAGG
TGGAAGCGGAGGCGGAGGAAGCGGTGGCGGCGGATCTCAAGTTCAGCTGGTTCAGTCTGGCCCTGGCCTCGTGAAGCCTTCTCAGA
CCCTGTCTCTGACCTGCACCGTGTCTGGCTTCTCCCTGACCAATTACGGCGTGCACTGGATCAGACAGGCCCCTGGCAAAGGACTG
GAATGGCTGGGAGTGATTTGGAGCGGCGGCAACACCGACTACAACACCCCTTTCACCAGCCGGTTCACCATCACCAAGGACAACTC
CAAGAACCAGGTGTACTTCAAGCTGAGATCCGTGCGGAGCGAGGACACCGCCGTGTACTATTGTGCTAGAGCCCTGACCTACTACG
ACTACGAGTTCGCCTATTGGGGCCAGGGCACACTGGTCACAGTCTCTTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 79: SI-79SF11 amino acid sequence
EIQLTQSPSTLSVSPGERATFSCRASQSIGTNIHWYQQKTGQPPRLLIKYASESISGIPDRFSGSGSGTEFTLSITSVQSEDFAVY
YCQQNNNWPTTFGPGTKLEILGGGGSGGGGSGGGGSGGGGSQVQLLQSGPGLVKPSETLSLTCTVSGFSLTNYGVHWIRQAPGKGL
EWIGVIWSGGNTDYNTPFTSRFTISKDNSKNQVYFKLRSLTSEDTAIYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 80: SI-79SF11 nucleotide sequence
GAGATCCAGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAAACCGGCCAGCCTCCTCGGCTGCTGATTAAGTACGCCTCCGAGTCCATCAGCGGCA
TCCCTGACAGATTCTCCGGCTCTGGCTCTGGCACCGAGTTCACCCTGTCTATCACCTCCGTGCAGTCCGAGGACTTCGCCGTGTAC
TACTGCCAGCAGAACAACAACTGGCCCACCACCTTTGGACCCGGCACCAAGCTGGAAATTCTTGGCGGCGGAGGATCTGGCGGAGG
TGGAAGCGGAGGCGGAGGAAGCGGTGGCGGCGGATCTCAAGTTCAGCTGTTGCAGTCTGGCCCTGGCCTGGTCAAGCCTTCCGAAA
CACTGTCTCTGACCTGCACCGTGTCCGGCTTCTCCCTGACCAATTATGGCGTGCACTGGATCAGACAGGCCCCTGGCAAAGGCCTG
GAATGGATCGGAGTGATTTGGAGCGGCGGCAACACCGACTACAACACCCCTTTCACCAGCCGGTTCACCATCTCCAAGGACAACTC
CAAGAACCAGGTGTACTTCAAGCTGCGGTCCCTGACCTCTGAGGACACCGCCATCTACTACTGCGCTAGAGCCCTGACCTACTACG
ACTACGAGTTCGCCTATTGGGGCCAGGGCACACTGGTCACAGTCTCTTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 81: SI-79SF12 amino acid sequence
EIVLTQSPSILSVSPGERATFSCRASQSIGTNIHWYQQRPGKPPRLLIKYASESISGIPSRFSGSGSGTEFTLTITSVQSEDIAVY
YCQQNNNWPTTFGPGTKLELKGGGGSGGGGSGGGGSGGGGSQVQLQQSGPGLVKPSQTLSITCTVSGFSLTNYGVHWIRQAPGKGL
EWLGVIWSGGNTDYNTPFTSRFTISKDNSKNQVYFKLRSLRAEDTAIYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 82: SI-79SF12 nucleotide sequence
GAGATCGTGCTGACCCAGTCTCCTTCCATCCTGTCTGTGTCTCCCGGCGAGAGAGCCACCTTCAGCTGTAGAGCCTCTCAGTCCAT
CGGCACCAACATCCACTGGTATCAGCAGCGGCCTGGCAAGCCTCCTCGGCTGCTGATTAAGTACGCCTCCGAGTCTATCAGCGGCA
TCCCCTCCAGATTCTCCGGCTCTGGATCTGGCACCGAGTTCACCCTGACCATCACCTCCGTGCAGTCCGAGGATATCGCCGTGTAC
TACTGCCAGCAGAACAACAACTGGCCCACCACCTTTGGACCCGGCACCAAGCTGGAATTGAAAGGCGGCGGAGGATCTGGCGGAGG
TGGAAGCGGAGGCGGAGGAAGCGGTGGCGGCGGATCTCAAGTTCAGTTGCAGCAGTCTGGCCCTGGCCTCGTGAAGCCTTCTCAGA
CCCTGTCTATCACCTGTACCGTGTCCGGCTTCTCCCTGACCAATTACGGCGTGCACTGGATCAGACAGGCCCCTGGCAAAGGACTG
GAATGGCTGGGAGTGATTTGGAGCGGCGGCAACACCGACTACAACACCCCTTTCACCAGCCGGTTCACCATCTCCAAGGACAACTC
CAAGAACCAGGTGTACTTCAAGCTGCGGTCCCTGAGAGCCGAGGACACCGCCATCTACTACTGTGCTAGAGCCCTGACCTACTACG
ACTACGAGTTCGCCTATTGGGGCCAGGGCACACTGGTCACAGTCTCTTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 83: SI-79SF13 amino acid sequence
DIVLTQSPATLSVSPGERATLSCRASQSIGTNIHWYQQKPGQAPRLLIKYASESISGIPSRFSGSGSGTDFTLTISSVQSEDFAVY
YCQQNNNWPTTFGQGTKLTVLGGGGSGGGGSGGGGSGGGGSQVQLQESGPGLVKPSESLSLTCTVSGFSLTNYGVHWVRQPPGKGL
EWIGVIWSGGNTDYNTPFTSRVTISKDNSKNQVSLKMNSLTAADTAVYYCARALTYYDYEFAYWGQGTLVTVSSGSGGSPVAGPSV
FLFPPKPKDTLMISRTPEVTCVVVDVSHEDPEVKFNWYVDGVEVHNAKTKPREEQYASTYRVVSVLTVLHQDWLNGKEYKCKVSNK
ALPAPIEKTISKAKGQPREPQVYTLPPSRDELTKNQVSLRCHVKGFYPSDIAVEWESNGQPENNYKTTKPVLDSDGSFFLYSTLTV
DKSRWQQGNVFSCSVLHEALHNHYTQKSLSLSPGK
>Sequence ID 84: SI-79SF13 nucleotide sequence
GACATCGTGCTGACCCAGTCTCCAGCCACACTGAGTGTGTCTCCAGGCGAGAGAGCTACCCTGTCCTGTAGAGCCTCTCAGTCCAT
CGGCACCAACATCCACTGGTATCAGCAGAAGCCTGGACAGGCCCCTCGGCTGCTGATTAAGTACGCCTCCGAGTCTATCAGCGGCA
TCCCCTCCAGATTCTCCGGCTCTGGCTCTGGCACAGACTTTACCCTGACCATCTCCTCCGTGCAGTCCGAGGATTTCGCCGTGTAC
TACTGCCAGCAGAACAACAACTGGCCCACCACCTTTGGCCAGGGCACCAAACTGACAGTTCTTGGCGGCGGAGGATCTGGCGGAGG
TGGAAGCGGAGGCGGAGGAAGCGGTGGCGGCGGATCTCAAGTTCAGCTGCAAGAGTCTGGCCCTGGCCTGGTCAAGCCTTCCGAAT
CTCTGTCTCTGACCTGCACCGTGTCCGGCTTCTCCCTGACCAATTATGGCGTGCACTGGGTTCGACAGCCTCCAGGCAAAGGCCTG
GAATGGATCGGAGTGATTTGGAGCGGCGGCAACACCGACTACAACACCCCTTTCACCTCTAGAGTGACCATCAGCAAGGACAACTC
CAAGAACCAGGTGTCCCTGAAGATGAACAGCCTGACCGCTGCCGACACCGCTGTGTACTATTGTGCTAGAGCCCTGACCTACTACG
ACTACGAGTTCGCCTATTGGGGCCAGGGAACCCTGGTCACAGTCTCCTCTGGATCCGGCGGCTCTCCCGTCGCTGGACCGTCAGTC
TTCCTCTTCCCCCCAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAGGAGCAGTACGCTA
GCACGTACCGTGTGGTCAGCGTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTGTACACCCTGCCCCCATCCCG
GGATGAGCTGACCAAGAACCAGGTCAGCCTGAGATGCCACGTCAAAGGCTTCTATCCCAGCGACATCGCCGTGGAGTGGGAGAGCA
ATGGGCAGCCGGAGAACAACTACAAGACCACGAAGCCCGTGCTGGACTCCGACGGCTCCTTCTTCCTCTACAGCACCCTCACCGTG
GACAAGAGCAGGTGGCAGCAGGGGAACGTCTTCTCATGCTCCGTGCTCCATGAGGCTCTGCACAACCACTACACACAGAAGAGCCT
CTCCCTGTCTCCGGGCAAATGA
>Sequence ID 85: SI-55P3 heavy chain amino acid sequence
EIVMTQSPSTLSASVGDRVIITCRASQSIGTNIHWYQQKPGKAPKLLIYYASESISGIPSRFSGSGSGAEFTLTISSLQPDDFATY
YCQQNNNWPTTFGQGTKLTVLGGGGSGGGGSGGGGSGGGGSEVQLVESGGGLVQPGGSLRLSCSVSGFSLTNYGVHWVRQAPGKGL
EWVGVIWSGGNTDYNTPFTSRFTISRDTSKNTVYLQMNSLRAEDTAVYYCARALTYYDYEFAYWGQGTLVTVSSGGGGSGGGGSQV
QLQESGGRLVQPGEPLSLTCKTSGIDLSSNAIGWVRQAPGKGLEWIGVIFGSGNTYYASWAKGRFTISRSTSTVYLKMNSLRSEDT
AIYYCARGGYSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGAPSVFLFPPKPKDTLMISRTPEVTCVVV
DVSHEDPEVKFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSVMHEALHNHY
TQKSLSLSPGGGGGSGGGGSEVQLLESGGGLVQPGGSLRLSCAASGFSFSSGYDMCWVRQAPGKGLEWIACIAAGSAGITYDANWA
KGRFTISRDNSKNTLYLQMNSLRAEDTAVYYCARSAFSFDYAMDLWGQGTLVTVSSGGGGSGGGGSGGGGSGGGGSDIQMTQSPST
LSASVGDRVTITCQASQSISSHLNWYQQKPGKAPKLLIYKASTLASGVPSRFSGSGSGTEFTLTISSLQPDDFATYYCQQGYSWGN
VDNVFGGGTKVEIKGGGGSGGGGSGRSLVESGGGLVQPGGSLRLSCTASGFTISSYHMQWVRQAPGKGLEYIGTISSGGNVYYASS
ARGRFTISRPSSKNTVDLQMNSLRAEDTAVYYCARDSGYSDPMWGQGTLVTVSSGGGGSGGGGSGGGGSGGGGSDVVMTQSPSSVS
ASVGDRVTITCQASQNIRTYLSWYQQKPGKAPKLLIYAAANLASGVPSRFSGSGSGTDFTLTISDLEPGDAATYYCQSTYLGTDYV
GGAFGGGTKVEIK
>Sequence ID 86: SI-55P3 heavy chain nucleotide sequence
GAAATCGTTATGACACAGTCCCCATCCACTCTTAGCGCTTCTGTAGGGGATCGAGTGATTATCACATGCCGGGCCTCCCAATCCAT
AGGAACCAACATACACTGGTATCAACAAAAACCAGGCAAAGCGCCAAAACTGCTTATCTACTACGCCTCCGAGAGTATTTCTGGAA
TCCCGAGTCGCTTCTCAGGTTCTGGAAGCGGCGCTGAGTTTACCCTCACAATTTCTTCACTCCAACCGGATGACTTCGCTACATAT
TACTGCCAACAAAACAATAATTGGCCGACGACCTTTGGCCAGGGCACGAAACTTACGGTACTTGGCGGTGGCGGTAGTGGGGGAGG
CGGTTCTGGCGGCGGAGGGTCCGGCGGTGGAGGATCAGAAGTACAGCTTGTCGAGTCCGGTGGGGGGCTTGTTCAGCCAGGGGGTT
CCTTGAGGCTTTCCTGCTCCGTCTCTGGGTTTAGCTTGACGAATTACGGCGTTCACTGGGTTAGACAAGCACCGGGGAAGGGGCTG
GAATGGGTCGGTGTGATATGGTCCGGGGGTAATACGGATTACAATACACCTTTCACGTCACGCTTTACGATTAGCAGGGACACGTC
AAAAAATACAGTCTACTTGCAGATGAACTCTCTTAGGGCGGAAGATACTGCAGTTTATTACTGCGCAAGGGCTCTGACATACTACG
ATTATGAATTTGCATATTGGGGCCAGGGGACTTTGGTCACGGTCTCGAGCGGCGGTGGAGGGTCCGGCGGTGGTGGATCACAAGTG
CAGTTGCAAGAAAGTGGTGGTAGACTGGTTCAGCCTGGTGAACCCTTGTCACTGACGTGTAAAACAAGCGGCATTGATCTGTCCTC
TAACGCCATCGGATGGGTCCGACAGGCCCCAGGAAAAGGTCTGGAGTGGATCGGAGTTATCTTCGGGAGCGGCAATACATACTACG
CAAGCTGGGCAAAAGGGCGATTTACGATATCACGGAGCACCTCTACAGTTTATTTGAAAATGAACTCCCTCCGGTCCGAGGATACC
GCGATATATTACTGTGCCAGAGGGGGGTACTCCTCTGATATCTGGGGGCAGGGTACACTGGTTACAGTTTCATCCGCTAGCACCAA
GGGCCCATCGGTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCACACATGCCCACCGTGCCCAGCACCTGAAGCCGCGG
GGGCACCGTCAGTCTTCCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAATGCCAAGACAAAGCCGCGGGA
GGAGCAGTACAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTGGCGGTGGAGGGTCCGGCGGTGGTGGATCCGAGGTGCAGCTGTTGGAGTCTGGGGG
AGGCTTGGTACAGCCTGGGGGGTCCCTGAGACTCTCCTGTGCAGCCTCTGGATTCTCCTTCAGTAGCGGGTACGACATGTGCTGGG
TCCGCCAGGCTCCAGGGAAGGGGCTGGAGTGGATCGCATGCATTGCTGCTGGTAGTGCTGGTATCACTTACGACGCGAACTGGGCG
AAAGGCCGGTTCACCATCTCCAGAGACAATTCCAAGAACACGCTGTATCTGCAAATGAACAGCCTGAGAGCCGAGGACACGGCCGT
ATATTACTGTGCGAGATCGGCGTTTTCGTTCGACTACGCCATGGACCTCTGGGGCCAGGGAACCCTGGTCACCGTGTCGAGCGGTG
GAGGCGGATCTGGCGGAGGTGGTTCCGGCGGTGGCGGCTCCGGTGGAGGCGGCTCTGACATCCAGATGACCCAGTCTCCTTCCACC
CTGTCTGCATCTGTAGGAGACAGAGTCACCATCACTTGCCAGGCCAGTCAGAGCATTAGTTCCCACTTAAACTGGTATCAGCAGAA
ACCAGGGAAAGCCCCTAAGCTCCTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCAACAGGGTTATAGTTGGGGTAAT
GTTGATAATGTTTTCGGCGGAGGGACCAAGGTGGAGATCAAAGGCGGTGGAGGGTCCGGCGGTGGTGGCTCCGGACGGTCGCTGGT
GGAGTCTGGGGGAGGCTTGGTCCAGCCTGGGGGGTCCCTGAGACTCTCCTGTACTGCCTCTGGATTCACCATCAGTAGCTACCACA
TGCAGTGGGTCCGCCAGGCTCCAGGGAAGGGGCTGGAGTACATCGGAACCATTAGTAGTGGTGGTAATGTATACTACGCAAGCTCC
GCTAGAGGCAGATTCACCATCTCCAGACCCTCGTCCAAGAACACGGTGGATCTTCAAATGAACAGCCTGAGAGCCGAGGACACGGC
TGTGTATTACTGTGCGAGAGACTCTGGTTATAGTGATCCTATGTGGGGCCAGGGAACCCTGGTCACCGTCTCTTCAGGCGGTGGCG
GTAGTGGGGGAGGCGGTTCTGGCGGCGGAGGGTCCGGCGGTGGAGGATCAGACGTTGTGATGACCCAGTCTCCATCTTCCGTGTCT
GCATCTGTAGGAGACAGAGTCACCATCACCTGTCAGGCCAGTCAGAACATTAGGACTTACTTATCCTGGTATCAGCAGAAACCAGG
GAAAGCCCCTAAGCTCCTGATCTATGCTGCAGCCAATCTGGCATCTGGGGTCCCATCAAGGTTCAGCGGCAGTGGATCTGGGACAG
ATTTCACTCTCACCATCAGCGACCTGGAGCCTGGCGATGCTGCAACTTACTATTGTCAGTCTACCTATCTTGGTACTGATTATGTT
GGCGGTGCTTTCGGCGGAGGGACCAAGGTGGAGATCAAATGA
>Sequence ID 87: SI-55P3 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDP
VLTQSPSSLSASVGDRVTISCQSSQSVAKNNNLAWFQQKPGQAPKLLIYSASTLAAGVPSRFSGSGSGTDFTLTISSVQPEDFATY
YCSARDSGNIQSFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSENRGEC
>Sequence ID 88: SI-55P3 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACGGTACTGGGTGGAGGCGGTTCAGGCGGAGGTGG
TTCCGGCGGTGGCGGCTCCGGTGGAGGCGGCTCTCAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCCGATCCA
GTTCTGACACAAAGTCCATCCAGCCTGTCTGCCTCAGTCGGCGACAGAGTGACCATCAGTTGCCAGAGCTCACAGTCTGTGGCTAA
GAACAACAACTTGGCGTGGTTCCAACAGAAACCTGGACAGGCTCCGAAATTGCTGATCTATTCTGCTTCCACGCTTGCTGCTGGTG
TTCCTTCCCGCTTTTCAGGTAGTGGTAGCGGGACAGACTTCACTTTGACTATAAGCAGCGTGCAGCCTGAAGATTTTGCGACCTAC
TATTGTTCTGCTAGAGACAGTGGAAATATTCAGTCCTTTGGGGGGGGAACGAAGGTCGAAATAAAGCGTACGGTGGCTGCACCATC
TGTCTTCATCTTCCCGCCATCTGATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTTGA
>Sequence ID 89: SI-55P4 heavy chain amino acid sequence
EIVMTQSPSTLSASVGDRVIITCRASQSIGTNIHWYQQKPGKAPKLLIYYASESISGIPSRFSGSGSGAEFTLTISSLQPDDFATY
YCQQNNNWPTTFGQGTKLTVLGGGGSGGGGSGGGGSGGGGSEVQLVESGGGLVQPGGSLRLSCSVSGFSLTNYGVHWVRQAPGKGL
EWVGVIWSGGNTDYNTPFTSRFTISRDTSKNTVYLQMNSLRAEDTAVYYCARALTYYDYEFAYWGQGTLVTVSSGGGGSGGGGSQV
QLQESGGRLVQPGEPLSLTCKTSGIDLSSNAIGWVRQAPGKGLEWIGVIFGSGNTYYASWAKGRFTISRSTSTVYLKMNSLRSEDT
AIYYCARGGYSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGAPSVFLFPPKPKDTLMISRTPEVTCVVV
DVSHEDPEVKFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSVMHEALHNHY
TQKSLSLSPGGGGGSGGGGSEVQLLESGGGLVQPGGSLRLSCAASGFSFSSGYDMCWVRQAPGKGLEWIACIAAGSAGITYDANWA
KGRFTISRDNSKNTLYLQMNSLRAEDTAVYYCARSAFSFDYAMDLWGQGTLVTVSSGGGGSGGGGSGGGGSGGGGSDIQMTQSPST
LSASVGDRVTITCQASQSISSHLNWYQQKPGKAPKLLIYKASTLASGVPSRFSGSGSGTEFTLTISSLQPDDFATYYCQQGYSWGN
VDNVFGGGTKVEIKGGGGGGGGSGREGPELSPDDPAGLLDLRQGMFAQLVAQNVLLIDGPLSWYSDPGLAGVSLTGGLSYKEDTK
ELVVAKAGVYYVFFQLELRRVVAGEGSGSVSLALHLQPLRSAAGAAALALTVDLPPASSEARNSAFGFQGRLLHLSAGQRLGVHLH
TEARARHAWQLTQGATVLGLFRVTPEIPAGLGSTGSGSKPGSGEGSTKGREGPELSPDDPAGLLDLRQGMFAQLVAQNVLLIDGPL
SWYSDPGLAGVSLTGGLSYKEDTKELVVAKAGVYYVFFQLELRRVVAGEGSGSVSLALHLQPLRSAAGAAALALTVDLPPASSEAR
NSAFGFQGRLLHLSAGQRLGVHLHTEARARHAWQLTQGATVLGLFRVTPEIPAGLGGGGSGGGGSREGPELSPDDPAGLLDLRQGM
FAQLVAQNVLLIDGPLSWYSDPGLAGVSLTGGLSYKEDTKELVVAKAGVYYVFFQLELRRVVAGEGSGSVSLALHLQPLRSAAGAA
ALALTVDLPPASSEARNSAFGFQGRLLHLSAGQRLGVHLHTEARARHAWQLTQGATVLGLFRVTPEIPAGL
>Sequence ID 90: SI-55P4 heavy chain nucleotide sequence
GAAATCGTTATGACACAGTCCCCATCCACTCTTAGCGCTTCTGTAGGGGATCGAGTGATTATCACATGCCGGGCCTCCCAATCCAT
AGGAACCAACATACACTGGTATCAACAAAAACCAGGCAAAGCGCCAAAACTGCTTATCTACTACGCCTCCGAGAGTATTTCTGGAA
TCCCGAGTCGCTTCTCAGGTTCTGGAAGCGGCGCTGAGTTTACCCTCACAATTTCTTCACTCCAACCGGATGACTTCGCTACATAT
TACTGCCAACAAAACAATAATTGGCCGACGACCTTTGGCCAGGGCACGAAACTTACGGTACTTGGCGGTGGCGGTAGTGGGGGAGG
CGGTTCTGGCGGCGGAGGGTCCGGCGGTGGAGGATCAGAAGTACAGCTTGTCGAGTCCGGTGGGGGGCTTGTTCAGCCAGGGGGTT
CCTTGAGGCTTTCCTGCTCCGTCTCTGGGTTTAGCTTGACGAATTACGGCGTTCACTGGGTTAGACAAGCACCGGGGAAGGGGCTG
GAATGGGTCGGTGTGATATGGTCCGGGGGTAATACGGATTACAATACACCTTTCACGTCACGCTTTACGATTAGCAGGGACACGTC
AAAAAATACAGTCTACTTGCAGATGAACTCTCTTAGGGCGGAAGATACTGCAGTTTATTACTGCGCAAGGGCTCTGACATACTACG
ATTATGAATTTGCATATTGGGGCCAGGGGACTTTGGTCACGGTCTCGAGCGGCGGTGGAGGGTCCGGCGGTGGTGGATCACAAGTG
CAGTTGCAAGAAAGTGGTGGTAGACTGGTTCAGCCTGGTGAACCCTTGTCACTGACGTGTAAAACAAGCGGCATTGATCTGTCCTC
TAACGCCATCGGATGGGTCCGACAGGCCCCAGGAAAAGGTCTGGAGTGGATCGGAGTTATCTTCGGGAGCGGCAATACATACTACG
CAAGCTGGGCAAAAGGGCGATTTACGATATCACGGAGCACCTCTACAGTTTATTTGAAAATGAACTCCCTCCGGTCCGAGGATACC
GCGATATATTACTGTGCCAGAGGGGGGTACTCCTCTGATATCTGGGGGCAGGGTACACTGGTTACAGTTTCATCCGCTAGCACCAA
GGGCCCATCGGTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCACACATGCCCACCGTGCCCAGCACCTGAAGCCGCGG
GGGCACCGTCAGTCTTCCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAATGCCAAGACAAAGCCGCGGGA
GGAGCAGTACAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTGGCGGTGGAGGGTCCGGCGGTGGTGGATCCGAGGTGCAGCTGTTGGAGTCTGGGGG
AGGCTTGGTACAGCCTGGGGGGTCCCTGAGACTCTCCTGTGCAGCCTCTGGATTCTCCTTCAGTAGCGGGTACGACATGTGCTGGG
TCCGCCAGGCTCCAGGGAAGGGGCTGGAGTGGATCGCATGCATTGCTGCTGGTAGTGCTGGTATCACTTACGACGCGAACTGGGCG
AAAGGCCGGTTCACCATCTCCAGAGACAATTCCAAGAACACGCTGTATCTGCAAATGAACAGCCTGAGAGCCGAGGACACGGCCGT
ATATTACTGTGCGAGATCGGCGTTTTCGTTCGACTACGCCATGGACCTCTGGGGCCAGGGAACCCTGGTCACCGTGTCGAGCGGTG
GAGGCGGATCTGGCGGAGGTGGTTCCGGCGGTGGCGGCTCCGGTGGAGGCGGCTCTGACATCCAGATGACCCAGTCTCCTTCCACC
CTGTCTGCATCTGTAGGAGACAGAGTCACCATCACTTGCCAGGCCAGTCAGAGCATTAGTTCCCACTTAAACTGGTATCAGCAGAA
ACCAGGGAAAGCCCCTAAGCTCCTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCAACAGGGTTATAGTTGGGGTAAT
GTTGATAATGTTTTCGGCGGAGGGACCAAGGTGGAGATCAAAGGCGGTGGAGGGTCCGGCGGTGGTGGCTCCGGACGAGAGGGCCC
CGAGCTGTCTCCTGATGACCCAGCAGGCCTCTTGGACTTGCGGCAGGGTATGTTCGCTCAACTTGTGGCTCAGAATGTTCTGCTCA
TTGATGGACCACTCTCTTGGTATAGTGACCCCGGTCTGGCCGGGGTGAGTCTGACCGGCGGGCTCTCTTATAAAGAGGATACTAAG
GAACTGGTCGTAGCAAAAGCGGGCGTTTATTACGTTTTTTTTCAGCTGGAGCTCAGGCGCGTGGTGGCCGGCGAGGGCAGTGGCTC
TGTGTCCCTGGCCCTGCACTTACAGCCCTTGAGAAGCGCTGCAGGTGCTGCCGCACTGGCTTTAACTGTTGACCTCCCTCCGGCCT
CTTCTGAAGCTAGAAACAGCGCTTTCGGCTTCCAAGGGCGCCTGCTGCACCTGAGCGCAGGCCAGCGCTTAGGTGTGCACCTTCAT
ACAGAGGCCAGGGCCCGACACGCTTGGCAGCTCACACAGGGTGCCACGGTTCTCGGACTTTTCCGCGTTACTCCCGAGATCCCCGC
TGGCCTCGGAAGTACTGGTTCTGGGTCTAAACCCGGTTCCGGCGAAGGTAGTACTAAAGGACGAGAAGGGCCAGAGTTAAGTCCAG
ATGACCCTGCTGGGCTTTTGGACCTGCGGCAGGGCATGTTCGCTCAACTGGTGGCTCAGAACGTGCTGCTGATCGATGGCCCCCTG
AGTTGGTACAGCGATCCCGGGCTGGCAGGCGTGTCACTTACAGGGGGCCTCTCTTACAAGGAAGACACCAAGGAGTTAGTGGTCGC
TAAGGCTGGCGTGTATTACGTGTTCTTCCAACTGGAGCTGAGAAGGGTTGTGGCAGGAGAGGGTAGCGGCAGCGTGTCTTTAGCCC
TTCACTTGCAGCCCCTGAGGTCTGCTGCAGGTGCAGCCGCTCTCGCGCTCACCGTGGATCTCCCCCCAGCCTCATCTGAAGCTAGG
AACAGTGCATTTGGCTTTCAGGGACGCTTGCTGCACCTCTCCGCTGGACAGAGGCTGGGCGTGCACCTTCACACAGAGGCCCGTGC
CAGGCATGCATGGCAGCTCACTCAGGGGGCAACAGTGCTGGGTCTCTTCCGCGTGACTCCTGAAATACCAGCTGGACTTGGCGGTG
GAGGCAGCGGCGGAGGAGGATCTCGTGAGGGGCCAGAACTGTCCCCCGATGACCCAGCCGGACTGCTCGATCTCAGACAGGGCATG
TTCGCTCAGCTTGTAGCCCAAAATGTCCTCCTGATTGACGGCCCTTTGAGCTGGTATAGTGATCCCGGCTTGGCCGGGGTATCTCT
GACCGGAGGCCTCTCCTACAAGGAAGACACCAAAGAGCTGGTGGTGGCAAAAGCGGGGGTGTATTATGTGTTCTTTCAGCTCGAGC
TGCGGAGAGTTGTGGCCGGGGAAGGGTCTGGGAGCGTATCTCTTGCACTTCACCTGCAGCCCCTGCGCAGCGCCGCTGGAGCCGCC
GCCCTTGCTCTTACTGTGGATCTGCCTCCTGCTTCCTCAGAAGCACGCAACAGCGCCTTCGGCTTTCAAGGACGTCTCCTGCACTT
GTCCGCAGGACAGAGGTTGGGCGTCCATTTACACACTGAGGCACGGGCACGGCACGCTTGGCAGCTTACCCAGGGAGCCACCGTGC
TGGGACTCTTTAGAGTGACACCCGAGATCCCCGCTGGCTTGTGA
>Sequence ID 91: SI-55P4 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDP
VLTQSPSSLSASVGDRVTISCQSSQSVAKNNNLAWFQQKPGQAPKLLIYSASTLAAGVPSRFSGSGSGTDFTLTISSVQPEDFATY
YCSARDSGNIQSFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSENRGEC
>Sequence ID 92: SI-55P4 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACGGTACTGGGTGGAGGCGGTTCAGGCGGAGGTGG
TTCCGGCGGTGGCGGCTCCGGTGGAGGCGGCTCTCAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCCGATCCA
GTTCTGACACAAAGTCCATCCAGCCTGTCTGCCTCAGTCGGCGACAGAGTGACCATCAGTTGCCAGAGCTCACAGTCTGTGGCTAA
GAACAACAACTTGGCGTGGTTCCAACAGAAACCTGGACAGGCTCCGAAATTGCTGATCTATTCTGCTTCCACGCTTGCTGCTGGTG
TTCCTTCCCGCTTTTCAGGTAGTGGTAGCGGGACAGACTTCACTTTGACTATAAGCAGCGTGCAGCCTGAAGATTTTGCGACCTAC
TATTGTTCTGCTAGAGACAGTGGAAATATTCAGTCCTTTGGGGGGGGAACGAAGGTCGAAATAAAGCGTACGGTGGCTGCACCATC
TGTCTTCATCTTCCCGCCATCTGATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTTGA
>Sequence ID 93: SI-79P2 heavy chain amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLELKGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGSGGGGSQV
QLQESGGRLVQPGEPLSLTCKTSGIDLSSNAIGWVRQAPGKGLEWIGVIFGSGNTYYASWAKGRFTISRSTSTVYLKMNSLRSEDT
AIYYCARGGYSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGAPSVFLFPPKPKDTLMISRTPEVTCVVV
DVSHEDPEVKFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSVMHEALHNHY
TQKSLSLSPGGGGGSGGGGSEVQLLESGGGLVQPGGSLRLSCAASGFSFSSGYDMCWVRQAPGKGLEWIACIAAGSAGITYDANWA
KGRFTISRDNSKNTLYLQMNSLRAEDTAVYYCARSAFSFDYAMDLWGQGTLVTVSSGGGGSGGGGSGGGGSGGGGSDIQMTQSPST
LSASVGDRVTITCQASQSISSHLNWYQQKPGKAPKLLIYKASTLASGVPSRFSGSGSGTEFTLTISSLQPDDFATYYCQQGYSWGN
VDNVFGGGTKVEIKGGGGSGGGGSGRSLVESGGGLVQPGGSLRLSCTASGFTISSYHMQWVRQAPGKGLEYIGTISSGGNVYYASS
ARGRFTISRPSSKNTVDLQMNSLRAEDTAVYYCARDSGYSDPMWGQGTLVTVSSGGGGSGGGGSGGGGSGGGGSDVVMTQSPSSVS
ASVGDRVTITCQASQNIRTYLSWYQQKPGKAPKLLIYAAANLASGVPSRFSGSGSGTDFTLTISDLEPGDAATYYCQSTYLGTDYV
GGAFGGGTKVEIK
>Sequence ID 94: SI-79P2 heavy chain nucleotide sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGTTGGAACTGAAAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGCGGTGGAGGGTCCGGCGGTGGTGGATCACAAGTG
CAGTTGCAAGAAAGTGGTGGTAGACTGGTTCAGCCTGGTGAACCCTTGTCACTGACGTGTAAAACAAGCGGCATTGATCTGTCCTC
TAACGCCATCGGATGGGTCCGACAGGCCCCAGGAAAAGGTCTGGAGTGGATCGGAGTTATCTTCGGGAGCGGCAATACATACTACG
CAAGCTGGGCAAAAGGGCGATTTACGATATCACGGAGCACCTCTACAGTTTATTTGAAAATGAACTCCCTCCGGTCCGAGGATACC
GCGATATATTACTGTGCCAGAGGGGGGTACTCCTCTGATATCTGGGGGCAGGGTACACTGGTTACAGTTTCATCCGCTAGCACCAA
GGGCCCATCGGTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCACACATGCCCACCGTGCCCAGCACCTGAAGCCGCGG
GGGCACCGTCAGTCTTCCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAATGCCAAGACAAAGCCGCGGGA
GGAGCAGTACAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTGGCGGTGGAGGGTCCGGCGGTGGTGGATCCGAGGTGCAGCTGTTGGAGTCTGGGGG
AGGCTTGGTACAGCCTGGGGGGTCCCTGAGACTCTCCTGTGCAGCCTCTGGATTCTCCTTCAGTAGCGGGTACGACATGTGCTGGG
TCCGCCAGGCTCCAGGGAAGGGGCTGGAGTGGATCGCATGCATTGCTGCTGGTAGTGCTGGTATCACTTACGACGCGAACTGGGCG
AAAGGCCGGTTCACCATCTCCAGAGACAATTCCAAGAACACGCTGTATCTGCAAATGAACAGCCTGAGAGCCGAGGACACGGCCGT
ATATTACTGTGCGAGATCGGCGTTTTCGTTCGACTACGCCATGGACCTCTGGGGCCAGGGAACCCTGGTCACCGTGTCGAGCGGTG
GAGGCGGATCTGGCGGAGGTGGTTCCGGCGGTGGCGGCTCCGGTGGAGGCGGCTCTGACATCCAGATGACCCAGTCTCCTTCCACC
CTGTCTGCATCTGTAGGAGACAGAGTCACCATCACTTGCCAGGCCAGTCAGAGCATTAGTTCCCACTTAAACTGGTATCAGCAGAA
ACCAGGGAAAGCCCCTAAGCTCCTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCAACAGGGTTATAGTTGGGGTAAT
GTTGATAATGTTTTCGGCGGAGGGACCAAGGTGGAGATCAAAGGCGGTGGAGGGTCCGGCGGTGGTGGCTCCGGACGGTCGCTGGT
GGAGTCTGGGGGAGGCTTGGTCCAGCCTGGGGGGTCCCTGAGACTCTCCTGTACTGCCTCTGGATTCACCATCAGTAGCTACCACA
TGCAGTGGGTCCGCCAGGCTCCAGGGAAGGGGCTGGAGTACATCGGAACCATTAGTAGTGGTGGTAATGTATACTACGCAAGCTCC
GCTAGAGGCAGATTCACCATCTCCAGACCCTCGTCCAAGAACACGGTGGATCTTCAAATGAACAGCCTGAGAGCCGAGGACACGGC
TGTGTATTACTGTGCGAGAGACTCTGGTTATAGTGATCCTATGTGGGGCCAGGGAACCCTGGTCACCGTCTCTTCAGGCGGTGGCG
GTAGTGGGGGAGGCGGTTCTGGCGGCGGAGGGTCCGGCGGTGGAGGATCAGACGTTGTGATGACCCAGTCTCCATCTTCCGTGTCT
GCATCTGTAGGAGACAGAGTCACCATCACCTGTCAGGCCAGTCAGAACATTAGGACTTACTTATCCTGGTATCAGCAGAAACCAGG
GAAAGCCCCTAAGCTCCTGATCTATGCTGCAGCCAATCTGGCATCTGGGGTCCCATCAAGGTTCAGCGGCAGTGGATCTGGGACAG
ATTTCACTCTCACCATCAGCGACCTGGAGCCTGGCGATGCTGCAACTTACTATTGTCAGTCTACCTATCTTGGTACTGATTATGTT
GGCGGTGCTTTCGGCGGAGGGACCAAGGTGGAGATCAAATGA
>Sequence ID 95: SI-79P2 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDP
VLTQSPSSLSASVGDRVTISCQSSQSVAKNNNLAWFQQKPGQAPKLLIYSASTLAAGVPSRFSGSGSGTDFTLTISSVQPEDFATY
YCSARDSGNIQSFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSENRGEC
>Sequence ID 96: SI-79P2 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACGGTACTGGGTGGAGGCGGTTCAGGCGGAGGTGG
TTCCGGCGGTGGCGGCTCCGGTGGAGGCGGCTCTCAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCCGATCCA
GTTCTGACACAAAGTCCATCCAGCCTGTCTGCCTCAGTCGGCGACAGAGTGACCATCAGTTGCCAGAGCTCACAGTCTGTGGCTAA
GAACAACAACTTGGCGTGGTTCCAACAGAAACCTGGACAGGCTCCGAAATTGCTGATCTATTCTGCTTCCACGCTTGCTGCTGGTG
TTCCTTCCCGCTTTTCAGGTAGTGGTAGCGGGACAGACTTCACTTTGACTATAAGCAGCGTGCAGCCTGAAGATTTTGCGACCTAC
TATTGTTCTGCTAGAGACAGTGGAAATATTCAGTCCTTTGGGGGGGGAACGAAGGTCGAAATAAAGCGTACGGTGGCTGCACCATC
TGTCTTCATCTTCCCGCCATCTGATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTTGA
>Sequence ID 97: SI-79P3 heavy chain amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLELKGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGSGGGGSQV
QLQESGGRLVQPGEPLSLTCKTSGIDLSSNAIGWVRQAPGKGLEWIGVIFGSGNTYYASWAKGRFTISRSTSTVYLKMNSLRSEDT
AIYYCARGGYSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGAPSVFLFPPKPKDTLMISRTPEVTCVVV
DVSHEDPEVKFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSVMHEALHNHY
TQKSLSLSPGGGGGSGGGGSEVQLLESGGGLVQPGGSLRLSCAASGFSFSSGYDMCWVRQAPGKGLEWIACIAAGSAGITYDANWA
KGRFTISRDNSKNTLYLQMNSLRAEDTAVYYCARSAFSFDYAMDLWGQGTLVTVSSGGGGSGGGGSGGGGSGGGGSDIQMTQSPST
LSASVGDRVTITCQASQSISSHLNWYQQKPGKAPKLLIYKASTLASGVPSRFSGSGSGTEFTLTISSLQPDDFATYYCQQGYSWGN
VDNVFGGGTKVEIKGGGGSGGGGSGREGPELSPDDPAGLLDLRQGMFAQLVAQNVLLIDGPLSWYSDPGLAGVSLTGGLSYKEDTK
ELVVAKAGVYYVFFQLELRRVVAGEGSGSVSLALHLQPLRSAAGAAALALTVDLPPASSEARNSAFGFQGRLLHLSAGQRLGVHLH
TEARARHAWQLTQGATVLGLFRVTPEIPAGLGSTGSGSKPGSGEGSTKGREGPELSPDDPAGLLDLRQGMFAQLVAQNVLLIDGPL
SWYSDPGLAGVSLTGGLSYKEDTKELVVAKAGVYYVFFQLELRRVVAGEGSGSVSLALHLQPLRSAAGAAALALTVDLPPASSEAR
NSAFGFQGRLLHLSAGQRLGVHLHTEARARHAWQLTQGATVLGLFRVTPEIPAGLGGGGSGGGGSREGPELSPDDPAGLLDLRQGM
FAQLVAQNVLLIDGPLSWYSDPGLAGVSLTGGLSYKEDTKELVVAKAGVYYVFFQLELRRVVAGEGSGSVSLALHLQPLRSAAGAA
ALALTVDLPPASSEARNSAFGFQGRLLHLSAGQRLGVHLHTEARARHAWQLTQGATVLGLFRVTPEIPAGL
>Sequence ID 98: SI-79P3 heavy chain nucleotide sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGTTGGAACTGAAAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGCGGTGGAGGGTCCGGCGGTGGTGGATCACAAGTG
CAGTTGCAAGAAAGTGGTGGTAGACTGGTTCAGCCTGGTGAACCCTTGTCACTGACGTGTAAAACAAGCGGCATTGATCTGTCCTC
TAACGCCATCGGATGGGTCCGACAGGCCCCAGGAAAAGGTCTGGAGTGGATCGGAGTTATCTTCGGGAGCGGCAATACATACTACG
CAAGCTGGGCAAAAGGGCGATTTACGATATCACGGAGCACCTCTACAGTTTATTTGAAAATGAACTCCCTCCGGTCCGAGGATACC
GCGATATATTACTGTGCCAGAGGGGGGTACTCCTCTGATATCTGGGGGCAGGGTACACTGGTTACAGTTTCATCCGCTAGCACCAA
GGGCCCATCGGTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCACACATGCCCACCGTGCCCAGCACCTGAAGCCGCGG
GGGCACCGTCAGTCTTCCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAATGCCAAGACAAAGCCGCGGGA
GGAGCAGTACAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTGGCGGTGGAGGGTCCGGCGGTGGTGGATCCGAGGTGCAGCTGTTGGAGTCTGGGGG
AGGCTTGGTACAGCCTGGGGGGTCCCTGAGACTCTCCTGTGCAGCCTCTGGATTCTCCTTCAGTAGCGGGTACGACATGTGCTGGG
TCCGCCAGGCTCCAGGGAAGGGGCTGGAGTGGATCGCATGCATTGCTGCTGGTAGTGCTGGTATCACTTACGACGCGAACTGGGCG
AAAGGCCGGTTCACCATCTCCAGAGACAATTCCAAGAACACGCTGTATCTGCAAATGAACAGCCTGAGAGCCGAGGACACGGCCGT
ATATTACTGTGCGAGATCGGCGTTTTCGTTCGACTACGCCATGGACCTCTGGGGCCAGGGAACCCTGGTCACCGTGTCGAGCGGTG
GAGGCGGATCTGGCGGAGGTGGTTCCGGCGGTGGCGGCTCCGGTGGAGGCGGCTCTGACATCCAGATGACCCAGTCTCCTTCCACC
CTGTCTGCATCTGTAGGAGACAGAGTCACCATCACTTGCCAGGCCAGTCAGAGCATTAGTTCCCACTTAAACTGGTATCAGCAGAA
ACCAGGGAAAGCCCCTAAGCTCCTGATCTATAAGGCATCCACTCTGGCATCTGGGGTCCCATCAAGGTTCAGCGGCAGTGGATCTG
GGACAGAATTTACTCTCACCATCAGCAGCCTGCAGCCTGATGATTTTGCAACTTATTACTGCCAACAGGGTTATAGTTGGGGTAAT
GTTGATAATGTTTTCGGCGGAGGGACCAAGGTGGAGATCAAAGGCGGTGGAGGGTCCGGCGGTGGTGGCTCCGGACGAGAGGGCCC
CGAGCTGTCTCCTGATGACCCAGCAGGCCTCTTGGACTTGCGGCAGGGTATGTTCGCTCAACTTGTGGCTCAGAATGTTCTGCTCA
TTGATGGACCACTCTCTTGGTATAGTGACCCCGGTCTGGCCGGGGTGAGTCTGACCGGCGGGCTCTCTTATAAAGAGGATACTAAG
GAACTGGTCGTAGCAAAAGCGGGCGTTTATTACGTTTTTTTTCAGCTGGAGCTCAGGCGCGTGGTGGCCGGCGAGGGCAGTGGCTC
TGTGTCCCTGGCCCTGCACTTACAGCCCTTGAGAAGCGCTGCAGGTGCTGCCGCACTGGCTTTAACTGTTGACCTCCCTCCGGCCT
CTTCTGAAGCTAGAAACAGCGCTTTCGGCTTCCAAGGGCGCCTGCTGCACCTGAGCGCAGGCCAGCGCTTAGGTGTGCACCTTCAT
ACAGAGGCCAGGGCCCGACACGCTTGGCAGCTCACACAGGGTGCCACGGTTCTCGGACTTTTCCGCGTTACTCCCGAGATCCCCGC
TGGCCTCGGAAGTACTGGTTCTGGGTCTAAACCCGGTTCCGGCGAAGGTAGTACTAAAGGACGAGAAGGGCCAGAGTTAAGTCCAG
ATGACCCTGCTGGGCTTTTGGACCTGCGGCAGGGCATGTTCGCTCAACTGGTGGCTCAGAACGTGCTGCTGATCGATGGCCCCCTG
AGTTGGTACAGCGATCCCGGGCTGGCAGGCGTGTCACTTACAGGGGGCCTCTCTTACAAGGAAGACACCAAGGAGTTAGTGGTCGC
TAAGGCTGGCGTGTATTACGTGTTCTTCCAACTGGAGCTGAGAAGGGTTGTGGCAGGAGAGGGTAGCGGCAGCGTGTCTTTAGCCC
TTCACTTGCAGCCCCTGAGGTCTGCTGCAGGTGCAGCCGCTCTCGCGCTCACCGTGGATCTCCCCCCAGCCTCATCTGAAGCTAGG
AACAGTGCATTTGGCTTTCAGGGACGCTTGCTGCACCTCTCCGCTGGACAGAGGCTGGGCGTGCACCTTCACACAGAGGCCCGTGC
CAGGCATGCATGGCAGCTCACTCAGGGGGCAACAGTGCTGGGTCTCTTCCGCGTGACTCCTGAAATACCAGCTGGACTTGGCGGTG
GAGGCAGCGGCGGAGGAGGATCTCGTGAGGGGCCAGAACTGTCCCCCGATGACCCAGCCGGACTGCTCGATCTCAGACAGGGCATG
TTCGCTCAGCTTGTAGCCCAAAATGTCCTCCTGATTGACGGCCCTTTGAGCTGGTATAGTGATCCCGGCTTGGCCGGGGTATCTCT
GACCGGAGGCCTCTCCTACAAGGAAGACACCAAAGAGCTGGTGGTGGCAAAAGCGGGGGTGTATTATGTGTTCTTTCAGCTCGAGC
TGCGGAGAGTTGTGGCCGGGGAAGGGTCTGGGAGCGTATCTCTTGCACTTCACCTGCAGCCCCTGCGCAGCGCCGCTGGAGCCGCC
GCCCTTGCTCTTACTGTGGATCTGCCTCCTGCTTCCTCAGAAGCACGCAACAGCGCCTTCGGCTTTCAAGGACGTCTCCTGCACTT
GTCCGCAGGACAGAGGTTGGGCGTCCATTTACACACTGAGGCACGGGCACGGCACGCTTGGCAGCTTACCCAGGGAGCCACCGTGC
TGGGACTCTTTAGAGTGACACCCGAGATCCCCGCTGGCTTGTGA
>Sequence ID 99: SI-79P3 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGGGGGSDP
VLTQSPSSLSASVGDRVTISCQSSQSVAKNNNLAWFQQKPGQAPKLLIYSASTLAAGVPSRFSGSGSGTDFTLTISSVQPEDFATY
YCSARDSGNIQSFGGGTKVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSENRGEC
>Sequence ID 100: SI-79P3 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACGGTACTGGGTGGAGGCGGTTCAGGCGGAGGTGG
TTCCGGCGGTGGCGGCTCCGGTGGAGGCGGCTCTCAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCCGATCCA
GTTCTGACACAAAGTCCATCCAGCCTGTCTGCCTCAGTCGGCGACAGAGTGACCATCAGTTGCCAGAGCTCACAGTCTGTGGCTAA
GAACAACAACTTGGCGTGGTTCCAACAGAAACCTGGACAGGCTCCGAAATTGCTGATCTATTCTGCTTCCACGCTTGCTGCTGGTG
TTCCTTCCCGCTTTTCAGGTAGTGGTAGCGGGACAGACTTCACTTTGACTATAAGCAGCGTGCAGCCTGAAGATTTTGCGACCTAC
TATTGTTCTGCTAGAGACAGTGGAAATATTCAGTCCTTTGGGGGGGGAACGAAGGTCGAAATAAAGCGTACGGTGGCTGCACCATC
TGTCTTCATCTTCCCGCCATCTGATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTTGA
>Sequence ID 101: SI-55P9 heavy chain amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVLGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGGGGGSEV
QLVESGGGLVQPGGSLRLSCAASGFTISTNAMSWVRQAPGKGLEWIGVITGRDITYYASWAKGRFTISRDNSKNTLYLQMNSLRAE
DTAVYYCARDGGSSAITSNNIWGQGTLVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPAPEAAGAPSVFLFPPKPKDTLMISRTP
EVTCVVVDVSHEDPEVKFNWYVDGVEVHNAKTKPREEQYNSTYRVVSVLTVLHQDWLNGKEYKCAVSNKALPAPIEKTISKAKGQP
REPQVYTLPPSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIACIAAGSAGI
TYDANWAKGRFTISRDNSKNTLYLQMNSLRAEDTAVYYCARSAFSFDYAMDLWGQGTLVTVSSGGGGSGGGGSGGGGSGGGGSDIQ
MTQSPSTLSASVGDRVTITCQASQSISSHLNWYQQKPGKAPKLLIYKASTLASGVPSRFSGSGSGTEFTLTISSLQPDDFATYYCQ
QGYSWGNVDNVFGGGTKVEIKGGGGSGGGGSGRSLVESGGGLVQPGGSLRLSCTASGFTISSYHMQWVRQAPGKGLEYIGTISSGG
NVYYASSARGRFTISRPSSKNTVDLQMNSLRAEDTAVYYCARDSGYSDPMWGQGTLVTVSSGGGGSGGGGSGGGGSGGGGSDVVMT
QSPSSVSASVGDRVTITCQASQNIRTYLSWYQQKPGKAPKLLIYAAANLASGVPSRFSGSGSGTDFTLTISDLEPGDAATYYCQST
YLGTDYVGGAFGGGTKVEIK
>Sequence ID 102: SI-55P9 heavy chain nucleotide sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGCTGACCGTTTTAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGCGGTGGAGGGTCCGGCGGTGGTGGATCAGAGGTG
CAGCTGGTGGAGTCTGGGGGAGGCTTGGTCCAGCCTGGGGGGTCCCTGAGACTCTCCTGTGCAGCCTCTGGATTCACCATCAGTAC
CAATGCAATGAGCTGGGTCCGCCAGGCTCCAGGGAAGGGGCTGGAGTGGATCGGAGTCATTACTGGTCGTGATATCACATACTACG
CGAGCTGGGCGAAAGGCAGATTCACCATCTCCAGAGACAATTCCAAGAACACGCTGTATCTTCAAATGAACAGCCTGAGAGCCGAG
GACACGGCTGTGTATTACTGTGCGAGAGACGGTGGTTCTTCTGCTATTACTAGTAACAACATTTGGGGCCAGGGAACCCTGGTCAC
CGTGTCCTCAGCTAGCACCAAGGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAGAGTTGAGCCCAAATCTTGTGACAAAACTCACACATGCCCACCGT
GCCCAGCACCTGAAGCCGCGGGGGCACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCGTCCTGCACCAGGACTGGCTGA
ATGGCAAGGAGTACAAGTGCGCGGTCTCCAACAAAGCCCTCCCAGCCCCCATCGAGAAAACCATCTCCAAAGCCAAAGGGCAGCCC
CGAGAACCACAGGTGTACACCCTGCCCCCATCCCGGGATGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGACCACGCCTCCCGTGCTGGACTCCG
ACGGCTCCTTCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGGATCCGAGGT
GCAGCTGTTGGAGTCTGGGGGAGGCTTGGTACAGCCTGGGGGGTCCCTGAGACTCTCCTGTGCAGCCTCTGGATTCTCCTTCAGTA
GCGGGTACGACATGTGCTGGGTCCGCCAGGCTCCAGGGAAGGGGCTGGAGTGGATCGCATGCATTGCTGCTGGTAGTGCTGGTATC
ACTTACGACGCGAACTGGGCGAAAGGCCGGTTCACCATCTCCAGAGACAATTCCAAGAACACGCTGTATCTGCAAATGAACAGCCT
GAGAGCCGAGGACACGGCCGTATATTACTGTGCGAGATCGGCGTTTTCGTTCGACTACGCCATGGACCTCTGGGGCCAGGGAACCC
TGGTCACCGTGTCGAGCGGTGGAGGCGGATCTGGCGGAGGTGGTTCCGGCGGTGGCGGCTCCGGTGGAGGCGGCTCTGACATCCAG
ATGACCCAGTCTCCTTCCACCCTGTCTGCATCTGTAGGAGACAGAGTCACCATCACTTGCCAGGCCAGTCAGAGCATTAGTTCCCA
CTTAAACTGGTATCAGCAGAAACCAGGGAAAGCCCCTAAGCTCCTGATCTATAAGGCATCCACTCTGGCATCTGGGGTCCCATCAA
GGTTCAGCGGCAGTGGATCTGGGACAGAATTTACTCTCACCATCAGCAGCCTGCAGCCTGATGATTTTGCAACTTATTACTGCCAA
CAGGGTTATAGTTGGGGTAATGTTGATAATGTTTTCGGCGGAGGGACCAAGGTGGAGATCAAAGGCGGTGGAGGGTCCGGCGGTGG
TGGCTCCGGACGGTCGCTGGTGGAGTCTGGGGGAGGCTTGGTCCAGCCTGGGGGGTCCCTGAGACTCTCCTGTACTGCCTCTGGAT
TCACCATCAGTAGCTACCACATGCAGTGGGTCCGCCAGGCTCCAGGGAAGGGGCTGGAGTACATCGGAACCATTAGTAGTGGTGGT
AATGTATACTACGCAAGCTCCGCTAGAGGCAGATTCACCATCTCCAGACCCTCGTCCAAGAACACGGTGGATCTTCAAATGAACAG
CCTGAGAGCCGAGGACACGGCTGTGTATTACTGTGCGAGAGACTCTGGTTATAGTGATCCTATGTGGGGCCAGGGAACCCTGGTCA
CCGTCTCTTCAGGCGGTGGCGGTAGTGGGGGAGGCGGTTCTGGCGGCGGAGGGTCCGGCGGTGGAGGATCAGACGTTGTGATGACC
CAGTCTCCATCTTCCGTGTCTGCATCTGTAGGAGACAGAGTCACCATCACCTGTCAGGCCAGTCAGAACATTAGGACTTACTTATC
CTGGTATCAGCAGAAACCAGGGAAAGCCCCTAAGCTCCTGATCTATGCTGCAGCCAATCTGGCATCTGGGGTCCCATCAAGGTTCA
GCGGCAGTGGATCTGGGACAGATTTCACTCTCACCATCAGCGACCTGGAGCCTGGCGATGCTGCAACTTACTATTGTCAGTCTACC
TATCTTGGTACTGATTATGTTGGCGGTGCTTTCGGCGGAGGGACCAAGGTGGAGATCAAATGA
>Sequence ID 103: SI-55P9 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLRLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDV
VMTQSPSTLSASVGDRVTINCQASESISSWLAWYQQKPGKAPKLLIYEASKLASGVPSRFSGSGSGTEFTLTISSLQPDDFATYYC
QGYFYFISRTYVNSFGGGTKVEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSENRGEC
>Sequence ID 104: SI-55P9 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACGGTACTGGGTGGAGGCGGTTCAGGCGGAGGTGG
TTCCGGCGGTGGCGGCTCCGGTGGAGGCGGCTCTCAGGTCACATTGAAGGAATCTGGCCCCGGCCTTGTTCAGCCAGGACAGACCC
TTAGGCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGCGGCGGTGGAGGGTCCGGCGGTGGTGGATCAGACGTC
GTGATGACCCAGTCTCCTTCCACCCTGTCTGCATCTGTAGGAGACAGAGTCACCATCAATTGCCAAGCCAGTGAGAGCATTAGCAG
TTGGTTAGCCTGGTATCAGCAGAAACCAGGGAAAGCCCCTAAGCTCCTGATCTATGAAGCATCCAAACTGGCATCTGGGGTCCCAT
CAAGGTTCAGCGGCAGTGGATCTGGGACAGAATTCACTCTCACCATCAGCAGCCTGCAGCCTGATGATTTTGCAACTTATTACTGC
CAAGGCTATTTTTATTTTATTAGTCGTACTTATGTAAATTCTTTCGGCGGAGGGACCAAGGTGGAGATCAAACGTACGGTGGCTGC
ACCATCTGTCTTCATCTTCCCGCCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTTGA
>Sequence ID 105: SI-77P1 heavy chain amino acid sequence
EIVMTQSPSTLSASVGDRVIITCQASESISSWLAWYQQKPGKAPKLLIYEASKLASGVPSRFSGSGSGAEFTLTISSLQPDDFATY
YCQGYFYFISRTYVNSFGQGTKLTVLGGGGSGGGGSGGGGSGGGGSEVQLVESGGGLVQPGGSLRLSCAASGFTISTNAMSWVRQA
PGKGLEWVGVITGRDITYYASWAKGRFTISRDTSKNTVYLQMNSLRAEDTAVYYCARDGGSSAITSNNIWGQGTLVTVSSGGGGSG
GGGSQVQLQQSGPGLVKPSETLSITCTVSGFSLTNYGVHWIRQAPGKGLEWLGVIWSGGNTDYNTPFTSRFTITKDNSKNQVYFKL
RSVRADDTAIYYCARALTYYDYEFAYWGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPCPAPEAAGAPSVFLFPPKPKDTLM
ISRTPEVTCVVVDVSHEDPEVKFNWYVDGVEVHNAKTKPREEQYNSTYRVVSVLTVLHQDWLNGKEYKCAVSNKALPAPIEKTISK
AKGQPREPQVYTLPPSRDELTKNQVSLTCLVKGFYPSDIAVEWESNGQPENNYKTTPPVLDSDGSFFLYSKLTVDKSRWQQGNVFS
CSVMHEALHNHYTQKSLSLSPGGGGGSGGGGSEVQLLESGGGLVQPGGSLRLSCAASGFSFSSGYDMCWVRQAPGKGLEWIACIAA
GSAGITYDANWAKGRFTISRDNSKNTLYLQMNSLRAEDTAVYYCARSAFSFDYAMDLWGQGTLVTVSSGGGGSGGGGSGGGGSGGG
GSDIQMTQSPSTLSASVGDRVTITCQASQSISSHLNWYQQKPGKAPKLLIYKASTLASGVPSRFSGSGSGTEFTLTISSLQPDDFA
TYYCQQGYSWGNVDNVFGGGTKVEIKGGGGSGGGGSGRSLVESGGGLVQPGGSLRLSCTASGFTISSYHMQWVRQAPGKGLEYIGT
ISSGGNVYYASSARGRFTISRPSSKNTVDLQMNSLRAEDTAVYYCARDSGYSDPMWGQGTLVTVSSGGGGSGGGGSGGGGSGGGGS
DVVMTQSPSSVSASVGDRVTITCQASQNIRTYLSWYQQKPGKAPKLLIYAAANLASGVPSRFSGSGSGTDFTLTISDLEPGDAATY
YCQSTYLGTDYVGGAFGGGTKVEIK
>Sequence ID 106: SI-77P1 heavy chain nucleotide sequence
GAAATCGTTATGACGCAGAGTCCCTCCACGCTCTCCGCTAGTGTCGGGGATCGCGTCATTATCACATGCCAGGCCTCCGAGTCAAT
CAGCAGCTGGCTTGCATGGTATCAACAGAAGCCGGGAAAAGCTCCTAAATTGCTGATCTATGAAGCGTCAAAATTGGCGTCTGGTG
TCCCATCTAGGTTCTCCGGCTCTGGGTCTGGTGCGGAATTTACTTTGACAATCTCCAGTCTTCAACCAGACGATTTCGCTACCTAC
TACTGCCAAGGGTATTTCTATTTTATAAGCCGGACATATGTAAACTCCTTCGGCCAAGGAACAAAGTTGACTGTTCTTGGTGGCGG
AGGCAGTGGTGGCGGGGGCAGCGGAGGTGGTGGTTCAGGGGGTGGTGGGAGCGAAGTCCAATTGGTAGAAAGTGGCGGTGGTCTGG
TGCAACCTGGTGGATCTCTTCGCCTCTCATGCGCCGCTAGTGGCTTTACTATTTCAACTAATGCGATGAGCTGGGTTCGCCAGGCC
CCCGGCAAAGGACTTGAGTGGGTCGGCGTCATCACCGGCAGGGACATTACATACTATGCGAGTTGGGCAAAGGGCAGGTTCACGAT
TAGCCGCGATACTTCAAAGAATACCGTTTACCTTCAAATGAATAGCTTGAGGGCGGAAGACACAGCTGTGTATTACTGCGCGAGGG
ATGGAGGTAGTTCCGCCATAACTTCCAACAACATATGGGGACAAGGCACGCTGGTTACTGTCTCGAGTGGCGGTGGAGGGTCCGGC
GGTGGTGGATCACAAGTACAGTTGCAGCAATCCGGTCCCGGTCTCGTCAAACCGAGTGAGACGCTTAGTATAACGTGTACTGTTTC
AGGCTTTAGCCTTACGAACTATGGAGTTCACTGGATTCGGCAGGCACCCGGCAAAGGTTTGGAATGGCTGGGTGTTATTTGGTCAG
GTGGAAATACAGACTATAACACCCCCTTTACAAGTCGGTTCACAATTACGAAAGATAATTCCAAAAATCAAGTTTATTTCAAGTTG
AGATCCGTCCGCGCGGACGACACTGCGATCTACTATTGTGCGAGGGCACTGACCTACTACGATTACGAATTTGCGTATTGGGGGCA
AGGGACTCTTGTAACAGTCTCCAGTGCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGACAAGAGAGTTGAGCCCAAATCTTGTGACAAAACTC
ACACATGCCCACCGTGCCCAGCACCTGAAGCCGCGGGGGCACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGTGTGGTCAGCGTCCTCACCGTCCTGC
ACCAGGACTGGCTGAATGGCAAGGAGTACAAGTGCGCGGTCTCCAACAAAGCCCTCCCAGCCCCCATCGAGAAAACCATCTCCAAA
GCCAAAGGGCAGCCCCGAGAACCACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAGAACAACTACAAGACCACGCCTC
CCGTGCTGGACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTGCAGCCTCTG
GATTCTCCTTCAGTAGCGGGTACGACATGTGCTGGGTCCGCCAGGCTCCAGGGAAGGGGCTGGAGTGGATCGCATGCATTGCTGCT
GGTAGTGCTGGTATCACTTACGACGCGAACTGGGCGAAAGGCCGGTTCACCATCTCCAGAGACAATTCCAAGAACACGCTGTATCT
GCAAATGAACAGCCTGAGAGCCGAGGACACGGCCGTATATTACTGTGCGAGATCGGCGTTTTCGTTCGACTACGCCATGGACCTCT
GGGGCCAGGGAACCCTGGTCACCGTGTCGAGCGGTGGAGGCGGATCTGGCGGAGGTGGTTCCGGCGGTGGCGGCTCCGGTGGAGGC
GGCTCTGACATCCAGATGACCCAGTCTCCTTCCACCCTGTCTGCATCTGTAGGAGACAGAGTCACCATCACTTGCCAGGCCAGTCA
GAGCATTAGTTCCCACTTAAACTGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAGCCTGCAGCCTGATGATTTTGCA
ACTTATTACTGCCAACAGGGTTATAGTTGGGGTAATGTTGATAATGTTTTCGGCGGAGGGACCAAGGTGGAGATCAAAGGCGGTGG
AGGGTCCGGCGGTGGTGGCTCCGGACGGTCGCTGGTGGAGTCTGGGGGAGGCTTGGTCCAGCCTGGGGGGTCCCTGAGACTCTCCT
GTACTGCCTCTGGATTCACCATCAGTAGCTACCACATGCAGTGGGTCCGCCAGGCTCCAGGGAAGGGGCTGGAGTACATCGGAACC
ATTAGTAGTGGTGGTAATGTATACTACGCAAGCTCCGCTAGAGGCAGATTCACCATCTCCAGACCCTCGTCCAAGAACACGGTGGA
TCTTCAAATGAACAGCCTGAGAGCCGAGGACACGGCTGTGTATTACTGTGCGAGAGACTCTGGTTATAGTGATCCTATGTGGGGCC
AGGGAACCCTGGTCACCGTCTCTTCAGGCGGTGGCGGTAGTGGGGGAGGCGGTTCTGGCGGCGGAGGGTCCGGCGGTGGAGGATCA
GACGTTGTGATGACCCAGTCTCCATCTTCCGTGTCTGCATCTGTAGGAGACAGAGTCACCATCACCTGTCAGGCCAGTCAGAACAT
TAGGACTTACTTATCCTGGTATCAGCAGAAACCAGGGAAAGCCCCTAAGCTCCTGATCTATGCTGCAGCCAATCTGGCATCTGGGG
TCCCATCAAGGTTCAGCGGCAGTGGATCTGGGACAGATTTCACTCTCACCATCAGCGACCTGGAGCCTGGCGATGCTGCAACTTAC
TATTGTCAGTCTACCTATCTTGGTACTGATTATGTTGGCGGTGCTTTCGGCGGAGGGACCAAGGTGGAGATCAAA
>Sequence ID 107: SI-77P1 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSEI
VLTQSPSTLSVSPGERATFSCRASQSIGTNIHWYQQKPGKPPRLLIKYASESISGIPDRFSGSGSGTEFTLTISSVQSEDFAVYYC
QQNNNWPTTFGPGTKLTVLRTVAAPSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSENRGEC
>Sequence ID 108: SI-77P1 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCAGAAATC
GTCCTTACACAATCTCCTAGCACACTGAGTGTGAGCCCCGGCGAACGCGCGACTTTCTCTTGCAGGGCAAGTCAATCCATAGGGAC
TAATATACATTGGTATCAACAAAAGCCAGGTAAACCACCCAGGCTTTTGATTAAGTATGCAAGTGAGTCTATTTCCGGTATCCCTG
ACCGCTTCTCTGGATCAGGCAGTGGCACAGAGTTCACACTCACCATATCTAGTGTGCAATCAGAGGACTTCGCCGTGTATTACTGC
CAACAGAATAATAACTGGCCGACTACCTTCGGACCCGGTACAAAGCTGACCGTTTTACGTACGGTGGCTGCACCATCTGTCTTCAT
CTTCCCGCCATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAGGGCCTGAGCTC
GCCCGTCACAAAGAGCTTCAACAGGGGAGAGTGTTAG
>Sequence ID 109: SI-77H5 heavy chain amino acid sequence
EIVMTQSPSTLSASVGDRVIITCQASESISSWLAWYQQKPGKAPKLLIYEASKLASGVPSRFSGSGSGAEFTLTISSLQPDDFATY
YCQGYFYFISRTYVNSFGQGTKLTVLGGGGSGGGGSGGGGSGGGGSEVQLVESGGGLVQPGGSLRLSCAASGFTISTNAMSWVRQA
PGKGLEWVGVITGRDITYYASWAKGRFTISRDTSKNTVYLQMNSLRAEDTAVYYCARDGGSSAITSNNIWGQGTLVTVSSGGGGSG
GGGSQVQLKQSGPGLVQPSQSLSITCTVSGFSLTNYGVHWVRQSPGKCLEWLGVIWSGGNTDYNTPFTSRLSINKDNSKSQVFFKM
NSLQSNDTAIYYCARALTYYDYEFAYWGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPCPAPEAAGAPSVFLFPPKPKDTLM
ISRTPEVTCVVVDVSHEDPEVKFNWYVDGVEVHNAKTKPREEQYNSTYRVVSVLTVLHQDWLNGKEYKCAVSNKALPAPIEKTISK
AKGQPREPQVYTLPPSRDELTKNQVSLTCLVKGFYPSDIAVEWESNGQPENNYKTTPPVLDSDGSFFLYSKLTVDKSRWQQGNVFS
CSVMHEALHNHYTQKSLSLSPGGGGGSGGGGSEVQLLESGGGLVQPGGSLRLSCAASGFSFSSGYDMCWVRQAPGKGLEWIACIAA
GSAGITYDANWAKGRFTISRDNSKNTLYLQMNSLRAEDTAVYYCARSAFSFDYAMDLWGQGTLVTVSSGGGGSGGGGSGGGGSGGG
GSDIQMTQSPSTLSASVGDRVTITCQASQSISSHLNWYQQKPGKAPKLLIYKASTLASGVPSRFSGSGSGTEFTLTISSLQPDDFA
TYYCQQGYSWGNVDNVFGGGTKVEIKGGGGSGGGGSGRSLVESGGGLVQPGGSLRLSCTASGFTISSYHMQWVRQAPGKGLEYIGT
ISSGGNVYYASSARGRFTISRPSSKNTVDLQMNSLRAEDTAVYYCARDSGYSDPMWGQGTLVTVSSGGGGSGGGGSGGGGSGGGGS
DVVMTQSPSSVSASVGDRVTITCQASQNIRTYLSWYQQKPGKAPKLLIYAAANLASGVPSRFSGSGSGTDFTLTISDLEPGDAATY
YCQSTYLGTDYVGGAFGGGTKVEIK
>Sequence ID 110: SI-77H5 heavy chain nucleotide sequence
GAAATCGTTATGACGCAGAGTCCCTCCACGCTCTCCGCTAGTGTCGGGGATCGCGTCATTATCACATGCCAGGCCTCCGAGTCAAT
CAGCAGCTGGCTTGCATGGTATCAACAGAAGCCGGGAAAAGCTCCTAAATTGCTGATCTATGAAGCGTCAAAATTGGCGTCTGGTG
TCCCATCTAGGTTCTCCGGCTCTGGGTCTGGTGCGGAATTTACTTTGACAATCTCCAGTCTTCAACCAGACGATTTCGCTACCTAC
TACTGCCAAGGGTATTTCTATTTTATAAGCCGGACATATGTAAACTCCTTCGGCCAAGGAACAAAGTTGACTGTTCTTGGTGGCGG
AGGCAGTGGTGGCGGGGGCAGCGGAGGTGGTGGTTCAGGGGGTGGTGGGAGCGAAGTCCAATTGGTAGAAAGTGGCGGTGGTCTGG
TGCAACCTGGTGGATCTCTTCGCCTCTCATGCGCCGCTAGTGGCTTTACTATTTCAACTAATGCGATGAGCTGGGTTCGCCAGGCC
CCCGGCAAAGGACTTGAGTGGGTCGGCGTCATCACCGGCAGGGACATTACATACTATGCGAGTTGGGCAAAGGGCAGGTTCACGAT
TAGCCGCGATACTTCAAAGAATACCGTTTACCTTCAAATGAATAGCTTGAGGGCGGAAGACACAGCTGTGTATTACTGCGCGAGGG
ATGGAGGTAGTTCCGCCATAACTTCCAACAACATATGGGGACAAGGCACGCTGGTTACTGTCTCGAGTGGCGGTGGAGGGTCCGGC
GGTGGTGGATCACAGGTGCAGCTGAAGCAGTCAGGACCTGGCCTAGTGCAGCCCTCACAGAGCCTGTCCATCACCTGCACAGTCTC
TGGTTTCTCATTAACTAACTATGGTGTACACTGGGTTCGCCAGTCTCCAGGAAAGTGTCTGGAGTGGCTGGGAGTGATATGGAGTG
GTGGAAACACAGACTATAATACACCTTTCACATCCAGACTGAGCATCAACAAGGACAATTCCAAGAGCCAAGTTTTCTTTAAAATG
AACAGTCTGCAATCTAATGACACAGCCATATATTACTGTGCCAGAGCCCTCACCTACTATGATTACGAGTTTGCTTACTGGGGCCA
AGGGACTCTGGTCACTGTCTCGAGTGCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGACAAGAGAGTTGAGCCCAAATCTTGTGACAAAACTC
ACACATGCCCACCGTGCCCAGCACCTGAAGCCGCGGGGGCACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGTGTGGTCAGCGTCCTCACCGTCCTGC
ACCAGGACTGGCTGAATGGCAAGGAGTACAAGTGCGCGGTCTCCAACAAAGCCCTCCCAGCCCCCATCGAGAAAACCATCTCCAAA
GCCAAAGGGCAGCCCCGAGAACCACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAGAACAACTACAAGACCACGCCTC
CCGTGCTGGACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTGCAGCCTCTG
GATTCTCCTTCAGTAGCGGGTACGACATGTGCTGGGTCCGCCAGGCTCCAGGGAAGGGGCTGGAGTGGATCGCATGCATTGCTGCT
GGTAGTGCTGGTATCACTTACGACGCGAACTGGGCGAAAGGCCGGTTCACCATCTCCAGAGACAATTCCAAGAACACGCTGTATCT
GCAAATGAACAGCCTGAGAGCCGAGGACACGGCCGTATATTACTGTGCGAGATCGGCGTTTTCGTTCGACTACGCCATGGACCTCT
GGGGCCAGGGAACCCTGGTCACCGTGTCGAGCGGTGGAGGCGGATCTGGCGGAGGTGGTTCCGGCGGTGGCGGCTCCGGTGGAGGC
GGCTCTGACATCCAGATGACCCAGTCTCCTTCCACCCTGTCTGCATCTGTAGGAGACAGAGTCACCATCACTTGCCAGGCCAGTCA
GAGCATTAGTTCCCACTTAAACTGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAGCCTGCAGCCTGATGATTTTGCA
ACTTATTACTGCCAACAGGGTTATAGTTGGGGTAATGTTGATAATGTTTTCGGCGGAGGGACCAAGGTGGAGATCAAAGGCGGTGG
AGGGTCCGGCGGTGGTGGCTCCGGACGGTCGCTGGTGGAGTCTGGGGGAGGCTTGGTCCAGCCTGGGGGGTCCCTGAGACTCTCCT
GTACTGCCTCTGGATTCACCATCAGTAGCTACCACATGCAGTGGGTCCGCCAGGCTCCAGGGAAGGGGCTGGAGTACATCGGAACC
ATTAGTAGTGGTGGTAATGTATACTACGCAAGCTCCGCTAGAGGCAGATTCACCATCTCCAGACCCTCGTCCAAGAACACGGTGGA
TCTTCAAATGAACAGCCTGAGAGCCGAGGACACGGCTGTGTATTACTGTGCGAGAGACTCTGGTTATAGTGATCCTATGTGGGGCC
AGGGAACCCTGGTCACCGTCTCTTCAGGCGGTGGCGGTAGTGGGGGAGGCGGTTCTGGCGGCGGAGGGTCCGGCGGTGGAGGATCA
GACGTTGTGATGACCCAGTCTCCATCTTCCGTGTCTGCATCTGTAGGAGACAGAGTCACCATCACCTGTCAGGCCAGTCAGAACAT
TAGGACTTACTTATCCTGGTATCAGCAGAAACCAGGGAAAGCCCCTAAGCTCCTGATCTATGCTGCAGCCAATCTGGCATCTGGGG
TCCCATCAAGGTTCAGCGGCAGTGGATCTGGGACAGATTTCACTCTCACCATCAGCGACCTGGAGCCTGGCGATGCTGCAACTTAC
TATTGTCAGTCTACCTATCTTGGTACTGATTATGTTGGCGGTGCTTTCGGCGGAGGGACCAAGGTGGAGATCAAATGA
>Sequence ID 111: SI-77H5 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDI
LLTQSPVILSVSPGERVSFSCRASQSIGTNIHWYQQRINGSPRLLIKYASESISGIPSRFSGSGSGTDFTLSINSVESEDIADYYC
QQNNNWPTTFGCGTKLELKRTVAAPSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGECGGGGSGGGGSGGGGSQSALTQPASVSGSPGQSITISCTGTSSDVGG
YNFVSWYQQHPGKAPKLMIYDVSDRPSGVSDRFSGSKSGNTASLIISGLQADDEADYYCSSYGSSSTHVIFGGGTKVTVLGGGGSG
GGGSGGGGSGGGGSQVQLQESGGGLVKPGGSLSLSCAASGFTFSSYWMSWVRQAPGKGLEWVANINRDGSASYYVDSVKGRFTISR
DDAKNSLYLQMNSLRAEDTAVYYCARDRGVGYFDLWGRGTLVTVSS
>Sequence ID 112: SI-77H5 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCTCGGATGGAACTCTGGT
CTTACTACTTTGATTATTGGGGGCAGGGGACTCTCGTCACGGTCTCGAGTGGCGGTGGAGGGTCCGGCGGTGGTGGATCAGACATC
TTGCTGACTCAGTCTCCAGTCATCCTGTCTGTGAGTCCAGGAGAAAGAGTCAGTTTCTCCTGCAGGGCCAGTCAGAGTATTGGCAC
AAACATACACTGGTATCAGCAAAGAACAAATGGTTCTCCAAGGCTTCTCATAAAGTATGCTTCTGAGTCTATCTCTGGGATTCCTT
CCAGGTTTAGTGGCAGTGGATCAGGGACAGATTTTACTCTTAGCATCAACAGTGTGGAGTCTGAAGATATTGCAGATTATTACTGT
CAACAAAATAATAACTGGCCAACCACGTTCGGTTGTGGGACCAAGCTGGAGCTGAAACGTACGGTGGCTGCACCATCTGTCTTCAT
CTTCCCGCCATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAGGGCCTGAGCTC
GCCCGTCACAAAGAGCTTCAACAGGGGAGAGTGTGGCGGTGGCGGTAGCGGTGGCGGCGGAAGTGGTGGCGGAGGATCCCAGTCTG
CCCTGACTCAGCCTGCCTCCGTGTCTGGGTCTCCTGGACAGTCGATCACCATCTCCTGCACTGGAACCAGCAGTGACGTTGGTGGT
TATAACTTTGTCTCCTGGTACCAACAACACCCAGGCAAAGCCCCCAAACTCATGATCTATGATGTCAGTGATCGGCCCTCAGGGGT
GTCTGATCGCTTCTCCGGCTCCAAGTCTGGCAACACGGCCTCCCTGATCATCTCTGGCCTCCAGGCTGACGACGAGGCTGATTATT
ACTGCAGCTCATATGGGAGCAGCAGCACTCATGTGATTTTCGGCGGAGGGACCAAGGTGACCGTCCTAGGTGGAGGCGGTTCAGGC
GGAGGTGGTTCCGGCGGTGGCGGCTCCGGTGGAGGCGGCTCTCAGGTGCAATTGCAGGAGTCGGGGGGAGGCCTGGTCAAGCCTGG
AGGGTCCCTGAGTCTCTCCTGTGCAGCCTCTGGATTCACCTTTAGTAGTTATTGGATGAGCTGGGTCCGCCAGGCTCCAGGGAAGG
GGCTGGAGTGGGTGGCCAACATAAACCGCGATGGAAGTGCGAGTTACTATGTGGACTCTGTGAAGGGCCGATTCACCATCTCCAGA
GACGACGCCAAGAACTCACTGTATCTGCAAATGAACAGCCTGAGAGCTGAGGACACGGCTGTGTATTACTGTGCGAGAGATCGTGG
GGTGGGCTACTTCGATCTCTGGGGCCGTGGCACCCTGGTCACCGTCTCTAGCTGA
>Sequence ID 113: SI-55H11 heavy chain amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVLGGGGGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGSGGGGSGG
GGSGGGGSEVQLVESGGGLVQPGGSLRLSCAASGFTISTNAMSWVREAPGKCLEWIGVITGRDITYYASWAKGRFTISRDNSKNTL
YLQMNSLRAEDTAVYYCARDGGSSAITSNNIWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPKSCDKTHTCPPCPAPEAAGAPSVFLFPPKP
KDTLMISRTPEVTCVVVDVSHEDPEVKFNWYVDGVEVHNAKTKPREEQYNSTYRVVSVLTVLHQDWLNGKEYKCAVSNKALPAPIE
KTISKAKGQPREPQVYTLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGGGGGSGGGGSEVQLLESGGGLVQPGGSLRLSCAASGFSFSSGYDMCWVRQAPGKGLEWI
ACIAAGSAGITYDANWAKGRFTISRDNSKNTLYLQMNSLRAEDTAVYYCARSAFSFDYAMDLWGQGTLVTVSSGGGGSGGGGSGGG
GSGGGGSDIQMTQSPSTLSASVGDRVTITCQASQSISSHLNWYQQKPGKAPKLLIYKASTLASGVPSRFSGSGSGTEFTLTISSLQ
PDDFATYYCQQGYSWGNVDNVFGGGTKVEIKGGGGSGGGGSGRSLVESGGGLVQPGGSLRLSCTASGFTISSYHMQWVRQAPGKGL
EYIGTISSGGNVYYASSARGRFTISRPSSKNTVDLQMNSLRAEDTAVYYCARDSGYSDPMWGQGTLVTVSSGGGGSGGGGSGGGGS
GGGGSDVVMTQSPSSVSASVGDRVTITCQASQNIRTYLSWYQQKPGKAPKLLIYAAANLASGVPSRFSGSGSGTDFTLTISDLEPG
DAATYYCQSTYLGTDYVGGAFGGGTKVEIK
>Sequence ID 114: SI-55H11 heavy chain nucleotide sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGCTGACCGTTTTAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGTGGAGGCGGATCTGGCGGAGGTGGTTCCGGCGGT
GGCGGCTCCGGTGGAGGCGGCTCTGAGGTGCAGCTGGTGGAGTCTGGGGGAGGCTTGGTCCAGCCTGGGGGGTCCCTGAGACTCTC
CTGTGCAGCCTCTGGATTCACCATCAGTACCAATGCAATGAGCTGGGTCCGCGAGGCTCCAGGGAAGTGTCTGGAGTGGATCGGAG
TCATTACTGGTCGTGATATCACATACTACGCGAGCTGGGCGAAAGGCAGATTCACCATCTCCAGAGACAATTCCAAGAACACGCTG
TATCTTCAAATGAACAGCCTGAGAGCCGAGGACACGGCTGTGTATTACTGTGCGAGAGACGGTGGTTCTTCTGCTATTACTAGTAA
CAACATTTGGGGCCAGGGAACCCTGGTCACCGTGTCCTCAGCTAGCACCAAGGGCCCATCGGTCTTCCCCCTGGCACCCTCCTCCA
AGAGCACCTCTGGGGGCACAGCGGCCCTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCCCTCAGCAGCGTGGTGACCGTGCCCTC
CAGCAGCTTGGGCACCCAGACCTACATCTGCAACGTGAATCACAAGCCCAGCAACACCAAGGTGGACAAGAGAGTTGAGCCCAAAT
CTTGTGACAAAACTCACACATGCCCACCGTGCCCAGCACCTGAAGCCGCGGGGGCACCGTCAGTCTTCCTCTTCCCCCCAAAACCC
AAGGACACCCTCATGATCTCCCGGACCCCTGAGGTCACATGCGTGGTGGTGGACGTGAGCCACGAAGACCCTGAGGTCAAGTTCAA
CTGGTACGTGGACGGCGTGGAGGTGCATAATGCCAAGACAAAGCCGCGGGAGGAGCAGTACAACAGCACGTACCGTGTGGTCAGCG
TCCTCACCGTCCTGCACCAGGACTGGCTGAATGGCAAGGAGTACAAGTGCGCGGTCTCCAACAAAGCCCTCCCAGCCCCCATCGAG
AAAACCATCTCCAAAGCCAAAGGGCAGCCCCGAGAACCACAGGTGTACACCCTGCCCCCATCCCGGGATGAGCTGACCAAGAACCA
GGTCAGCCTGACCTGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTCTCCCTGTCTCCGGGTGGCGG
TGGAGGGTCCGGCGGTGGTGGATCCGAGGTGCAGCTGTTGGAGTCTGGGGGAGGCTTGGTACAGCCTGGGGGGTCCCTGAGACTCT
CCTGTGCAGCCTCTGGATTCTCCTTCAGTAGCGGGTACGACATGTGCTGGGTCCGCCAGGCTCCAGGGAAGGGGCTGGAGTGGATC
GCATGCATTGCTGCTGGTAGTGCTGGTATCACTTACGACGCGAACTGGGCGAAAGGCCGGTTCACCATCTCCAGAGACAATTCCAA
GAACACGCTGTATCTGCAAATGAACAGCCTGAGAGCCGAGGACACGGCCGTATATTACTGTGCGAGATCGGCGTTTTCGTTCGACT
ACGCCATGGACCTCTGGGGCCAGGGAACCCTGGTCACCGTGTCGAGCGGTGGAGGCGGATCTGGCGGAGGTGGTTCCGGCGGTGGC
GGCTCCGGTGGAGGCGGCTCTGACATCCAGATGACCCAGTCTCCTTCCACCCTGTCTGCATCTGTAGGAGACAGAGTCACCATCAC
TTGCCAGGCCAGTCAGAGCATTAGTTCCCACTTAAACTGGTATCAGCAGAAACCAGGGAAAGCCCCTAAGCTCCTGATCTATAAGG
CATCCACTCTGGCATCTGGGGTCCCATCAAGGTTCAGCGGCAGTGGATCTGGGACAGAATTTACTCTCACCATCAGCAGCCTGCAG
CCTGATGATTTTGCAACTTATTACTGCCAACAGGGTTATAGTTGGGGTAATGTTGATAATGTTTTCGGCGGAGGGACCAAGGTGGA
GATCAAAGGCGGTGGAGGGTCCGGCGGTGGTGGCTCCGGACGGTCGCTGGTGGAGTCTGGGGGAGGCTTGGTCCAGCCTGGGGGGT
CCCTGAGACTCTCCTGTACTGCCTCTGGATTCACCATCAGTAGCTACCACATGCAGTGGGTCCGCCAGGCTCCAGGGAAGGGGCTG
GAGTACATCGGAACCATTAGTAGTGGTGGTAATGTATACTACGCAAGCTCCGCTAGAGGCAGATTCACCATCTCCAGACCCTCGTC
CAAGAACACGGTGGATCTTCAAATGAACAGCCTGAGAGCCGAGGACACGGCTGTGTATTACTGTGCGAGAGACTCTGGTTATAGTG
ATCCTATGTGGGGCCAGGGAACCCTGGTCACCGTCTCTTCAGGCGGTGGCGGTAGTGGGGGAGGCGGTTCTGGCGGCGGAGGGTCC
GGCGGTGGAGGATCAGACGTTGTGATGACCCAGTCTCCATCTTCCGTGTCTGCATCTGTAGGAGACAGAGTCACCATCACCTGTCA
GGCCAGTCAGAACATTAGGACTTACTTATCCTGGTATCAGCAGAAACCAGGGAAAGCCCCTAAGCTCCTGATCTATGCTGCAGCCA
ATCTGGCATCTGGGGTCCCATCAAGGTTCAGCGGCAGTGGATCTGGGACAGATTTCACTCTCACCATCAGCGACCTGGAGCCTGGC
GATGCTGCAACTTACTATTGTCAGTCTACCTATCTTGGTACTGATTATGTTGGCGGTGCTTTCGGCGGAGGGACCAAGGTGGAGAT
CAAATGA
>Sequence ID 115: SI-55H11 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDV
VMTQSPSTLSASVGDRVTINCQASESISSWLAWYQQKPGKAPKLLIYEASKLASGVPSRFSGSGSGTEFTLTISSLQPDDFATYYC
QGYFYFISRTYVNSFGCGTKVEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGECGGGGSGGGGSGGGGSQSALTQPASVSGSPGQSITISCTGTS
SDVGGYNFVSWYQQHPGKAPKLMIYDVSDRPSGVSDRFSGSKSGNTASLIISGLQADDEADYYCSSYGSSSTHVIFGGGTKVTVLG
GGGSGGGGSGGGGSGGGGSQVQLQESGGGLVKPGGSLSLSCAASGFTFSSYWMSWVRQAPGKGLEWVANINRDGSASYYVDSVKGR
FTISRDDAKNSLYLQMNSLRAEDTAVYYCARDRGVGYFDLWGRGTLVTVSS
>Sequence ID 116: SI-55H11 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCTCGGATGGAACTCTGGT
CTTACTACTTTGATTATTGGGGGCAGGGGACTCTCGTCACGGTCTCGAGTGGCGGTGGAGGGTCCGGCGGTGGTGGATCAGACGTC
GTGATGACCCAGTCTCCTTCCACCCTGTCTGCATCTGTAGGAGACAGAGTCACCATCAATTGCCAAGCCAGTGAGAGCATTAGCAG
TTGGTTAGCCTGGTATCAGCAGAAACCAGGGAAAGCCCCTAAGCTCCTGATCTATGAAGCATCCAAACTGGCATCTGGGGTCCCAT
CAAGGTTCAGCGGCAGTGGATCTGGGACAGAATTTACTCTCACCATCAGCAGCCTGCAGCCTGATGATTTTGCAACTTATTACTGC
CAAGGCTATTTTTATTTTATTAGTCGTACTTATGTAAATTCTTTCGGCTGTGGGACCAAGGTGGAGATCAAACGTACGGTGGCTGC
ACCATCTGTCTTCATCTTCCCGCCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTGGCGGTGGCGGTAGCGGTGGCGGCGGAAGTGGTGGCG
GAGGATCCCAGTCTGCCCTGACTCAGCCTGCCTCCGTGTCTGGGTCTCCTGGACAGTCGATCACCATCTCCTGCACTGGAACCAGC
AGTGACGTTGGTGGTTATAACTTTGTCTCCTGGTACCAACAACACCCAGGCAAAGCCCCCAAACTCATGATCTATGATGTCAGTGA
TCGGCCCTCAGGGGTGTCTGATCGCTTCTCCGGCTCCAAGTCTGGCAACACGGCCTCCCTGATCATCTCTGGCCTCCAGGCTGACG
ACGAGGCTGATTATTACTGCAGCTCATATGGGAGCAGCAGCACTCATGTGATTTTCGGCGGAGGGACCAAGGTGACCGTCCTAGGT
GGAGGCGGTTCAGGCGGAGGTGGTTCCGGCGGTGGCGGCTCCGGTGGAGGCGGCTCTCAGGTGCAATTGCAGGAGTCGGGGGGAGG
CCTGGTCAAGCCTGGAGGGTCCCTGAGTCTCTCCTGTGCAGCCTCTGGATTCACCTTTAGTAGTTATTGGATGAGCTGGGTCCGCC
AGGCTCCAGGGAAGGGGCTGGAGTGGGTGGCCAACATAAACCGCGATGGAAGTGCGAGTTACTATGTGGACTCTGTGAAGGGCCGA
TTCACCATCTCCAGAGACGACGCCAAGAACTCACTGTATCTGCAAATGAACAGCCTGAGAGCTGAGGACACGGCTGTGTATTACTG
TGCGAGAGATCGTGGGGTGGGCTACTTCGATCTCTGGGGCCGTGGCACCCTGGTCACCGTCTCTAGCTGA
>Sequence ID 117: SI-77H4 heavy chain amino acid sequence
EIVMTQSPSTLSASVGDRVIITCQASESISSWLAWYQQKPGKAPKLLIYEASKLASGVPSRFSGSGSGAEFTLTISSLQPDDFATY
YCQGYFYFISRTYVNSFGQGTKLTVLGGGGGGGGSGGGGSGGGGSEVQLVESGGGLVQPGGSLRLSCAASGFTISTNAMSWVRQA
PGKGLEWVGVITGRDITYYASWAKGRFTISRDTSKNTVYLQMNSLRAEDTAVYYCARDGGSSAITSNNIWGQGTLVTVSSGGGGSG
GGGSQVQLQQSGPGLVKPSETLSITCTVSGFSLTNYGVHWIRQAPGKCLEWLGVIWSGGNTDYNTPFTSRFTITKDNSKNQVYFKL
RSVRADDTAIYYCARALTYYDYEFAYWGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPCPAPEAAGAPSVFLFPPKPKDTLM
ISRTPEVTCVVVDVSHEDPEVKFNWYVDGVEVHNAKTKPREEQYNSTYRVVSVLTVLHQDWLNGKEYKCAVSNKALPAPIEKTISK
AKGQPREPQVYTLPPSRDELTKNQVSLTCLVKGFYPSDIAVEWESNGQPENNYKTTPPVLDSDGSFFLYSKLTVDKSRWQQGNVES
CSVMHEALHNHYTQKSLSLSPGGGGGSGGGGSEVQLLESGGGLVQPGGSLRLSCAASGFSFSSGYDMCWVRQAPGKGLEWIACIAA
GSAGITYDANWAKGRFTISRDNSKNTLYLQMNSLRAEDTAVYYCARSAFSFDYAMDLWGQGTLVTVSSGGGGSGGGGGGGGSGGG
GSDIQMTQSPSTLSASVGDRVTITCQASQSISSHLNWYQQKPGKAPKLLIYKASTLASGVPSRFSGSGSGTEFTLTISSLQPDDFA
TYYCQQGYSWGNVDNVFGGGTKVEIKGGGGGGGGSGRSLVESGGGLVQPGGSLRLSCTASGFTISSYHMQWVRQAPGKGLEYIGT
ISSGGNVYYASSARGRFTISRPSSKNTVDLQMNSLRAEDTAVYYCARDSGYSDPMWGQGTLVTVSSGGGGSGGGGSGGGGSGGGGS
DVVMTQSPSSVSASVGDRVTITCQASQNIRTYLSWYQQKPGKAPKLLIYAAANLASGVPSRFSGSGSGTDFTLTISDLEPGDAATY
YCQSTYLGTDYVGGAFGGGTKVEIK
>Sequence ID 118: SI-77H4 heavy chain nucleotide sequence
GAAATCGTTATGACGCAGAGTCCCTCCACGCTCTCCGCTAGTGTCGGGGATCGCGTCATTATCACATGCCAGGCCTCCGAGTCAAT
CAGCAGCTGGCTTGCATGGTATCAACAGAAGCCGGGAAAAGCTCCTAAATTGCTGATCTATGAAGCGTCAAAATTGGCGTCTGGTG
TCCCATCTAGGTTCTCCGGCTCTGGGTCTGGTGCGGAATTTACTTTGACAATCTCCAGTCTTCAACCAGACGATTTCGCTACCTAC
TACTGCCAAGGGTATTTCTATTTTATAAGCCGGACATATGTAAACTCCTTCGGCCAAGGAACAAAGTTGACTGTTCTTGGTGGCGG
AGGCAGTGGTGGCGGGGGCAGCGGAGGTGGTGGTTCAGGGGGTGGTGGGAGCGAAGTCCAATTGGTAGAAAGTGGCGGTGGTCTGG
TGCAACCTGGTGGATCTCTTCGCCTCTCATGCGCCGCTAGTGGCTTTACTATTTCAACTAATGCGATGAGCTGGGTTCGCCAGGCC
CCCGGCAAAGGACTTGAGTGGGTCGGCGTCATCACCGGCAGGGACATTACATACTATGCGAGTTGGGCAAAGGGCAGGTTCACGAT
TAGCCGCGATACTTCAAAGAATACCGTTTACCTTCAAATGAATAGCTTGAGGGCGGAAGACACAGCTGTGTATTACTGCGCGAGGG
ATGGAGGTAGTTCCGCCATAACTTCCAACAACATATGGGGACAAGGCACGCTGGTTACTGTCTCGAGTGGCGGTGGAGGGTCCGGC
GGTGGTGGATCACAAGTACAGTTGCAGCAATCCGGTCCCGGTCTCGTCAAACCGAGTGAGACGCTTAGTATAACGTGTACTGTTTC
AGGCTTTAGCCTTACGAACTATGGAGTTCACTGGATTCGGCAGGCACCCGGCAAATGTTTGGAATGGCTGGGTGTTATTTGGTCAG
GTGGAAATACAGACTATAACACCCCCTTTACAAGTCGGTTCACAATTACGAAAGATAATTCCAAAAATCAAGTTTATTTCAAGTTG
AGATCCGTCCGCGCGGACGACACTGCGATCTACTATTGTGCGAGGGCACTGACCTACTACGATTACGAATTTGCGTATTGGGGGCA
AGGGACTCTTGTAACAGTCTCCAGTGCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGACAAGAGAGTTGAGCCCAAATCTTGTGACAAAACTC
ACACATGCCCACCGTGCCCAGCACCTGAAGCCGCGGGGGCACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGTGTGGTCAGCGTCCTCACCGTCCTGC
ACCAGGACTGGCTGAATGGCAAGGAGTACAAGTGCGCGGTCTCCAACAAAGCCCTCCCAGCCCCCATCGAGAAAACCATCTCCAAA
GCCAAAGGGCAGCCCCGAGAACCACAGGTGTACACCCTGCCCCCATCCCGGGATGAGCTGACCAAGAACCAGGTCAGCCTGACCTG
CCTGGTCAAAGGCTTCTATCCCAGCGACATCGCCGTGGAGTGGGAGAGCAATGGGCAGCCGGAGAACAACTACAAGACCACGCCTC
CCGTGCTGGACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTGCAGCCTCTG
GATTCTCCTTCAGTAGCGGGTACGACATGTGCTGGGTCCGCCAGGCTCCAGGGAAGGGGCTGGAGTGGATCGCATGCATTGCTGCT
GGTAGTGCTGGTATCACTTACGACGCGAACTGGGCGAAAGGCCGGTTCACCATCTCCAGAGACAATTCCAAGAACACGCTGTATCT
GCAAATGAACAGCCTGAGAGCCGAGGACACGGCCGTATATTACTGTGCGAGATCGGCGTTTTCGTTCGACTACGCCATGGACCTCT
GGGGCCAGGGAACCCTGGTCACCGTGTCGAGCGGTGGAGGCGGATCTGGCGGAGGTGGTTCCGGCGGTGGCGGCTCCGGTGGAGGC
GGCTCTGACATCCAGATGACCCAGTCTCCTTCCACCCTGTCTGCATCTGTAGGAGACAGAGTCACCATCACTTGCCAGGCCAGTCA
GAGCATTAGTTCCCACTTAAACTGGTATCAGCAGAAACCAGGGAAAGCCCCTAAGCTCCTGATCTATAAGGCATCCACTCTGGCAT
CTGGGGTCCCATCAAGGTTCAGCGGCAGTGGATCTGGGACAGAATTTACTCTCACCATCAGCAGCCTGCAGCCTGATGATTTTGCA
ACTTATTACTGCCAACAGGGTTATAGTTGGGGTAATGTTGATAATGTTTTCGGCGGAGGGACCAAGGTGGAGATCAAAGGCGGTGG
AGGGTCCGGCGGTGGTGGCTCCGGACGGTCGCTGGTGGAGTCTGGGGGAGGCTTGGTCCAGCCTGGGGGGTCCCTGAGACTCTCCT
GTACTGCCTCTGGATTCACCATCAGTAGCTACCACATGCAGTGGGTCCGCCAGGCTCCAGGGAAGGGGCTGGAGTACATCGGAACC
ATTAGTAGTGGTGGTAATGTATACTACGCAAGCTCCGCTAGAGGCAGATTCACCATCTCCAGACCCTCGTCCAAGAACACGGTGGA
TCTTCAAATGAACAGCCTGAGAGCCGAGGACACGGCTGTGTATTACTGTGCGAGAGACTCTGGTTATAGTGATCCTATGTGGGGCC
AGGGAACCCTGGTCACCGTCTCTTCAGGCGGTGGCGGTAGTGGGGGAGGCGGTTCTGGCGGCGGAGGGTCCGGCGGTGGAGGATCA
GACGTTGTGATGACCCAGTCTCCATCTTCCGTGTCTGCATCTGTAGGAGACAGAGTCACCATCACCTGTCAGGCCAGTCAGAACAT
TAGGACTTACTTATCCTGGTATCAGCAGAAACCAGGGAAAGCCCCTAAGCTCCTGATCTATGCTGCAGCCAATCTGGCATCTGGGG
TCCCATCAAGGTTCAGCGGCAGTGGATCTGGGACAGATTTCACTCTCACCATCAGCGACCTGGAGCCTGGCGATGCTGCAACTTAC
TATTGTCAGTCTACCTATCTTGGTACTGATTATGTTGGCGGTGCTTTCGGCGGAGGGACCAAGGTGGAGATCAAATGA
>Sequence ID 119: SI-77H4 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSEI
VLTQSPSTLSVSPGERATFSCRASQSIGTNIHWYQQKPGKPPRLLIKYASESISGIPDRFSGSGSGTEFTLTISSVQSEDFAVYYC
QQNNNWPTTFGCGTKLTVLRTVAAPSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGECGGGGSGGGGSGGGGSQSALTQPASVSGSPGQSITISCTGTSSDVGG
YNFVSWYQQHPGKAPKLMIYDVSDRPSGVSDRFSGSKSGNTASLIISGLQADDEADYYCSSYGSSSTHVIFGGGTKVTVLGGGGSG
GGGSGGGGSGGGGSQVQLQESGGGLVKPGGSLSLSCAASGFTFSSYWMSWVRQAPGKGLEWVANINRDGSASYYVDSVKGRFTISR
DDAKNSLYLQMNSLRAEDTAVYYCARDRGVGYFDLWGRGTLVTVSS
>Sequence ID 120: SI-77H4 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCTCGGATGGAACTCTGGT
CTTACTACTTTGATTATTGGGGGCAGGGGACTCTCGTCACGGTCTCGAGTGGCGGTGGAGGGTCCGGCGGTGGTGGATCAGAAATC
GTCCTTACACAATCTCCTAGCACACTGAGTGTGAGCCCCGGCGAACGCGCGACTTTCTCTTGCAGGGCAAGTCAATCCATAGGGAC
TAATATACATTGGTATCAACAAAAGCCAGGTAAACCACCCAGGCTTTTGATTAAGTATGCAAGTGAGTCTATTTCCGGTATCCCTG
ACCGCTTCTCTGGATCAGGCAGTGGCACAGAGTTCACACTCACCATATCTAGTGTGCAATCAGAGGACTTCGCCGTGTATTACTGC
CAACAGAATAATAACTGGCCGACTACCTTCGGATGCGGTACAAAGCTGACCGTTTTACGTACGGTGGCTGCACCATCTGTCTTCAT
CTTCCCGCCATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAGGGCCTGAGCTC
GCCCGTCACAAAGAGCTTCAACAGGGGAGAGTGTGGCGGTGGCGGTAGCGGTGGCGGCGGAAGTGGTGGCGGAGGATCCCAGTCTG
CCCTGACTCAGCCTGCCTCCGTGTCTGGGTCTCCTGGACAGTCGATCACCATCTCCTGCACTGGAACCAGCAGTGACGTTGGTGGT
TATAACTTTGTCTCCTGGTACCAACAACACCCAGGCAAAGCCCCCAAACTCATGATCTATGATGTCAGTGATCGGCCCTCAGGGGT
GTCTGATCGCTTCTCCGGCTCCAAGTCTGGCAACACGGCCTCCCTGATCATCTCTGGCCTCCAGGCTGACGACGAGGCTGATTATT
ACTGCAGCTCATATGGGAGCAGCAGCACTCATGTGATTTTCGGCGGAGGGACCAAGGTGACCGTCCTAGGTGGAGGCGGTTCAGGC
GGAGGTGGTTCCGGCGGTGGCGGCTCCGGTGGAGGCGGCTCTCAGGTGCAATTGCAGGAGTCGGGGGGAGGCCTGGTCAAGCCTGG
AGGGTCCCTGAGTCTCTCCTGTGCAGCCTCTGGATTCACCTTTAGTAGTTATTGGATGAGCTGGGTCCGCCAGGCTCCAGGGAAGG
GGCTGGAGTGGGTGGCCAACATAAACCGCGATGGAAGTGCGAGTTACTATGTGGACTCTGTGAAGGGCCGATTCACCATCTCCAGA
GACGACGCCAAGAACTCACTGTATCTGCAAATGAACAGCCTGAGAGCTGAGGACACGGCTGTGTATTACTGTGCGAGAGATCGTGG
GGTGGGCTACTTCGATCTCTGGGGCCGTGGCACCCTGGTCACCGTCTCTAGCTGA
>Sequence ID 121: SI-68P7 heavy chain amino acid sequence
EIVMTQSPSTLSASVGDRVIITCRASQSIGTNIHWYQQKPGKAPKLLIYYASESISGIPSRFSGSGSGAEFTLTISSLQPDDFATY
YCQQNNNWPTTFGQGTKLTVLGGGGSGGGGSGGGGSGGGGSEVQLVESGGGLVQPGGSLRLSCSVSGFSLTNYGVHWVRQAPGKGL
EWVGVIWSGGNTDYNTPFTSRFTISRDTSKNTVYLQMNSLRAEDTAVYYCARALTYYDYEFAYWGQGTLVTVSSGGGGSGGGGSQV
QLQESGGRLVQPGEPLSLTCKTSGIDLSSNAIGWVRQAPGKGLEWIGVIFGSGNTYYASWAKGRFTISRSTSTVYLKMNSLRSEDT
AIYYCARGGYSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYKCAVSNKALPAPIEKTISKAKGQPREPQVYT
LPPSRDELTKNQVSLSCAVKGFYPSDIAVEWESNGQPENNYKTTPPVLDSDGSFFLVSKLTVDKSRWQQGNVFSCSVMHEALHNHY
TQKSLSLSPGGGGGSGGGGSREGPELSPDDPAGLLDLRQGMFAQLVAQNVLLIDGPLSWYSDPGLAGVSLTGGLSYKEDTKELVVA
KAGVYYVFFQLELRRVVAGEGSGSVSLALHLQPLRSAAGAAALALTVDLPPASSEARNSAFGFQGRLLHLSAGQRLGVHLHTEARA
RHAWQLTQGATVLGLFRVTPEIPAGLGSTGSGSKPGSGEGSTKGREGPELSPDDPAGLLDLRQGMFAQLVAQNVLLIDGPLSWYSD
PGLAGVSLTGGLSYKEDTKELVVAKAGVYYVFFQLELRRVVAGEGSGSVSLALHLQPLRSAAGAAALALTVDLPPASSEARNSAFG
FQGRLLHLSAGQRLGVHLHTEARARHAWQLTQGATVLGLFRVTPEIPAGLGGGGSGGGGSREGPELSPDDPAGLLDLRQGMFAQLV
AQNVLLIDGPLSWYSDPGLAGVSLTGGLSYKEDTKELVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGATVLGLFRVTPEIPAGL
>Sequence ID 122: SI-68P7 heavy chain nucleotide sequence
GAAATCGTTATGACACAGTCCCCATCCACTCTTAGCGCTTCTGTAGGGGATCGAGTGATTATCACATGCCGGGCCTCCCAATCCAT
AGGAACCAACATACACTGGTATCAACAAAAACCAGGCAAAGCGCCAAAACTGCTTATCTACTACGCCTCCGAGAGTATTTCTGGAA
TCCCGAGTCGCTTCTCAGGTTCTGGAAGCGGCGCTGAGTTTACCCTCACAATTTCTTCACTCCAACCGGATGACTTCGCTACATAT
TACTGCCAACAAAACAATAATTGGCCGACGACCTTTGGCCAGGGCACGAAACTTACGGTACTTGGCGGTGGCGGTAGTGGGGGAGG
CGGTTCTGGCGGCGGAGGGTCCGGCGGTGGAGGATCAGAAGTACAGCTTGTCGAGTCCGGTGGGGGGCTTGTTCAGCCAGGGGGTT
CCTTGAGGCTTTCCTGCTCCGTCTCTGGGTTTAGCTTGACGAATTACGGCGTTCACTGGGTTAGACAAGCACCGGGGAAGGGGCTG
GAATGGGTCGGTGTGATATGGTCCGGGGGTAATACGGATTACAATACACCTTTCACGTCACGCTTTACGATTAGCAGGGACACGTC
AAAAAATACAGTCTACTTGCAGATGAACTCTCTTAGGGCGGAAGATACTGCAGTTTATTACTGCGCAAGGGCTCTGACATACTACG
ATTATGAATTTGCATATTGGGGCCAGGGGACTTTGGTCACGGTCTCGAGCGGCGGTGGAGGGTCCGGCGGTGGTGGATCACAAGTG
CAGTTGCAAGAAAGTGGTGGTAGACTGGTTCAGCCTGGTGAACCCTTGTCACTGACGTGTAAAACAAGCGGCATTGATCTGTCCTC
TAACGCCATCGGATGGGTCCGACAGGCCCCAGGAAAAGGTCTGGAGTGGATCGGAGTTATCTTCGGGAGCGGCAATACATACTACG
CAAGCTGGGCAAAAGGGCGATTTACGATATCACGGAGCACCTCTACAGTTTATTTGAAAATGAACTCCCTCCGGTCCGAGGATACC
GCGATATATTACTGTGCCAGAGGGGGGTACTCCTCTGATATCTGGGGGCAGGGTACACTGGTTACAGTTTCATCCGCTAGCACCAA
GGGCCCATCGGTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCACACATGCCCACCGTGCCCAGCACCTGAAGCCGCGG
GGGGACCGTCAGTCTTCCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAATGCCAAGACAAAGCCGCGGGA
GGAGCAGTACAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAGGTGTATACC
CTGCCCCCATCCCGGGATGAGCTGACCAAGAACCAGGTCAGCCTGTCCTGCGCCGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCTTCCTCGTGA
GCAAGCTCACCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTGGCGGCGGCGGTTCAGGTGGTGGGGGATCCCGAGAGGGCCCCGAGCTGTCTCCTGA
TGACCCAGCAGGCCTCTTGGACTTGCGGCAGGGTATGTTCGCTCAACTTGTGGCTCAGAATGTTCTGCTCATTGATGGACCACTCT
CTTGGTATAGTGACCCCGGTCTGGCCGGGGTGAGTCTGACCGGCGGGCTCTCTTATAAAGAGGATACTAAGGAACTGGTCGTAGCA
AAAGCGGGCGTTTATTACGTTTTTTTTCAGCTGGAGCTCAGGCGCGTGGTGGCCGGCGAGGGCAGTGGCTCTGTGTCCCTGGCCCT
GCACTTACAGCCCTTGAGAAGCGCTGCAGGTGCTGCCGCACTGGCTTTAACTGTTGACCTCCCTCCGGCCTCTTCTGAAGCTAGAA
ACAGCGCTTTCGGCTTCCAAGGGCGCCTGCTGCACCTGAGCGCAGGCCAGCGCTTAGGTGTGCACCTTCATACAGAGGCCAGGGCC
CGACACGCTTGGCAGCTCACACAGGGTGCCACGGTTCTCGGACTTTTCCGCGTTACTCCCGAGATCCCCGCTGGCCTCGGAAGTAC
TGGTTCTGGGTCTAAACCCGGTTCCGGCGAAGGTAGTACTAAAGGACGAGAAGGGCCAGAGTTAAGTCCAGATGACCCTGCTGGGC
TTTTGGACCTGCGGCAGGGCATGTTCGCTCAACTGGTGGCTCAGAACGTGCTGCTGATCGATGGCCCCCTGAGTTGGTACAGCGAT
CCCGGGCTGGCAGGCGTGTCACTTACAGGGGGCCTCTCTTACAAGGAAGACACCAAGGAGTTAGTGGTCGCTAAGGCTGGCGTGTA
TTACGTGTTCTTCCAACTGGAGCTGAGAAGGGTTGTGGCAGGAGAGGGTAGCGGCAGCGTGTCTTTAGCCCTTCACTTGCAGCCCC
TGAGGTCTGCTGCAGGTGCAGCCGCTCTCGCGCTCACCGTGGATCTCCCCCCAGCCTCATCTGAAGCTAGGAACAGTGCATTTGGC
TTTCAGGGACGCTTGCTGCACCTCTCCGCTGGACAGAGGCTGGGCGTGCACCTTCACACAGAGGCCCGTGCCAGGCATGCATGGCA
GCTCACTCAGGGGGCAACAGTGCTGGGTCTCTTCCGCGTGACTCCTGAAATACCAGCTGGACTTGGCGGTGGAGGCAGCGGCGGAG
GAGGATCTCGTGAGGGGCCAGAACTGTCCCCCGATGACCCAGCCGGACTGCTCGATCTCAGACAGGGCATGTTCGCTCAGCTTGTA
GCCCAAAATGTCCTCCTGATTGACGGCCCTTTGAGCTGGTATAGTGATCCCGGCTTGGCCGGGGTATCTCTGACCGGAGGCCTCTC
CTACAAGGAAGACACCAAAGAGCTGGTGGTGGCAAAAGCGGGGGTGTATTATGTGTTCTTTCAGCTCGAGCTGCGGAGAGTTGTGG
CCGGGGAAGGGTCTGGGAGCGTATCTCTTGCACTTCACCTGCAGCCCCTGCGCAGCGCCGCTGGAGCCGCCGCCCTTGCTCTTACT
GTGGATCTGCCTCCTGCTTCCTCAGAAGCACGCAACAGCGCCTTCGGCTTTCAAGGACGTCTCCTGCACTTGTCCGCAGGACAGAG
GTTGGGCGTCCATTTACACACTGAGGCACGGGCACGGCACGCTTGGCAGCTTACCCAGGGAGCCACCGTGCTGGGACTCTTTAGAG
TGACACCCGAGATCCCCGCTGGCTTGTGA
>Sequence ID 123: SI-68P7 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDP
QLTQSPSSLSATVGQRVTINCQSSQSVAKNNNLAWFQQKPGKPPKLLIYSASTLAAGVPSRFSGSGSGTQFTLTITRVQSEDFATY
YCSARDSGNIQSFGGGTRVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGECGGDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCAVSNKALPAPIEKTISKAKGQPREPQ
VYTLPPSRDELTKNQVSLWCLVKGFYPSDIAVEWESNGQPENNYKTTPPVLDSDGSFFLYSKLTVDKSRWQQGNVFSCSVMHEALH
NRFTQKSLSLSPGGGGGSGGGGSDIQMTQSPSTLSASVGDRVTITCQASQSISSHLNWYQQKPGKAPKLLIYKASTLASGVPSRFS
GSGSGTEFTLTISSLQPDDFATYYCQQGYSWGNVDNVFGGGTKVTVLGGGGSGGGGSGGGGSGGGGSEVQLLESGGGLVQPGGSLS
LSCAASGFSFSSGYDMCWVRQAPGKGLEWIACIAAGSAGITYDANWAKGRFTISRDNSKNTLYLQMNSLRAEDTAVYYCARSAFSF
DYAMDLWGQGTLVTVSS
>Sequence ID 124: SI-68P7 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCAGATCCT
CAATTGACCCAGTCCCCTAGTAGCCTTTCTGCAACCGTGGGCCAGAGGGTAACAATCAATTGCCAGTCAAGCCAGTCCGTAGCGAA
GAATAATAATTTGGCATGGTTCCAACAAAAACCGGGTAAACCTCCTAAACTCCTTATCTACTCAGCTTCAACCCTTGCCGCGGGTG
TGCCCAGCCGGTTCTCAGGCTCAGGTTCCGGGACTCAATTTACACTTACCATCACTCGCGTCCAAAGTGAGGATTTTGCAACCTAC
TATTGTTCTGCACGGGATTCCGGGAACATACAGTCCTTTGGGGGTGGAACTCGGGTGGAGATAAAACGTACGGTGGCTGCACCATC
TGTCTTCATCTTCCCGCCATCTGATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTGGAGGCGACAAAACTCACACATGCCCACCGTGCCCAGCACCTG
AAGCCGCGGGGGGACCGTCAGTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACCAGGACTGGCTGAATGGCAAGGAGT
ACAAGTGCGCGGTCTCCAACAAAGCCCTCCCAGCCCCCATCGAGAAAACCATCTCCAAAGCCAAAGGGCAGCCCCGAGAACCACAG
GTGTACACCCTGCCCCCAAGCCGGGATGAGCTGACCAAGAACCAGGTCAGCCTGTGGTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACGGCTCCTTCT
TCCTCTATAGCAAGCTCACCGTGGACAAGAGCAGGTGGCAGCAGGGGAACGTCTTCTCATGCTCCGTGATGCATGAGGCTCTGCAC
AACAGATTCACGCAGAAGAGCCTCTCCCTGTCTCCGGGTGGCGGTGGAGGGTCCGGCGGTGGTGGATCCGACATTCAGATGACACA
GTCACCGTCTACGCTCTCAGCCTCAGTAGGCGATAGGGTGACTATCACCTGCCAAGCCTCCCAATCTATAAGTTCACACCTCAACT
GGTATCAACAGAAACCCGGTAAAGCACCGAAGCTGCTCATCTACAAGGCATCAACTCTTGCCTCAGGTGTGCCTAGTCGCTTCTCC
GGGAGCGGCTCCGGTACTGAGTTTACTTTGACGATATCTTCCCTCCAACCCGACGACTTCGCCACATACTATTGTCAGCAGGGTTA
TAGTTGGGGAAATGTTGACAATGTGTTTGGAGGGGGAACGAAAGTGACTGTGTTGGGAGGGGGAGGAAGTGGCGGCGGCGGAAGCG
GAGGTGGTGGCTCCGGCGGGGGTGGAAGCGAAGTGCAATTGCTCGAAAGTGGTGGCGGACTTGTGCAACCTGGTGGAAGCCTCTCA
CTCTCTTGCGCGGCGAGTGGTTTTAGCTTCAGTAGCGGCTACGATATGTGCTGGGTAAGACAGGCTCCTGGTAAAGGCCTTGAGTG
GATTGCGTGTATCGCCGCCGGGTCTGCTGGTATAACATACGATGCAAACTGGGCGAAAGGGCGCTTCACAATCTCCAGGGACAACT
CAAAAAATACATTGTACCTCCAGATGAATAGCCTGAGAGCCGAAGACACCGCAGTCTACTATTGCGCAAGGTCAGCATTTAGCTTC
GATTATGCAATGGATCTTTGGGGCCAGGGGACGCTCGTCACCGTCTCATCTTGA
>Sequence ID 125: SI-79P1 heavy chain amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLELKGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGSGGGGSQV
QLQESGGRLVQPGEPLSLTCKTSGIDLSSNAIGWVRQAPGKGLEWIGVIFGSGNTYYASWAKGRFTISRSTSTVYLKMNSLRSEDT
AIYYCARGGYSSDIWGQGTLVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYKCAVSNKALPAPIEKTISKAKGQPREPQVYT
LPPSRDELTKNQVSLSCAVKGFYPSDIAVEWESNGQPENNYKTTPPVLDSDGSFFLVSKLTVDKSRWQQGNVFSCSVMHEALHNHY
TQKSLSLSPGGGGGSGGGGSDVVMTQSPSSVSASVGDRVTITCQASQNIRTYLSWYQQKPGKAPKLLIYAAANLASGVPSRFSGSG
SGTDFTLTISDLEPGDAATYYCQSTYLGTDYVGGAFGGGTKLTVLGGGGSGGGGSGGGGSGGGGSEVQLVESGGGLVQPGGSLRLS
CTASGFTISSYHMQWVRQAPGKGLEYIGTISSGGNVYYASSARGRFTISRPSSKNTVDLQMNSLRAEDTAVYYCARDSGYSDPMWG
QGTLVTVSS
>Sequence ID 126: SI-79P1 heavy chain nucleotide sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGTTGGAACTGAAAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGCGGTGGAGGGTCCGGCGGTGGTGGATCACAAGTG
CAGTTGCAAGAAAGTGGTGGTAGACTGGTTCAGCCTGGTGAACCCTTGTCACTGACGTGTAAAACAAGCGGCATTGATCTGTCCTC
TAACGCCATCGGATGGGTCCGACAGGCCCCAGGAAAAGGTCTGGAGTGGATCGGAGTTATCTTCGGGAGCGGCAATACATACTACG
CAAGCTGGGCAAAAGGGCGATTTACGATATCACGGAGCACCTCTACAGTTTATTTGAAAATGAACTCCCTCCGGTCCGAGGATACC
GCGATATATTACTGTGCCAGAGGGGGGTACTCCTCTGATATCTGGGGGCAGGGTACACTGGTTACAGTTTCATCCGCTAGCACCAA
GGGCCCATCGGTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCTGCAACGTGAATCACAAGCCCAG
CAACACCAAGGTGGACAAGAGAGTTGAGCCCAAATCTTGTGACAAAACTCACACATGCCCACCGTGCCCAGCACCTGAAGCCGCGG
GGGGACCGTCAGTCTTCCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAATGCCAAGACAAAGCCGCGGGA
GGAGCAGTACAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAGGTGTATACC
CTGCCCCCATCCCGGGATGAGCTGACCAAGAACCAGGTCAGCCTGTCCTGCGCCGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCTTCCTCGTGA
GCAAGCTCACCGTGGACAAGAGCAGGTGGCAGCAGGGGAACGTCTTCTCATGCTCCGTGATGCATGAGGCTCTGCACAACCACTAC
ACGCAGAAGAGCCTCTCCCTGTCTCCGGGTGGCGGCGGCGGTTCAGGTGGTGGGGGATCCGACGTTGTGATGACGCAGTCTCCTAG
TTCAGTATCAGCGTCTGTTGGGGATCGAGTCACGATTACATGCCAGGCGAGCCAAAATATTAGGACGTATCTGTCTTGGTATCAAC
AAAAGCCTGGTAAGGCTCCAAAACTCCTCATTTACGCCGCTGCCAACTTGGCTAGTGGAGTACCTTCACGCTTTAGTGGGTCAGGT
TCTGGAACAGATTTTACGTTGACGATCTCCGATTTGGAACCAGGGGACGCTGCCACGTATTATTGCCAGTCCACCTACCTCGGGAC
TGACTACGTTGGTGGGGCGTTTGGCGGAGGAACAAAACTCACTGTACTTGGTGGTGGTGGTTCAGGCGGGGGGGGTTCCGGCGGAG
GCGGTTCTGGGGGTGGAGGTTCCGAGGTTCAGTTGGTTGAAAGTGGGGGGGGACTCGTTCAGCCTGGAGGCTCTTTGCGACTTTCT
TGTACCGCTTCTGGGTTCACTATAAGTTCATATCATATGCAATGGGTAAGACAGGCGCCTGGTAAGGGACTTGAATACATAGGAAC
AATAAGCTCTGGTGGTAACGTCTATTACGCCTCATCCGCGCGGGGGAGGTTTACAATTTCCAGGCCTTCTAGCAAAAACACGGTAG
ATCTGCAAATGAACTCTCTTCGCGCTGAAGATACAGCAGTCTACTACTGCGCCAGGGATTCAGGGTATTCTGACCCCATGTGGGGG
CAGGGCACATTGGTTACCGTGTCCTCTTGA
>Sequence ID 127: SI-79P1 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDP
QLTQSPSSLSATVGQRVTINCQSSQSVAKNNNLAWFQQKPGKPPKLLIYSASTLAAGVPSRFSGSGSGTQFTLTITRVQSEDFATY
YCSARDSGNIQSFGGGTRVEIKRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGECGGDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQDWLNGKEYKCAVSNKALPAPIEKTISKAKGQPREPQ
VYTLPPSRDELTKNQVSLWCLVKGFYPSDIAVEWESNGQPENNYKTTPPVLDSDGSFFLYSKLTVDKSRWQQGNVFSCSVMHEALH
NRFTQKSLSLSPGGGGGSGGGGSDIQMTQSPSTLSASVGDRVTITCQASQSISSHLNWYQQKPGKAPKLLIYKASTLASGVPSRFS
GSGSGTEFTLTISSLQPDDFATYYCQQGYSWGNVDNVFGGGTKVTVLGGGGSGGGGSGGGGSGGGGSEVQLLESGGGLVQPGGSLS
LSCAASGFSFSSGYDMCWVRQAPGKGLEWIACIAAGSAGITYDANWAKGRFTISRDNSKNTLYLQMNSLRAEDTAVYYCARSAFSF
DYAMDLWGQGTLVTVSS
>Sequence ID 128: SI-79P1 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCAGATCCT
CAATTGACCCAGTCCCCTAGTAGCCTTTCTGCAACCGTGGGCCAGAGGGTAACAATCAATTGCCAGTCAAGCCAGTCCGTAGCGAA
GAATAATAATTTGGCATGGTTCCAACAAAAACCGGGTAAACCTCCTAAACTCCTTATCTACTCAGCTTCAACCCTTGCCGCGGGTG
TGCCCAGCCGGTTCTCAGGCTCAGGTTCCGGGACTCAATTTACACTTACCATCACTCGCGTCCAAAGTGAGGATTTTGCAACCTAC
TATTGTTCTGCACGGGATTCCGGGAACATACAGTCCTTTGGGGGTGGAACTCGGGTGGAGATAAAACGTACGGTGGCTGCACCATC
TGTCTTCATCTTCCCGCCATCTGATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCACCCATCAGGG
CCTGAGCTCGCCCGTCACAAAGAGCTTCAACAGGGGAGAGTGTGGAGGCGACAAAACTCACACATGCCCACCGTGCCCAGCACCTG
AAGCCGCGGGGGGACCGTCAGTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCCTGCACCAGGACTGGCTGAATGGCAAGGAGT
ACAAGTGCGCGGTCTCCAACAAAGCCCTCCCAGCCCCCATCGAGAAAACCATCTCCAAAGCCAAAGGGCAGCCCCGAGAACCACAG
GTGTACACCCTGCCCCCAAGCCGGGATGAGCTGACCAAGAACCAGGTCAGCCTGTGGTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACGGCTCCTTCT
TCCTCTATAGCAAGCTCACCGTGGACAAGAGCAGGTGGCAGCAGGGGAACGTCTTCTCATGCTCCGTGATGCATGAGGCTCTGCAC
AACAGATTCACGCAGAAGAGCCTCTCCCTGTCTCCGGGTGGCGGTGGAGGGTCCGGCGGTGGTGGATCCGACATTCAGATGACACA
GTCACCGTCTACGCTCTCAGCCTCAGTAGGCGATAGGGTGACTATCACCTGCCAAGCCTCCCAATCTATAAGTTCACACCTCAACT
GGTATCAACAGAAACCCGGTAAAGCACCGAAGCTGCTCATCTACAAGGCATCAACTCTTGCCTCAGGTGTGCCTAGTCGCTTCTCC
GGGAGCGGCTCCGGTACTGAGTTTACTTTGACGATATCTTCCCTCCAACCCGACGACTTCGCCACATACTATTGTCAGCAGGGTTA
TAGTTGGGGAAATGTTGACAATGTGTTTGGAGGGGGAACGAAAGTGACTGTGTTGGGAGGGGGAGGAAGTGGCGGCGGCGGAAGCG
GAGGTGGTGGCTCCGGCGGGGGTGGAAGCGAAGTGCAATTGCTCGAAAGTGGTGGCGGACTTGTGCAACCTGGTGGAAGCCTCTCA
CTCTCTTGCGCGGCGAGTGGTTTTAGCTTCAGTAGCGGCTACGATATGTGCTGGGTAAGACAGGCTCCTGGTAAAGGCCTTGAGTG
GATTGCGTGTATCGCCGCCGGGTCTGCTGGTATAACATACGATGCAAACTGGGCGAAAGGGCGCTTCACAATCTCCAGGGACAACT
CAAAAAATACATTGTACCTCCAGATGAATAGCCTGAGAGCCGAAGACACCGCAGTCTACTATTGCGCAAGGTCAGCATTTAGCTTC
GATTATGCAATGGATCTTTGGGGCCAGGGGACGCTCGTCACCGTCTCATCTTGA
>Sequence ID 129: SI-68P13 heavy chain amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVLGGGGSGGGGSGGGGSGGGGSQVQLQQSGPGLVKPSETLSITCTVSGFSLTNYGVHWIRQAPGKGL
EWLGVIWSGGNTDYNTPFTSRFTITKDNSKNQVYFKLRSVRADDTAIYYCARALTYYDYEFAYWGQGTLVTVSSGGGGSGGGGSEV
QLVESGGGLVQPGGSLRLSCAASGFTISTNAMSWVRQAPGKGLEWIGVITGRDITYYASWAKGRFTISRDNSKNTLYLQMNSLRAE
DTAVYYCARDGGSSAITSNNIWGQGTLVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWINGKEYKCAVSNKALPAPIEKTISKAKGQP
REPQVYTLPPSRDELTKNQVSLSCAVKGFYPSDIAVEWESNGQPENNYKTTPPVLDSDGSFFLVSKLTVDKSRWQQGNVFSCSVMH
EALHNHYTQKSLSLSPGGGGGSGGGGSDVVMTQSPSSVSASVGDRVTITCQASQNIRTYLSWYQQKPGKAPKLLIYAAANLASGVP
SRFSGSGSGTDFTLTISDLEPGDAATYYCQSTYLGTDYVGGAFGGGTKLTVLGGGGSGGGGSGGGGSGGGGSEVQLVESGGGLVQP
GGSLRLSCTASGFTISSYHMQWVRQAPGKGLEYIGTISSGGNVYYASSARGRFTISRPSSKNTVDLQMNSLRAEDTAVYYCARDSG
YSDPMWGQGTLVTVSS
>Sequence ID 130: SI-68P13 heavy chain nucleotide sequence
GAAATCGTCCTTACACAATCTCCTAGCACACTGAGTGTGAGCCCCGGCGAACGCGCGACTTTCTCTTGCAGGGCAAGTCAATCCAT
AGGGACTAATATACATTGGTATCAACAAAAGCCAGGTAAACCACCCAGGCTTTTGATTAAGTATGCAAGTGAGTCTATTTCCGGTA
TCCCTGACCGCTTCTCTGGATCAGGCAGTGGCACAGAGTTCACACTCACCATATCTAGTGTGCAATCAGAGGACTTCGCCGTGTAT
TACTGCCAACAGAATAATAACTGGCCGACTACCTTCGGACCCGGTACAAAGCTGACCGTTTTAGGCGGTGGCGGTAGTGGGGGAGG
CGGTTCTGGCGGCGGAGGGTCCGGCGGTGGAGGATCACAAGTACAGTTGCAGCAATCCGGTCCCGGTCTCGTCAAACCGAGTGAGA
CGCTTAGTATAACGTGTACTGTTTCAGGCTTTAGCCTTACGAACTATGGAGTTCACTGGATTCGGCAGGCACCCGGCAAAGGTTTG
GAATGGCTGGGTGTTATTTGGTCAGGTGGAAATACAGACTATAACACCCCCTTTACAAGTCGGTTCACAATTACGAAAGATAATTC
CAAAAATCAAGTTTATTTCAAGTTGAGATCCGTCCGCGCGGACGACACTGCGATCTACTATTGTGCGAGGGCACTGACCTACTACG
ATTACGAATTTGCGTATTGGGGGCAAGGGACTCTTGTAACAGTCTCGAGCGGCGGTGGAGGGTCCGGCGGTGGTGGATCAGAGGTG
CAGCTGGTGGAGTCTGGGGGAGGCTTGGTCCAGCCTGGGGGGTCCCTGAGACTCTCCTGTGCAGCCTCTGGATTCACCATCAGTAC
CAATGCAATGAGCTGGGTCCGCCAGGCTCCAGGGAAGGGGCTGGAGTGGATCGGAGTCATTACTGGTCGTGATATCACATACTACG
CGAGCTGGGCGAAAGGCAGATTCACCATCTCCAGAGACAATTCCAAGAACACGCTGTATCTTCAAATGAACAGCCTGAGAGCCGAG
GACACGGCTGTGTATTACTGTGCGAGAGACGGTGGTTCTTCTGCTATTACTAGTAACAACATTTGGGGCCAGGGAACCCTGGTCAC
CGTGTCCTCAGCTAGCACCAAGGGCCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAGAGTTGAGCCCAAATCTTGTGACAAAACTCACACATGCCCACCGT
GCCCAGCACCTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCGTCCTGCACCAGGACTGGCTGA
ATGGCAAGGAGTACAAGTGCGCGGTCTCCAACAAAGCCCTCCCAGCCCCCATCGAGAAAACCATCTCCAAAGCCAAAGGGCAGCCC
CGAGAACCACAGGTGTATACCCTGCCCCCATCCCGGGATGAGCTGACCAAGAACCAGGTCAGCCTGTCCTGCGCCGTCAAAGGCTT
CTATCCCAGCGACATCGCCGTGGAGTGGGAGAGCAATGGGCAGCCGGAGAACAACTACAAGACCACGCCTCCCGTGCTGGACTCCG
ACGGCTCCTTCTTCCTCGTGAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGCGGCGGTTCAGGTGGTGGGGGATCCGACGT
TGTGATGACGCAGTCTCCTAGTTCAGTATCAGCGTCTGTTGGGGATCGAGTCACGATTACATGCCAGGCGAGCCAAAATATTAGGA
CGTATCTGTCTTGGTATCAACAAAAGCCTGGTAAGGCTCCAAAACTCCTCATTTACGCCGCTGCCAACTTGGCTAGTGGAGTACCT
TCACGCTTTAGTGGGTCAGGTTCTGGAACAGATTTTACGTTGACGATCTCCGATTTGGAACCAGGGGACGCTGCCACGTATTATTG
CCAGTCCACCTACCTCGGGACTGACTACGTTGGTGGGGCGTTTGGCGGAGGAACAAAACTCACTGTACTTGGTGGTGGTGGTTCAG
GCGGGGGGGGTTCCGGCGGAGGCGGTTCTGGGGGTGGAGGTTCCGAGGTTCAGTTGGTTGAAAGTGGGGGGGGACTCGTTCAGCCT
GGAGGCTCTTTGCGACTTTCTTGTACCGCTTCTGGGTTCACTATAAGTTCATATCATATGCAATGGGTAAGACAGGCGCCTGGTAA
GGGACTTGAATACATAGGAACAATAAGCTCTGGTGGTAACGTCTATTACGCCTCATCCGCGCGGGGGAGGTTTACAATTTCCAGGC
CTTCTAGCAAAAACACGGTAGATCTGCAAATGAACTCTCTTCGCGCTGAAGATACAGCAGTCTACTACTGCGCCAGGGATTCAGGG
TATTCTGACCCCATGTGGGGGCAGGGCACATTGGTTACCGTGTCCTCTTGA
>Sequence ID 131: SI-68P13 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSDV
VMTQSPSTLSASVGDRVTINCQASESISSWLAWYQQKPGKAPKLLIYEASKLASGVPSRFSGSGSGTEFTLTISSLQPDDFATYYC
QGYFYFISRTYVNSFGGGTKVEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGECGGDKTHTCPPCPAPEAAGGPSVFLFPPKPKDTLMISRTPEV
TCVVVDVSHEDPEVKFNWYVDGVEVHNAKTKPREEQYNSTYRVVSVLTVLHQDWLNGKEYKCAVSNKALPAPIEKTISKAKGQPRE
PQVYTLPPSRDELTKNQVSLWCLVKGFYPSDIAVEWESNGQPENNYKTTPPVLDSDGSFFLYSKLTVDKSRWQQGNVFSCSVMHEA
LHNRFTQKSLSLSPGGGGGSGGGGSDIQMTQSPSTLSASVGDRVTITCQASQSISSHLNWYQQKPGKAPKLLIYKASTLASGVPSR
FSGSGSGTEFTLTISSLQPDDFATYYCQQGYSWGNVDNVFGGGTKVTVLGGGGSGGGGSGGGGSGGGGSEVQLLESGGGLVQPGGS
LSLSCAASGFSFSSGYDMCWVRQAPGKGLEWIACIAAGSAGITYDANWAKGRFTISRDNSKNTLYLQMNSLRAEDTAVYYCARSAF
SFDYAMDLWGQGTLVTVSS
>Sequence ID 132: SI-68P13 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGCGGCGGTGGAGGGTCCGGCGGTGGTGGATCAGACGTC
GTGATGACCCAGTCTCCTTCCACCCTGTCTGCATCTGTAGGAGACAGAGTCACCATCAATTGCCAAGCCAGTGAGAGCATTAGCAG
TTGGTTAGCCTGGTATCAGCAGAAACCAGGGAAAGCCCCTAAGCTCCTGATCTATGAAGCATCCAAACTGGCATCTGGGGTCCCAT
CAAGGTTCAGCGGCAGTGGATCTGGGACAGAATTCACTCTCACCATCAGCAGCCTGCAGCCTGATGATTTTGCAACTTATTACTGC
CAAGGCTATTTTTATTTTATTAGTCGTACTTATGTAAATTCTTTCGGCGGAGGGACCAAGGTGGAGATCAAACGTACGGTGGCTGC
ACCATCTGTCTTCATCTTCCCGCCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTGGAGGCGACAAAACTCACACATGCCCACCGTGCCCAG
CACCTGAAGCCGCGGGGGGACCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGGCGTGGAGGTGCATAATGCCAA
GACAAAGCCGCGGGAGGAGCAGTACAACAGCACGTACCGTGTGGTCAGCGTCCTCACCGTCCTGCACCAGGACTGGCTGAATGGCA
AGGAGTACAAGTGCGCGGTCTCCAACAAAGCCCTCCCAGCCCCCATCGAGAAAACCATCTCCAAAGCCAAAGGGCAGCCCCGAGAA
CCACAGGTGTACACCCTGCCCCCAAGCCGGGATGAGCTGACCAAGAACCAGGTCAGCCTGTGGTGCCTGGTCAAAGGCTTCTATCC
CAGCGACATCGCCGTGGAGTGGGAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTCATGCTCCGTGATGCATGAGGCT
CTGCACAACAGATTCACGCAGAAGAGCCTCTCCCTGTCTCCGGGTGGCGGTGGAGGGTCCGGCGGTGGTGGATCCGACATTCAGAT
GACACAGTCACCGTCTACGCTCTCAGCCTCAGTAGGCGATAGGGTGACTATCACCTGCCAAGCCTCCCAATCTATAAGTTCACACC
TCAACTGGTATCAACAGAAACCCGGTAAAGCACCGAAGCTGCTCATCTACAAGGCATCAACTCTTGCCTCAGGTGTGCCTAGTCGC
TTCTCCGGGAGCGGCTCCGGTACTGAGTTTACTTTGACGATATCTTCCCTCCAACCCGACGACTTCGCCACATACTATTGTCAGCA
GGGTTATAGTTGGGGAAATGTTGACAATGTGTTTGGAGGGGGAACGAAAGTGACTGTGTTGGGAGGGGGAGGAAGTGGCGGCGGCG
GAAGCGGAGGTGGTGGCTCCGGCGGGGGTGGAAGCGAAGTGCAATTGCTCGAAAGTGGTGGCGGACTTGTGCAACCTGGTGGAAGC
CTCTCACTCTCTTGCGCGGCGAGTGGTTTTAGCTTCAGTAGCGGCTACGATATGTGCTGGGTAAGACAGGCTCCTGGTAAAGGCCT
TGAGTGGATTGCGTGTATCGCCGCCGGGTCTGCTGGTATAACATACGATGCAAACTGGGCGAAAGGGCGCTTCACAATCTCCAGGG
ACAACTCAAAAAATACATTGTACCTCCAGATGAATAGCCTGAGAGCCGAAGACACCGCAGTCTACTATTGCGCAAGGTCAGCATTT
AGCTTCGATTATGCAATGGATCTTTGGGGCCAGGGGACGCTCGTCACCGTCTCATCTTGA
>Sequence ID 133: SI-68P17 heavy chain amino acid sequence
DVVMTQSPSTLSASVGDRVTINCQASESISSWLAWYQQKPGKAPKLLIYEASKLASGVPSRFSGSGSGTEFTLTISSLQPDDFATY
YCQGYFYFISRTYVNSFGGGTKVEIKGGGGSGGGGSGGGGSGGGGSEVQLVESGGGLVQPGGSLRLSCAASGFTISTNAMSWVRQA
PGKGLEWIGVITGRDITYYASWAKGRFTISRDNSKNTLYLQMNSLRAEDTAVYYCARDGGSSAITSNNIWGQGTLVTVSSGGGGSG
GGGSQVQLQQSGPGLVKPSETLSITCTVSGFSLTNYGVHWIRQAPGKGLEWLGVIWSGGNTDYNTPFTSRFTITKDNSKNQVYFKL
RSVRADDTAIYYCARALTYYDYEFAYWGQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTCPPCPAPEAAGGPSVFLFPPKPKDTLM
ISRTPEVTCVVVDVSHEDPEVKFNWYVDGVEVHNAKTKPREEQYNSTYRVVSVLTVLHQDWLNGKEYKCAVSNKALPAPIEKTISK
AKGQPREPQVYTLPPSRDELTKNQVSLSCAVKGFYPSDIAVEWESNGQPENNYKTTPPVLDSDGSFFLVSKLTVDKSRWQQGNVES
CSVMHEALHNHYTQKSLSLSPGGGGGSGGGGSDVVMTQSPSSVSASVGDRVTITCQASQNIRTYLSWYQQKPGKAPKLLIYAAANL
ASGVPSRFSGSGSGTDFTLTISDLEPGDAATYYCQSTYLGTDYVGGAFGGGTKLTVLGGGGSGGGGGGGGSGGGGSEVQLVESGG
GLVQPGGSLRLSCTASGFTISSYHMQWVRQAPGKGLEYIGTISSGGNVYYASSARGRFTISRPSSKNTVDLQMNSLRAEDTAVYYC
ARDSGYSDPMWGQGTLVTVSS
>Sequence ID 134: SI-68P17 heavy chain nucleotide sequence
GACGTCGTGATGACCCAGTCTCCTTCCACCCTGTCTGCATCTGTAGGAGACAGAGTCACCATCAATTGCCAAGCCAGTGAGAGCAT
TAGCAGTTGGTTAGCCTGGTATCAGCAGAAACCAGGGAAAGCCCCTAAGCTCCTGATCTATGAAGCATCCAAACTGGCATCTGGGG
TCCCATCAAGGTTCAGCGGCAGTGGATCTGGGACAGAATTTACTCTCACCATCAGCAGCCTGCAGCCTGATGATTTTGCAACTTAT
TACTGCCAAGGCTATTTTTATTTTATTAGTCGTACTTATGTAAATTCTTTCGGCGGAGGGACCAAGGTGGAGATCAAAGGCGGTGG
CGGTAGTGGGGGAGGCGGTTCTGGCGGCGGAGGGTCCGGCGGTGGAGGATCAGAGGTGCAGCTGGTGGAGTCTGGGGGAGGCTTGG
TCCAGCCTGGGGGGTCCCTGAGACTCTCCTGTGCAGCCTCTGGATTCACCATCAGTACCAATGCAATGAGCTGGGTCCGCCAGGCT
CCAGGGAAGGGGCTGGAGTGGATCGGAGTCATTACTGGTCGTGATATCACATACTACGCGAGCTGGGCGAAAGGCAGATTCACCAT
CTCCAGAGACAATTCCAAGAACACGCTGTATCTTCAAATGAACAGCCTGAGAGCCGAGGACACGGCTGTGTATTACTGTGCGAGAG
ACGGTGGTTCTTCTGCTATTACTAGTAACAACATTTGGGGCCAGGGAACCCTGGTCACCGTCTCGAGTGGCGGTGGAGGGTCCGGC
GGTGGTGGATCACAAGTACAGTTGCAGCAATCCGGTCCCGGTCTCGTCAAACCGAGTGAGACGCTTAGTATAACGTGTACTGTTTC
AGGCTTTAGCCTTACGAACTATGGAGTTCACTGGATTCGGCAGGCACCCGGCAAAGGTTTGGAATGGCTGGGTGTTATTTGGTCAG
GTGGAAATACAGACTATAACACCCCCTTTACAAGTCGGTTCACAATTACGAAAGATAATTCCAAAAATCAAGTTTATTTCAAGTTG
AGATCCGTCCGCGCGGACGACACTGCGATCTACTATTGTGCGAGGGCACTGACCTACTACGATTACGAATTTGCGTATTGGGGGCA
AGGGACTCTTGTAACAGTCTCCAGTGCTAGCACCAAGGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGACAAGAGAGTTGAGCCCAAATCTTGTGACAAAACTC
ACACATGCCCACCGTGCCCAGCACCTGAAGCCGCGGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGTGTGGTCAGCGTCCTCACCGTCCTGC
ACCAGGACTGGCTGAATGGCAAGGAGTACAAGTGCGCGGTCTCCAACAAAGCCCTCCCAGCCCCCATCGAGAAAACCATCTCCAAA
GCCAAAGGGCAGCCCCGAGAACCACAGGTGTATACCCTGCCCCCATCCCGGGATGAGCTGACCAAGAACCAGGTCAGCCTGTCCTG
CGCCGTCAAAGGCTTCTATCCCAGCGACATCGCCGTGGAGTGGGAGAGCAATGGGCAGCCGGAGAACAACTACAAGACCACGCCTC
CCGTGCTGGACTCCGACGGCTCCTTCTTCCTCGTGAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCGGCGGCGGTTCAGGTGG
TGGGGGATCCGACGTTGTGATGACGCAGTCTCCTAGTTCAGTATCAGCGTCTGTTGGGGATCGAGTCACGATTACATGCCAGGCGA
GCCAAAATATTAGGACGTATCTGTCTTGGTATCAACAAAAGCCTGGTAAGGCTCCAAAACTCCTCATTTACGCCGCTGCCAACTTG
GCTAGTGGAGTACCTTCACGCTTTAGTGGGTCAGGTTCTGGAACAGATTTTACGTTGACGATCTCCGATTTGGAACCAGGGGACGC
TGCCACGTATTATTGCCAGTCCACCTACCTCGGGACTGACTACGTTGGTGGGGCGTTTGGCGGAGGAACAAAACTCACTGTACTTG
GTGGTGGTGGTTCAGGCGGGGGGGGTTCCGGCGGAGGCGGTTCTGGGGGTGGAGGTTCCGAGGTTCAGTTGGTTGAAAGTGGGGGG
GGACTCGTTCAGCCTGGAGGCTCTTTGCGACTTTCTTGTACCGCTTCTGGGTTCACTATAAGTTCATATCATATGCAATGGGTAAG
ACAGGCGCCTGGTAAGGGACTTGAATACATAGGAACAATAAGCTCTGGTGGTAACGTCTATTACGCCTCATCCGCGCGGGGGAGGT
TTACAATTTCCAGGCCTTCTAGCAAAAACACGGTAGATCTGCAAATGAACTCTCTTCGCGCTGAAGATACAGCAGTCTACTACTGC
GCCAGGGATTCAGGGTATTCTGACCCCATGTGGGGGCAGGGCACATTGGTTACCGTGTCCTCTTGA
>Sequence ID 135: SI-68P17 light chain amino acid sequence
ENVLTQSPASLSASPGERVTITCSASSSVSYMHWYQQKPGQAPKLWIYDTSKLASGVPSRFSGSGSGNDHTLTISSMEPEDFATYY
CFQGSVYPFTFGQGTKVTVLGGGGSGGGGSGGGGSGGGGSQVTLKESGPGLVQPGQTLSLTCAFSGFSLSTSGMGVGWIRQPPGKG
LEWLAHIWWDDDKRYNPALKSRLTISKDTSKNQVYLQMNSLDAEDTAVYYCARMELWSYYFDYWGQGTLVTVSSGGGGSGGGGSEI
VLTQSPSTLSVSPGERATFSCRASQSIGTNIHWYQQKPGKPPRLLIKYASESISGIPDRFSGSGSGTEFTLTISSVQSEDFAVYYC
QQNNNWPTTFGPGTKLTVLRTVAAPSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGECGGDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYKCAVSNKALPAPIEKTISKAKGQPREPQVYT
LPPSRDELTKNQVSLWCLVKGFYPSDIAVEWESNGQPENNYKTTPPVLDSDGSFFLYSKLTVDKSRWQQGNVFSCSVMHEALHNRF
TQKSLSLSPGGGGGSGGGGSDIQMTQSPSTLSASVGDRVTITCQASQSISSHLNWYQQKPGKAPKLLIYKASTLASGVPSRFSGSG
SGTEFTLTISSLQPDDFATYYCQQGYSWGNVDNVFGGGTKVTVLGGGGSGGGGSGGGGSGGGGSEVQLLESGGGLVQPGGSLSLSC
AASGFSFSSGYDMCWVRQAPGKGLEWIACIAAGSAGITYDANWAKGRFTISRDNSKNTLYLQMNSLRAEDTAVYYCARSAFSFDYA
MDLWGQGTLVTVSS
>Sequence ID 136: SI-68P17 light chain nucleotide sequence
GAAAATGTATTGACACAGAGCCCCGCCTCCCTCAGTGCCTCACCTGGGGAAAGGGTAACTATCACTTGCTCTGCATCAAGCAGCGT
CTCATACATGCATTGGTATCAACAAAAGCCTGGACAGGCCCCCAAGCTCTGGATATACGATACGAGCAAGCTGGCTTCCGGCGTAC
CTAGCCGCTTCAGTGGTTCCGGCTCAGGCAACGATCACACCCTTACGATTTCCAGTATGGAACCCGAAGATTTTGCAACTTATTAT
TGTTTCCAGGGGAGCGTGTACCCATTCACTTTCGGGCAGGGGACAAAAGTGACCGTCCTAGGCGGTGGCGGTAGTGGGGGAGGCGG
TTCTGGCGGCGGAGGGTCCGGCGGTGGAGGATCACAGGTCACATTGAAGGAATCTGGCCCCGGCCTTGTTCAGCCAGGACAGACCC
TTAGCCTCACCTGTGCCTTCAGTGGTTTTTCTCTTAGCACTAGCGGTATGGGGGTCGGCTGGATTCGGCAGCCTCCCGGCAAAGGT
CTTGAGTGGTTGGCTCACATTTGGTGGGACGACGACAAACGGTATAATCCTGCCTTGAAAAGTCGGCTGACCATTAGTAAGGATAC
CTCAAAAAATCAAGTGTACTTGCAAATGAATAGCCTTGACGCCGAGGATACGGCTGTATATTATTGCGCGCGGATGGAACTCTGGT
CTTACTACTTTGATTATTGGGGGCAGGGGACTCTCGTCACGGTCTCGAGTGGCGGTGGAGGGTCCGGCGGTGGTGGATCAGAAATC
GTCCTTACACAATCTCCTAGCACACTGAGTGTGAGCCCCGGCGAACGCGCGACTTTCTCTTGCAGGGCAAGTCAATCCATAGGGAC
TAATATACATTGGTATCAACAAAAGCCAGGTAAACCACCCAGGCTTTTGATTAAGTATGCAAGTGAGTCTATTTCCGGTATCCCTG
ACCGCTTCTCTGGATCAGGCAGTGGCACAGAGTTCACACTCACCATATCTAGTGTGCAATCAGAGGACTTCGCCGTGTATTACTGC
CAACAGAATAATAACTGGCCGACTACCTTCGGACCCGGTACAAAGCTGACCGTTTTACGTACGGTGGCTGCACCATCTGTCTTCAT
CTTCCCGCCATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAGGGCCTGAGCTC
GCCCGTCACAAAGAGCTTCAACAGGGGAGAGTGTGGAGGCGACAAAACTCACACATGCCCACCGTGCCCAGCACCTGAAGCCGCGG
GGGGACCGTCAGTCTTCCTCTTCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAATGCCAAGACAAAGCCGCGGGA
GGAGCAGTACAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAGGTGTACACC
CTGCCCCCAAGCCGGGATGAGCTGACCAAGAACCAGGTCAGCCTGTGGTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCACAACAGATTC
ACGCAGAAGAGCCTCTCCCTGTCTCCGGGTGGCGGTGGAGGGTCCGGCGGTGGTGGATCCGACATTCAGATGACACAGTCACCGTC
TACGCTCTCAGCCTCAGTAGGCGATAGGGTGACTATCACCTGCCAAGCCTCCCAATCTATAAGTTCACACCTCAACTGGTATCAAC
AGAAACCCGGTAAAGCACCGAAGCTGCTCATCTACAAGGCATCAACTCTTGCCTCAGGTGTGCCTAGTCGCTTCTCCGGGAGCGGC
TCCGGTACTGAGTTTACTTTGACGATATCTTCCCTCCAACCCGACGACTTCGCCACATACTATTGTCAGCAGGGTTATAGTTGGGG
AAATGTTGACAATGTGTTTGGAGGGGGAACGAAAGTGACTGTGTTGGGAGGGGGAGGAAGTGGCGGCGGCGGAAGCGGAGGTGGTG
GCTCCGGCGGGGGTGGAAGCGAAGTGCAATTGCTCGAAAGTGGTGGCGGACTTGTGCAACCTGGTGGAAGCCTCTCACTCTCTTGC
GCGGCGAGTGGTTTTAGCTTCAGTAGCGGCTACGATATGTGCTGGGTAAGACAGGCTCCTGGTAAAGGCCTTGAGTGGATTGCGTG
TATCGCCGCCGGGTCTGCTGGTATAACATACGATGCAAACTGGGCGAAAGGGCGCTTCACAATCTCCAGGGACAACTCAAAAAATA
CATTGTACCTCCAGATGAATAGCCTGAGAGCCGAAGACACCGCAGTCTACTATTGCGCAAGGTCAGCATTTAGCTTCGATTATGCA
ATGGATCTTTGGGGCCAGGGGACGCTCGTCACCGTCTCATCTTGA
>Sequence ID 137: SI-79C1 HC and SI-79X1 HC1 amino acid sequence
QVQLKQSGPGLVQPSQSLSITCTVSGFSLTNYGVHWVRQSPGKGLEWLGVIWSGGNTDYNTPFTSRLSINKDNSKSQVFFKMNSLQ
SNDTAIYYCARALTYYDYEFAYWGQGTLVTVSAASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPPCPAPEAAGAPSVFLFPPKPKDTLMISRT
PEVTCVVVDVSHEDPEVKFNWYVDGVEVHNAKTKPREEQYNSTYRVVSVLTVLHQDWLNGKEYKCAVSNKALPAPIEKTISKAKGQ
PREPQVYTLPPSRDELTKNQVSLTCLVKGFYPSDIAVEWESNGQPENNYKTTPPVLDSDGSFFLYSRLTVDKSRWQQGNVFSCSVM
HEALHNHYTQKSLSLSPG
>Sequence ID 138: SI-79C1 HC and SI-79X1 HC1 nucleotide sequence
CAAGTTCAGCTCAAGCAGTCTGGCCCTGGCCTGGTTCAGCCTTCTCAGAGCCTGAGCATCACCTGTACCGTGTCCGGCTTCTCCCT
GACCAATTACGGCGTGCACTGGGTTCGACAGAGCCCTGGCAAAGGACTGGAATGGCTGGGAGTGATTTGGAGCGGCGGCAACACCG
ACTACAACACCCCTTTCACCTCTCGGCTGTCTATCAACAAGGACAACTCCAAGAGCCAGGTGTTCTTCAAGATGAACTCCCTGCAG
TCCAACGACACCGCCATCTACTACTGTGCTCGGGCCCTGACCTACTACGACTACGAGTTTGCTTACTGGGGCCAGGGCACCCTGGT
CACAGTTTCTGCTGCTAGCACCAAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGCGTCCTCACCGTCCTGCACCAGGACTGGC
TGAATGGCAAGGAGTACAAGTGCGCGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAGGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTTGA
>Sequence ID 139: SI-79C1 LC, SI-79C5 LC, SI-79X1 LC1, and SI-79X5 LC1 amino acid
sequence
DILLTQSPVILSVSPGERVSFSCRASQSIGTNIHWYQQRTNGSPRLLIKYASESISGIPSRFSGSGSGTDFTLSINSVESEDIADY
YCQQNNNWPTTFGAGTKLELKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
>Sequence ID 140: SI-79C1 LC, SI-79C5 LC, SI-79X1 LC1, and SI-79X5 LC1 nucleotide
sequence
GACATCCTGCTGACCCAGTCTCCAGTGATCCTGTCCGTGTCTCCTGGCGAGAGAGTGTCCTTCAGCTGCAGAGCCTCTCAGTCCAT
CGGCACCAACATCCACTGGTATCAGCAGCGGACCAACGGCTCCCCTCGGCTGCTGATTAAGTACGCCTCCGAGTCTATCAGCGGCA
TCCCCTCCAGATTCTCCGGCTCTGGCTCTGGCACCGACTTCACCCTGTCCATCAACTCCGTGGAATCCGAGGATATCGCCGACTAC
TACTGCCAGCAGAACAACAACTGGCCCACCACCTTTGGCGCTGGCACCAAGCTGGAATTGAAACGTACGGTGGCTGCACCATCTGT
CTTCATCTTCCCGCCATCTGATGAGCAGTTGAAATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTCTACGCCTGCGAAGTCACCCATCAGGGCCT
GAGCTCGCCCGTCACAAAGAGCTTCAACAGGGGAGAGTGTTAG
>Sequence ID 141: SI-79C2 HC, SI-79C3 HC, SI-79X2 HC1, SI-79X3 HC1 amino acid sequence
QVQLQQSGPGLVKPSETLSITCTVSGFSLTNYGVHWIRQAPGKGLEWLGVIWSGGNTDYNTPFTSRFTITKDNSKNQVYFKLRSVR
ADDTAIYYCARALTYYDYEFAYWGQGTLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPPCPAPEAAGAPSVFLFPPKPKDTLMISRT
PEVTCVVVDVSHEDPEVKFNWYVDGVEVHNAKTKPREEQYNSTYRVVSVLTVLHQDWLNGKEYKCAVSNKALPAPIEKTISKAKGQ
PREPQVYTLPPSRDELTKNQVSLTCLVKGFYPSDIAVEWESNGQPENNYKTTPPVLDSDGSFFLYSRLTVDKSRWQQGNVFSCSVM
HEALHNHYTQKSLSLSPG
>Sequence ID 142: SI-79C2 HC, SI-79C3 HC, SI-79X2 HC1, SI-79X3 HC1 nucleotide sequence
CAAGTTCAGTTGCAGCAGTCTGGCCCTGGCCTGGTCAAGCCTTCTGAGACACTGTCCATCACCTGTACCGTGTCCGGCTTCTCCCT
GACCAATTACGGCGTGCACTGGATCAGACAGGCCCCTGGCAAAGGACTGGAATGGCTGGGAGTGATTTGGAGCGGCGGCAACACCG
ACTACAACACCCCTTTCACCAGCCGGTTCACCATCACCAAGGACAACTCCAAGAACCAGGTGTACTTCAAGCTGCGGAGCGTGCGG
GCTGATGACACCGCCATCTACTACTGTGCTCGGGCCCTGACCTACTACGACTACGAGTTTGCTTACTGGGGCCAGGGCACCCTGGT
CACAGTTTCTTCTGCTAGCACCAAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGCGTCCTCACCGTCCTGCACCAGGACTGGC
TGAATGGCAAGGAGTACAAGTGCGCGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAGGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTTGA
>Sequence ID 143: SI-79C2 LC and SI-79X2 LC1 amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLELKRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSENRGEC
>Sequence ID 144: SI-79C2 LC and SI-79X2 LC1 nucleotide sequence
GAGATCGTGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAGCCCGGCAAGCCTCCTCGGCTGCTGATTAAGTACGCCTCCGAGTCCATCAGCGGCA
TCCCTGACAGATTCTCCGGCTCTGGCTCTGGCACCGAGTTTACCCTGACCATCTCCTCCGTGCAGTCCGAGGATTTCGCCGTGTAC
TACTGCCAGCAGAACAACAACTGGCCCACCACCTTTGGACCCGGCACCAAGCTGGAATTGAAACGTACGGTGGCTGCACCATCTGT
CTTCATCTTCCCGCCATCTGATGAGCAGTTGAAATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTCTACGCCTGCGAAGTCACCCATCAGGGCCT
GAGCTCGCCCGTCACAAAGAGCTTCAACAGGGGAGAGTGTTAG
>Sequence ID 145: SI-9C21 HC, SI-79X1 HC2, SI-79X2 HC2, SI-79X3 HC2, and SI-79X5 HC2
amino acid sequence
EVQLVESGGGLVQPGGSLRLSCTASGFTISTNAMSWVRQAPGKGLEWVGVITGRDITYYASWAKGRFTISRDTSKNTVYLQMNSLR
AEDTAVYYCARDGGSSAITSNNIWGQGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCPPCPAPEAAGAPSVFLFPPKPKDTLMISR
TPEVTCVVVDVSHEDPEVKFNWYVDGVEVHNAKTKPREEQYNSTYRVVSVLTVLHQDWLNGKEYKCAVSNKALPAPIEKTISKAKG
QPREPQVYTLPPSRDELTKNQVSLTCLVKGFYPSDIAVEWESNGQPENNYKTTPPVLDSDGSFLLYSKLTVDKSRWQQGNVFSCSV
MHEALHNHYTQKSLSLSPG
>Sequence ID 146: SI-9C21 HC, SI-79X1 HC2, SI-79X2 HC2, SI-79X3 HC2, and SI-79X5 HC2
nucleotide sequence
GAAGTCCAATTGGTAGAAAGTGGCGGTGGTCTGGTGCAACCTGGTGGATCTCTTCGCCTCTCATGCACGGCTAGTGGCTTTACTAT
TTCAACTAATGCGATGAGCTGGGTTCGCCAGGCCCCCGGCAAAGGACTTGAGTGGGTCGGCGTCATCACCGGCAGGGACATTACAT
ACTATGCGAGTTGGGCAAAGGGCAGGTTCACGATTAGCCGCGATACTTCAAAGAATACCGTTTACCTTCAAATGAATAGCTTGCGC
GCGGAAGACACAGCTGTGTATTACTGCGCGCGGGATGGAGGTAGTTCCGCCATAACTTCCAACAACATATGGGGACAAGGCACGCT
GGTTACTGTGTCGTCAGCTAGCACCAAGGGCCCATCGGTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGTGACCGTGCCCTCCAGCAGCTTGGGCACCCAGACCTA
CATCTGCAACGTGAATCACAAGCCCAGCAACACCAAGGTGGACAAGAGAGTTGAGCCCAAATCTTGTGACAAAACTCACACATGCC
CACCGTGCCCAGCACCTGAAGCCGCGGGGGCACCGTCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAGGTCAAGTTCAACTGGTACGTGGACGGCGTGGAGGT
GCATAATGCCAAGACAAAGCCGCGGGAGGAGCAGTACAACAGCACGTACCGTGTGGTCAGCGTCCTCACCGTCCTGCACCAGGACT
GGCTGAATGGCAAGGAGTACAAGTGCGCGGTCTCCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGACCAAGAACCAGGTCAGCCTGACCTGCCTGGTCAA
AGGCTTCTATCCCAGCGACATCGCCGTGGAGTGGGAGAGCAATGGGCAGCCGGAGAACAACTACAAGACCACGCCTCCCGTGCTGG
ACTCCGACGGCTCCTTCCTGCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTTGA
>Sequence ID 147: SI-9C21 LC, SI-79X1 LC2, SI-79X2 LC2, SI-79X3 LC2, and SI-79X5 LC2
amino acid sequence
EIVMTQSPSTLSASVGDRVIITCQASESISSWLAWYQQKPGKAPKLLIYEASKLASGVPSRFSGSGSGAEFTLTISSLQPDDFATY
YCQGYFYFISRTYVNSFGQGTKLTVLRTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSENRGEC
>Sequence ID 148: SI-9C21 LC, SI-79X1 LC2, SI-79X2 LC2, SI-79X3 LC2, and SI-79X5 LC2
nucleotide sequence
GAAATCGTTATGACGCAGAGTCCCTCCACGCTCTCCGCTAGTGTCGGGGATCGCGTCATTATCACATGCCAGGCCTCCGAGTCAAT
CAGCAGCTGGCTTGCATGGTATCAACAGAAGCCGGGAAAAGCTCCTAAATTGCTGATCTATGAAGCGTCAAAATTGGCGTCTGGTG
TCCCATCTAGGTTCTCCGGCTCTGGGTCTGGTGCGGAATTTACTTTGACAATCTCCAGTCTTCAACCAGACGATTTCGCTACCTAC
TACTGCCAAGGGTATTTCTATTTTATAAGCCGGACATATGTAAACTCCTTCGGCCAAGGAACAAAGTTGACTGTTCTTCGTACGGT
GGCTGCACCATCTGTCTTCATCTTCCCGCCATCTGATGAGCAGTTGAAATCTGGAACTGCCTCTGTTGTGTGCCTGCTGAATAACT
TCTATCCCAGAGAGGCCAAAGTACAGTGGAAGGTGGATAACGCCCTCCAATCGGGTAACTCCCAGGAGAGTGTCACAGAGCAGGAC
AGCAAGGACAGCACCTACAGCCTCAGCAGCACCCTGACGCTGAGCAAAGCAGACTACGAGAAACACAAAGTCTACGCCTGCGAAGT
CACCCATCAGGGCCTGAGCTCGCCCGTCACAAAGAGCTTCAACAGGGGAGAGTGTTAG
>Sequence ID 149: SI-79C3 LC and SI-79X3 LC1 amino acid sequence
EIVLTQSPSTLSVSPGERATFSCRASQSIGTNIHWYQQKPGKPPRLLIKYASESISGIPDRFSGSGSGTEFTLTISSVQSEDFAVY
YCQQNNNWPTTFGPGTKLTVLRTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSENRGEC
>Sequence ID 150: SI-79C3 LC and SI-79X3 LC1 nucleotide sequence
GAGATCGTGCTGACCCAGTCTCCTTCCACACTGTCTGTGTCTCCCGGCGAGAGAGCCACCTTCAGCTGTAGAGCCTCTCAGTCCAT
CGGCACCAACATCCACTGGTATCAGCAGAAGCCCGGCAAGCCTCCTCGGCTGCTGATTAAGTACGCCTCCGAGTCCATCAGCGGCA
TCCCTGACAGATTCTCCGGCTCTGGCTCTGGCACCGAGTTTACCCTGACCATCTCCTCCGTGCAGTCCGAGGATTTCGCCGTGTAC
TACTGCCAGCAGAACAACAACTGGCCCACCACCTTTGGACCCGGCACCAAGCTGACCGTGCTGCGTACGGTGGCTGCACCATCTGT
CTTCATCTTCCCGCCATCTGATGAGCAGTTGAAATCTGGAACTGCCTCTGTTGTGTGCCTGCTGAATAACTTCTATCCCAGAGAGG
CCAAAGTACAGTGGAAGGTGGATAACGCCCTCCAATCGGGTAACTCCCAGGAGAGTGTCACAGAGCAGGACAGCAAGGACAGCACC
TACAGCCTCAGCAGCACCCTGACGCTGAGCAAAGCAGACTACGAGAAACACAAAGTCTACGCCTGCGAAGTCACCCATCAGGGCCT
GAGCTCGCCCGTCACAAAGAGCTTCAACAGGGGAGAGTGTTAG
>Sequence ID 151: SI-79C5 HC and SI-79X5 HC1 amino acid sequence
QVQLKQSGPGLVQPSQSLSITCTVSGFSLTNYGVHWVRQSPGKGLEWLGVIWSGGNTDYNTPFTSRLSINKDNSKSQVFFKMNSLQ
SEDTAIYYCARALTYYDYEFAYWGQGTLVTVSAASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPPCPAPEAAGAPSVFLFPPKPKDTLMISRT
PEVTCVVVDVSHEDPEVKFNWYVDGVEVHNAKTKPREEQYNSTYRVVSVLTVLHQDWLNGKEYKCAVSNKALPAPIEKTISKAKGQ
PREPQVYTLPPSRDELTKNQVSLTCLVKGFYPSDIAVEWESNGQPENNYKTTPPVLDSDGSFFLYSRLTVDKSRWQQGNVFSCSVM
HEALHNHYTQKSLSLSPG
>Sequence ID 152: SI-79C5 HC and SI-79X5 HC1 nucleotide sequence
CAAGTTCAGCTCAAGCAGTCTGGCCCTGGCCTGGTTCAGCCTTCTCAGAGCCTGAGCATCACCTGTACCGTGTCCGGCTTCTCCCT
GACCAATTACGGCGTGCACTGGGTTCGACAGAGCCCTGGCAAAGGACTGGAATGGCTGGGAGTGATTTGGAGCGGCGGCAACACCG
ACTACAACACCCCTTTCACCTCTCGGCTGTCTATCAACAAGGACAACTCCAAGAGCCAGGTGTTCTTCAAGATGAACTCCCTGCAG
TCCGAGGACACCGCCATCTACTACTGTGCTCGGGCCCTGACCTACTACGACTACGAGTTTGCTTACTGGGGCCAGGGCACCCTGGT
CACAGTTTCTGCTGCTAGCACCAAGGGCCCATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCAGCTTGGGCACCCAGACCTACAT
CTGCAACGTGAATCACAAGCCCAGCAACACCAAGGTGGACAAGAGAGTTGAGCCCAAATCTTGTGACAAAACTCACACATGCCCAC
CGTGCCCAGCACCTGAAGCCGCGGGGGCACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTACGTGGACGGCGTGGAGGTGCA
TAATGCCAAGACAAAGCCGCGGGAGGAGCAGTACAACAGCACGTACCGTGTGGTCAGCGTCCTCACCGTCCTGCACCAGGACTGGC
TGAATGGCAAGGAGTACAAGTGCGCGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGATGAGCTGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAGGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTTGA
>Sequence ID 153: Consensus-VH
QVQLQQSGPGLVKPSESLSLTCTVSGFSLTNYGVHWVRQAPGKGLEWLGVIWSGGNTDYNTPFTSRFTISKDNSKNQVYFKMNSLR
AEDTAXYYCARALTYYDYEFAYWGQGTLVTVSS
>Sequence ID 154: Consensus-VL
EIVLTQSPSTLSVSPGERVTFSCRASQSIGTNIHWYQQKPGQPPRLLIKYASESISGIPSRFSGSGSGTEFTLTISSVQSEDFAVY
YCQQNNNWPTTFGQGTKLELK

### REFERENCES

1. Graham J, Muhsin M, Kirkpatrick P. Cetuximab. Nat Rev Drug Discov 2004; 3:549-50.
2. Bou-Assaly W, Mukherji S. Cetuximab (Erbitux). Am J Neuroradiol 2010; 31:626-7.
3. Hanck-Silva G, Fatori Trevizan L N, Petrilli R, de Lima F T, Eloy J O, Chorilli M. A Critical Review of Properties and Analytical/Bioanalytical Methods for Characterization of Cetuximab. Crit Rev Anal Chem [Internet] 2020; 50:125-35. Available from: https://doi.org/10.1080/10408347.2019.1581984
4. García-Foncillas J, Sunakawa Y, Aderka D, Wainberg Z, Ronga P, Witzler P, Stintzing S. Distinguishing Features of Cetuximab and Panitumumab in Colorectal Cancer and Other Solid Tumors. Front Oncol 2019; 9:1-16.
5. Kaplon H, Reichert JM. Antibodies to watch in 2021. MAbs [Internet] 2021; 13:1-34. Available from: https://doi.org/10.1080/19420862.2020.1860476
6. Hwang WYK, Foote J. Immunogenicity of engineered antibodies. Methods 2005; 36:3-10.
7. Park S S, Kim J, Brandts J F, Hong H J. Stability of murine, chimeric and humanized antibodies against pre-S2 surface antigen of hepatitis B virus. Biologicals 2003; 31:295-302.
8. Ayoub D, Jabs W, Resemann A, Evers W, Evans C, Main L, Baessmann C, Wagner-Rousset E, Suckau D, Beck A. Correct primary structure assessment and extensive glyco-profiling of cetuximab by a combination of intact, middle-up, middle-down and bottom-up ESI and MALDI mass spectrometry techniques. MAbs 2013; 5:699-710.
9. van de Bovenkamp F S, Hafkenscheid L, Rispens T, Rombouts Y. The Emerging Importance of IgG Fab Glycosylation in Immunity. J Immunol 2016; 196:1435-41.
10 Chung C H, Mirakhur B, Chan E, Le Q-T, Berlin J, Morse M, Murphy B A, Satinover S M, Hosen J, Mauro D, et al. Cetuximab-Induced Anaphylaxis and IgE Specific for Galactose-α-1,3-Galactose. N Engl J Med 2008; 358:1109-17.
11. Kuriakose A, Chirmule N, Nair P. Immunogenicity of Biotherapeutics: Causes and Association with Posttranslational Modifications. J Immunol Res 2016; 2016.
12. Zhang P, Woen S, Wang T, Liau B, Zhao S, Chen C, Yang Y, Song Z, Wormald M R, Yu C, et al. Challenges of glycosylation analysis and control: An integrated approach to producing optimal and consistent therapeutic drugs. Drug Discov Today [Internet] 2016; 21:740-65. Available from: http://dx.doi.org/10.1016/j.drudis.2016.01.006
13. Borras L, Gunde T, Tietz J, Bauer U, Hulmann-Cottier V, Grimshaw J P A, Urech D M. Generic approach for the generation of stable humanized single-chain Fv fragments from rabbit monoclonal antibodies. J Biol Chem 2010; 285:9054-66.
14. Li S, Schmitz K R, Jeffrey P D, Wiltzius J J W, Kussie P, Ferguson K M. Structural basis for inhibition of the epidermal growth factor receptor by cetuximab. Cancer Cell 2005; 7:301-11.
15. Ewert S, Huber T, Honegger A, Pluckthun A. Biophysical properties of human antibody variable domains. J Mol Biol 2003; 325:531-53.
16 Nieba L, Honegger A, Krebber C, Pluckthun A. Disrupting the hydrophobic patches at the antibody variable/constant domain interface: Improved in vivo folding and physical characterization of an engineered scFv fragment. Protein Eng 1997; 10:435-44.
17. Gao S H, Huang K, Tu H, Adler A S. Monoclonal antibody humanness score and its applications. BMC Biotechnol 2013; 13.
18 Racle J, Michaux J, Rockinger G A, Arnaud M, Bobisse S, Chong C, Guillaume P, Coukos G, Harari A, Jandus C, et al. Robust prediction of HLA class IIepitopes by deep motif deconvolution of immunopeptidomes. Nat Biotechnol [Internet] 2019; 37:1283-6. Available from: http://dx.doi.org/10.1038/s41587-019-0289-6
19. Wang C, Wu Y, Wang L, Hong B, Jin Y, Hu D, Chen G, Kong Y, Huang A, Hua G, et al. Engineered soluble monomeric IgG1 Fc with significantly decreased non-specific binding. Front Immunol 2017; 8:1-8.
20. Ulitzka M, Carrara S, Grzeschik J, Kornmann H, Hock B, Kolmar H. Engineering therapeutic antibodies for patient safety: Tackling the immunogenicity problem. Protein Eng Des Sel 2020; 33:1-12.
21 Banisadr A, Safdari Y, Kianmehr A, Pourafshar M. Production of a germline-humanized cetuximab scFv and evaluation of its activity in recognizing EGFR-overexpressing cancer cells. Hum Vaccines Immunother [Internet] 2018; 14:856-63. Available from: https://doi.org/10.1080/21645515.2017.1407482
22. Choi Y, Ndong C, Griswold K E, Bailey-Kellogg C. Computationally driven antibody engineering enables simultaneous humanization and thermostabilization. Protein Eng Des Sel 2016; 29:419-26.
23 Giddens J P, Lomino J V., DiLillo D J, Ravetch J V., Wang L X. Site-selective chemoenzymatic glycoengineering of Fab and Fc glycans of a therapeutic antibody. Proc Natl Acad Sci USA 2018; 115:12023-7.

What is claimed is:

1. A human epithelium growth factor receptor (EGFR) binding peptide having a binding specificity to human EGFR, comprising an amino acid sequence having at least 98% sequence identity to SEQ ID NO. 1, 3, 5, 7, 9, 11, 13, 15, 17, 19, 21, 23, 25, 27, 29, 31, 33, 35, 37, 39, 41, 43, 57, 61, 63, 65, 67, 69, 71, 73, 75, 77, 79, 81, or 83.

2. The EGFR binding peptide of claim 1, comprising a variable heavy (VH) chain and a variable light (VL) chain,
wherein the VH chain comprises an amino acid sequence having at least 98% sequence identify to SEQ ID NO. 1, 5, 9, 13, 17, 21, 25, 29, 33, 37, or 41; and
wherein the VL chain comprises an amino acid sequence having at least 98% sequence identity to SEQ ID NO. 3, 7, 11, 15, 19, 23, 27, 31, 35, 39, or 43.

3. The EGFR binding peptide of claim 2, comprising a scFv domain, wherein the scFv domain comprises the VH chain and the VL chain, and wherein the scFv domain comprises an amino acid sequence having at least 98% sequence identity to SEQ ID NO. 57, 61, 63, 65, 67, 69, 71, 73, 75, 77, 79, 81, or 83.

4. The EGFR binding peptide of claim 3, comprising a histidine residue linked to at least one end of the scFv domain, and wherein the human EGFR binding peptide comprises an amino acid sequence having at least 98% sequence identity to SEQ ID NO. 57.

5. The EGFR binding peptide of claim 2, comprising a Fab domain, wherein the Fab domain comprises the VH chain and the VL chain.

6. The EGFR binding peptide of claim 5, further comprising a Fc domain linked to the Fab domain to provide a Fab-monoFc fusion protein, wherein the Fc domain comprises an amino acid sequence having at least 98% sequence identity to SEQ ID NO. 45 or 47.

7. An antibody-like protein having a binding specificity to human EGFR, comprising an EGFR binding domain having a variable heavy (VH) chain and a variable light (VL) chain,
wherein the VH chain comprises an amino acid sequence having at least 98% sequence identify to SEQ ID NO. 1, 5, 9, 13, 17, 21, 25, 29, 33, 37, or 41; and
wherein the VL chain comprises an amino acid sequence having at least 98% sequence identity to SEQ ID NO. 3, 7, 11, 15, 19, 23, 27, 31, 35, 39, or 43.

8. The antibody-like protein of claim 7, comprising a scFv domain having an amino acid sequence having at least 98% sequence identity to SEQ ID NO. 57, 59, 61, 63, 65, 67, 69, 71, 73, 75, 77, 79, 81, or 83.

9. The antibody-like protein of claim 7, wherein the antibody-like protein is a monospecific antibody comprising an amino acid sequence having at least 98% sequence identity to SEQ ID NO. 137, 139; 141, 143; 141, 149, 151,139; 145, or 147.

10. The antibody-like protein of claim 7, wherein the antibody-like protein is a bispecific antibody comprising an amino acid sequence having at least 98% sequence identity to SEQ ID NO. 137, 145, 139, 147, 141, 145, 143, 147, 141, 145, 149, 147, 151, 145, 139, or 147.

11. The antibody-like protein of claim 7, wherein the antibody-like protein is a penta-specific antibody comprising an amino acid sequence having at least 98% sequence identity to SEQ ID NO. 85, 87; 89, 91; 93, 95, 97, 99, 101, 103, 105, or 107.

12. The antibody-like protein of claim 7, wherein the antibody-like protein is a hexa-specific antibody comprising an amino acid sequence having at least 98% sequence identity to SEQ ID NO. 109, 111, 113, 115, 117, or 119.

13. The antibody-like protein of claim 7, comprising a heavy chain (HC) and a light chain (LC),
wherein the HC comprises an amino acid sequence having at least 98% sequence identity to SEQ ID NO. 85, 89, 93, 97, 101, 105, 109, 113, 117, 137, 141, 145, or 151; and
wherein the LC comprises an amino acid sequence having at least 98% sequence identity to SEQ ID NO. 87, 91, 95, 99, 103, 107, 111, 115, 119, 139, 143, 147, or 149.

14. The antibody-like protein of claim 7 comprising a heavy chain monomer and a light chain monomer, wherein the heavy chain monomer having a N-terminus and a C-terminus, comprising in tandem from the N-terminal to the C-terminal,
an optional first binding domain (D1) at the N-terminal,
a Fab domain as a second binding domain (D2) comprising a light chain,
a Fc domain,
an optional third binding domain (D3), and
an optional fourth binding domain (D4) at the C-terminal,
wherein the light chain comprises an optional fifth binding domain (D5) covalently attached to the C-terminus, an optional sixth binding domain (D6) covalently attached to the N-terminus, or a combination thereof, and wherein at least one of D1, D2, D3, D4, D5 and D6 comprises the EGFR binding domain.

15. The antibody-like protein of claim 14, wherein at least one of the D1 or D2 comprises the EGFR binding domain.

16. The antibody-like protein of claim 14, wherein each of the D3, D4, D5 and D6 comprises the EGFR binding domain.

17. The antibody-like protein of claim 14, wherein antibody-like protein is a bispecific antibody comprising an amino acid sequence having at least 98% sequence identity to SEQ ID NO. 137, 145, 139, 147; 141, 145, 143, 147; 141, 145, 149, 147; 151, 145, 139, or 147.

18. The antibody-like protein of claim 17, wherein the bispecific antibody is asymmetric with the D2 comprising the EGFR binding domain and the D3 has a binding specificity to CD3.

19. The antibody-like protein of claim 7 having a N-terminus and a C-terminus, comprising,
a first monomer, comprising, from the N-terminus to the C-terminus, a first binding domain (mD1), a variable heavy (VH) chain, a CH1 domain, a first hinge, a first CH2 domain, a first CH3 domain, and a fourth binding domain (mD4),
a second monomer, comprising, from the N-terminus to the C-terminus, a second binding domain (mD2), a variable light (VL) chain, a CL domain, a second hinge, a second CH2 domain, and a second CH3 domain, and a fifth binding domain (mD5),
wherein the CH chain and CL chain forms a third binding domain (mD3),
wherein the first monomer and the second monomer are covalently paired through at least one disulfide bond between the CH1 domain and the CL domain and at least one disulfide bond between the first hinge and the second hinge,
wherein the multi-specific antibody-like protein is at least bi-specific, and
wherein at least one of the mD1, mD2, mD3, mD4, and mD5 comprises the EGFR binding domain.

20. (canceled)

21. The antibody-like protein of claim 19, wherein at least one of the mD3 or mD2 domain comprises the EGFR binding domain.

22. The antibody-like protein of claim 19, wherein the mD2, mD4, mD5 each comprises the EGFR binding domain.

23. The antibody-like protein of claim 19, comprising an amino acid sequence having at least 98% sequence identity to SEQ ID NO. 121, 123, 125, 127, 129, 131, 133, 135, or a combination thereof.

24-25. (canceled)

26. An isolated nucleic acid sequence encoding the antibody-like protein of claim 7.

27. An expression vector comprising the isolated nucleic acid sequences of claim 26.

28. A host cell comprising the isolated nucleic acid sequence of claim 26.

29. A pharmaceutical composition, comprising the antibody-like protein of claim 7 and a pharmaceutically acceptable carrier.

30. An immunoconjugate comprising the antibody-like protein of claim 7 and a cytotoxic agent.

31. A pharmaceutical composition, comprising the immunoconjugate of claim 30 and a pharmaceutically acceptable carrier.

32. A method for treating or preventing a cancer, an autoimmune disease, or an infectious disease in a subject, said method comprising administering to the subject a pharmaceutical composition comprising a purified antibody-like protein of claim 7.

33. A method for producing the antibody-like protein of claim 7, comprising
culturing a host cell such that the DNA sequence encoding the antibody-like protein of claim 9 is expressed, and
purifying said multi-specific antibody-like protein.

34. (canceled)
